(19) United States
(12) Patent Application Publication
(10) Pub. No.: US 2023/0035072 A1
(43) Pub. Date: Feb. 2, 2023

(54) METHODS OF USE OF ANTI-CD33 ANTIBODIES

(71) Applicant: Alector LLC, South San Francisco, CA (US)

(72) Inventors: Robert PAUL, San Francisco, CA (US); Michael F. WARD, San Francisco, CA (US); Hua LONG, San Carlos, CA (US); Shiao-Ping LU, Los Altos, CA (US); Omer Rizwan SIDDIQUI, Redwood City, CA (US); Arnon ROSENTHAL, Woodside, CA (US)

(73) Assignee: Alector LLC, South San Francisco, CA (US)

(21) Appl. No.: 17/784,579

(22) PCT Filed: Dec. 11, 2020
(86) PCT No.: PCT/US2020/064461

§ 371 Date: Jun. 10, 2022

Related U.S. Application Data
(60) Provisional application No. 62/947,455, filed on Dec. 12, 2019.

(51) Int. Cl. C07K 16/28; A61P 25/28

(52) U.S. Cl. CPC C07K 16/2803; A61P 25/28; A61K 2039/505

(57) ABSTRACT

The present disclosure is generally directed to antibodies, e.g., monoclonal, chimeric, humanized antibodies, antibody fragments, etc., that specifically bind one or more epitopes within a CD33 protein, e.g., human CD33 or a mammalian CD33, and have improved and/or enhanced functional characteristics. The present disclosure is further directed to the methods of treating and/or delaying the progression of a disease or injury in an individual by administering such antibodies.

### CROSS-REFERENCE TO RELATED APPLICATIONS

This application claims the benefit of U.S. Provisional Application No. 62/947,455 filed Dec. 12, 2019, which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 735022003340SEQLIST.TXT, date recorded: Dec. 9, 2020, size: 180 KB).

### FIELD OF THE INVENTION

This present disclosure relates to anti-CD33 antibodies, and therapeutic uses of such antibodies.

### BACKGROUND

Myeloid cell surface antigen CD33 precursor (CD33), also known as Siglec-3, is a type 1, immunoglobulin-like, transmembrane protein expressed on immune and hematopoietic cells, including immature and mature myeloid cells, dendritic cells, and microglial cells. (Crocker et al. (2007) Nat Rev Immunol. 7:255-266; McMillan and Crocker (2008) Carbohydr Res. 343:2050-2056; Von Gunten and Bochner (2008) Ann NY Acad Sci. 1143:61-82; Handgretinger et al. (1993) Immunol Lett. 37:223-228; and Hernández-Caselles et al. (2006) J Leukoc Biol. 79:46-58). CD33 contains an Ig-like C2-type (immunoglobulin-like) and an Ig-like V-type (immunoglobulin-like) extracellular domain, as well as two ITIM-like motifs in its cytoplasmic domain. Three alternatively spliced forms (isoforms) of CD33 have been identified, including a higher molecular weight variant, named CD33M and a smaller isoform CD33m that lacks the Ig-like V-type domain (the ligand-binding site), and the disulfide bond linking the V and C domains.

Genome-wide association studies (GWAS) performed on extended cohorts (e.g., thousands of individuals) have identified single nucleotide polymorphisms (SNPs) rs3865444^{CC} (AKA rs3826656) and rs3865444^{AA} in CD33 as genetic modulators of risk for late onset Alzheimer's disease (AD). In addition, carriers of the 2459419^{TT} allele, as well as carriers of the rs12459419^{CT} allele, which show over 25% reduction in expression of full-length CD33, display reduced Alzheimer's disease risk (Malik M. et al. (2015) Human Molecular Genetics, 1-14). This suggests that reduced expression or functionality of CD33 may be beneficial in Alzheimer's disease and cancer.

Accordingly, there is a need for therapeutic anti-CD33 antibodies to treat diseases, disorders, and conditions associated with undesired CD33 activity.

Novel therapeutic antibodies targeting CD33 are one solution to treating diseases associated with CD33 activity, such as Alzheimer's disease. Systemically administered monoclonal antibodies normally exhibit a biphasic pharmacokinetic profile, being first distributed relatively quickly and then eliminated more slowly (Ovacik, M and Lin, L, (2018) Clin Transl Sci 11, 540-552). Circulation of systemically administered antibodies is typically confined to the vasculature and interstitial space (Ovacik, M and Lin, L, (2018) Clin Transl Sci 11, 540-552). This is because of their size, polarity, recycling and clearance kinetics, and typically relatively long half-lives, which are often 11-30 days in humans (Ovacik, M and Lin, L, (2018) Clin Transl Sci 11, 540-552).

Administration of monoclonal antibodies presents a challenge for therapeutic use. Monoclonal antibodies have limited oral bioavailability, so they are typically administered intravenously, subcutaneously, or intramuscularly (Ovacik, M and Lin, L, (2018) Clin Transl Sci 11, 540-552). Intravenous administration is particularly challenging for patients with neurodegenerative diseases, such as Alzheimer's disease. These diseases affect patients for long periods of time and thus require regular treatment over the course of many years. As intravenous administration cannot be done at home, patients must be transported to infusion centers on a regular basis, which is a burden on both the patient and caregiver. Finally, the memory loss, mood swings, aggression, and other behavioral symptoms of these diseases make patient compliance difficult.

Accordingly, there is a further need for identifying methods of treating patients with the appropriate dose and frequency of administration of anti-CD33 antibodies for treating and/or delaying the progression of a disease or injury, such as Alzheimer's disease, while facilitating patient compliance.

All references cited herein, including patents, patent applications and publications, are hereby incorporated by reference in their entirety.

### SUMMARY

In one aspect, provided herein is a method of treating and/or delaying the progression of a disease or injury in an individual, including administering to the individual an anti-CD33 antibody intravenously at a dose of at least about 1.6 mg/kg, wherein the antibody is administered about once every twelve weeks or more frequently; and wherein the antibody includes: a heavy chain variable region that includes an HVR-H1 including the amino acid sequence GYTFTDYNLH (SEQ ID NO: 105), an HVR-H2 including the amino acid sequence FIYPSNRITG (SEQ ID NO: 119), and an HVR-H3 including the amino acid sequence SDVDYFDY (SEQ ID NO: 122); and a light chain variable region that includes an HVR-L1 including the amino acid sequence RASQSVSTSTYSYMH (SEQ ID NO: 127), an HVR-L2 including the amino acid sequence YASNLES (SEQ ID NO: 135), and an HVR-L3 including the amino acid sequence QHSWEIPLT (SEQ ID NO: 146). In some embodiments, the anti-CD33 antibody is administered at a dose of between about 1.6 mg/kg and about 15 mg/kg. In some embodiments, the anti-CD33 antibody is administered at a dose of about 1.6 mg/kg, about 5 mg/kg, about 7.5 mg/kg, about 10 mg/kg, or about 15 mg/kg. In some embodiments, the anti-CD33 antibody is administered once every two weeks, once every four weeks, once every five weeks, once every six weeks, once every seven weeks, once every eight weeks, once every nine weeks, once every ten weeks, once every eleven weeks, or once every twelve weeks. In some embodiments, the anti-CD33 antibody is administered once every two weeks at a dose of about 1.6 mg/kg. In some embodiments, the anti-CD33 antibody is administered once every four weeks at a dose of about 1.6 mg/kg. In some embodiments, the anti-CD33 antibody is administered once every four weeks at a dose of about 15 mg/kg. In some embodiments, the anti-CD33 antibody is administered once every five weeks at a dose of about 15 mg/kg. In some embodiments, the anti-CD33 antibody is administered once every six weeks at a dose of about 15 mg/kg. In some embodiments, the anti-CD33 antibody is administered once every seven weeks at a dose of about 15 mg/kg. In some embodiments, the anti-CD33 antibody is administered once every eight weeks at a dose of about 15 mg/kg.

In some embodiments, which may be combined with any of the preceding embodiments, the cell surface level of CD33 is reduced by at least about 70% compared to the cell surface level of CD33 prior to administration of the anti-CD33 antibody. In some embodiments, the cell surface level of CD33 is reduced by at least about 80% compared to the cell surface level of CD33 prior to administration of the anti-CD33 antibody. In some embodiments, the cell surface level of CD33 is reduced by at least about 85% compared to the cell surface level of CD33 prior to administration of the anti-CD33 antibody. In some embodiments, the cell surface level of CD33 is reduced by at least about 90% compared to the cell surface level of CD33 prior to administration of the anti-CD33 antibody. In some embodiments, the reduction in the cell surface level of CD33 is present for at least about 12 days after administration of the anti-CD33 antibody. In some embodiments, the reduction in the cell surface level of CD33 is present for at least about 17 days after administration of the anti-CD33 antibody. In some embodiments, the reduction in the cell surface level of CD33 is present for at least about 29 days after administration of the anti-CD33 antibody. In some embodiments, the reduction in the cell surface level of CD33 is present for at least about 42 days after administration of the anti-CD33 antibody. In some embodiments, the reduction in the cell surface level of CD33 is present for at least about 56 days after administration of the anti-CD33 antibody. In some embodiments, the reduction in cell surface level of CD33 includes a reduction in the cell surface level of CD33 on peripheral blood monocytes of the individual.

In some embodiments, which may be combined with any of the preceding embodiments, the antibody includes a heavy chain variable region including the amino acid sequence of SEQ ID NO: 59 and a light chain variable region including the amino acid sequence of SEQ ID NO: 86. In some embodiments, the antibody has an IgG2 isotype. In some embodiments, the antibody includes a heavy chain including the amino acid sequence of SEQ ID NO: 180 or SEQ ID NO: 201, and a light chain including the amino acid sequence of SEQ ID NO: 185.

In some embodiments, which may be combined with any of the preceding embodiments, the terminal half-life of the anti-CD33 antibody in plasma is about 4 days, about 5 days, about 6 days, about 7 days, about 8 days, about 9 days, about 10 days, about 11 days, or about 12 days. In some embodiments, the terminal half-life of the anti-CD33 antibody in plasma is about 10 days.

In some embodiments, which may be combined with any of the preceding embodiments, the disease or injury is selected from dementia, frontotemporal dementia, Alzheimer's disease, vascular dementia, mixed dementia, taupathy disease, infections, and cancer. In some embodiments, the disease or injury is Alzheimer's disease.

In some embodiments, which may be combined with any of the preceding embodiments, the individual is diagnosed with Alzheimer's disease, or has a clinical diagnosis of probable Alzheimer's disease dementia. In some embodiments, the individual has a Mini-Mental State Examination (MMSE) score of between about 16 points to about 28 points. In some embodiments, the individual has a Clinical Dementia Rating-Global Score (CDR-GS) of about 0.5, about 1.0, or about 2.0. In some embodiments, the individual has a positive amyloid-PET scan. In some embodiments, the individual is taking a stable dose of a cholinesterase inhibitor and/or a memantine therapy for Alzheimer's disease. In some embodiments, the individual does not carry two copies of the rs12459419^{T} allele.

In some embodiments, which may be combined with any of the preceding embodiments, the disease or injury is Alzheimer's disease, and treatment and/or delay of the progression of Alzheimer's disease is assessed using one or more clinical assessments selected from the Mini-Mental State Examination (MMSE), the Repeatable Battery for the Assessment of Neuropsychological Status (RBANS), the Clinical Dementia Rating (CDR) assessment, amyloid brain positron emission tomography (PET), translocator protein (TSPO)-PET imaging, and any combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the effect of anti-CD33 antibody AB-64.1.2 on CD33 levels on peripheral monocytes over time following a single intravenous administration of the antibody at a dose of 1.6 mg/kg or 15 mg/kg.

### DETAILED DESCRIPTION

### General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### Definitions

As used herein, the term “preventing” includes providing prophylaxis with respect to occurrence or recurrence of a particular disease, disorder, or condition in an individual. An individual may be predisposed to, susceptible to a particular disease, disorder, or condition, or at risk of developing such a disease, disorder, or condition, but has not yet been diagnosed with the disease, disorder, or condition.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations. An effective amount herein may vary according to factors such as the disease state, age, sex, and weight of the individual, and the ability of the treatment to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, administration “in conjunction” with another compound or composition includes simultaneous administration and/or administration at different times. Administration in conjunction also encompasses administration as a co-formulation or administration as separate compositions, including at different dosing frequencies or intervals, and using the same route of administration or different routes of administration.

An “individual” for purposes of treatment, prevention, or reduction of risk refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. Preferably, the individual is human.

The term “immunoglobulin” (Ig) is used interchangeably with “antibody” herein. The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

The basic 4-chain antibody unit is a heterotetrameric glycoprotein composed of two identical light (L) chains and two identical heavy (H) chains. The pairing of a V_{H} and V_{L} together forms a single antigen-binding site. For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”) and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three dimensional configurations of different classes of immunoglobulins are well known and described generally in, for example, Abbas et al., Cellular and Molecular Immunology, 4^{th} ed. (W. B. Saunders Co., 2000).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

An “isolated” antibody, such as an anti-CD33 antibody of the present disclosure, is one that has been identified, separated and/or recovered from a component of its production environment (e.g., naturally or recombinantly). Preferably, the isolated polypeptide is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant T cells since at least one component of the antibody's natural environment will not be present. Ordinarily, however, an isolated polypeptide or antibody will be prepared by at least one purification step.

The “variable region” or “variable domain” of an antibody, such as an anti-CD33 antibody of the present disclosure, refers to the amino-terminal domains of the heavy or light chain of the antibody. The variable domains of the heavy chain and light chain may be referred to as “V_{H}” and “VL”, respectively. These domains are generally the most variable parts of the antibody (relative to other antibodies of the same class) and contain the antigen binding sites.

The term “variable” refers to the fact that certain segments of the variable domains differ extensively in sequence among antibodies, such as anti-CD33 antibodies of the present disclosure. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent-cellular toxicity.

The term “monoclonal antibody” as used herein refers to an antibody, such as an anti-CD33 antibody of the present disclosure, obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against one or more antigenic sites. In some embodiments, a monoclonal antibody of the present disclosure can be a bispecific antibody. In contrast to polyclonal antibody preparations which typically include different antibodies directed against different determinants (epitopes), each monoclonal antibody is directed against a single determinant on the one or more antigenic sites. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including, for example, phage-display technologies (see, e.g., Clackson et al., Nature, 352:624-628 (1991); Marks et al., J. Mol. Biol. 222:581-597 (1992); Sidhu et al., J. Mol. Biol. 338(2): 299-310 (2004); Lee et al., J. Mol. Biol. 340(5):1073-1093 (2004); Fellouse, Proc. Nat'l Acad. Sci. USA 101(34):12467-472 (2004); and Lee et al., J. Immunol. Methods 284(1-2):119-132 (2004), the hybridoma method (e.g., Kohler and Milstein., Nature, 256:495-97 (1975); Hongo et al., Hybridoma, 14 (3):253-260 (1995), Harlow et al., Antibodies: A Laboratory Manual, (Cold Spring Harbor Laboratory Press, 2d ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), yeast presentation technologies (see, e.g., WO2009/036379A2; WO2010105256; WO2012009568, and Xu et al., Protein Eng. Des. Sel., 26(10): 663-70 (2013), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat'l Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

The terms “full-length antibody,” “intact antibody” or “whole antibody” are used interchangeably to refer to an antibody, such as an anti-CD33 antibody of the present disclosure, in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10):1057-1062 (1995)); single-chain antibody molecules and multispecific antibodies formed from antibody fragments.

Papain digestion of antibodies, such as anti-CD33 antibodies of the present disclosure, produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of antibodies, such as anti-CD33 antibodies of the present disclosure, comprise a portion of an intact antibody, generally including the antigen binding or variable region of the intact antibody or the F region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Nat'l Acad. Sci. USA 90:6444-48 (1993).

As used herein, a “chimeric antibody” refers to an antibody (immunoglobulin), such as an anti-CD33 antibody of the present disclosure, in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Nat'l Acad. Sci. USA, 81:6851-55 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies, such as anti-CD33 antibodies of the present disclosure, are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, and the like. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino-acid sequence corresponding to that of an antibody, such as an anti-CD33 antibody of the present disclosure, produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Nat'l Acad. Sci. USA, 103:3557-3562 (2006) regarding human antibodies generated via a human B-cell hybridoma technology. Alternatively, human antibodies can also be prepared by employing yeast libraries and methods as disclosed in, for example, WO2009/036379A2; WO2010105256; WO2012009568; and Xu et al., Protein Eng. Des. Sel., 26(10): 663-70 (2013).

The term “hypervariable region,” “HVR,” or “HV,” when used herein refers to the regions of an antibody-variable domain, such as that of an anti-CD33 antibody of the present disclosure, that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu in Methods in Molecular Biology 248:1-25 (Lo, ed., Human Press, Totowa, N.J., 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. In some embodiments, the HVRs may be Kabat complementarity-determining regions (CDRs) based on sequence variability and are the most commonly used (Kabat et al., supra). In some embodiments, the HVRs may be Chothia CDRs. Chothia refers instead to the location of the structural loops (Chothia and Lesk J. Mol. Biol. 196:901-917 (1987)). In some embodiments, the HVRs may be AbM HVRs. The AbM HVRs represent a compromise between the Kabat CDRs and Chothia structural loops, and are used by Oxford Molecular's AbM antibody-modeling software. In some embodiments, the HVRs may be “contact” HVRs. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat numbering) |
| H1 | H31-H35 | H26-H35 | H26-H32 | H30-H35 (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to EU or Kabat et al., supra, for each of these extended-HVR definitions.

“Framework” or “FR” residues are those variable-domain residues other than the HVR residues as herein defined.

The phrase “variable-domain residue-numbering as in EU or Kabat” or “amino-acid-position numbering as in EU or Kabat,” and variations thereof, refers to the numbering system used for heavy-chain variable domains or light-chain variable domains of the compilation of antibodies in EU or Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g., residues 82a, 82b, and 82c, etc. according to Kabat) after heavy-chain FR residue 82. The EU or Kabat numbering of residues may be determined for a given antibody by alignment at regions of homology of the sequence of the antibody with a “standard” Kabat numbered sequence.

The EU or Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU or Kabat numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU or Kabat numbering system (e.g., see United States Patent Publication No. 2010-280227).

An “acceptor human framework” as used herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferable those changes occur at only three, two, or one of positions 71H, 73H and 78H; for instance, the amino acid residues at those positions may by 71A, 73T and/or 78A. In one embodiment, the VL acceptor human framework is identical in sequence to the VL human immunoglobulin framework sequence or human consensus framework sequence.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, Md. (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra.

An “amino-acid modification” at a specified position, e.g., of an anti-CD33 antibody of the present disclosure, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody, such as an anti-CD33 antibody of the present disclosure, is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As use herein, the term “specifically recognizes” or “specifically binds” refers to measurable and reproducible interactions such as attraction or binding between a target and an antibody, such as an anti-CD33 antibody of the present disclosure, that is determinative of the presence of the target in the presence of a heterogeneous population of molecules including biological molecules. For example, an antibody, such as an anti-CD33 antibody of the present disclosure, that specifically or preferentially binds to a target or an epitope is an antibody that binds this target or epitope with greater affinity, avidity, more readily, and/or with greater duration than it binds to other targets or other epitopes of the target. It is also understood by reading this definition that, for example, an antibody (or a moiety) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³ M⁻¹ or 10⁴ M⁻¹, sometimes about 10⁵ M⁻¹ or 10⁶ M⁻¹, in other instances about 10⁶M⁻¹ or 10⁷ M⁻¹, about 10⁸ M⁻¹ to 10⁹M⁻¹, or about 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

As used herein, an “interaction” between a CD33 protein and a second protein encompasses, without limitation, protein-protein interaction, a physical interaction, a chemical interaction, binding, covalent binding, and ionic binding. As used herein, an antibody “inhibits interaction” between two proteins when the antibody disrupts, reduces, or completely eliminates an interaction between the two proteins. An antibody of the present disclosure, or fragment thereof, “inhibits interaction” between two proteins when the antibody or fragment thereof binds to one of the two proteins.

An “agonist” antibody or an “activating” antibody is an antibody, such as an agonist anti-CD33 antibody of the present disclosure, that induces (e.g., increases) one or more activities or functions of the antigen after the antibody binds the antigen.

A “blocking” antibody, an “antagonist” antibody, or an “inhibitory” antibody is an antibody, such as an anti-CD33 antibody of the present disclosure, that inhibits or reduces (e.g., decreases) antigen binding to one or more ligand after the antibody binds the antigen, and/or that inhibits or reduces (e.g., decreases) one or more activities or functions of the antigen after the antibody binds the antigen. In some embodiments, blocking antibodies, antagonist antibodies, or inhibitory antibodies substantially or completely inhibit antigen binding to one or more ligand and/or one or more activities or functions of the antigen.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU or Kabat numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. (see, e.g., M. Daëron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126: 330-41 (1995). Other FcRs, including those to be identified in the future, are encompassed by the term “FcR” herein. FcRs can also increase the serum half-life of antibodies.

Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants with improved or diminished binding to FcRs. See also, e.g., Shields et al., J. Biol. Chem. 9(2):6591-6604 (2001).

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full length of the sequences being compared.

An “isolated” nucleic acid molecule encoding an antibody, such as an anti-CD33 antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of the present disclosure.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspect and embodiments of the present disclosure described herein include “comprising,” “consisting,” and “consisting essentially of” aspects and embodiments.

### Overview

The present disclosure relates to methods of treating and/or delaying the progression of a disease or injury, e.g., Alzheimer's disease, in an individual by administering an anti-CD33 antibody to the individual. As described below, the methods of the present disclosure meet the need in the art for treating patients with the correct dose and frequency of administration of an anti-CD33 antibody and of administering that dose in ways that ease patient compliance.

An anti-CD33 antibody of the present disclosure exhibits a relatively short terminal half-life in serum of between about 4 days to about 10 days (see, e.g., Example 1). The short terminal half-life of the anti-CD33 antibody compared to other therapeutic antibodies (Ovacik, M and Lin, L, (2018) Clin Transl Sci 11, 540-552) suggests that the antibody may not be expected to be useful therapeutically. However, unexpectedly, a single intravenous administration of the anti-CD33 antibody resulted in a decrease of CD33 cell surface levels of at least 70% that persisted for at least between about 17 days and about 56 days (see, e.g., Example 2).

Thus, despite the relatively short terminal half-life of the anti-CD33 antibody of the present disclosure, the methods provided herein permit relatively infrequent administration of the anti-CD33 antibody, which is particularly beneficial for patients with neurodegenerative diseases, such as Alzheimer's disease.

Accordingly, in some embodiments, the present disclosure further relates to methods of treating and/or delaying the progression of a disease or injury, e.g., Alzheimer's Disease, in an individual by administering an anti-CD33 antibody to the individual at a dose of between about 1.6 mg/kg and about 15 mg/kg once every twelve weeks or more frequently (see, e.g., Example 3).

All references cited herein, including patents, patent applications and publications, are hereby incorporated by reference in their entirety.

### Therapeutic Uses

As disclosed herein, anti-CD33 antibodies of the present disclosure may be used for preventing, reducing risk, or treating dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, and cancer including bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express CD33, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and/or Haemophilus influenza. In some embodiments, the CD33 antibodies are agonist antibodies. In some embodiments, the antibodies are inert antibodies. In some embodiments, the antibodies are antagonist antibodies.

In some embodiments, the present disclosure provides methods of preventing, reducing risk, or treating dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express CD33, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and/or Haemophilus influenza, by administering to an individual in need thereof a therapeutically effective amount of an antibody of the present disclosure that decreases cellular levels of CD33, inhibits interaction between CD33 and one or more CD33 ligands, or both.

In some embodiments, the present disclosure provides methods of preventing, reducing risk, or treating cancer, by administering to an individual in need thereof, a therapeutically effective amount of an antibody of the present disclosure that decreases cellular levels of CD33, inhibits interaction between CD33 and one or more CD33 ligands, or both. In some embodiments, the antibody inhibits one or more CD33 activities selected from: (a) promoting proliferation, maturation, migration, differentiation, and/or functionality of one or more of immunosuppressor dendritic cells, immunosuppressor macrophages, immunosuppressor neutrophils, non-tumorigenic myeloid derived suppressor cells, tumor-associated macrophages, non-tumorigenic CD14⁺ myeloid cells, and regulatory T cells; (b) enhancing infiltration of one or more of immunosuppressor dendritic cells, immunosuppressor macrophages, immunosuppressor neutrophils, non-tumorigenic myeloid derived suppressor cells, tumor-associated macrophages, and regulatory T cells into tumors; (c) increasing number of tumor-promoting myeloid/granulocytic immune-suppressive cells and/or non-tumorigenic CD14⁺ myeloid cells in a tumor, in peripheral blood, or other lymphoid organ; (d) decreasing activation of tumor-specific T lymphocytes with tumor killing potential; (e) decreasing infiltration of tumor-specific T lymphocytes with tumor killing potential; (f) increasing tumor volume; (g) increasing tumor growth rate; (h) increasing metastasis; (i) increasing rate of tumor recurrence; (j) increasing expression of one or more PD-1 ligands; (k) decreasing efficacy of one or more immune-therapies that modulate anti-tumor T cell responses, optionally wherein the one or more immune-therapies are immune-therapies that target one or more proteins selected from the group consisting of CD40, OX40, ICOS, CD28, CD137/4-1BB, CD27, GITR, PD-L1, CTLA4, PD-L2, PD-1, B7-H3, B7-H4, HVEM, LIGHT, BTLA, CD38, TIGIT, VISTA, KIR, GAL9, TIM1, TIM3, TIM4, A2AR, LAG3, DR5, CD39, CD70, CD73, TREM1, TREM2, Siglec-5, Siglec-7, Siglec-9, Siglec-11, SirpA, CD47, CSF-1 receptor, and any combination thereof, or of one or more cancer vaccines; and (1) decreasing efficacy of one or more chemotherapy agents, optionally wherein the one or more of the chemotherapy agents are gemcitabine, capecitabine, anthracyclines, doxorubicin (Adriamycin®), epirubicin (Ellence®), taxanes, paclitaxel (Taxol®), docetaxel (Taxotere®), 5-fluorouracil (5-FU), cyclophosphamide (Cytoxan®), carboplatin (Paraplatin®), oxaliplatin (Elotaxin®), leucovorin, temozolmide (Temodar®), and any combination thereof. In some embodiments, the antibody exhibits one or more activities selected from: (a) increasing the number of tumor infiltrating CD3⁺ T cells; (b) decreasing cellular levels of CD33 in non-tumorigenic CD14⁺ myeloid cells, optionally wherein the non-tumorigenic CD14⁺ myeloid cells are tumor infiltrating cells or optionally wherein the non-tumorigenic CD14⁺ myeloid cells are present in blood; (c) reducing the number of non-tumorigenic CD14⁺ myeloid cells, optionally wherein the non-tumorigenic CD14⁺ myeloid cells are tumor infiltrating cells or optionally wherein the non-tumorigenic CD14⁺ myeloid cells are present in blood; (d) reducing PD-L1 levels in one or more cells, optionally wherein the one or more cells are non-tumorigenic myeloid-derived suppressor cells (MDSC); (e) decreasing tumor growth rate of solid tumors; (f) reducing tumor volume; (g) increasing efficacy of one or more PD-1 inhibitors; (h) increasing efficacy of one or more checkpoint inhibitor therapies and/or immune-modulating therapies, optionally wherein the one or more checkpoint inhibitor therapies and/or immune-modulating therapies target one or more of CTL4, the adenosine pathway, PD-L1, PD-L2, OX40, TIM3, LAG3, or any combination thereof; (i) increasing efficacy of one or more chemotherapy agents, optionally wherein the one or more of the chemotherapy agents are gemcitabine, capecitabine, anthracyclines, doxorubicin (Adriamycin®), epirubicin (Ellence®), taxanes, paclitaxel (Taxol®), docetaxel (Taxotere®), 5-fluorouracil (5-FU), cyclophosphamide (Cytoxan®), carboplatin (Paraplatin®), oxaliplatin (Elotaxin®), leucovorin, temozolmide (Temodar®), and any combination thereof; and (j) killing CD33-expressing immunosuppressor myeloid cells and/or CD14-expressing cells in solid tumors and associated blood vessels when conjugated to a chemical or radioactive toxin.

As disclosed herein, anti-CD33 antibodies of the present disclosure may also be used for inducing and/or promoting the survival maturation, functionality, migration, or proliferation of one or more immune cells (e.g., innate immune cells or adaptive immune cells). In some embodiments, the present disclosure provides methods of inducing or promoting the survival, maturation, functionality, migration, or proliferation of one or more immune cells in an individual in need thereof, by administering to the individual a therapeutically effective amount of an antibody of the present disclosure that decreases cellular levels of CD33, inhibits interaction between CD33 and one or more CD33 ligands, or both. In some embodiments, the one or more immune cells are selected from dendritic cells, macrophages, microglia, neutrophils, T cells, T helper cells, cytotoxic T cells, and any combination thereof.

In some embodiments, the antibody is an agonist anti-CD33 antibody. In some embodiments, the antibody is a transient agonist anti-CD33 antibody of the present disclosure that initially acts as an agonist and then acts as a long-term antagonist antibody. In some embodiments, the antibody is an inert anti-CD33 antibody. In some embodiments, the antibody is an antagonist anti-CD33 antibody. In some embodiments, the anti-CD33 antibody reduces cellular (e.g., cell surface, intracellular, or total) levels of CD33. In some embodiments, the anti-CD33 antibody induces degradation of CD33. In some embodiments, the anti-CD33 antibody induces cleavage of CD33. In some embodiments, the anti-CD33 antibody induces internalization of CD33. In some embodiments, the anti-CD33 antibody induces shedding of CD33. In some embodiments, the anti-CD33 antibody induces downregulation of CD33 expression. In some embodiments, the anti-CD33 antibody inhibits interaction (e.g., binding) between CD33 and one or more CD33 ligands. In some embodiments, the anti-CD33 antibody transiently activates and then induces degradation of CD33. In some embodiments, the anti-CD33 antibody transiently activates and then induces cleavage of CD33. In some embodiments, the anti-CD33 antibody transiently activates and then induces internalization of CD33. In some embodiments, the anti-CD33 antibody transiently activates and then induces shedding of CD33. In some embodiments, the anti-CD33 antibody transiently activates and then induces downregulation of CD33 expression. In some embodiments, the anti-CD33 antibody transiently activates and then induces decreased expression of CD33. In certain embodiments, the individual has a CD33 variant allele having single nucleotide polymorphisms (SNPs) rs3865444 CC or AC. In certain embodiments, the individual has a CD33 variant allele having single nucleotide polymorphisms (SNPs) 2459419 CC or CT.

As disclosed herein, anti-CD33 antibodies of the present disclosure may further be used for decreasing the activity, functionality, or survival of regulatory T cells, tumor-imbedded immunosuppressor dendritic cells, tumor-imbedded immunosuppressor macrophages, myeloid-derived suppressor cells, tumor-associated macrophages, acute myeloid leukemia (AML) cells, chronic lymphocytic leukemia (CLL) cell, and/or chronic myeloid leukemia (CML) cells. In some embodiments, the present disclosure provides methods of decreasing the activity, functionality, or survival of regulatory T cells, tumor-imbedded immunosuppressor dendritic cells, tumor-imbedded immunosuppressor macrophages, myeloid-derived suppressor cells, tumor-associated macrophages, acute myeloid leukemia (AML) cells, chronic lymphocytic leukemia (CLL) cell, or chronic myeloid leukemia (CML) cells in an individual in need thereof, by administering to the individual a therapeutically effective amount of an antibody that binds or interacts with CD33. In some embodiments, the antibody is selected from an antagonist antibody, an inert antibody, or an agonist antibody. In some embodiments, the antibody is an isolated anti-CD33 antibody or anti-CD33 antibody conjugate of the present disclosure. In some embodiments, the anti-CD33 antibody conjugate comprises an anti-CD33 antibody conjugated to a detectable marker, a toxin, or a therapeutic agent.

As disclosed herein, anti-CD33 antibodies of the present disclosure may be used for decreasing cellular levels of CD33, inhibiting interaction between CD33 and one or more CD33 ligands, or both on one or more cells in vitro or in vivo. In some embodiments, the present disclosure provides methods of decreasing cellular levels of CD33, inhibiting interaction between CD33 and one or more CD33 ligands, or both on one or more cells in an individual in need thereof, by administering to the individual a therapeutically effective amount of an isolated anti-CD33 antibody of the present disclosure. In some embodiments, the anti-CD33 antibody decreases cellular levels of CD33 in vivo.

As disclosed herein, anti-CD33 antibodies of the present disclosure may be used for decreasing cellular levels of CD33 on one or more cells, including without limitation, dendritic cells, bone marrow-derived dendritic cells, monocytes, peripheral blood monocytes, granulocytes, microglia, T cells, macrophages, and/or cell lines. In some embodiments, the present disclosure provides methods of decreasing cellular levels of CD33 on one or more cells in an individual in need thereof, by administering to the individual a therapeutically effective amount of an anti-CD33 antibody of the present disclosure. In some embodiments, the one or more cells are selected from dendritic cells, bone marrow-derived dendritic cells, monocytes, peripheral blood monocytes, granulocytes, microglia, T cells, and macrophages, and any combination thereof. In some embodiments, the anti-CD33 antibody decreases cellular levels of CD33 in vivo. Cellular levels of CD33 may refer to, without limitation, cell surface levels of CD33, intracellular levels of CD33, and total levels of CD33. In some embodiments, a decrease in cellular levels of CD33 comprises decrease in cell surface levels of CD33. As used herein, cell surface levels of CD33 may be measured by any in vitro cell-based assays or suitable in vivo model described herein or known in the art. In some embodiments, a decrease in cellular levels of CD33 comprises a decrease in intracellular levels of CD33. As used herein, intracellular levels of CD33 may be measured by any in vitro cell-based assays or suitable in vivo model described herein or known in the art. In some embodiments, a decrease in cellular levels of CD33 comprises a decrease in total levels of CD33. As used herein, total levels of CD33 may be measured by any in vitro cell-based assays or suitable in vivo model described herein or known in the art. In some embodiments, the anti-CD33 antibodies induce CD33 degradation, CD33 cleavage, CD33 internalization, CD33 shedding, and/or downregulation of CD33 expression. In some embodiments, cellular levels of CD33 are measured on primary cells (e.g., dendritic cells, bone marrow-derived dendritic cells, monocytes, peripheral blood monocytes, granulocytes, microglia, T cells, and macrophages) or on cell lines utilizing an in vitro cell assay.

Other aspects of the present disclosure relate to a method of selecting a subject in need thereof for treatment with an anti-CD33 antibody, the method comprising: a. obtaining a sample (e.g., blood sample) from the subject; b. detecting the CD33 alleles present in the subject; and c. selecting the subject for treatment with the antibody that binds or interacts with CD33 is the subject has one or more CD33 alleles, wherein the one or more CD33 alleles are selected from the group consisting of rs3865444^{AC}, and rs3865444^{CC}. Other aspects of the present disclosure relate to a method of assessing responsiveness of a subject in need thereof to an antibody that binds or interacts with CD33, the method comprising: a. measuring the expression levels of CD45⁺ and CD14⁺ on non-tumorigenic myeloid cells in a blood sample obtained from the subject prior to administering to the subject an anti-CD33 antibody; b. administering to the subject a therapeutically effective amount of the antibody; and c. measuring the expression levels of CD45⁺ and CD14⁺ on non-tumorigenic myeloid cells in a blood sample obtained from the subject after administration of the anti-CD33 antibody, wherein a reduction in the levels of CD45⁺ CD14⁺ on non-tumorigenic myeloid cells after administration of the anti-CD33 antibody indicates the subject is responsive to the agent. Any suitable methods for obtaining a sample, such as a blood sample, may be used. Further, it will be appreciated that any known method of detecting CD33 variants and/or alleles, such as SNP analysis, may be used. In some embodiments, the method of assessing responsiveness further comprises administering one or more additional therapeutically effective amounts of the antibody. In some embodiments, the subject is human.

In some embodiments the individual has a variant of CD33. In some embodiments, the variant includes, without limitation, one or more polymorphisms selected from: (a) SNP rs3865444^{A}c; (b) SNP rs3865444^{CC}; (c) SNP rs35112940^{GG, AA, AG}; and (d) SNP rs12459419^{CC, CT or TT} and any combinations thereof.

In some embodiments, the individual is not a carrier of two copies of the minor allele rs12459419^{T}.

In some embodiments, the individual has a clinical diagnosis of probable Alzheimer's disease dementia based on National Institute on Aging Alzheimer's Association criteria. In some embodiments, the individual has an Mini-Mental State Examination (MMSE) score of 16-28 points, inclusive. In some embodiments, the individual has a Clinical Dementia Rating-Global Score (CDR-GS) of 0.5, 1.0, or 2.0. In some embodiments, the individual has a positive amyloid-PET scan by qualitative read. In some embodiments, the individual is taking a cholinesterase inhibitor and/or memantine therapy for Alzheimer's disease, on a stable dose for at least 4 weeks prior to administration of the anti-CD33 antibody. As used herein, “a stable dose” refers to a dose that has not changed significantly over the specified time period and is not expected or intended to change.

In some embodiments, the methods of the present disclosure may further involve the coadministration of anti-CD33 antibodies or bispecific anti-CD33 antibodies, with antibodies that bind to pattern recognition receptors, antibodies that bind to Toll-like receptors, antibodies that bind to damage-associated molecular pattern (DAMP) receptors, and/or antibodies that bind to cytokine or antibodies to interleukins).

In some embodiments, the methods of the present disclosure may further include administering to the individual at least one antibody that specifically binds to an inhibitory checkpoint molecule, and/or one or more standard or investigational anti-cancer therapies. In some embodiments, the at least one antibody that specifically binds to an inhibitory checkpoint molecule is administered in combination with the anti-CD33 antibody. In some embodiments, the at least one antibody that specifically binds to an inhibitory checkpoint molecule is selected from an anti-PD-L1 antibody, an anti-CTLA4 antibody, an anti-PD-L2 antibody, an anti-PD-1 antibody, an anti-B7-H3 antibody, an anti-B7-H4 antibody, and anti-HVEM antibody, an anti-B- and T-lymphocyte attenuator (BTLA) antibody, an anti-Killer inhibitory receptor (KIR) antibody, an anti-GAL9 antibody, an anti-TIM3 antibody, an anti-A2AR antibody, an anti-LAG-3 antibody, an anti-phosphatidylserine antibody, an anti-CD27 antibody, an anti-TNFa antibody, an anti-Siglec-5 antibody, an anti-Siglec-7 antibody, an anti-Siglec-9 antibody, an anti-Siglec-11 antibody, an antagonistic anti-TREM1 antibody, an antagonistic anti-TREM2 antibody, and any combination thereof. In some embodiments, the one or more standard or investigational anti-cancer therapies are selected from radiotherapy, cytotoxic chemotherapy, targeted therapy, imatinib therapy, trastuzumab therapy, etanercept therapy, adoptive cell transfer (ACT) therapy, chimeric antigen receptor T cell transfer (CAR-T) therapy, vaccine therapy, and cytokine therapy.

In some embodiments, the methods of the present disclosure may further include administering to the individual at least one antibody that specifically binds to an inhibitory cytokine. In some embodiments, the at least one antibody that specifically binds to an inhibitory cytokine is administered in combination with the anti-CD33 antibody. In some embodiments, the at least one antibody that specifically binds to an inhibitory cytokine is selected from an anti-CCL2 antibody, an anti-CSF-1 antibody, an anti-IL-2 antibody, and any combination thereof.

In some embodiments, the methods of the present disclosure may further include administering to the individual at least one agonistic antibody that specifically binds to a stimulatory checkpoint protein. In some embodiments, the at least one agonistic antibody that specifically binds to a stimulatory checkpoint protein is administered in combination with the anti-CD33 antibody. In some embodiments, the at least one agonistic antibody that specifically binds to a stimulatory checkpoint protein is selected from an agonist anti-CD40 antibody, an agonist anti-OX40 antibody, an agonist anti-ICOS antibody, an agonist anti-CD28 antibody, an agonistic anti-TREM1 antibody, an agonistic anti-TREM2 antibody, an agonist anti-CD137/4-1BB antibody, an agonist anti-CD27 antibody, an agonist anti-glucocorticoid-induced TNFR-related protein GITR antibody, and any combination thereof.

In some embodiments, the methods of the present disclosure may further include administering to the individual at least one stimulatory cytokine. In some embodiments, the at least one stimulatory cytokine is administered in combination with the anti-CD33 antibody. In some embodiments, the at least one stimulatory cytokine is selected from IFN-a4, IFN-b, IL-1β, TNF-α, IL-6, IL-8, CRP, IL-20 family members, LIF, IFN-gamma, OSM, CNTF, GM-CSF, IL-11, IL-12, IL-17, IL-18, IL-23, CXCL10, IL-33, CRP, IL-33, MCP-1, MIP-1-beta, and any combination thereof.

In some embodiments, a subject or individual is a mammal. Mammals include, without limitation, domesticated animals (e.g., cows, sheep, cats, dogs, and horses), primates (e.g., humans and non-human primates such as monkeys), rabbits, and rodents (e.g., mice and rats). In some embodiments, the subject or individual is a human.

Dementia

Dementia is a non-specific syndrome (i.e., a set of signs and symptoms) that presents as a serious loss of global cognitive ability in a previously unimpaired person, beyond what might be expected from normal ageing. Dementia may be static as the result of a unique global brain injury. Alternatively, dementia may be progressive, resulting in long-term decline due to damage or disease in the body. While dementia is much more common in the geriatric population, it can also occur before the age of 65. Cognitive areas affected by dementia include, without limitation, memory, attention span, language, and problem solving. Generally, symptoms must be present for at least six months to before an individual is diagnosed with dementia.

Exemplary forms of dementia include, without limitation, frontotemporal dementia, Alzheimer's disease, vascular dementia, semantic dementia, and dementia with Lewy bodies.

In some embodiments, administering an anti-CD33 antibody of the present disclosure can prevent, reduce the risk, and/or treat dementia. In some embodiments, an anti-CD33 antibody may modulate one or more CD33 activities in an individual having dementia.

Frontotemporal Dementia

Frontotemporal dementia (FTD) is a condition resulting from the progressive deterioration of the frontal lobe of the brain. Over time, the degeneration may advance to the temporal lobe. Second only to Alzheimer's disease (AD) in prevalence, FTD accounts for 20% of pre-senile dementia cases. The clinical features of FTD include memory deficits, behavioral abnormalities, personality changes, and language impairments (Cruts, M. & Van Broeckhoven, C., Trends Genet. 24:186-194 (2008); Neary, D., et al., Neurology 51:1546-1554 (1998); Ratnavalli, E., Brayne, C., Dawson, K. & Hodges, J. R., Neurology 58:1615-1621 (2002)).

A substantial portion of FTD cases are inherited in an autosomal dominant fashion, but even in one family, symptoms can span a spectrum from FTD with behavioral disturbances, to Primary Progressive Aphasia, to Cortico-Basal Ganglionic Degeneration. FTD, like most neurodegenerative diseases, can be characterized by the pathological presence of specific protein aggregates in the diseased brain. Historically, the first descriptions of FTD recognized the presence of intraneuronal accumulations of hyperphosphorylated Tau protein in neurofibrillary tangles or Pick bodies. A causal role for the microtubule associated protein Tau was supported by the identification of mutations in the gene encoding the Tau protein in several families (Hutton, M., et al., Nature 393:702-705 (1998). However, the majority of FTD brains show no accumulation of hyperphosphorylated Tau but do exhibit immunoreactivity to ubiquitin (Ub) and TAR DNA binding protein (TDP43) (Neumann, M., et al., Arch. Neurol. 64:1388-1394 (2007)). A majority of those FTD cases with Ub inclusions (FTD-U) were shown to carry mutations in the Progranulin gene.

In some embodiments, administering an anti-CD33 antibody of the present disclosure, can prevent, reduce the risk, and/or treat FTD. In some embodiments, administering an anti-CD33 antibody, may modulate one or more CD33 activities in an individual having FTD.

Alzheimer's Disease

Alzheimer's disease (AD) is the most common form of dementia. There is no cure for the disease, which worsens as it progresses, and eventually leads to death. Most often, AD is diagnosed in people over 65 years of age. However, the less-prevalent early-onset Alzheimer's can occur much earlier.

Common symptoms of Alzheimer's disease include, behavioral symptoms, such as difficulty in remembering recent events; cognitive symptoms, confusion, irritability and aggression, mood swings, trouble with language, and long-term memory loss. As the disease progresses bodily functions are lost, ultimately leading to death. Alzheimer's disease develops for an unknown and variable amount of time before becoming fully apparent, and it can progress undiagnosed for years.

In some embodiments, administering an anti-CD33 antibody of the present disclosure can prevent, reduce the risk, and/or treat Alzheimer's disease. In some embodiments, administering an anti-CD33 antibody may modulate one or more CD33 activities in an individual having Alzheimer's disease.

In some embodiments, treatment and/or delay of the progression of Alzheimer's disease in an individual administered an anti-CD33 antibody according to the methods provided herein is assessed according to any method known in the art. In some embodiments, treatment and/or delay of the progression of Alzheimer's disease in an individual administered an anti-CD33 antibody according to the methods provided herein is assessed using the Clinical Dementia Rating Sum (CDR) Sum of Boxes (CDR-SB) score, the Mini-Mental State Examination (MMSE) score, the Repeatable Battery for the Assessment of Neuropsychological Status (RBANS) score, amyloid brain positron emission tomography (PET), or translocator protein (TSPO)-PET imaging, and any combination thereof. In some embodiments, administration of an anti-CD33 antibody according to the methods provided herein results in an improvement of at least about 10%, at least about 20%, at least about 30%, at least about 40%, at least about 50%, at least about 60%, at least about 70%, at least about 80%, at least about 90%, at least about 95%, at least about 99%, or about 100% in the Clinical Dementia Rating Sum (CDR) Sum of Boxes (CDR-SB) score, the Mini-Mental State Examination (MMSE) score, the Repeatable Battery for the Assessment of Neuropsychological Status (RBANS) score, amyloid brain positron emission tomography (PET), or translocator protein (TSPO)-PET imaging, and any combination thereof.

Parkinson's Disease

Parkinson's disease, which may be referred to as idiopathic or primary parkinsonism, hypokinetic rigid syndrome (HRS), or paralysis agitans, is a neurodegenerative brain disorder that affects motor system control. The progressive death of dopamine-producing cells in the brain leads to the major symptoms of Parkinson's. Most often, Parkinson's disease is diagnosed in people over 50 years of age. Parkinson's disease is idiopathic (having no known cause) in most people. However, genetic factors also play a role in the disease.

Symptoms of Parkinson's disease include, without limitation, tremors of the hands, arms, legs, jaw, and face, muscle rigidity in the limbs and trunk, slowness of movement (bradykinesia), postural instability, difficulty walking, neuropsychiatric problems, changes in speech or behavior, depression, anxiety, pain, psychosis, dementia, hallucinations, and sleep problems.

In some embodiments, administering an anti-CD33 antibody of the present disclosure can prevent, reduce the risk, and/or treat Parkinson's disease. In some embodiments, administering an anti-CD33 antibody, may modulate one or more CD33 activities in an individual having Parkinson's disease.

Amyotrophic Lateral Sclerosis (ALS)

As used herein, amyotrophic lateral sclerosis (ALS) or, motor neuron disease or, Lou Gehrig's disease are used interchangeably and refer to a debilitating disease with varied etiology characterized by rapidly progressive weakness, muscle atrophy and fasciculations, muscle spasticity, difficulty speaking (dysarthria), difficulty swallowing (dysphagia), and difficulty breathing (dyspnea).

It has been shown that Progranulin plays a role in ALS (Schymick, J C et al., (2007) J Neurol Neurosurg Psychiatry; 78:754-6) and protects again the damage caused by ALS causing proteins such as TDP-43 (Laird, A S et al., (2010). PLoS ONE 5: e13368). It was also demonstrated that pro-NGF induces p75 mediated death of oligodendrocytes and corticospinal neurons following spinal cord injury (Beatty et al., Neuron (2002), 36, pp. 375-386; Giehl et al, Proc. Natl. Acad. Sci USA (2004), 101, pp 6226-30).

In some embodiments, administering an anti-CD33 antibody of the present disclosure can prevent, reduce the risk, and/or treat ALS. In some embodiments, administering an anti-CD33 antibody, may modulate one or more CD33 activities in an individual having amyotrophic lateral sclerosis.

Huntington's Disease

Huntington's disease (HD) is an inherited neurodegenerative disease caused by an autosomal dominant mutation in the Huntingtin gene (HTT). Expansion of a cytokine-adenine-guanine (CAG) triplet repeat within the Huntingtin gene results in production of a mutant form of the Huntingtin protein (Htt) encoded by the gene. This mutant Huntingtin protein (mHtt) is toxic and contributes to neuronal death. Symptoms of Huntington's disease most commonly appear between the ages of 35 and 44, although they can appear at any age.

Symptoms of Huntington's disease, include, without limitation, motor control problems, jerky, random movements (chorea), abnormal eye movements, impaired balance, seizures, difficulty chewing, difficulty swallowing, cognitive problems, altered speech, memory deficits, thinking difficulties, insomnia, fatigue, dementia, changes in personality, depression, anxiety, and compulsive behavior.

In some embodiments, administering as an anti-CD33 antibody of the present disclosure can prevent, reduce the risk, and/or treat Huntington's disease (HD). In some embodiments, administering an anti-CD33 antibody, may modulate one or more CD33 activities in an individual having Huntington's disease.

Taupathy Disease

Taupathy diseases, or Tauopathies, are a class of neurodegenerative disease caused by aggregation of the microtubule-associated protein tau within the brain. Alzheimer's disease (AD) is the most well-known taupathy disease, and involves an accumulation of tau protein within neurons in the form of insoluble neurofibrillary tangles (NFTs). Other taupathy diseases and disorders include progressive supranuclear palsy, dementia pugilistica (chromic traumatic encephalopathy), frontotemporal dementia and parkinsonism linked to chromosome 17, Lytico-Bodig disease (Parkinson-dementia complex of Guam), Tangle-predominant dementia, Ganglioglioma and gangliocytoma, Meningioangiomatosis, Subacute sclerosing panencephalitis, lead encephalopathy, tuberous sclerosis, Hallervorden-Spatz disease, lipofuscinosis, Pick's disease, corticobasal degeneration, Argyrophilic grain disease (AGD), Huntington's disease, and frontotemporal lobar degeneration.

In some embodiments, administering an anti-CD33 antibody of the present disclosure, can prevent, reduce the risk, and/or treat taupathy disease. In some embodiments, administering an anti-CD33 antibody, may modulate one or more CD33 activities in an individual having a taupathy disease.

Multiple Sclerosis

Multiple sclerosis (MS) can also be referred to as disseminated sclerosis or encephalomyelitis disseminata. MS is an inflammatory disease in which the fatty myelin sheaths around the axons of the brain and spinal cord are damaged, leading to demyelination and scarring as well as a broad spectrum of signs and symptoms. MS affects the ability of nerve cells in the brain and spinal cord to communicate with each other effectively. Nerve cells communicate by sending electrical signals called action potentials down long fibers called axons, which are contained within an insulating substance called myelin. In MS, the body's own immune system attacks and damages the myelin. When myelin is lost, the axons can no longer effectively conduct signals. MS onset usually occurs in young adults, and is more common in women (see, e.g., http://en(dot)wikipedia(dot)org/wiki/Multiple_sclerosis-cite_note-pmid18970977-1

Symptoms of MS include, without limitation, changes in sensation, such as loss of sensitivity or tingling; pricking or numbness, such as hypoesthesia and paresthesia; muscle weakness; clonus; muscle spasms; difficulty in moving; difficulties with coordination and balance, such as ataxia; problems in speech, such as dysarthria, or in swallowing, such as dysphagia; visual problems, such as nystagmus, optic neuritis including phosphenes, and diplopia; fatigue; acute or chronic pain; and bladder and bowel difficulties; cognitive impairment of varying degrees; emotional symptoms of depression or unstable mood; Uhthoffs phenomenon, which is an exacerbation of extant symptoms due to an exposure to higher than usual ambient temperatures; and Lhermitte's sign, which is an electrical sensation that runs down the back when bending the neck.

In some embodiments, administering an anti-CD33 antibody of the present disclosure can prevent, reduce the risk, and/or treat multiple sclerosis. In some embodiments, administering an anti-CD33 antibody may modulate one or more CD33 activities in an individual having multiple sclerosis.

Cancer

Further aspects of the present disclosure provide methods for preventing, reducing risk, or treating cancer, by administering to an individual in need thereof a therapeutically effective amount of an isolated anti-CD33 antibody of the present disclosure. Any of the isolated antibodies of the present disclosure may be used in these methods. In some embodiments, the isolated antibody is an agonist antibody of the present disclosure. In other embodiments, the isolated antibody is an antagonist antibody of the present disclosure. In other embodiments, the isolated antibody is an inert antibody of the present disclosure. In other embodiments, the isolated antibody is an antibody conjugate of the present disclosure.

As disclosed herein, the tumor microenvironment is known to contain a heterogeneous immune infiltrate, which includes T lymphocytes, macrophages and cells of myeloid/granulocytic lineage. The presence and activity of T-regulatory cells, tumor-imbedded immunosuppressor myeloid cells, and/or M2-macrophages in tumors is associated with poor prognosis. In contrast, the presence and activity of cytotoxic T cells is beneficial for cancer therapy. Therapies that directly or indirectly enhance the activity of cytotoxic T cells and reduce the number and activity of the various immunosuppressor cells, are expected to provide significant therapeutic benefit. A seminal preclinical study has shown synergies between drugs that target immunosuppressor cells (e.g., CSF1/CSF1R blocking antibodies) and immune checkpoint blocking antibodies that activate cytotoxic T cells, indicating that manipulating both cell types shows efficacy in tumor models where individual therapies are poorly effective (Zhu Y; Cancer Res. 2014 Sep. 15; 74(18):5057-69). Therefore, in some embodiments, blocking CD33, which is expressed on myeloid cells, subset of T cells, and tumor-associated immune cells, may stimulate beneficial anti-tumor immune response, resulting in a therapeutic anti-tumor immune response.

In some embodiments, the methods for preventing, reducing risk, or treating an individual having cancer further include administering to the individual at least one antibody that specifically binds to an inhibitory checkpoint molecule. Examples of antibodies that specifically bind to an inhibitory checkpoint molecule include, without limitation, an anti-PD-L1 antibody, an anti-CTLA4 antibody, an anti-PD-L2 antibody, an anti-PD-1 antibody, an anti-B7-H3 antibody, an anti-B7-H4 antibody, and anti-HVEM antibody, an anti-BTLA antibody, an anti-GAL9 antibody, an anti-TIM3 antibody, an anti-A2AR antibody, an anti-LAG-3 antibody, an anti-phosphatidylserine antibody, and any combination thereof. In some embodiments, the at least one antibody that specifically binds to an inhibitory checkpoint molecule is administered in combination with an antagonist anti-CD33 antibody of the present disclosure.

In some embodiments, a cancer to be prevented or treated by the methods of the present disclosure includes, without limitation, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B cell lymphoma; chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumors), Meigs' syndrome, brain, as well as head and neck cancer, and associated metastases. In some embodiments, the cancer is colorectal cancer. In some embodiments, the cancer is selected from non-small cell lung cancer, glioblastoma, neuroblastoma, renal cell carcinoma, bladder cancer, ovarian cancer, melanoma, breast carcinoma, gastric cancer, and hepatocellular carcinoma. In some embodiments, the cancer is triple-negative breast carcinoma. In some embodiments, the cancer may be an early stage cancer or a late stage cancer. In some embodiments, the cancer may be a primary tumor. In some embodiments, the cancer may be a metastatic tumor at a second site derived from any of the above types of cancer.

In some embodiments, anti-CD33 antibodies of the present disclosure may be used for preventing, reducing risk, or treating cancer, including, without limitation, bladder cancer breast cancer, colon and rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, and thyroid cancer.

In some embodiments, the present disclosure provides methods of preventing, reducing risk, or treating an individual having cancer, by administering to the individual a therapeutically effective amount of an anti-CD33 antibody of the present disclosure.

In some embodiments, the method further includes administering to the individual at least one antibody that specifically binds to an inhibitory immune checkpoint molecule, and/or another standard or investigational anti-cancer therapy. In some embodiments, the at least one antibody that specifically binds to an inhibitory checkpoint molecule is administered in combination with the anti-CD33 antibody of the present disclosure. In some embodiments, the at least one antibody that specifically binds to an inhibitory checkpoint molecule is selected from an anti-PD-L1 antibody, an anti-CTLA4 antibody, an anti-PD-L2 antibody, an anti-PD-1 antibody, an anti-B7-H3 antibody, an anti-B7-H4 antibody, and anti-HVEM antibody, an anti-B- and T-lymphocyte attenuator (BTLA) antibody, an anti-Killer inhibitory receptor (KIR) antibody, an anti-GAL9 antibody, an anti-TIM3 antibody, an anti-A2AR antibody, an anti-LAG-3 antibody, an anti-phosphatidylserine antibody, an anti-CD27 antibody, and any combination thereof. In some embodiments, the standard or investigational anti-cancer therapy is one or more therapies selected from radiotherapy, cytotoxic chemotherapy, targeted therapy, imatinib (Gleevec®), trastuzumab (Herceptin®), adoptive cell transfer (ACT), chimeric antigen receptor T cell transfer (CAR-T), vaccine therapy, and cytokine therapy.

In some embodiments, the method further includes administering to the individual at least one antibody that specifically binds to an inhibitory cytokine. In some embodiments, the at least one antibody that specifically binds to an inhibitory cytokine is administered in combination with the anti-CD33 antibody of the present disclosure. In some embodiments, the at least one antibody that specifically binds to an inhibitory cytokine is selected from an anti-CCL2 antibody, an anti-CSF-1 antibody, an anti-IL-2 antibody, and any combination thereof.

In some embodiments, the method further includes administering to the individual at least one agonistic antibody that specifically binds to a stimulatory immune checkpoint protein. In some embodiments, the at least one agonistic antibody that specifically binds to a stimulatory checkpoint protein is administered in combination with the anti-CD33 antibody of the present disclosure. In some embodiments, the at least one agonistic antibody that specifically binds to a stimulatory checkpoint protein is selected from an agonist anti-CD40 antibody, an agonist anti-OX40 antibody, an agonist anti-ICOS antibody, an agonist anti-CD28 antibody, an agonist anti-CD137/4-1BB antibody, an agonist anti-CD27 antibody, an agonist anti-glucocorticoid-induced TNFR-related protein GITR antibody, and any combination thereof.

In some embodiments, the method further includes administering to the individual at least one stimulatory cytokine. In some embodiments, the at least one stimulatory cytokine is administered in combination with the anti-CD33 antibody of the present disclosure. In some embodiments, the at least one stimulatory cytokine is selected from TNF-α, IL-6, IL-8, CRP, IL-20 family member, LIF, OSM, CNTF, IL-11, IL-12, IL-17, IL-8, CRP, IFN-α, IFN-$, IL-2, IL-18, GM-CSF, G-CSF, and any combination thereof.

### Diagnostic Uses

The isolated antibodies of the present disclosure (e.g., an anti-CD33 antibody described herein) also have diagnostic utility. This disclosure therefore provides for methods of using the antibodies of this disclosure, or functional fragments thereof, for diagnostic purposes, such as the detection of a CD33 protein in an individual or in tissue samples derived from an individual.

In some embodiments, the individual is a human. In some embodiments, the individual is a human patient suffering from, or at risk for developing a disease, disorder, or injury of the present disclosure. In some embodiments, the diagnostic methods involve detecting a CD33 protein in a biological sample, such as a biopsy specimen, a tissue, or a cell. An anti-CD33 antibody described herein is contacted with the biological sample and antigen-bound antibody is detected. For example, a biopsy specimen may be stained with an anti-CD33 antibody described herein in order to detect and/or quantify disease-associated cells. The detection method may involve quantification of the antigen-bound antibody. Antibody detection in biological samples may occur with any method known in the art, including immunofluorescence microscopy, immunocytochemistry, immunohistochemistry, ELISA, FACS analysis, immunoprecipitation, or micro-positron emission tomography. In certain embodiments, the antibody is radiolabeled, for example with ¹⁸F and subsequently detected utilizing micro-positron emission tomography analysis. Antibody-binding may also be quantified in a patient by non-invasive techniques such as positron emission tomography (PET), X-ray computed tomography, single-photon emission computed tomography (SPECT), computed tomography (CT), and computed axial tomography (CAT).

In other embodiments, an isolated antibody of the present disclosure (e.g., an anti-CD33 antibody described herein) may be used to detect and/or quantify, for example, microglia in a brain specimen taken from a preclinical disease model (e.g., a non-human disease model). As such, an isolated antibody of the present disclosure (e.g., an anti-CD33 antibody described herein) may be useful in evaluating therapeutic response after treatment in a model for a nervous system disease or injury such as frontotemporal dementia, Alzheimer's disease, vascular dementia, seizures, retinal dystrophy, atherosclerotic vascular diseases, Nasu-Hakola disease, or multiple sclerosis, as compared to a control.

### CD33 Antibodies

Certain aspects of the present disclosure are based, at least in part, on the identification of anti-CD33 antibodies that exhibit one or more improved and/or enhanced functional characteristics (e.g., relative to an anti-CD33 antibody having a heavy chain variable region comprising the sequence of SEQ ID NO: 103 and a light chain variable region comprising the sequence of SEQ ID NO: 104), including, an improved/enhanced ability to decrease cell surface levels of CD33 on cells, resulting in the reduction, neutralization, prevention, or curbing of one or more CD33 activities, including, without limitation, reducing cell growth of monocytes, macrophages, T cells, dendritic cells and/or microglia; reducing T cell proliferation induced by dendritic cells, bone marrow-derived dendritic cells, monocytes, microglia, M1 microglia, activated M1 microglia, M2 microglia, macrophages, M1 macrophages, activated M1 macrophages, and/or M2 macrophages; decreasing survival of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; decreasing proliferation of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; inhibiting migration of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; decreasing one or more functions of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; reducing proliferation of monocytes, macrophages, T cells, dendritic cells, neutrophils, and/or microglia; reducing the overall functionality of monocytes, macrophages, T cells, dendritic cells, neutrophils, and/or microglia; inhibition of beneficial immune response to different types of cancer selected from bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, acute myeloid leukemia, pancreatic cancer, prostate cancer, ovarian cancer, fibrosarcoma, and thyroid cancer; inhibition of beneficial immune response to different types of neurological disorders selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, essential tremor, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, Sarcoidosis, diseases of aging, seizures, spinal cord injury, traumatic brain injury, age related macular degeneration, glaucoma, retinitis pigmentosa, retinal degeneration, and multiple sclerosis; binding to CD33 ligand on tumor cells; binding to CD33 ligand on dendritic cells, bone marrow-derived dendritic cells, monocytes, microglia, T cells, neutrophils, and/or macrophages; inhibition of tumor cell killing by one or more of microglia, macrophages, dendritic cells, bone marrow-derived dendritic cells, neutrophils, T cells, T helper cells, or cytotoxic T cells; inhibition of anti-tumor cell proliferation activity of one or more of microglia, macrophages, dendritic cells, bone marrow-derived dendritic cells, neutrophils, T cells, T helper cells, or cytotoxic T cells; inhibition of anti-tumor cell metastasis activity of one or more of microglia, macrophages, dendritic cells, bone marrow-derived dendritic cells, neutrophils, T cells, T helper cells, or cytotoxic T cells; modulated expression of one or more inflammatory receptors, such as CD86, expressed on one or more of microglia, macrophages, dendritic cells, bone marrow-derived dendritic cells, neutrophils, T cells, T helper cells, or cytotoxic T cells; enhancing infiltration of one or more of immunosuppressor dendritic cells, immunosuppressor macrophages, myeloid derived suppressor cells, tumor-associated macrophages, immunosuppressor neutrophils, and regulatory T cells into tumors; increasing number of tumor-promoting myeloid/granulocytic immune-suppressive cells in a tumor, in peripheral blood, or other lymphoid organ; enhancing tumor-promoting activity of myeloid-derived suppressor cells; decreasing activation of tumor-specific T lymphocytes with tumor killing potential; decreasing infiltration of tumor-specific T lymphocytes with tumor killing potential; increasing tumor growth rate; increasing rate of tumor recurrence; decreasing efficacy of one or more immune-therapies that modulate anti-tumor T cell responses, optionally wherein the one or more immune-therapies are immune-therapies that target one or more proteins selected from CD40, OX40, ICOS, CD28, CD137/4-1BB, CD27, GITR, PD-L1, CTLA4, PD-L2, PD-1, B7-H3, B7-H4, HVEM, LIGHT, BTLA, VISTA, KIR, GAL9, TIM1, TIM3, TIM4, A2AR, LAG3, DR-5, CD39, CD70, TREM1, TREM2, Siglec-5, Siglec-7, Siglec-9, Siglec-11, SirpA, CD447, CSF-1 receptor, and any combination thereof, or of one or chemotherapy agents and/or more cancer vaccines.

In some embodiments, treatment of cancer with anti-CD33 antibodies as described herein may: (i) increase the number of tumor infiltrating CD3⁺ T cells; (ii) decrease cellular levels of CD33 in non-tumorigenic CD14⁺ myeloid cells, optionally wherein the non-tumorigenic CD14⁺ myeloid cells are tumor infiltrating cells or optionally wherein the non-tumorigenic CD14⁺ myeloid cells are present in blood; (iii) reduce the number of non-tumorigenic CD14⁺ myeloid cells, optionally wherein the non-tumorigenic CD14⁺ myeloid cells are tumor infiltrating cells or optionally wherein the non-tumorigenic CD14⁺ myeloid cells are present in blood; (iv) reduce PD-L1, PD-L2, B7-H7, B7-H3, CD200R, CD163, and/or CD206 levels in one or more cells, optionally wherein the one or more cells are non-tumorigenic myeloid-derived suppressor cells (MDSC); (v) decrease tumor growth rate of solid tumors; (vi) reducing tumor volume; (vii) increase efficacy of one or more PD-1 inhibitors; (viii) increase efficacy of one or more checkpoint inhibitor therapies and/or immune-modulating therapies, optionally wherein the one or more checkpoint inhibitor therapies and/or immune-modulating therapies target one or more of CTL4, the adenosine pathway, PD-L1, PD-L2, OX40, TIM3, LAG3, or any combination thereof; (ix) increase efficacy of one or more chemotherapy agents, optionally wherein the one or more of the chemotherapy agents are gemcitabine, capecitabine, anthracyclines, doxorubicin (Adriamycin®), epirubicin (Ellence®), taxanes, paclitaxel (Taxol®), docetaxel (Taxotere®), 5-fluorouracil (5-FU), cyclophosphamide (Cytoxan®), carboplatin (Paraplatin®), and any combination thereof; (x) i increase proliferation of T cells in the presence of non-tumorigenic myeloid-derived suppressor cells (MDSC); (xi) inhibit differentiation, survival, and/or one or more functions of non-tumorigenic myeloid-derived suppressor cells (MDSC); and (xii) kil CD33-expressing immunosuppressor non-tumorigenic myeloid cells and/or non-tumorigenic CD14-expressing cells in solid tumors and associated blood vessels when conjugated to a chemical or radioactive toxin.

In some embodiments, myeloid cells of the present disclosure include, without limitation, CD45+CD14+ myeloid cells, CD14+ myeloid cells, and myeloid-derived suppressor cells (MDSC). In some embodiments, myeloid cells of the present disclosure are non-tumorigenic myeloid cells. Immunosuppressor cells are sometimes also referred to as myeloid-derived suppressor cells (MDSC). In humans, MDSCs can be defined by one of the following combination of markers: (1) CD14+ HLA-DRlow/−, (2) CD14+IL4Rα+, (3) CD14+ HLA-DR−IL4Rα+, (4) CD34+CD14+CD11b+CD33+, (5) CD11b+ C D14+ CD33+, (6) CD33+ HLA-DR−, (7) Lin− HLA-DR−, (8) Lin− HLA-DR− CD33+, (9) Lin− HLA-DR− CD33+CD11b+, (10) Lin− CD33+ CD11b+ CD15+, (11) Lin− HLA-DR− CD33+ CD11b+ CD14− CD15+, (12) CD11b+ CD14− CD33+, (13) CD11b+ CD14− HLA-DR− CD33+CD15+, (14) CD33+ HLA-DR− CD15+, (15) CD15+IL4Rα+, (16) CD11b+ CD15+CD66b+, (17) CD15+ FSClow SSChigh, (18) CD15high CD33+, (19) CD11b+ CD14− CD15+, (20) CD66b+ SSChigh, and (21) CD11b+ CD15+ (see also Solito S et al. Annals of the NY Academy of Sciences, 2014). In mice, MDSCs can be defined by the expression of the surface markers CD45+, CD11b+, Gr1+, and/or I14Ra+. Additional exemplary immunosuppressive monocytic lineages are CD45+, CD11b+, Gr1low; and CD45+, CD11c+.

Certain aspects of the present disclosure relate to anti-CD33 antibodies comprising one or more improved and/or enhanced functional characteristics. In some embodiments, anti-CD33 antibodies of the present disclosure comprise one or more improved and/or enhanced functional characteristics relative to a control antibody (e.g., a control anti-CD33 antibody comprising a heavy chain variable region comprising the amino acid sequence of SEQ ID NO: 34 and a light chain variable region comprising the amino acid sequence of SEQ ID NO: 77; a control anti-CD33 antibody comprising a heavy chain variable region comprising the amino acid sequence of SEQ ID NO: 40 and a light chain variable region comprising the amino acid sequence of SEQ ID NO: 86; and control anti-CD33 antibody comprising a heavy chain variable region comprising the amino acid sequence of SEQ ID NO: 52 and a light chain variable region comprising the amino acid sequence of SEQ ID NO: 86; and/or a control anti-CD33 antibody comprising a heavy chain variable region comprising the amino acid sequence of SEQ ID NO: 103 and a light chain variable region comprising the amino acid sequence of SEQ ID NO: 104). In some embodiments, anti-CD33 antibodies of the present disclosure have an affinity for CD33 (e.g., human CD33) that is higher than that of a control anti-CD33 antibody (e.g., a control anti-CD33 antibody comprising a heavy chain variable region comprising the amino acid sequence of SEQ ID NO: 34 and a light chain variable region comprising the amino acid sequence of SEQ ID NO: 77; a control anti-CD33 antibody comprising a heavy chain variable region comprising the amino acid sequence of SEQ ID NO: 40 and a light chain variable region comprising the amino acid sequence of SEQ ID NO: 86; and control anti-CD33 antibody comprising a heavy chain variable region comprising the amino acid sequence of SEQ ID NO: 52 and a light chain variable region comprising the amino acid sequence of SEQ ID NO: 86; and/or a control anti-CD33 antibody comprising a heavy chain variable region comprising the amino acid sequence of SEQ ID NO: 103 and a light chain variable region comprising the amino acid sequence of SEQ ID NO: 104). In some embodiments, anti-CD33 antibodies of the present disclosure bind to human cells, such as dendritic cells, with a half-maximal effective concentration (EC₅₀) that is lower than that of a control antibody (e.g., a control anti-CD33 antibody comprising a heavy chain variable region comprising the amino acid sequence of SEQ ID NO: 34 and a light chain variable region comprising the amino acid sequence of SEQ ID NO: 77; a control anti-CD33 antibody comprising a heavy chain variable region comprising the amino acid sequence of SEQ ID NO: 40 and a light chain variable region comprising the amino acid sequence of SEQ ID NO: 86; and control anti-CD33 antibody comprising a heavy chain variable region comprising the amino acid sequence of SEQ ID NO: 52 and a light chain variable region comprising the amino acid sequence of SEQ ID NO: 86; and/or a control anti-CD33 antibody comprising a heavy chain variable region comprising the amino acid sequence of SEQ ID NO: 103 and a light chain variable region comprising the amino acid sequence of SEQ ID NO: 104). In some embodiments, anti-CD33 antibodies of the present disclosure decrease cellular levels (e.g., cell surface levels) of CD33 with a half-maximal effective concentration (EC₅₀) that is lower than that of a control antibody (e.g., a control anti-CD33 antibody comprising a heavy chain variable region comprising the sequence of SEQ ID NO: 103 and a light chain variable region comprising the sequence of SEQ ID NO: 104).

Cellular levels of CD33 may refer to, without limitation, cell surface levels of CD33, intracellular levels of CD33, and total levels of CD33. In some embodiments, a decrease in cellular levels of CD33 comprises decrease in cell surface levels of CD33. In some embodiments, anti-CD33 antibodies of the present disclosure that decrease cellular levels of CD33 (e.g., cell surface levels of CD33) have one or more of the following characteristics: (1) inhibits or reduces one or more CD33 activities; (2) the ability to inhibit or reduce binding of a CD33 to one or more of its ligands; (3) the ability to reduce CD33 expression in CD33-expressing cells; (4) the ability to interact, bind, or recognize a CD33 protein; (5) the ability to specifically interact with or bind to a CD33 protein; and (6) the ability to treat, ameliorate, or prevent any aspect of a disease or disorder described or contemplated herein.

Anti-CD33 antibodies of the present disclosure may have nanomolar or even picomolar affinities for the target antigen (e.g., human CD33). In certain embodiments, the dissociation constant (K_{D}) of the antibody is from about 0.001 to about 100 nM. In certain embodiments, the K_{D} of the antibody is about 0.01 to about 10 nM. In certain embodiments, the K_{D} of the antibody is about 0.202 to about 8.57 nM. In some embodiments, the K_{D} of the antibody is less than about or equal to about 100 nM, 90 nM, 80 nM, 70 nM, 60 nM, 50 nM, 40 nM, 30 nM, 20 nM, 10 nM, 9.5 nM, 9 nM, 8.5 nM, 8 nM, 7.5 nM, 7 nM, 6.5 nM, 6 nM, 5.5 nM, 5 nM, 4.5 nM, 4 nM, 3.5 nM, 3 nM, 2.5 nM, 2 nM, 1.5 nM, 1 nM, 0.9 nM, 0.8 nM, 0.7 nM, 0.6 nM, 0.5 nM, 0.4 nM, 0.3 nM, 0.2 nM, 0.1 nM, 0.05 nM, 0.01 nM, or 0.005 nM. In some embodiments, the K_{D} of the antibody is less than about 5.22 nM. In some embodiments, the K_{D} of the antibody is greater than about or equal to about 0.001 nM, 0.005 nM, 0.01 nM, 0.05 nM, 0.1 nM, 0.2 nM, 0.3 nM, 0.4 nM, 0.5 nM, 0.6 nM, 0.7 nM, 0.8 nM, 0.9 nM, 1 nM, 1.5 nM, 2 nM, 2.5 nM, 3 nM 3.5 nM, 4 nM, 4.5 nM, 5 nM, 5.5 nM, 6 nM, 6.5 nM, 7 nM, 7.5 nM, 8 nM, 8.5 nM, 9 nM, 9.5 nM, 10 nM. 20 nM, 30 nM, 40 nM, 50 nM, 60 nM, 70 nM, 80 nM, or 90 nM. That is, the K_{D} of the antibody can be any of a range of affinities having an upper limit of about 100 nM, 90 nM, 80 nM, 70 nM, 60 nM, 50 nM, 40 nM, 30 nM, 20 nM, 10 nM, 9.5 nM, 9 nM, 8.5 nM, 8 nM, 7.5 nM, 7 nM, 6.5 nM, 6 nM, 5.5 nM, 5 nM, 4.5 nM, 4 nM, 3.5 nM, 3 nM, 2.5 nM, 2 nM, 1.5 nM, 1 nM, 0.9 nM, 0.8 nM, 0.7 nM, 0.6 nM, 0.5 nM, 0.4 nM, 0.3 nM, 0.2 nM, 0.1 nM, 0.05 nM, 0.01 nM, or 0.005 nM, and an independently selected lower limit of about 0.001 nM, 0.005 nM, 0.01 nM, 0.05 nM, 0.1 nM, 0.2 nM, 0.3 nM, 0.4 nM, 0.5 nM, 0.6 nM, 0.7 nM, 0.8 nM, 0.9 nM, 1 nM, 1.5 nM, 2 nM, 2.5 nM, 3 nM 3.5 nM, 4 nM, 4.5 nM, 5 nM, 5.5 nM, 6 nM, 6.5 nM, 7 nM, 7.5 nM, 8 nM, 8.5 nM, 9 nM, 9.5 nM, 10 nM. 20 nM, 30 nM, 40 nM, 50 nM, 60 nM, 70 nM, 80 nM, or 90 nM, wherein the lower limit is less than the upper limit. In some embodiments, the K_{D} of the antibody is any of about 10 nM, about 9 nM, about 8 nM, about 7 nM, about 6 nM, about 5 nM, about 4 nM, about 3 nM, about 2 nM, about 1 nM, about 900 pM, about 800 pM, about 700 pM, about 600 pM, about 500 pM, about 400 pM, about 300 pM, about 200 pM, or about 100 pM. Various methods of measuring antibody affinity are known in the art, including, for example, using surface plasmon resonance or BioLayer Interferometry (See e.g., Example 1 below). In some embodiments, the K_{D} for CD33 is determined at a temperature of approximately 25° C. In some embodiments, the K_{D} for CD33 is determined at a temperature of approximately 4° C. In some embodiments, the K_{D} is determined using a monovalent antibody (e.g., a Fab) or a full-length antibody in a monovalent form. In some embodiments, the K_{D} is determined using a bivalent antibody and monomeric recombinant CD33 protein.

In some embodiments, anti-CD33 antibodies of the present disclosure have a lower dissociation constant (K_{D}) for CD33 than a control anti-CD33 antibody (e.g., a control anti-CD33 antibody comprising a heavy chain variable region comprising the amino acid sequence of SEQ ID NO: 34 and a light chain variable region comprising the amino acid sequence of SEQ ID NO: 77; a control anti-CD33 antibody comprising a heavy chain variable region comprising the amino acid sequence of SEQ ID NO: 40 and a light chain variable region comprising the amino acid sequence of SEQ ID NO: 86; and control anti-CD33 antibody comprising a heavy chain variable region comprising the amino acid sequence of SEQ ID NO: 52 and a light chain variable region comprising the amino acid sequence of SEQ ID NO: 86; and/or a control anti-CD33 antibody comprising a heavy chain variable region comprising the amino acid sequence of SEQ ID NO: 103 and a light chain variable region comprising the amino acid sequence of SEQ ID NO: 104). In some embodiments, anti-CD33 antibodies of the present disclosure have a K_{D} for a target (e.g., human CD33) that is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% lower than the K_{D} of a control anti-CD33 antibody for the target (e.g., a control anti-CD33 antibody comprising a heavy chain variable region comprising the amino acid sequence of SEQ ID NO: 34 and a light chain variable region comprising the amino acid sequence of SEQ ID NO: 77; a control anti-CD33 antibody comprising a heavy chain variable region comprising the amino acid sequence of SEQ ID NO: 40 and a light chain variable region comprising the amino acid sequence of SEQ ID NO: 86; and control anti-CD33 antibody comprising a heavy chain variable region comprising the amino acid sequence of SEQ ID NO: 52 and a light chain variable region comprising the amino acid sequence of SEQ ID NO: 86; and/or a control anti-CD33 antibody comprising a heavy chain variable region comprising the amino acid sequence of SEQ ID NO: 103 and a light chain variable region comprising the amino acid sequence of SEQ ID NO: 104). In some embodiments, anti-CD33 antibodies of the present disclosure have a K_{D} for a target (e.g., human CD33) that is at least about 1-fold, at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 17.5-fold, at least about 20-fold, at least about 22.5-fold, at least about 25-fold, at least about 27.5-fold, at least about 30-fold, at least about 50-fold, or at least about 100-fold lower than the K_{D} of a control anti-CD33 antibody for the target (e.g., a control anti-CD33 antibody comprising a heavy chain variable region comprising the amino acid sequence of SEQ ID NO: 34 and a light chain variable region comprising the amino acid sequence of SEQ ID NO: 77; a control anti-CD33 antibody comprising a heavy chain variable region comprising the amino acid sequence of SEQ ID NO: 40 and a light chain variable region comprising the amino acid sequence of SEQ ID NO: 86; and control anti-CD33 antibody comprising a heavy chain variable region comprising the amino acid sequence of SEQ ID NO: 52 and a light chain variable region comprising the amino acid sequence of SEQ ID NO: 86; and/or a control anti-CD33 antibody comprising a heavy chain variable region comprising the amino acid sequence of SEQ ID NO: 103 and a light chain variable region comprising the amino acid sequence of SEQ ID NO: 104). In some embodiments, anti-CD33 antibodies of the present disclosure have a K_{D} for human CD33 that is at least 9-fold greater than an anti-CD33 antibody having a heavy chain variable region comprising the amino acid sequence of SEQ ID NO: 34 and a light chain variable region comprising the amino acid sequence of SEQ ID NO: 77. In some embodiments, anti-CD33 antibodies of the present disclosure have a K_{D} for human CD33 that is at least 3-fold greater than an anti-CD33 antibody having a heavy chain variable region comprising the amino acid sequence of SEQ ID NO: 40 and a light chain variable region comprising the amino acid sequence of SEQ ID NO: 86. In some embodiments, anti-CD33 antibodies of the present disclosure have a K_{D} for human CD33 that is at least 3-fold greater than an anti-CD33 antibody having a heavy chain variable region comprising the amino acid sequence of SEQ ID NO: 52 and a light chain variable region comprising the amino acid sequence of SEQ ID NO: 86. In some embodiments, the affinity is measured by surface plasmon resonance. In some embodiments, the affinity is measured at a temperature of approximately 25° C. In some embodiments, the affinity is measured at a temperature of approximately 4° C. In some embodiments, the affinity is measured using the experimental approach as described in Example 1 below.

Anti-CD33 antibodies of the present disclosure may decrease cellular levels (e.g., cell surface levels) of CD33 with a half-maximal effective concentration (EC₅₀) (e.g., when measured in vitro using primary human dendritic cells) in the picomolar range. In certain embodiments, the EC₅₀ of the antibody is about 0.1 to about 500 pM. In certain embodiments, the EC₅₀ of the antibody is about 1 to about 250 pM. In certain embodiments, the EC₅₀ of the antibody is about 4.1 to about 151.1 pM. In some embodiments, the EC₅₀ of the antibody is less than about or equal to about 500 pM, 400 pM, 300 pM, 250 pM, 225 pM, 200 pM, 175 pM, 150 pM, 125 pM, 100 pM, 75 pM, 50 pM, 25 pM, 10 pM, 1 pM, or 0.5 pM. In some embodiments, the EC₅₀ of the antibody is less than about 74.3 pM. In some embodiments, the EC₅₀ of the antibody is greater than about or equal to about 0.1 pM, 0.5 pM, 1 pM, 10 pM, 25 pM, 50 pM, 75 pM, 100 pM, 125 pM, 150 pM, 175 pM, 200 pM, 225 pM, 250 pM, 300 pM, or 400 pM. That is, the EC₅₀ of the antibody can be any of a range having an upper limit of about 500 pM, 400 pM, 300 pM, 250 pM, 225 pM, 200 pM, 175 pM, 150 pM, 125 pM, 100 pM, 75 pM, 50 pM, 25 pM, 10 pM, 1 pM, or 0.5 pM, and an independently selected lower limit of about 0.1 pM, 0.5 pM, 1 pM, 10 pM, 25 pM, 50 pM, 75 pM, 100 pM, 125 pM, 150 pM, 175 pM, 200 pM, 225 pM, 250 pM, 300 pM, or 400 pM, wherein the lower limit is less than the upper limit. In some embodiments, the EC₅₀ of the antibody is any of about 1 pM, 2 pM, 3 pM, 4 pM, 5 pM, 6 pM, 7 pM, 8 pM, 9 pM, 10 pM, 15 pM, 20 pM, 25 pM, 30 pM, 35 pM, 40 pM, 45 pM, 5 pM, 55 pM, 60 pM, 65 pM, 70 pM, 75 pM, 80 pM, 85 pM, 90 pM, 95 pM, 100 pM, 105 pM, 110 pM, 115 pM, 120 pM, 125 pM, 130 pM, 135 pM, 140 pM, 145 pM, 150 pM, 155 pM, 160 pM, 165 pM, 170 pM, 175 pM, 180 pM, 185 pM, 190 pM, 195 pM, or 200 pM. Various methods of measuring antibody EC₅₀ values are known in the art, including, for example, by flow cytometry (See e.g., Example 2 below). In some embodiments, the EC₅₀ is measured in vitro using primary human dendritic cells. In some embodiments, the EC₅₀ is measured in vitro using primary human monocytes. In some embodiments, the EC₅₀ is measured in vitro using primary human macrophages. In some embodiments, the EC₅₀ is measured in vitro using cultured cells transfected with human CD33. In some embodiments, the EC₅₀ is measured at a temperature of approximately 4° C. In some embodiments, the EC₅₀ is measured at a temperature of approximately 25° C. In some embodiments, the EC₅₀ is measured at a temperature of approximately 35° C. In some embodiments, the EC₅₀ is measured at a temperature of approximately 37° C. In some embodiments, the EC₅₀ is determined using a monovalent antibody (e.g., a Fab) or a full-length antibody in a monovalent form. In some embodiments, the EC₅₀ is determined using antibodies containing constant regions that demonstrate enhanced Fc receptor binding. In some embodiments, the EC₅₀ is determined using antibodies containing constant regions that demonstrate reduced Fc receptor binding.

In some embodiments, anti-CD33 antibodies of the present disclosure decrease cellular levels (e.g., cell surface levels) of CD33 with a lower EC₅₀ (e.g., as measured in vitro using primary human dendritic cells) than a control anti-CD33 antibody (e.g., a control anti-CD33 antibody comprising a heavy chain variable region comprising the amino acid sequence of SEQ ID NO: 34 and a light chain variable region comprising the amino acid sequence of SEQ ID NO: 77; a control anti-CD33 antibody comprising a heavy chain variable region comprising the amino acid sequence of SEQ ID NO: 40 and a light chain variable region comprising the amino acid sequence of SEQ ID NO: 86; and control anti-CD33 antibody comprising a heavy chain variable region comprising the amino acid sequence of SEQ ID NO: 52 and a light chain variable region comprising the amino acid sequence of SEQ ID NO: 86; and/or a control anti-CD33 antibody comprising a heavy chain variable region comprising the amino acid sequence of SEQ ID NO: 103 and a light chain variable region comprising the amino acid sequence of SEQ ID NO: 104). In some embodiments, anti-CD33 antibodies of the present disclosure decrease cellular levels (e.g., cell surface levels) of CD33 with an EC₅₀ that is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% lower than the EC₅₀ of a control anti-CD33 antibody (e.g., a control anti-CD33 antibody comprising a heavy chain variable region comprising the amino acid sequence of SEQ ID NO: 34 and a light chain variable region comprising the amino acid sequence of SEQ ID NO: 77; a control anti-CD33 antibody comprising a heavy chain variable region comprising the amino acid sequence of SEQ ID NO: 40 and a light chain variable region comprising the amino acid sequence of SEQ ID NO: 86; and control anti-CD33 antibody comprising a heavy chain variable region comprising the amino acid sequence of SEQ ID NO: 52 and a light chain variable region comprising the amino acid sequence of SEQ ID NO: 86; and/or a control anti-CD33 antibody comprising a heavy chain variable region comprising the amino acid sequence of SEQ ID NO: 103 and a light chain variable region comprising the amino acid sequence of SEQ ID NO: 104). In some embodiments, anti-CD33 antibodies of the present disclosure decrease cellular levels (e.g., cell surface levels) of CD33 with an EC₅₀ that is at least about 1-fold, at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 17.5-fold, at least about 20-fold, at least about 22.5-fold, at least about 25-fold, at least about 27.5-fold, at least about 30-fold, at least about 50-fold, or at least about 100-fold lower than the EC₅₀ of a control anti-CD33 antibody (e.g., a control anti-CD33 antibody comprising a heavy chain variable region comprising the amino acid sequence of SEQ ID NO: 34 and a light chain variable region comprising the amino acid sequence of SEQ ID NO: 77; a control anti-CD33 antibody comprising a heavy chain variable region comprising the amino acid sequence of SEQ ID NO: 40 and a light chain variable region comprising the amino acid sequence of SEQ ID NO: 86; and control anti-CD33 antibody comprising a heavy chain variable region comprising the amino acid sequence of SEQ ID NO: 52 and a light chain variable region comprising the amino acid sequence of SEQ ID NO: 86; and/or a control anti-CD33 antibody comprising a heavy chain variable region comprising the amino acid sequence of SEQ ID NO: 103 and a light chain variable region comprising the amino acid sequence of SEQ ID NO: 104). In some embodiments, anti-CD33 antibodies of the present disclosure have an EC₅₀ that is at least 1.6-fold lower than an anti-CD33 antibody having a heavy chain variable region comprising the amino acid sequence of SEQ ID NO: 34 and a light chain variable region comprising the amino acid sequence of SEQ ID NO: 77. In some embodiments, anti-CD33 antibodies of the present disclosure have an EC₅₀ that is at least 1.05-fold lower than an anti-CD33 antibody having a heavy chain variable region comprising the amino acid sequence of SEQ ID NO: 40 and a light chain variable region comprising the amino acid sequence of SEQ ID NO: 86. In some embodiments, anti-CD33 antibodies of the present disclosure have an EC₅₀ that is at least 1.07-fold lower than an anti-CD33 antibody having a heavy chain variable region comprising the amino acid sequence of SEQ ID NO: 52 and a light chain variable region comprising the amino acid sequence of SEQ ID NO: 86. In some embodiments, anti-CD33 antibodies of the present disclosure have an EC₅₀ that is at least 1.2-fold lower than an anti-CD33 antibody having a heavy chain variable region comprising the amino acid sequence of SEQ ID NO: 103 and a light chain variable region comprising the amino acid sequence of SEQ ID NO: 104. In some embodiments, the EC₅₀ is measured in vitro using primary human dendritic cells. In some embodiments, the EC₅₀ is measured in vitro using primary human monocytes. In some embodiments, the EC₅₀ is measured in vitro using primary human macrophages. In some embodiments, the EC₅₀ is measured in vitro using cultured cells transfected with human CD33. In some embodiments, the EC₅₀ is measured by flow cytometry. In some embodiments, the EC₅₀ is measured at a temperature of approximately 25° C. In some embodiments, the EC₅₀ is measured at a temperature of approximately 35° C. In some embodiments, the EC₅₀ is measured at a temperature of approximately 37° C. In some embodiments, the EC₅₀ is determined using antibodies containing constant regions that demonstrate enhanced Fc receptor binding. In some embodiments, the EC₅₀ is determined using antibodies containing constant regions that demonstrate reduced Fc receptor binding. In some embodiments, the EC₅₀ is measured using the experimental approach as described in Example 2 below. Any in vitro cell-based assays or suitable in vivo model described herein or known in the art may be used to measure inhibition of interaction (e.g., binding) between CD33 and one or more CD33 ligands. In some embodiments, anti-CD33 antibodies of the present disclosure inhibit interaction (e.g., binding) between CD33 and one or more CD33 ligands by at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more at saturating antibody concentrations utilizing any in vitro assay or cell-based culture assay described herein or known in the art.

In some embodiments, anti-CD33 antibodies of the present disclosure inhibit cell surface clustering of CD33. In some embodiments, anti-CD33 antibodies of the present disclosure inhibit one or more activities of a CD33 protein, including, without limitation, counteracting one or more of phosphorylation of Tyr-340 and Tyr-358 by a Src family tyrosine kinase, such as LCK and FYN; recruitment of and binding to the tyrosine-specific protein phosphatases SHP1 and SHP2; recruitment of and binding to PLC-gamma1, which acts as a guanine nucleotide exchange factor for Dynamini-1; recruitment of and binding to SH2-domain containing protein (e.g., Crk1); recruitment of and binding to the spleen tyrosine kinase Syk; recruitment of and binding to SH3-SH2-SH3 growth factor receptor-bound protein 2 (Grb2); recruitment of and binding to multiple SH2-containing proteins; phosphorylation of Ser-307 and Ser-342 by protein kinase C; modulated expression of one or more anti-inflammatory cytokines, IL-4, IL-10, IL-13, IL-35, IL-16, TGF-beta, IL-1Ra, G-CSF, and soluble receptors for TNF, IFN-beta1a, IFN-beta1b, or IL-6 in monocytes, macrophages, T cells, dendritic cells neutrophils, and/or microglia; decreasing intracellular calcium mobilization; modulated expression of one or more pro-inflammatory cytokines IFN-α4, IFN-b, IL-1β, TNF-α, IL-6, IL-8, CRP, IL-20 family members, LIF, IFN-gamma, OSM, CNTF, GM-CSF, IL-11, IL-12, IL-17, IL-18, IL-23, CXCL10, IL-33, CRP, IL-33, MCP-1, and MIP-1-beta in monocytes, macrophages, T cells, dendritic cells, neutrophils, and/or microglia; modulated expression of one or more proteins selected from C1qa, C1qB, C1qC, C1s, C1R, C4, C2, C3, ITGB2, HMOX1, LAT2, CASP1, CSTA, VSIG4, MS4A4A, C3AR1, GPX1, TyroBP, ALOX5AP, ITGAM, SLC7A7, CD4, ITGAX, PYCARD, CD14, CD16, HLA-DR, and CCR2; inhibition of extracellular signal-regulated kinase (ERK) phosphorylation; decreasing tyrosine phosphorylation on multiple cellular proteins; modulated expression of C-C chemokine receptor 7 (CCR7); inhibition of microglial cell chemotaxis toward CCL19 and CCL21 expressing cells; activation of phosphoinositide 3-kinase; reducing cell growth of monocytes, macrophages, T cells, dendritic cells and/or microglia; reducing T cell proliferation induced by dendritic cells, bone marrow-derived dendritic cells, monocytes, microglia, M1 microglia, activated M1 microglia, M2 microglia, macrophages, M1 macrophages, activated M1 macrophages, and/or M2 macrophages; inhibition of osteoclast production, decreased rate of osteoclastogenesis, or both; decreasing survival of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; decreasing proliferation of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; inhibiting migration of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; decreasing one or more functions of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; inhibiting maturation of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; increasing cell death and apoptosis of monocytes, macrophages, T cells, dendritic cells, neutrophils, and/or microglia; reducing phagocytic activity of monocytes, macrophages, T cells, dendritic cells, neutrophils, and/or microglia; reducing proliferation of monocytes, macrophages, T cells, dendritic cells, neutrophils, and/or microglia; reducing the overall functionality of monocytes, macrophages, T cells, dendritic cells, neutrophils, and/or microglia, phosphorylation of an ITAM containing receptor; phosphorylation of a signaling molecules that mediates ITAM signaling; reducing the activation of pattern recognition receptors; reducing the activation of Toll-like receptors; reducing the activation of damage-associated of clearance of cellular and protein debris; interaction between CD33 and one or more of its ligands; interaction between CD33 and a co-receptor such as CD64; reducing one or more types of clearance selected from apoptotic neuron clearance, nerve tissue debris clearance, dysfunctional synapse clearance, non-nerve tissue debris clearance, bacteria or other foreign body clearance, disease-causing protein clearance, and tumor cell clearance; inhibition of phagocytosis of one or more of apoptotic neurons, nerve tissue debris, non-nerve tissue debris, bacteria, other foreign bodies, disease-causing proteins, disease-causing peptides, disease-causing nucleic acid, disease-causing lipids, or tumor cells; inhibition of clearance of a disease-causing nucleic acid, such as the disease-causing nucleic acid is antisense GGCCCC (G2C4) repeat-expansion RNA; activation of clearance of, a disease-causing protein selected from amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides; inhibition of beneficial immune response to different types of cancer selected from bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, acute myeloid leukemia, pancreatic cancer, prostate cancer, ovarian cancer, fibrosarcoma, and thyroid cancer; inhibition of beneficial immune response to different types of neurological disorders selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, essential tremor, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, Sarcoidosis, diseases of aging, seizures, spinal cord injury, traumatic brain injury, age related macular degeneration, glaucoma, retinitis pigmentosa, retinal degeneration, and multiple sclerosis; inhibition of beneficial immune response-to different types of inflammatory and infectious disorders selected from lupus, acute and chronic colitis, wound healing, Crohn's disease, inflammatory bowel disease, ulcerative colitis, obesity, malaria, respiratory tract infection, sepsis, eye infection, systemic infection, lupus, arthritis, low bone density, osteoporosis, osteogenesis, osteopetrotic disease, and Paget's disease of bone; binding to CD33 ligand on tumor cells; binding to CD33 ligand on dendritic cells, bone marrow-derived dendritic cells, monocytes, microglia, T cells, neutrophils, and/or macrophages; inhibition of tumor cell killing by one or more of microglia, macrophages, dendritic cells, bone marrow-derived dendritic cells, neutrophils, T cells, T helper cells, or cytotoxic T cells; inhibition of anti-tumor cell proliferation activity of one or more of microglia, macrophages, dendritic cells, bone marrow-derived dendritic cells, neutrophils, T cells, T helper cells, or cytotoxic T cells; inhibition of anti-tumor cell metastasis activity of one or more of microglia, macrophages, dendritic cells, bone marrow-derived dendritic cells, neutrophils, T cells, T helper cells, or cytotoxic T cells; promotion of immunosuppressor dendritic cells, immunosuppressor macrophages, myeloid-derived suppressor cells, tumor-associated macrophages, or regulatory T cells; inhibition of one or more ITAM motif containing receptors, such as TREM1, TREM2, FcgR, DAP10, and DAP12; inhibition of one or more receptors containing the motif D/Ex0-2YxxL/IX6-8YxxL/I (SEQ ID NO: 165); inhibition of signaling by one or more pattern recognition receptors (PRRs), such as receptors that identify pathogen-associated molecular patterns (PAMPs), and receptors that identify damage-associated molecular patterns (DAMPs); inhibition of signaling by one or more Toll-like receptors; inhibition of the JAK-STAT signaling pathway; inhibition of nuclear factor kappa-light-chain-enhancer of activated B cells (NFκB); inhibition of PLCγ/PKC/calcium mobilization; inhibition of PI3K/Akt, Ras/MAPK signaling; reduced expression of one or more inflammatory receptors, such as CD86, expressed on one or more of microglia, macrophages, dendritic cells, bone marrow-derived dendritic cells, neutrophils, T cells, T helper cells, or cytotoxic T cells; increasing expression of one or more CD33-dependent genes; normalization of disrupted CD33-dependent gene expression; and decreasing expression of one or more ITAM-dependent genes, such as NFAT transcription factors.

In some embodiments, anti-CD33 antibodies of the present disclosure exhibit one or more activities of a CD33 protein, including, without limitation, increasing the number of tumor infiltrating CD3⁺ T cells; decreasing cellular levels of CD33 in CD14⁺ myeloid cells, such as tumor infiltrating CD14⁺ myeloid cells and CD14⁺ myeloid cells present in blood; reducing the number of CD14⁺ myeloid cells, such as tumor infiltrating CD14⁺ myeloid cells and CD14⁺ myeloid cells present in blood; reducing PD-L1, PD-L2, B7-H7, B7-H3, CD200R, CD163, and/or CD206 levels in one or more cells, such as myeloid-derived suppressor cells (MDSC); decreasing tumor growth rate of solid tumors; reducing tumor volume; increasing efficacy of one or more PD-1 inhibitors; increasing efficacy of one or more checkpoint inhibitor therapies and/or immune-modulating therapies, such as checkpoint inhibitor therapies and/or immune-modulating therapies that target one or more of CTL4, the adenosine pathway, PD-L1, PD-L2, OX40, TIM3, LAG3, or any combination thereof; increasing efficacy of one or more chemotherapy agents, optionally wherein the one or more of the chemotherapy agents are gemcitabine, capecitabine, anthracyclines, doxorubicin (Adriamycin®), epirubicin (Ellence®), taxanes, paclitaxel (Taxol®), docetaxel (Taxotere®), 5-fluorouracil (5-FU), cyclophosphamide (Cytoxan®), carboplatin (Paraplatin®), oxaliplatin (Elotaxin®), leucovorin, temazolamide (Temodar®), and any combination thereof; increasing proliferation of T cells in the presence of myeloid-derived suppressor cells (MDSC); inhibiting differentiation, survival, and/or one or more functions of myeloid-derived suppressor cells (MDSC); and killing CD33-expressing immunosuppressor non-tumorigenic myeloid cells and/or non-tumorigenic CD14-expressing cells in solid tumors and associated blood vessels when conjugated to a chemical or radioactive toxin.

In some embodiments, the anti-CD33 antibodies inhibit interaction (e.g., binding) between a CD33 protein of the present disclosure and one or more CD33 ligands including, without limitation, CD33 ligands expressed on red blood cells, CD33 ligands expressed on bacterial cells, CD33 ligands expressed on apoptotic cells, CD33 ligands expressed on tumor cells, CD33 ligands expressed on viruses, CD33 ligands expressed on dendritic cells, CD33 ligands expressed on nerve cells, CD33 ligands expressed on glial cells, CD33 ligands expressed on microglia, CD33 ligands expressed on astrocytes, CD33 ligands on beta amyloid plaques, CD33 ligands on Tau tangles, CD33 ligands on disease-causing proteins, CD33 ligands on disease-causing peptides, CD33 ligands expressed on macrophages, CD33 ligands expressed on natural killer cells, CD33 ligands expressed on T cells, CD33 ligands expressed on T helper cells, CD33 ligands expressed on cytotoxic T cells, CD33 ligands expressed on B cells, CD33 ligands expressed on tumor-imbedded immunosuppressor dendritic cells, CD33 ligands expressed on tumor-imbedded immunosuppressor macrophages, CD33 ligands expressed on myeloid-derived suppressor cells, CD33 ligands expressed on regulatory T cells, secreted mucins, sialic acid, sialic acid-containing glycolipids, sialic acid-containing glycoproteins, alpha-2,6-linked sialic acid-containing glycolipids, alpha-2,6-linked sialic acid-containing glycoproteins, alpha-2,3-linked sialic acid-containing glycolipids, alpha-2,3-linked sialic acid-containing glycoproteins, alpha-1-acid glycoprotein (AGP), CD24 protein, and gangliosides.

In some embodiments, anti-CD33 antibodies of the present disclosure bind to a CD33 protein of the present disclosure expressed on the surface of cell and the naked antibodies inhibit interaction (e.g., binding) between the CD33 protein and one or more CD33 ligands. In some embodiments, anti-CD33 antibodies of the present disclosure that bind to a CD33 protein of the present inhibit interaction (e.g., binding) between the CD33 protein and one or more CD33 ligands by reducing the effective levels of CD33 that is available to interact with these proteins either on the cell surface or inside the cell. In some embodiments, anti-CD33 antibodies of the present disclosure that bind to a CD33 protein of the present inhibit interaction (e.g., binding) between the CD33 protein and one or more CD33 ligands by inducing degradation of CD33.

As used herein, levels of CD33 may refer to expression levels of the gene encoding CD33; to expression levels of one or more transcripts encoding CD33; to expression levels of CD33 protein; and/or to the amount of CD33 protein present within cells and/or on the cell surface. Any methods known in the art for measuring levels of gene expression, transcription, translation, and/or protein abundance or localization may be used to determine the levels of CD33.

Additionally, anti-CD33 antibodies of the present disclosure can be used to prevent, reduce risk of, or treat dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer including bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express CD33, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and/or Haemophilus influenza. In some embodiments, anti-CD33 antibodies of the present disclosure can be used for inducing or promoting the survival, maturation, functionality, migration, or proliferation of one or more immune cells in an individual in need thereof; or for decreasing the activity, functionality, or survival of regulatory T cells, tumor-imbedded immunosuppressor dendritic cells, tumor-imbedded immunosuppressor macrophages, myeloid-derived suppressor cells, tumor-associated macrophages, acute myeloid leukemia (AML) cells, chronic lymphocytic leukemia (CLL) cell, and/or chronic myeloid leukemia (CML) cell in an individual in need thereof. In some embodiments, anti-CD33 antibodies of the present disclosure are monoclonal antibodies.

In some embodiments, an isolated anti-CD33 antibody of the present disclosure decreases cellular levels of CD33 (e.g., cell surface levels, intracellular levels, and/or total levels). In some embodiments, an isolated anti-CD33 antibody of the present disclosure induces downregulation of CD33. In some embodiments, an isolated anti-CD33 antibody of the present disclosure induces cleavage of CD33. In some embodiments, an isolated anti-CD33 antibody of the present disclosure induces internalization of CD33. In some embodiments, an isolated anti-CD33 antibody of the present disclosure induces shedding of CD33. In some embodiments, an isolated anti-CD33 antibody of the present disclosure induces degradation of CD33. In some embodiments, an isolated anti-CD33 antibody of the present disclosure induces desensitization of CD33. In some embodiments, an isolated anti-CD33 antibody of the present disclosure acts as a ligand mimetic to transiently activate CD33. In some embodiments, an isolated anti-CD33 antibody of the present disclosure acts as a ligand mimetic and transiently activates CD33 before inducing a decrease in cellular levels of CD33 and/or inhibition of interaction (e.g., binding) between CD33 and one or more CD33 ligands. In some embodiments, an isolated anti-CD33 antibody of the present disclosure acts as a ligand mimetic and transiently activates CD33 before inducing degradation of CD33. In some embodiments, an isolated anti-CD33 antibody of the present disclosure acts as a ligand mimetic and transiently activates CD33 before inducing cleavage of CD33. In some embodiments, an isolated anti-CD33 antibody of the present disclosure acts as a ligand mimetic and transiently activates CD33 before inducing internalization of CD33. In some embodiments, an isolated anti-CD33 antibody of the present disclosure acts as a ligand mimetic and transiently activates CD33 before inducing shedding of CD33. In some embodiments, an isolated anti-CD33 antibody of the present disclosure acts as a ligand mimetic and transiently activates CD33 before inducing downregulation of CD33 expression. In some embodiments, an isolated anti-CD33 antibody of the present disclosure acts as a ligand mimetic and transiently activates CD33 before inducing desensitization of CD33.

In some embodiments, an isolated anti-CD33 antibody of the present disclosure is a human antibody, a humanized antibody, a bispecific antibody, a monoclonal antibody, a multivalent antibody, or a chimeric antibody. Exemplary descriptions of such antibodies are found throughout the present disclosure.

In some embodiments, anti-CD33 antibodies of the present disclosure bind to a human CD33, or a homolog thereof, including without limitation, a mammalian CD33 protein. In some embodiments, anti-CD33 antibodies of the present disclosure specifically bind to human CD33. In some embodiments, anti-CD33 antibodies of the present disclosure bind to human CD33 and are not cross-reactive with CD33 orthologs or homologs from other species.

In some embodiments, anti-CD33 antibodies of the present disclosure bind to a CD33 protein of the present disclosure expressed on the surface of a cell and modulate (e.g., induce or inhibit) one or more CD33 activities of the present disclosure after binding to the surface-expressed CD33 protein. In some embodiments, anti-CD33 antibodies of the present disclosure are inert antibodies.

Anti-CD33 Antibody-Binding Regions

In some embodiments, anti-CD33 antibodies of the present disclosure may bind a conformational epitope. In some embodiments, anti-CD33 antibodies of the present disclosure may bind a discontinuous CD33 epitope. In some embodiments, the discontinuous CD33 epitope comprises two or more peptides, three or more peptides, four or more peptides, five or more peptides, six or more peptides, seven or more peptides, eight or more peptides, nine or more peptides, or 10 or more peptides. In some embodiments, anti-CD33 antibodies of the present disclosure may bind a CD33 epitope comprising one or more peptides. As disclosed herein, CD33 epitopes may comprise one or more peptides comprising five or more, six or more, seven or more, eight or more, nine or more, 10 or more, 11 or more, 12 or more, 13 or more 14 or more, 15 or more, 16 or more, 17 or more, 18 or more, 19 or more, or 20 or more amino acid residues of the amino acid sequence of SEQ ID NO: 1, or five or more, six or more, seven or more, eight or more, nine or more, 10 or more, 11 or more, 12 or more, 13 or more 14 or more, 15 or more, 16 or more, 17 or more, 18 or more, 19 or more, or 20 or more amino acid residues on a mammalian CD33 protein corresponding to the amino acid sequence of SEQ ID NO: 1.

In some embodiments, anti-CD33 antibodies of the present disclosure bind to an epitope of human CD33 that is the same as or overlaps with the CD33 epitope bound by an anti-CD33 antibody comprising a heavy chain variable region comprising the amino acid sequence of SEQ ID NO: 103 and a light chain variable region comprising the amino acid sequence of SEQ ID NO: 104. In some embodiments, anti-CD33 antibodies of the present disclosure bind essentially the same CD33 epitope bound by an anti-CD33 antibody comprising a heavy chain variable region comprising the amino acid sequence of SEQ ID NO: 103 and a light chain variable region comprising the amino acid sequence of SEQ ID NO: 104.

In some embodiments, anti-CD33 antibodies of the present disclosure competitively inhibit binding of an anti-CD33 antibody comprising a heavy chain variable region comprising the amino acid sequence of SEQ ID NO: 103 and a light chain variable region comprising the amino acid sequence of SEQ ID NO: 104. In some embodiments, anti-CD33 antibodies of the present disclosure compete with an anti-CD33 antibody comprising a heavy chain variable region comprising the amino acid sequence of SEQ ID NO: 103 and a light chain variable region comprising the amino acid sequence of SEQ ID NO: 104 for binding to CD33.

In some embodiments, anti-CD33 antibodies of the present disclosure competitively inhibit binding of at least one antibody selected from any of the antibodies listed in Tables 1A-1C, 2A-3C, 3, 4, 5A-5D, and 6A-6D. In some embodiments, anti-CD33 antibodies of the present disclosure competitively inhibit binding of at least one antibody selected from AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, AB-64.1.15, AB-H2, AB-H9, AB-H14, AB-H15, AB-H63, AB-H64, AB-H65, AB-H66, and any combination thereof. In some embodiments, an anti-CD33 antibody of the present disclosure competes with one or more antibodies selected from AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, AB-64.1.15, AB-H2, AB-H9, AB-H14, AB-H15, AB-H63, AB-H64, AB-H65, AB-H66, and any combination thereof, for binding to CD33 when the anti-CD33 antibody reduces the binding of one or more antibodies selected from AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, AB-64.1.15, AB-H2, AB-H9, AB-H14, AB-H15, AB-H63, AB-H64, AB-H65, AB-H66, and any combination thereof to CD33 by an amount the ranges from about 50% to 100%, as compared to binding to CD33 in the absence of the anti-CD33 antibody. In some embodiments, an anti-CD33 antibody of the present disclosure competes with one or more antibodies selected from AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, AB-64.1.15, AB-H2, AB-H9, AB-H14, AB-H15, AB-H63, AB-H64, AB-H65, AB-H66, and any combination thereof for binding to CD33 when the anti-CD33 antibody reduces the binding of one or more antibodies selected from AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, AB-64.1.15, AB-H2, AB-H9, AB-H14, AB-H15, AB-H63, AB-H64, AB-H65, AB-H66, and any combination thereof to CD33 by at least 50%, at least 55%, by at least 60%, at least 65%, at least 70%, at least 75%, at least 80%, at least 85%, at least 90%, at least 95%, or 100%, as compared to binding to CD33 in the absence of the anti-CD33 antibody. In some embodiments, an anti-CD33 antibody of the present disclosure that reduces the binding of one or more antibodies selected from AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, AB-64.1.15, AB-H2, AB-H9, AB-H14, AB-H15, AB-H63, AB-H64, AB-H65, AB-H66, and any combination thereof to CD33 by 100% indicates that the anti-CD33 antibody essential completely blocks the binding of one or more antibodies selected from AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, AB-64.1.15, AB-H2, AB-H9, AB-H14, AB-H15, AB-H63, AB-H64, AB-H65, AB-H66, and any combination thereof to CD33. In some embodiments, the anti-CD33 antibody and the one or more antibodies selected from AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, AB-64.1.15, AB-H2, AB-H9, AB-H14, AB-H15, AB-H63, AB-H64, AB-H65, AB-H66, and any combination thereof are present in an amount that corresponds to a 10:1 ratio, 9:1 ratio, 8:1 ratio, 7:1 ratio, 6:1 ratio, 5:1 ratio, 4:1 ratio, 3:1 ratio, 2:1 ratio, 1:1 ratio, 0.75:1 ratio, 0.5:1 ratio, 0.25:1 ratio, 0.1:1 ratio, 0.075:1 ratio, 0.050:1 ratio, 0.025:1 ratio, 0.01:1 ratio, 0.0075:ratio, 0.0050:1 ratio, 0.0025:1 ratio, 0.001:ratio, 0.00075:1 ratio, 0.00050:1 ratio, 0.00025:1 ratio, 0.0001:ratio, 1:10 ratio, 1:9 ratio, 1:8 ratio, 1:7 ratio, 1:6 ratio, 1:5 ratio, 1:4 ratio, 1:3 ratio, 1:2 ratio, 1:0.75 ratio, 1:0.5 ratio, 1:0.25 ratio, 1:0.1 ratio, 1:0.075 ratio, 1:0.050 ratio, 1:0.025 ratio, 1:0.01 ratio, 1:0.0075 ratio, 1:0.0050 ratio, 1:0.0025 ratio, 1:0.001 ratio, 1:0.00075 ratio, 1:0.00050 ratio, 1:0.00025 ratio, or 1:0.0001 ratio of anti-CD33 antibody to one or more antibodies selected from AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, AB-64.1.15, AB-H2, AB-H9, AB-H14, AB-H15, AB-H63, AB-H64, AB-H65, AB-H66, and any combination thereof. In some embodiments, the anti-CD33 antibody is present in excess by an amount that ranges from about 1.5-fold to 100-fold, or greater than 100-fold compared to the amount of the one or more antibodies selected from AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, AB-64.1.15, AB-H2, AB-H9, AB-H14, AB-H15, AB-H63, AB-H64, AB-H65, AB-H66, and any combination thereof. In some embodiments, the anti-CD33 antibody is present in an amount that is about a 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold excess compared to the amount of the one or more antibodies selected from AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, AB-64.1.15, AB-H2, AB-H9, AB-H14, AB-H15, AB-H63, AB-H64, AB-H65, AB-H66, and any combination thereof.

In some embodiments, anti-CD33 antibodies of the present disclosure bind to an epitope of human CD33 that is the same as or overlaps with the CD33 epitope bound by at least one antibody selected from any of the antibodies listed in Tables 1A-1C, 2A-3C, 3, 4, 5A-5D, and 6A-6D. In some embodiments, anti-CD33 antibodies of the present disclosure bind to an epitope of human CD33 that is the same as or overlaps with the CD33 epitope bound by at least one antibody selected from AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, AB-64.1.15, AB-H2, AB-H9, AB-H14, AB-H15, AB-H63, AB-H64, AB-H65, AB-H66.

In some embodiments, anti-CD33 antibodies of the present disclosure bind essentially the same CD33 epitope bound by at least one antibody selected from any of the antibodies listed in Tables 1A-1C, 2A-3C, 3, 4, 5A-5D, and 6A-6D. In some embodiments, anti-CD33 antibodies of the present disclosure bind essentially the same CD33 epitope bound by at least one antibody selected from AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, AB-64.1.15, AB-H2, AB-H9, AB-H14, AB-H15, AB-H63, AB-H64, AB-H65, AB-H66. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In some embodiments, anti-CD33 antibodies of the present disclosure compete with one or more antibodies selected from AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, AB-64.1.15, AB-H2, AB-H9, AB-H14, AB-H15, AB-H63, AB-H64, AB-H65, AB-H66, and any combination thereof for binding to CD33.

Any suitable competition assay or CD33 binding assay known in the art, such as BIAcore analysis, ELISA assays, or flow cytometry, may be utilized to determine whether an anti-CD33 antibody competes with one or more antibodies selected from AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, AB-64.1.15, AB-H2, AB-H9, AB-H14, AB-H15, AB-H63, AB-H64, AB-H65, AB-H66, and any combination thereof for binding to CD33. In an exemplary competition assay, immobilized CD33 or cells expressing CD33 on the cell surface are incubated in a solution comprising a first labeled antibody that binds to CD33 (e.g., human or non-human primate) and a second unlabeled antibody that is being tested for its ability to compete with the first antibody for binding to CD33. The second antibody may be present in a hybridoma supernatant. As a control, immobilized CD33 or cells expressing CD33 is incubated in a solution comprising the first labeled antibody but not the second unlabeled antibody. After incubation under conditions permissive for binding of the first antibody to CD33, excess unbound antibody is removed, and the amount of label associated with immobilized CD33 or cells expressing CD33 is measured. If the amount of label associated with immobilized CD33 or cells expressing CD33 is substantially reduced in the test sample relative to the control sample, then that indicates that the second antibody is competing with the first antibody for binding to CD33. See, Harlow and Lane (1988) Antibodies: A Laboratory Manual ch. 14 (Cold Spring Harbor Laboratory, Cold Spring Harbor, N.Y.).

Anti-CD33 Antibody Light Chain and Heavy Chain Variable Regions

In some embodiments, anti-CD33 antibodies of the present disclosure comprise a heavy chain variable region comprising one or more (e.g., one or more, two or more, or all three) HVRs selected from HVR-H1, HVR-H2, and HVR-H3 (as shown in Tables 1A-1C). In some embodiments, the heavy chain variable region comprises an HVR-H1, an HVR-H2, and an HVR-H3 (as shown in Tables 1A-1C). In some embodiments, the antibody is not an antibody comprising a heavy chain variable region comprising an HVR-H1 comprising the sequence of GYTFTDYNLH (SEQ ID NO: 105), an HVR-H2 comprising the sequence of FIYPSNGITG (SEQ ID NO: 115), and an HVR-H3 comprising the sequence of STVDYFDY (SEQ ID NO: 121).

In some embodiments, the HVR-H1 comprises a sequence according to Formula I: GX₁X₂X₃TDYNX₄H (SEQ ID NO: 152), wherein X₁ is Y, A, or V, X₂ is T or A, X₃ is F, E, or H, and X₄ is L, F, Y, or N. In some embodiments, the HVR-H1 comprises a sequence selected from SEQ ID NOs: 105-114. In some embodiments, the HVR-H2 comprises a sequence according to Formula II: FIYPX₁NX₂IX₃G (SEQ ID NO: 153), wherein X₁ is S or A, X₂ is G, Q, R, or V, and X₃ is T or R. In some embodiments, the HVR-H2 comprises a sequence selected from SEQ ID NOs: 115-120. In some embodiments, the HVR-H3 comprises a sequence according to Formula III: SX₁VDYFDX₂ (SEQ ID NO: 154), wherein X₁ is T, D, F, or S, and X₂ is Y, D, or L. In some embodiments, the HVR-H3 comprises a sequence selected from SEQ ID NOs: 121-126.

In some embodiments, the heavy chain variable region comprises an HVR-H1 according to Formula I, an HVR-H2 according to Formula II, and an HVR-H3 according to Formula III, and the antibody is not an antibody comprising a heavy chain variable region comprising an HVR-H1 comprising the sequence of GYTFTDYNLH (SEQ ID NO: 105), an HVR-H2 comprising the sequence of FIYPSNGITG (SEQ ID NO: 115), and an HVR-H3 comprising the sequence of STVDYFDY (SEQ ID NO: 121). In some embodiments, the heavy chain variable region comprises an HVR-H1 comprising a sequence selected from SEQ ID NOs: 105-114, and HVR-H2 comprising a sequence selected from SEQ ID NOs: 115-120, and an HVR-H3 comprising a sequence selected from SEQ ID NOs: 121-126, and the antibody is not an antibody comprising a heavy chain variable region comprising an HVR-H1 comprising the sequence of GYTFTDYNLH (SEQ ID NO: 105), an HVR-H2 comprising the sequence of FIYPSNGITG (SEQ ID NO: 115), and an HVR-H3 comprising the sequence of STVDYFDY (SEQ ID NO: 121).

In some embodiments, the heavy chain variable region comprises the HVR-H1, HVR-H2, and HVR-H3 of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15, and any combination thereof (as shown in Tables 1A to 1C).

In some embodiments, anti-CD33 antibodies of the present disclosure comprise a heavy chain variable region, wherein the heavy chain variable region comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-H1 amino acid sequence of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-H2 amino acid sequence of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-H3 amino acid sequence of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15, and the antibody is not an antibody comprising a heavy chain variable region comprising an HVR-H1 comprising the sequence of GYTFTDYNLH (SEQ ID NO: 105), an HVR-H2 comprising the sequence of FIYPSNGITG (SEQ ID NO: 115), and an HVR-H3 comprising the sequence of STVDYFDY (SEQ ID NO: 121).

In some embodiments, anti-CD33 antibodies of the present disclosure comprise an HVR-H1 comprising the amino acid sequence GYTFTDYNLH (SEQ ID NO: 105), an HVR-H2 comprising the amino acid sequence FIYPSNRITG (SEQ ID NO: 119), and an HVR-H3 comprising the amino acid sequence SDVDYFDY (SEQ ID NO: 122). In some embodiments, anti-CD33 antibodies of the present disclosure comprise an HVR-H1 comprising the amino acid sequence GYTFTDYNLH (SEQ ID NO: 105), an HVR-H2 comprising the amino acid sequence FIYPSNQITG (SEQ ID NO: 118), and an HVR-H3 comprising the amino acid sequence SDVDYFDY (SEQ ID NO: 122).

In some embodiments, anti-CD33 antibodies of the present disclosure comprise a light chain variable region comprising one or more (e.g., one or more, two or more, or all three) HVRs selected from HVR-L1, HVR-L2, and HVR-L3 (as shown in Tables 2A-2C). In some embodiments, the light chain variable region comprises an HVR-L1, an HVR-L2, and an HVR-L3 (as shown in Tables 2A-2C). In some embodiments, the antibody is not an antibody comprising a light chain variable region comprising an HVR-L1 comprising the sequence of RASQSVSTSTYSYMH (SEQ ID NO: 127), an HVR-L2 comprising the sequence of YASNLES (SEQ ID NO: 135), and an HVR-L3 comprising the sequence of QHSWEIPLT (SEQ ID NO: 146).

In some embodiments, the HVR-L1 comprises a sequence according to Formula IV: X₁X₂SQX₃VX₄X₅STYSYMH (SEQ ID NO: 155), wherein X₁ is R or K, X₂ is A, G, or V, X₃ is S or D, X₄ is S, G, or H, and X₅ is T or A. In some embodiments, the HVR-L1 comprises a sequence selected from SEQ ID NOs: 127-134. In some embodiments, the HVR-L2 comprises a sequence according to Formula V: YX₁X₂X₃X₄X₅S (SEQ ID NO: 156), wherein X₁ is A, V, or E, X₂ is S, V, or F, X₃ is N, A, Y, or F, X₄ is L or V, and X₅ is E, G, or N. In some embodiments, the HVR-L2 comprises a sequence selected from SEQ ID NOs: 135-145. In some embodiments, the HVR-L3 comprises a sequence according to Formula VI: X₁HSX₂X₃X₄PLX₅ (SEQ ID NO: 157), wherein X₁ is Q or E, X₂ is W or E, X₃ is E or A, X₄ is I or L, and X₅ is T or E. In some embodiments, the HVR-13 comprises a sequence selected from SEQ ID NOs: 146-151.

In some embodiments, the light chain variable region comprises an HVR-L1 according to Formula IV, an HVR-L2 according to Formula V, and an HVR-L3 according to Formula VI, and the antibody is not an antibody comprising a light chain variable region comprising an HVR-L1 comprising the sequence of RASQSVSTSTYSYMH (SEQ ID NO: 127), an HVR-L2 comprising the sequence of YASNLES (SEQ ID NO: 135), and an HVR-L3 comprising the sequence of QHSWEIPLT (SEQ ID NO: 146). In some embodiments, the light chain variable region comprises an HVR-L1 comprising a sequence selected from SEQ ID NOs: 127-134, and HVR-L2 comprising a sequence selected from SEQ ID NOs: 135-145, and an HVR-L3 comprising a sequence selected from SEQ ID NOs: 146-151, and the antibody is not an antibody comprising a light chain variable region comprising an HVR-L1 comprising the sequence of RASQSVSTSTYSYMH (SEQ ID NO: 127), an HVR-L2 comprising the sequence of YASNLES (SEQ ID NO: 135), and an HVR-L3 comprising the sequence of QHSWEIPLT (SEQ ID NO: 146).

In some embodiments, the light chain variable region comprises the HVR-L1, HVR-L2, and HVR-L3 of antibody AB-14.3, AB-14.4, AB-14.6, AB-14.7, AB-14.8, AB-14.9, AB-14.10, AB-14.11, AB-63.6, AB-63.7, AB-63.8, AB-63.9, AB-63.10, AB-63.11, AB-63.12, AB-63.14, AB-63.15, AB-63.16, AB-63.17, AB-63.18, AB-64.2, AB-64.3, AB-64.4, AB-64.5, AB-64.6, AB-64.7, or AB-64.8, and any combination thereof (as shown in Tables 2A to 2C).

In some embodiments, anti-CD33 antibodies of the present disclosure comprise a light chain variable region, wherein the light chain variable region comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-L1 amino acid sequence of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-L2 amino acid sequence of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-L3 amino acid sequence of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15, and the antibody is not an antibody comprising a light chain variable region comprising an HVR-L1 comprising the sequence of RASQSVSTSTYSYMH (SEQ ID NO: 127), an HVR-L2 comprising the sequence of YASNLES (SEQ ID NO: 135), and an HVR-L3 comprising the sequence of QHSWEIPLT (SEQ ID NO: 146).

In some embodiments, anti-CD33 antibodies of the present disclosure comprise an HVR-L1 comprising the amino acid sequence RASQSVSTSTYSYMH (SEQ ID NO: 127), an HVR-L2 comprising the amino acid sequence YASNLES (SEQ ID NO: 135), and an HVR-L3 comprising the amino acid sequence QHSWEIPLT (SEQ ID NO: 146). In some embodiments, anti-CD33 antibodies of the present disclosure comprise an HVR-L1 comprising the amino acid sequence RASQSVSTSTYSYMH (SEQ ID NO: 127), an HVR-L2 comprising the amino acid sequence YASNLES (SEQ ID NO: 135), and an HVR-L3 comprising the amino acid sequence QHSWEIPLT (SEQ ID NO: 146).

In some embodiments, anti-CD33 antibodies of the present disclosure comprise a heavy chain variable region comprising one or more (e.g., one or more, two or more, or all three) HVRs selected from HVR-H1, HVR-H2, and HVR-H3 (as shown in Tables 1A-1C), and a light chain variable region comprising one or more (e.g., one or more, two or more, or all three) HVRs selected from HVR-L1, HVR-L2, and HVR-L3 (as shown in Tables 2A-2C). In some embodiments, the heavy chain variable region comprises an HVR-H1, an HVR-H2, and an HVR-H3 (as shown in Tables 1A-1C), and the light chain variable region comprises an HVR-L1, an HVR-L2, and an HVR-L3 (as shown in Tables 2A-2C). In some embodiments, the antibody is not an antibody comprising a heavy chain variable region comprising an HVR-H1 comprising the sequence of GYTFTDYNLH (SEQ ID NO: 105), an HVR-H2 comprising the sequence of FIYPSNGITG (SEQ ID NO: 115), and an HVR-H3 comprising the sequence of STVDYFDY (SEQ ID NO: 121), and a light chain variable region comprising an HVR-L1 comprising the sequence of RASQSVSTSTYSYMH (SEQ ID NO: 127), an HVR-L2 comprising the sequence of YASNLES (SEQ ID NO: 135), and an HVR-L3 comprising the sequence of QHSWEIPLT (SEQ ID NO: 146).

In some embodiments, the heavy chain variable region comprises an HVR-H1 according to Formula I, an HVR-H2 according to Formula II, and an HVR-H3 according to Formula III, and the light chain variable region comprises an HVR-L1 according to Formula IV, an HVR-L2 according to Formula V, and an HVR-L3 according to Formula VI, and the antibody is not an antibody comprising a heavy chain variable region comprising an HVR-H1 comprising the sequence of GYTFTDYNLH (SEQ ID NO: 105), an HVR-H2 comprising the sequence of FIYPSNGITG (SEQ ID NO: 115), and an HVR-H3 comprising the sequence of STVDYFDY (SEQ ID NO: 121), and a light chain variable region comprising an HVR-L1 comprising the sequence of RASQSVSTSTYSYMH (SEQ ID NO: 127), an HVR-L2 comprising the sequence of YASNLES (SEQ ID NO: 135), and an HVR-L3 comprising the sequence of QHSWEIPLT (SEQ ID NO: 146). In some embodiments, the heavy chain variable region comprises an HVR-H1 comprising a sequence selected from SEQ ID NOs: 105-114, and HVR-H2 comprising a sequence selected from SEQ ID NOs: 115-120, and an HVR-H3 comprising a sequence selected from SEQ ID NOs: 121-126, and the light chain variable region comprises an HVR-L1 comprising a sequence selected from SEQ ID NOs: 127-134, and HVR-L2 comprising a sequence selected from SEQ ID NOs: 135-145, and an HVR-L3 comprising a sequence selected from SEQ ID NOs: 146-151, and the antibody is not an antibody comprising a heavy chain variable region comprising an HVR-H1 comprising the sequence of GYTFTDYNLH (SEQ ID NO: 105), an HVR-H2 comprising the sequence of FIYPSNGITG (SEQ ID NO: 115), and an HVR-H3 comprising the sequence of STVDYFDY (SEQ ID NO: 121), and a light chain variable region comprising an HVR-L1 comprising the sequence of RASQSVSTSTYSYMH (SEQ ID NO: 127), an HVR-L2 comprising the sequence of YASNLES (SEQ ID NO: 135), and an HVR-L3 comprising the sequence of QHSWEIPLT (SEQ ID NO: 146).

In some embodiments, anti-CD33 antibodies of the present disclosure comprise a heavy chain variable region comprising the HVR-H1, HVR-H2, and HVR-H3 of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15, and any combination thereof (as shown in Tables 1A to 1C); and a light chain variable region comprising the HVR-L1, HVR-L2, and HVR-L3 of antibody AB-14.3, AB-14.4, AB-14.6, AB-14.7, AB-14.8, AB-14.9, AB-14.10, AB-14.11, AB-63.6, AB-63.7, AB-63.8, AB-63.9, AB-63.10, AB-63.11, AB-63.12, AB-63.14, AB-63.15, AB-63.16, AB-63.17, AB-63.18, AB-64.2, AB-64.3, AB-64.4, AB-64.5, AB-64.6, AB-64.7, or AB-64.8, and any combination thereof (as shown in Tables 2A to 2C). In some embodiments, anti-CD33 antibodies of the present disclosure comprise a heavy chain variable region comprising an HVR-H1, HVR-H2, and HVR-H3 and a light chain variable region comprising an HVR-L1, HVR-L2, and HVR-L3, wherein the antibody comprises the HVR-H1, HVR-H2, HVR-H3, HVR-L1, HVR-L2, and HVR-L3 of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15 (as shown in Tables 1A to 1C and 2A to 2C).

In some embodiments, anti-CD33 antibodies of the present disclosure comprise a heavy chain variable region and a light chain variable region, wherein the heavy chain variable region comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-H1 amino acid sequence of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-H2 amino acid sequence of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-H3 amino acid sequence of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15, and the antibody is not an antibody comprising a heavy chain variable region comprising an HVR-H1 comprising the sequence of GYTFTDYNLH (SEQ ID NO: 105), an HVR-H2 comprising the sequence of FIYPSNGITG (SEQ ID NO: 115), and an HVR-H3 comprising the sequence of STVDYFDY (SEQ ID NO: 121); and wherein the light chain variable region comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-L1 amino acid sequence of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-L2 amino acid sequence of antibody B-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-L3 amino acid sequence of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15, and the antibody is not an antibody comprising a light chain variable region comprising an HVR-L1 comprising the sequence of RASQSVSTSTYSYMH (SEQ ID NO: 127), an HVR-L2 comprising the sequence of YASNLES (SEQ ID NO: 135), and an HVR-L3 comprising the sequence of QHSWEIPLT (SEQ ID NO: 146).

In some embodiments, anti-CD33 antibodies of the present disclosure comprise an HVR-H1 comprising the amino acid sequence GYTFTDYNLH (SEQ ID NO: 105), an HVR-H2 comprising the amino acid sequence FIYPSNRITG (SEQ ID NO: 119), an HVR-H3 comprising the amino acid sequence SDVDYFDY (SEQ ID NO: 122), an HVR-L1 comprising the amino acid sequence RASQSVSTSTYSYMH (SEQ ID NO: 127), an HVR-L2 comprising the amino acid sequence YASNLES (SEQ ID NO: 135), and an HVR-L3 comprising the amino acid sequence QHSWEIPLT (SEQ ID NO: 146). In some embodiments, anti-CD33 antibodies of the present disclosure comprise an HVR-H1 comprising the amino acid sequence GYTFTDYNLH (SEQ ID NO: 105), an HVR-H2 comprising the amino acid sequence FIYPSNQITG (SEQ ID NO: 118), an HVR-H3 comprising the amino acid sequence SDVDYFDY (SEQ ID NO: 122), an HVR-L1 comprising the amino acid sequence RASQSVSTSTYSYMH (SEQ ID NO: 127), an HVR-L2 comprising the amino acid sequence YASNLES (SEQ ID NO: 135), and an HVR-L3 comprising the amino acid sequence QHSWEIPLT (SEQ ID NO: 146).

In some embodiments, anti-CD33 antibodies of the present disclosure comprise a heavy chain variable region comprising an amino acid sequence selected from SEQ ID NOs: 34-72. In some embodiments, the heavy chain variable region comprises the amino acid sequence of SEQ ID NO: 59. In some embodiments, the heavy chain variable region comprises the amino acid sequence of SEQ ID NO: 65. In some embodiments, anti-CD33 antibodies of the present disclosure comprise a heavy chain variable region of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15 (as shown in Table 3). In some embodiments, anti-CD33 antibodies of the present disclosure comprise a heavy chain variable region comprising an HVR-H1 comprising the amino acid sequence GYTFTDYNLH (SEQ ID NO: 105), an HVR-H2 comprising the amino acid sequence FIYPSNRITG (SEQ ID NO: 119), and an HVR-H3 comprising the amino acid sequence SDVDYFDY (SEQ ID NO: 122). In some embodiments, anti-CD33 antibodies of the present disclosure comprise a heavy chain variable region comprising an HVR-H1 comprising the amino acid sequence GYTFTDYNLH (SEQ ID NO: 105), an HVR-H2 comprising the amino acid sequence FIYPSNQITG (SEQ ID NO: 118), and an HVR-H3 comprising the amino acid sequence SDVDYFDY (SEQ ID NO: 122).

In some embodiments, anti-CD33 antibodies of the present disclosure comprise a light chain variable region comprising an amino acid sequence selected from SEQ ID NOs: 77-101. In some embodiments, the light chain variable region comprises the amino acid sequence of SEQ ID NO: 86. In some embodiments, anti-CD33 antibodies of the present disclosure comprise a light chain variable region of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15 (as shown in Table 4). In some embodiments, anti-CD33 antibodies of the present disclosure comprise a light chain variable region comprising an HVR-L1 comprising the amino acid sequence RASQSVSTSTYSYMH (SEQ ID NO: 127), an HVR-L2 comprising the amino acid sequence YASNLES (SEQ ID NO: 135), and an HVR-L3 comprising the amino acid sequence QHSWEIPLT (SEQ ID NO: 146). In some embodiments, anti-CD33 antibodies of the present disclosure comprise a light chain variable region comprising an HVR-L1 comprising the amino acid sequence RASQSVSTSTYSYMH (SEQ ID NO: 127), an HVR-L2 comprising the amino acid sequence YASNLES (SEQ ID NO: 135), and an HVR-L3 comprising the amino acid sequence QHSWEIPLT (SEQ ID NO: 146).

In some embodiments, anti-CD33 antibodies of the present disclosure comprise a heavy chain variable region comprising an amino acid sequence selected from SEQ ID NOs: 34-72 and a light chain variable region comprising an amino acid sequence selected from SEQ ID NOs: 77-101. In some embodiments, the heavy chain variable region comprises the amino acid sequence of SEQ ID NO: 59, and the light chain variable region comprises the amino acid sequence of SEQ ID NO: 86. In some embodiments, the heavy chain variable region comprises the amino acid sequence of SEQ ID NO: 65, and the light chain variable region comprises the amino acid sequence of SEQ ID NO: 86. In some embodiments, anti-CD33 antibodies of the present disclosure comprise a heavy chain variable region of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15 (as shown in Table 3) and a light chain variable region of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15 (as shown in Table 4). In some embodiments, anti-CD33 antibodies of the present disclosure comprise a heavy chain variable region comprising an HVR-H1 comprising the amino acid sequence GYTFTDYNLH (SEQ ID NO: 105), an HVR-H2 comprising the amino acid sequence FIYPSNRITG (SEQ ID NO: 119), an HVR-H3 comprising the amino acid sequence SDVDYFDY (SEQ ID NO: 122), and a light chain variable region comprising an HVR-L1 comprising the amino acid sequence RASQSVSTSTYSYMH (SEQ ID NO: 127), an HVR-L2 comprising the amino acid sequence YASNLES (SEQ ID NO: 135), and an HVR-L3 comprising the amino acid sequence QHSWEIPLT (SEQ ID NO: 146). In some embodiments, anti-CD33 antibodies of the present disclosure comprise a heavy chain variable region comprising an HVR-H1 comprising the amino acid sequence GYTFTDYNLH (SEQ ID NO: 105), an HVR-H2 comprising the amino acid sequence FIYPSNQITG (SEQ ID NO: 118), an HVR-H3 comprising the amino acid sequence SDVDYFDY (SEQ ID NO: 122), and a light chain variable region comprising an HVR-L1 comprising the amino acid sequence RASQSVSTSTYSYMH (SEQ ID NO: 127), an HVR-L2 comprising the amino acid sequence YASNLES (SEQ ID NO: 135), and an HVR-L3 comprising the amino acid sequence QHSWEIPLT (SEQ ID NO: 146).

In some embodiments, anti-CD33 antibodies of the present disclosure comprise a heavy chain variable region comprising an amino acid sequence selected from SEQ ID NOs: 34, 40, 42, 52, 53, and 73-76. In some embodiments, the antibody comprises a heavy chain variable region of AB-H2, AB-H9, AB-H14, AB-H15, AB-H63, AB-H64, AB-H65, or AB-H66 (as shown in Table 3). In some embodiments, anti-CD33 antibodies of the present disclosure comprise a light chain variable region comprising an amino acid sequence selected from SEQ ID NOs: 77, 86, and 102. In some embodiments, the antibody comprises a light chain variable region of antibody AB-H2, AB-H9, AB-H14, AB-H15, AB-H63, AB-H64, AB-H65, or AB-H66 (as shown in Table 4). In some embodiments, anti-CD33 antibodies of the present disclosure comprise a heavy chain variable region comprising an amino acid sequence selected from SEQ ID NOs: 34, 40, 42, 52, 53, and 73-76, and a light chain variable region comprising an amino acid sequence selected from SEQ ID NOs: 77, 86, and 102. In some embodiments, the antibody comprises a heavy chain variable region of AB-H2, AB-H9, AB-H14, AB-H15, AB-H63, AB-H64, AB-H65, or AB-H66 (as shown in Table 3), and a light chain variable region of antibody AB-H2, AB-H9, AB-H14, AB-H15, AB-H63, AB-H64, AB-H65, or AB-H66 (as shown in Table 4).

Any of the antibodies of the present disclosure may be produced by a cell line. In some embodiments, the cell line may be a mammalian cell line. In certain embodiments, the cell line may be a hybridoma cell line. In other embodiments, the cell line may be a yeast cell line. Any cell line known in the art suitable for antibody production may be used to produce an antibody of the present disclosure. Exemplary cell lines for antibody production are described throughout the present disclosure.

In some embodiments, the anti-CD33 antibody is an anti-CD33 monoclonal antibody selected from AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, AB-64.1.15, AB-H2, AB-H9, AB-H14, AB-H15, AB-H63, AB-H64, AB-H65, or AB-H66.

In some embodiments, anti-CD33 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1 or to the amino acid sequence of SEQ ID NO: 52; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1 or to the amino acid sequence of SEQ ID NO: 86. In some embodiments, anti-CD33 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1 or to the amino acid sequence of SEQ ID NO: 52, wherein the heavy chain variable domain comprises the HVR-H1, HVR-H2, and HVR-H3 amino acid sequences of antibody AB-64.1. In some embodiments, anti-CD33 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1 or to the amino acid sequence of SEQ ID NO: 86, wherein the light chain variable domain comprises the HVR-L1, HVR-L2, and HVR-L3 amino acid sequences of antibody AB-64.1. In some embodiments, the anti-CD33 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1 or to the amino acid sequence of SEQ ID NO: 52 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain variable domain amino acid sequence of antibody AB-64.1 or the amino acid sequence of SEQ ID NO: 52. In certain embodiments, a total of 1 to 5 amino acids have been substituted, inserted and/or deleted in the heavy chain variable domain amino acid sequence of antibody AB-64.1 or the amino acid sequence of SEQ ID NO: 52. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VH sequence of antibody CD33 or of SEQ ID NO: 52, including post-translational modifications of that sequence. In a particular embodiment, the VH comprises one, two or three HVRs selected from: (a) the HVR-H1 amino acid sequence of antibody AB-64.1, (b) the HVR-H2 amino acid sequence of antibody AB-64.1, and (c) the HVR-H3 amino acid sequence of antibody AB-64.1. In some embodiments, anti-CD33 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1 or to the amino acid sequence of SEQ ID NO: 86 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain variable domain amino acid sequence of antibody AB-64.1 or the amino acid sequence of SEQ ID NO: 86. In certain embodiments, a total of 1 to 5 amino acids have been substituted, inserted and/or deleted in the light chain variable domain amino acid sequence of antibody AB-64.1 or the amino acid sequence of SEQ ID NO: 86. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VL sequence of antibody AB-64.1 or of SEQ ID NO: 86, including post-translational modifications of that sequence. In a particular embodiment, the VL comprises one, two or three HVRs selected from: (a) the HVR-L1 amino acid sequence of antibody AB-64.1, (b) the HVR-L2 amino acid sequence of antibody AB-64.1, and (c) the HVR-L3 amino acid sequence of antibody AB-64.1.

In some embodiments, anti-CD33 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.1 or to the amino acid sequence of SEQ ID NO: 58; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.1 or to the amino acid sequence of SEQ ID NO: 86. In some embodiments, anti-CD33 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.1 or to the amino acid sequence of SEQ ID NO: 58, wherein the heavy chain variable domain comprises the HVR-H1, HVR-H2, and HVR-H3 amino acid sequences of antibody AB-64.1.1. In some embodiments, anti-CD33 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.1 or to the amino acid sequence of SEQ ID NO: 86, wherein the light chain variable domain comprises the HVR-L1, HVR-L2, and HVR-L3 amino acid sequences of antibody AB-64.1.1. In some embodiments, the anti-CD33 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.1 or to the amino acid sequence of SEQ ID NO: 58 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain variable domain amino acid sequence of antibody AB-64.1.1 or the amino acid sequence of SEQ ID NO: 58. In certain embodiments, a total of 1 to 5 amino acids have been substituted, inserted and/or deleted in the heavy chain variable domain amino acid sequence of antibody AB-64.1.1 or the amino acid sequence of SEQ ID NO: 58. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VH sequence of antibody CD33 or of SEQ ID NO: 58, including post-translational modifications of that sequence. In a particular embodiment, the VH comprises one, two or three HVRs selected from: (a) the HVR-H1 amino acid sequence of antibody AB-64.1.1, (b) the HVR-H2 amino acid sequence of antibody AB-64.1.1, and (c) the HVR-H3 amino acid sequence of antibody AB-64.1.1. In some embodiments, anti-CD33 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.1 or to the amino acid sequence of SEQ ID NO: 86 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain variable domain amino acid sequence of antibody AB-64.1.1 or the amino acid sequence of SEQ ID NO: 86. In certain embodiments, a total of 1 to 5 amino acids have been substituted, inserted and/or deleted in the light chain variable domain amino acid sequence of antibody AB-64.1.1 or the amino acid sequence of SEQ ID NO: 86. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VL sequence of antibody AB-64.1.1 or of SEQ ID NO: 86, including post-translational modifications of that sequence. In a particular embodiment, the VL comprises one, two or three HVRs selected from: (a) the HVR-L1 amino acid sequence of antibody AB-64.1.1, (b) the HVR-L2 amino acid sequence of antibody AB-64.1.1, and (c) the HVR-L3 amino acid sequence of antibody AB-64.1.1.

In some embodiments, anti-CD33 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.2 or to the amino acid sequence of SEQ ID NO: 59; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.2 or to the amino acid sequence of SEQ ID NO: 86. In some embodiments, anti-CD33 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.2 or to the amino acid sequence of SEQ ID NO: 59, wherein the heavy chain variable domain comprises the HVR-H1, HVR-H2, and HVR-H3 amino acid sequences of antibody AB-64.1.2. In some embodiments, anti-CD33 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.2 or to the amino acid sequence of SEQ ID NO: 86, wherein the light chain variable domain comprises the HVR-L1, HVR-L2, and HVR-L3 amino acid sequences of antibody AB-64.1.2. In some embodiments, the anti-CD33 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.2 or to the amino acid sequence of SEQ ID NO: 59 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain variable domain amino acid sequence of antibody AB-64.1.2 or the amino acid sequence of SEQ ID NO: 59. In certain embodiments, a total of 1 to 5 amino acids have been substituted, inserted and/or deleted in the heavy chain variable domain amino acid sequence of antibody AB-64.1.2 or the amino acid sequence of SEQ ID NO: 59. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VH sequence of antibody CD33 or of SEQ ID NO: 59, including post-translational modifications of that sequence. In a particular embodiment, the VH comprises one, two or three HVRs selected from: (a) the HVR-H1 amino acid sequence of antibody AB-64.1.2, (b) the HVR-H2 amino acid sequence of antibody AB-64.1.2, and (c) the HVR-H3 amino acid sequence of antibody AB-64.1.2. In some embodiments, anti-CD33 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.2 or to the amino acid sequence of SEQ ID NO: 86 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain variable domain amino acid sequence of antibody AB-64.1.2 or the amino acid sequence of SEQ ID NO: 86. In certain embodiments, a total of 1 to 5 amino acids have been substituted, inserted and/or deleted in the light chain variable domain amino acid sequence of antibody AB-64.1.2 or the amino acid sequence of SEQ ID NO: 86. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VL sequence of antibody AB-64.1.2 or of SEQ ID NO: 86, including post-translational modifications of that sequence. In a particular embodiment, the VL comprises one, two or three HVRs selected from: (a) the HVR-L1 amino acid sequence of antibody AB-64.1.2, (b) the HVR-L2 amino acid sequence of antibody AB-64.1.2, and (c) the HVR-L3 amino acid sequence of antibody AB-64.1.2.

In some embodiments, anti-CD33 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.3 or to the amino acid sequence of SEQ ID NO: 60; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.3 or to the amino acid sequence of SEQ ID NO: 86. In some embodiments, anti-CD33 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.3 or to the amino acid sequence of SEQ ID NO: 60, wherein the heavy chain variable domain comprises the HVR-H1, HVR-H2, and HVR-H3 amino acid sequences of antibody AB-64.1.3. In some embodiments, anti-CD33 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.3 or to the amino acid sequence of SEQ ID NO: 86, wherein the light chain variable domain comprises the HVR-L1, HVR-L2, and HVR-L3 amino acid sequences of antibody AB-64.1.3. In some embodiments, the anti-CD33 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.3 or to the amino acid sequence of SEQ ID NO: 60 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain variable domain amino acid sequence of antibody AB-64.1.3 or the amino acid sequence of SEQ ID NO: 60. In certain embodiments, a total of 1 to 5 amino acids have been substituted, inserted and/or deleted in the heavy chain variable domain amino acid sequence of antibody AB-64.1.3 or the amino acid sequence of SEQ ID NO: 60. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VH sequence of antibody CD33 or of SEQ ID NO: 60, including post-translational modifications of that sequence. In a particular embodiment, the VH comprises one, two or three HVRs selected from: (a) the HVR-H1 amino acid sequence of antibody AB-64.1.3, (b) the HVR-H2 amino acid sequence of antibody AB-64.1.3, and (c) the HVR-H3 amino acid sequence of antibody AB-64.1.3. In some embodiments, anti-CD33 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.3 or to the amino acid sequence of SEQ ID NO: 86 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain variable domain amino acid sequence of antibody AB-64.1.3 or the amino acid sequence of SEQ ID NO: 86. In certain embodiments, a total of 1 to 5 amino acids have been substituted, inserted and/or deleted in the light chain variable domain amino acid sequence of antibody AB-64.1.3 or the amino acid sequence of SEQ ID NO: 86. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VL sequence of antibody AB-64.1.3 or of SEQ ID NO: 86, including post-translational modifications of that sequence. In a particular embodiment, the VL comprises one, two or three HVRs selected from: (a) the HVR-L1 amino acid sequence of antibody AB-64.1.3, (b) the HVR-L2 amino acid sequence of antibody AB-64.1.3, and (c) the HVR-L3 amino acid sequence of antibody AB-64.1.3.

In some embodiments, anti-CD33 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.4 or to the amino acid sequence of SEQ ID NO: 61; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.4 or to the amino acid sequence of SEQ ID NO: 86. In some embodiments, anti-CD33 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.4 or to the amino acid sequence of SEQ ID NO: 61, wherein the heavy chain variable domain comprises the HVR-H1, HVR-H2, and HVR-H3 amino acid sequences of antibody AB-64.1.4. In some embodiments, anti-CD33 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.4 or to the amino acid sequence of SEQ ID NO: 86, wherein the light chain variable domain comprises the HVR-L1, HVR-L2, and HVR-L3 amino acid sequences of antibody AB-64.1.4. In some embodiments, the anti-CD33 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.4 or to the amino acid sequence of SEQ ID NO: 61 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain variable domain amino acid sequence of antibody AB-64.1.4 or the amino acid sequence of SEQ ID NO: 61. In certain embodiments, a total of 1 to 5 amino acids have been substituted, inserted and/or deleted in the heavy chain variable domain amino acid sequence of antibody AB-64.1.4 or the amino acid sequence of SEQ ID NO: 61. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VH sequence of antibody CD33 or of SEQ ID NO: 61, including post-translational modifications of that sequence. In a particular embodiment, the VH comprises one, two or three HVRs selected from: (a) the HVR-H1 amino acid sequence of antibody AB-64.1.4, (b) the HVR-H2 amino acid sequence of antibody AB-64.1.4, and (c) the HVR-H3 amino acid sequence of antibody AB-64.1.4. In some embodiments, anti-CD33 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.4 or to the amino acid sequence of SEQ ID NO: 86 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain variable domain amino acid sequence of antibody AB-64.1.4 or the amino acid sequence of SEQ ID NO: 86. In certain embodiments, a total of 1 to 5 amino acids have been substituted, inserted and/or deleted in the light chain variable domain amino acid sequence of antibody AB-64.1.4 or the amino acid sequence of SEQ ID NO: 86. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VL sequence of antibody AB-64.1.4 or of SEQ ID NO: 86, including post-translational modifications of that sequence. In a particular embodiment, the VL comprises one, two or three HVRs selected from: (a) the HVR-L1 amino acid sequence of antibody AB-64.1.4, (b) the HVR-L2 amino acid sequence of antibody AB-64.1.4, and (c) the HVR-L3 amino acid sequence of antibody AB-64.1.4.

In some embodiments, anti-CD33 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.5 or to the amino acid sequence of SEQ ID NO: 62; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.5 or to the amino acid sequence of SEQ ID NO: 86. In some embodiments, anti-CD33 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.5 or to the amino acid sequence of SEQ ID NO: 62, wherein the heavy chain variable domain comprises the HVR-H1, HVR-H2, and HVR-H3 amino acid sequences of antibody AB-64.1.5. In some embodiments, anti-CD33 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.5 or to the amino acid sequence of SEQ ID NO: 86, wherein the light chain variable domain comprises the HVR-L1, HVR-L2, and HVR-L3 amino acid sequences of antibody AB-64.1.5. In some embodiments, the anti-CD33 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.5 or to the amino acid sequence of SEQ ID NO: 62 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain variable domain amino acid sequence of antibody AB-64.1.5 or the amino acid sequence of SEQ ID NO: 62. In certain embodiments, a total of 1 to 5 amino acids have been substituted, inserted and/or deleted in the heavy chain variable domain amino acid sequence of antibody AB-64.1.5 or the amino acid sequence of SEQ ID NO: 62. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VH sequence of antibody CD33 or of SEQ ID NO: 62, including post-translational modifications of that sequence. In a particular embodiment, the VH comprises one, two or three HVRs selected from: (a) the HVR-H1 amino acid sequence of antibody AB-64.1.5, (b) the HVR-H2 amino acid sequence of antibody AB-64.1.5, and (c) the HVR-H3 amino acid sequence of antibody AB-64.1.5. In some embodiments, anti-CD33 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.5 or to the amino acid sequence of SEQ ID NO: 86 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain variable domain amino acid sequence of antibody AB-64.1.5 or the amino acid sequence of SEQ ID NO: 86. In certain embodiments, a total of 1 to 5 amino acids have been substituted, inserted and/or deleted in the light chain variable domain amino acid sequence of antibody AB-64.1.5 or the amino acid sequence of SEQ ID NO: 86. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VL sequence of antibody AB-64.1.5 or of SEQ ID NO: 86, including post-translational modifications of that sequence. In a particular embodiment, the VL comprises one, two or three HVRs selected from: (a) the HVR-L1 amino acid sequence of antibody AB-64.1.5, (b) the HVR-L2 amino acid sequence of antibody AB-64.1.5, and (c) the HVR-L3 amino acid sequence of antibody AB-64.1.5.

In some embodiments, anti-CD33 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.6 or to the amino acid sequence of SEQ ID NO: 63; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.6 or to the amino acid sequence of SEQ ID NO: 86. In some embodiments, anti-CD33 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.6 or to the amino acid sequence of SEQ ID NO: 63, wherein the heavy chain variable domain comprises the HVR-H1, HVR-H2, and HVR-H3 amino acid sequences of antibody AB-64.1.6. In some embodiments, anti-CD33 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.6 or to the amino acid sequence of SEQ ID NO: 86, wherein the light chain variable domain comprises the HVR-L1, HVR-L2, and HVR-L3 amino acid sequences of antibody AB-64.1.6. In some embodiments, the anti-CD33 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.6 or to the amino acid sequence of SEQ ID NO: 63 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain variable domain amino acid sequence of antibody AB-64.1.6 or the amino acid sequence of SEQ ID NO: 63. In certain embodiments, a total of 1 to 5 amino acids have been substituted, inserted and/or deleted in the heavy chain variable domain amino acid sequence of antibody AB-64.1.6 or the amino acid sequence of SEQ ID NO: 63. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VH sequence of antibody CD33 or of SEQ ID NO: 63, including post-translational modifications of that sequence. In a particular embodiment, the VH comprises one, two or three HVRs selected from: (a) the HVR-H1 amino acid sequence of antibody AB-64.1.6, (b) the HVR-H2 amino acid sequence of antibody AB-64.1.6, and (c) the HVR-H3 amino acid sequence of antibody AB-64.1.6. In some embodiments, anti-CD33 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.6 or to the amino acid sequence of SEQ ID NO: 86 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain variable domain amino acid sequence of antibody AB-64.1.6 or the amino acid sequence of SEQ ID NO: 86. In certain embodiments, a total of 1 to 5 amino acids have been substituted, inserted and/or deleted in the light chain variable domain amino acid sequence of antibody AB-64.1.6 or the amino acid sequence of SEQ ID NO: 86. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VL sequence of antibody AB-64.1.6 or of SEQ ID NO: 86, including post-translational modifications of that sequence. In a particular embodiment, the VL comprises one, two or three HVRs selected from: (a) the HVR-L1 amino acid sequence of antibody AB-64.1.6, (b) the HVR-L2 amino acid sequence of antibody AB-64.1.6, and (c) the HVR-L3 amino acid sequence of antibody AB-64.1.6.

In some embodiments, anti-CD33 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.7 or to the amino acid sequence of SEQ ID NO: 64; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.7 or to the amino acid sequence of SEQ ID NO: 86. In some embodiments, anti-CD33 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.7 or to the amino acid sequence of SEQ ID NO: 64, wherein the heavy chain variable domain comprises the HVR-H1, HVR-H2, and HVR-H3 amino acid sequences of antibody AB-64.1.7. In some embodiments, anti-CD33 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.7 or to the amino acid sequence of SEQ ID NO: 86, wherein the light chain variable domain comprises the HVR-L1, HVR-L2, and HVR-L3 amino acid sequences of antibody AB-64.1.7. In some embodiments, the anti-CD33 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.7 or to the amino acid sequence of SEQ ID NO: 64 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain variable domain amino acid sequence of antibody AB-64.1.7 or the amino acid sequence of SEQ ID NO: 64. In certain embodiments, a total of 1 to 5 amino acids have been substituted, inserted and/or deleted in the heavy chain variable domain amino acid sequence of antibody AB-64.1.7 or the amino acid sequence of SEQ ID NO: 64. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VH sequence of antibody CD33 or of SEQ ID NO: 64, including post-translational modifications of that sequence. In a particular embodiment, the VH comprises one, two or three HVRs selected from: (a) the HVR-H1 amino acid sequence of antibody AB-64.1.7, (b) the HVR-H2 amino acid sequence of antibody AB-64.1.7, and (c) the HVR-H3 amino acid sequence of antibody AB-64.1.7. In some embodiments, anti-CD33 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.7 or to the amino acid sequence of SEQ ID NO: 86 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain variable domain amino acid sequence of antibody AB-64.1.7 or the amino acid sequence of SEQ ID NO: 86. In certain embodiments, a total of 1 to 5 amino acids have been substituted, inserted and/or deleted in the light chain variable domain amino acid sequence of antibody AB-64.1.7 or the amino acid sequence of SEQ ID NO: 86. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VL sequence of antibody AB-64.1.7 or of SEQ ID NO: 86, including post-translational modifications of that sequence. In a particular embodiment, the VL comprises one, two or three HVRs selected from: (a) the HVR-L1 amino acid sequence of antibody AB-64.1.7, (b) the HVR-L2 amino acid sequence of antibody AB-64.1.7, and (c) the HVR-L3 amino acid sequence of antibody AB-64.1.7.

In some embodiments, anti-CD33 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.8 or to the amino acid sequence of SEQ ID NO: 65; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.8 or to the amino acid sequence of SEQ ID NO: 86. In some embodiments, anti-CD33 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.8 or to the amino acid sequence of SEQ ID NO: 65, wherein the heavy chain variable domain comprises the HVR-H1, HVR-H2, and HVR-H3 amino acid sequences of antibody AB-64.1.8. In some embodiments, anti-CD33 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.8 or to the amino acid sequence of SEQ ID NO: 86, wherein the light chain variable domain comprises the HVR-L1, HVR-L2, and HVR-L3 amino acid sequences of antibody AB-64.1.8. In some embodiments, the anti-CD33 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.8 or to the amino acid sequence of SEQ ID NO: 65 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain variable domain amino acid sequence of antibody AB-64.1.8 or the amino acid sequence of SEQ ID NO: 65. In certain embodiments, a total of 1 to 5 amino acids have been substituted, inserted and/or deleted in the heavy chain variable domain amino acid sequence of antibody AB-64.1.8 or the amino acid sequence of SEQ ID NO: 65. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VH sequence of antibody CD33 or of SEQ ID NO: 65, including post-translational modifications of that sequence. In a particular embodiment, the VH comprises one, two or three HVRs selected from: (a) the HVR-H1 amino acid sequence of antibody AB-64.1.8, (b) the HVR-H2 amino acid sequence of antibody AB-64.1.8, and (c) the HVR-H3 amino acid sequence of antibody AB-64.1.8. In some embodiments, anti-CD33 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.8 or to the amino acid sequence of SEQ ID NO: 86 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain variable domain amino acid sequence of antibody AB-64.1.8 or the amino acid sequence of SEQ ID NO: 86. In certain embodiments, a total of 1 to 5 amino acids have been substituted, inserted and/or deleted in the light chain variable domain amino acid sequence of antibody AB-64.1.8 or the amino acid sequence of SEQ ID NO: 86. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VL sequence of antibody AB-64.1.8 or of SEQ ID NO: 86, including post-translational modifications of that sequence. In a particular embodiment, the VL comprises one, two or three HVRs selected from: (a) the HVR-L1 amino acid sequence of antibody AB-64.1.8, (b) the HVR-L2 amino acid sequence of antibody AB-64.1.8, and (c) the HVR-L3 amino acid sequence of antibody AB-64.1.8.

In some embodiments, anti-CD33 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.9 or to the amino acid sequence of SEQ ID NO: 66; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.9 or to the amino acid sequence of SEQ ID NO: 86. In some embodiments, anti-CD33 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.9 or to the amino acid sequence of SEQ ID NO: 66, wherein the heavy chain variable domain comprises the HVR-H1, HVR-H2, and HVR-H3 amino acid sequences of antibody AB-64.1.9. In some embodiments, anti-CD33 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.9 or to the amino acid sequence of SEQ ID NO: 86, wherein the light chain variable domain comprises the HVR-L1, HVR-L2, and HVR-L3 amino acid sequences of antibody AB-64.1.9. In some embodiments, the anti-CD33 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.9 or to the amino acid sequence of SEQ ID NO: 66 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain variable domain amino acid sequence of antibody AB-64.1.9 or the amino acid sequence of SEQ ID NO: 66. In certain embodiments, a total of 1 to 5 amino acids have been substituted, inserted and/or deleted in the heavy chain variable domain amino acid sequence of antibody AB-64.1.9 or the amino acid sequence of SEQ ID NO: 66. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VH sequence of antibody CD33 or of SEQ ID NO: 66, including post-translational modifications of that sequence. In a particular embodiment, the VH comprises one, two or three HVRs selected from: (a) the HVR-H1 amino acid sequence of antibody AB-64.1.9, (b) the HVR-H2 amino acid sequence of antibody AB-64.1.9, and (c) the HVR-H3 amino acid sequence of antibody AB-64.1.9. In some embodiments, anti-CD33 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.9 or to the amino acid sequence of SEQ ID NO: 86 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain variable domain amino acid sequence of antibody AB-64.1.9 or the amino acid sequence of SEQ ID NO: 86. In certain embodiments, a total of 1 to 5 amino acids have been substituted, inserted and/or deleted in the light chain variable domain amino acid sequence of antibody AB-64.1.9 or the amino acid sequence of SEQ ID NO: 86. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VL sequence of antibody AB-64.1.9 or of SEQ ID NO: 86, including post-translational modifications of that sequence. In a particular embodiment, the VL comprises one, two or three HVRs selected from: (a) the HVR-L1 amino acid sequence of antibody AB-64.1.9, (b) the HVR-L2 amino acid sequence of antibody AB-64.1.9, and (c) the HVR-L3 amino acid sequence of antibody AB-64.1.9.

In some embodiments, anti-CD33 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.10 or to the amino acid sequence of SEQ ID NO: 67; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.10 or to the amino acid sequence of SEQ ID NO: 86. In some embodiments, anti-CD33 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.10 or to the amino acid sequence of SEQ ID NO: 67, wherein the heavy chain variable domain comprises the HVR-H1, HVR-H2, and HVR-H3 amino acid sequences of antibody AB-64.1.10. In some embodiments, anti-CD33 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.10 or to the amino acid sequence of SEQ ID NO: 86, wherein the light chain variable domain comprises the HVR-L1, HVR-L2, and HVR-L3 amino acid sequences of antibody AB-64.1.10. In some embodiments, the anti-CD33 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.10 or to the amino acid sequence of SEQ ID NO: 67 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain variable domain amino acid sequence of antibody AB-64.1.10 or the amino acid sequence of SEQ ID NO: 67. In certain embodiments, a total of 1 to 5 amino acids have been substituted, inserted and/or deleted in the heavy chain variable domain amino acid sequence of antibody AB-64.1.10 or the amino acid sequence of SEQ ID NO: 67. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VH sequence of antibody CD33 or of SEQ ID NO: 67, including post-translational modifications of that sequence. In a particular embodiment, the VH comprises one, two or three HVRs selected from: (a) the HVR-H1 amino acid sequence of antibody AB-64.1.10, (b) the HVR-H2 amino acid sequence of antibody AB-64.1.10, and (c) the HVR-H3 amino acid sequence of antibody AB-64.1.10. In some embodiments, anti-CD33 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.10 or to the amino acid sequence of SEQ ID NO: 86 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain variable domain amino acid sequence of antibody AB-64.1.10 or the amino acid sequence of SEQ ID NO: 86. In certain embodiments, a total of 1 to 5 amino acids have been substituted, inserted and/or deleted in the light chain variable domain amino acid sequence of antibody AB-64.1.10 or the amino acid sequence of SEQ ID NO: 86. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VL sequence of antibody AB-64.1.10 or of SEQ ID NO: 86, including post-translational modifications of that sequence. In a particular embodiment, the VL comprises one, two or three HVRs selected from: (a) the HVR-L1 amino acid sequence of antibody AB-64.1.10, (b) the HVR-L2 amino acid sequence of antibody AB-64.1.10, and (c) the HVR-L3 amino acid sequence of antibody AB-64.1.10.

In some embodiments, anti-CD33 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.11 or to the amino acid sequence of SEQ ID NO: 68; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.11 or to the amino acid sequence of SEQ ID NO: 86. In some embodiments, anti-CD33 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.11 or to the amino acid sequence of SEQ ID NO: 68, wherein the heavy chain variable domain comprises the HVR-H1, HVR-H2, and HVR-H3 amino acid sequences of antibody AB-64.1.11. In some embodiments, anti-CD33 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.11 or to the amino acid sequence of SEQ ID NO: 86, wherein the light chain variable domain comprises the HVR-L1, HVR-L2, and HVR-L3 amino acid sequences of antibody AB-64.1.11. In some embodiments, the anti-CD33 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.11 or to the amino acid sequence of SEQ ID NO: 68 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain variable domain amino acid sequence of antibody AB-64.1.11 or the amino acid sequence of SEQ ID NO: 68. In certain embodiments, a total of 1 to 5 amino acids have been substituted, inserted and/or deleted in the heavy chain variable domain amino acid sequence of antibody AB-64.1.11 or the amino acid sequence of SEQ ID NO: 68. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VH sequence of antibody CD33 or of SEQ ID NO: 68, including post-translational modifications of that sequence. In a particular embodiment, the VH comprises one, two or three HVRs selected from: (a) the HVR-H1 amino acid sequence of antibody AB-64.1.11, (b) the HVR-H2 amino acid sequence of antibody AB-64.1.11, and (c) the HVR-H3 amino acid sequence of antibody AB-64.1.11. In some embodiments, anti-CD33 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.11 or to the amino acid sequence of SEQ ID NO: 86 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain variable domain amino acid sequence of antibody AB-64.1.11 or the amino acid sequence of SEQ ID NO: 86. In certain embodiments, a total of 1 to 5 amino acids have been substituted, inserted and/or deleted in the light chain variable domain amino acid sequence of antibody AB-64.1.11 or the amino acid sequence of SEQ ID NO: 86. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VL sequence of antibody AB-64.1.11 or of SEQ ID NO: 86, including post-translational modifications of that sequence. In a particular embodiment, the VL comprises one, two or three HVRs selected from: (a) the HVR-L1 amino acid sequence of antibody AB-64.1.11, (b) the HVR-L2 amino acid sequence of antibody AB-64.1.11, and (c) the HVR-L3 amino acid sequence of antibody AB-64.1.11.

In some embodiments, anti-CD33 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.12 or to the amino acid sequence of SEQ ID NO: 69; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.12 or to the amino acid sequence of SEQ ID NO: 86. In some embodiments, anti-CD33 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.12 or to the amino acid sequence of SEQ ID NO: 69, wherein the heavy chain variable domain comprises the HVR-H1, HVR-H2, and HVR-H3 amino acid sequences of antibody AB-64.1.12. In some embodiments, anti-CD33 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.12 or to the amino acid sequence of SEQ ID NO: 86, wherein the light chain variable domain comprises the HVR-L1, HVR-L2, and HVR-L3 amino acid sequences of antibody AB-64.1.12. In some embodiments, the anti-CD33 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.12 or to the amino acid sequence of SEQ ID NO: 69 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain variable domain amino acid sequence of antibody AB-64.1.12 or the amino acid sequence of SEQ ID NO: 69. In certain embodiments, a total of 1 to 5 amino acids have been substituted, inserted and/or deleted in the heavy chain variable domain amino acid sequence of antibody AB-64.1.12 or the amino acid sequence of SEQ ID NO: 69. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VH sequence of antibody CD33 or of SEQ ID NO: 69, including post-translational modifications of that sequence. In a particular embodiment, the VH comprises one, two or three HVRs selected from: (a) the HVR-H1 amino acid sequence of antibody AB-64.1.12, (b) the HVR-H2 amino acid sequence of antibody AB-64.1.12, and (c) the HVR-H3 amino acid sequence of antibody AB-64.1.12. In some embodiments, anti-CD33 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.12 or to the amino acid sequence of SEQ ID NO: 86 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain variable domain amino acid sequence of antibody AB-64.1.12 or the amino acid sequence of SEQ ID NO: 86. In certain embodiments, a total of 1 to 5 amino acids have been substituted, inserted and/or deleted in the light chain variable domain amino acid sequence of antibody AB-64.1.12 or the amino acid sequence of SEQ ID NO: 86. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VL sequence of antibody AB-64.1.12 or of SEQ ID NO: 86, including post-translational modifications of that sequence. In a particular embodiment, the VL comprises one, two or three HVRs selected from: (a) the HVR-L1 amino acid sequence of antibody AB-64.1.12, (b) the HVR-L2 amino acid sequence of antibody AB-64.1.12, and (c) the HVR-L3 amino acid sequence of antibody AB-64.1.12.

In some embodiments, anti-CD33 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.13 or to the amino acid sequence of SEQ ID NO: 52; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.13 or to the amino acid sequence of SEQ ID NO: 86. In some embodiments, anti-CD33 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.13 or to the amino acid sequence of SEQ ID NO: 52, wherein the heavy chain variable domain comprises the HVR-H1, HVR-H2, and HVR-H3 amino acid sequences of antibody AB-64.1.13. In some embodiments, anti-CD33 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.13 or to the amino acid sequence of SEQ ID NO: 86, wherein the light chain variable domain comprises the HVR-L1, HVR-L2, and HVR-L3 amino acid sequences of antibody AB-64.1.13. In some embodiments, the anti-CD33 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.13 or to the amino acid sequence of SEQ ID NO: 52 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain variable domain amino acid sequence of antibody AB-64.1.13 or the amino acid sequence of SEQ ID NO: 52. In certain embodiments, a total of 1 to 5 amino acids have been substituted, inserted and/or deleted in the heavy chain variable domain amino acid sequence of antibody AB-64.1.13 or the amino acid sequence of SEQ ID NO: 52. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VH sequence of antibody CD33 or of SEQ ID NO: 52, including post-translational modifications of that sequence. In a particular embodiment, the VH comprises one, two or three HVRs selected from: (a) the HVR-H1 amino acid sequence of antibody AB-64.1.13, (b) the HVR-H2 amino acid sequence of antibody AB-64.1.13, and (c) the HVR-H3 amino acid sequence of antibody AB-64.1.13. In some embodiments, anti-CD33 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.13 or to the amino acid sequence of SEQ ID NO: 86 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain variable domain amino acid sequence of antibody AB-64.1.13 or the amino acid sequence of SEQ ID NO: 86. In certain embodiments, a total of 1 to 5 amino acids have been substituted, inserted and/or deleted in the light chain variable domain amino acid sequence of antibody AB-64.1.13 or the amino acid sequence of SEQ ID NO: 86. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VL sequence of antibody AB-64.1.13 or of SEQ ID NO: 86, including post-translational modifications of that sequence. In a particular embodiment, the VL comprises one, two or three HVRs selected from: (a) the HVR-L1 amino acid sequence of antibody AB-64.1.13, (b) the HVR-L2 amino acid sequence of antibody AB-64.1.13, and (c) the HVR-L3 amino acid sequence of antibody AB-64.1.13.

In some embodiments, anti-CD33 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.14 or to the amino acid sequence of SEQ ID NO: 71; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.14 or to the amino acid sequence of SEQ ID NO: 86. In some embodiments, anti-CD33 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.14 or to the amino acid sequence of SEQ ID NO: 71, wherein the heavy chain variable domain comprises the HVR-H1, HVR-H2, and HVR-H3 amino acid sequences of antibody AB-64.1.14. In some embodiments, anti-CD33 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.14 or to the amino acid sequence of SEQ ID NO: 86, wherein the light chain variable domain comprises the HVR-L1, HVR-L2, and HVR-L3 amino acid sequences of antibody AB-64.1.14. In some embodiments, the anti-CD33 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.14 or to the amino acid sequence of SEQ ID NO: 71 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain variable domain amino acid sequence of antibody AB-64.1.14 or the amino acid sequence of SEQ ID NO: 71. In certain embodiments, a total of 1 to 5 amino acids have been substituted, inserted and/or deleted in the heavy chain variable domain amino acid sequence of antibody AB-64.1.14 or the amino acid sequence of SEQ ID NO: 71. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VH sequence of antibody CD33 or of SEQ ID NO: 71, including post-translational modifications of that sequence. In a particular embodiment, the VH comprises one, two or three HVRs selected from: (a) the HVR-H1 amino acid sequence of antibody AB-64.1.14, (b) the HVR-H2 amino acid sequence of antibody AB-64.1.14, and (c) the HVR-H3 amino acid sequence of antibody AB-64.1.14. In some embodiments, anti-CD33 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.14 or to the amino acid sequence of SEQ ID NO: 86 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain variable domain amino acid sequence of antibody AB-64.1.14 or the amino acid sequence of SEQ ID NO: 86. In certain embodiments, a total of 1 to 5 amino acids have been substituted, inserted and/or deleted in the light chain variable domain amino acid sequence of antibody AB-64.1.14 or the amino acid sequence of SEQ ID NO: 86. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VL sequence of antibody AB-64.1.14 or of SEQ ID NO: 86, including post-translational modifications of that sequence. In a particular embodiment, the VL comprises one, two or three HVRs selected from: (a) the HVR-L1 amino acid sequence of antibody AB-64.1.14, (b) the HVR-L2 amino acid sequence of antibody AB-64.1.14, and (c) the HVR-L3 amino acid sequence of antibody AB-64.1.14.

In some embodiments, anti-CD33 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.15 or to the amino acid sequence of SEQ ID NO: 72; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.15 or to the amino acid sequence of SEQ ID NO: 86. In some embodiments, anti-CD33 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.15 or to the amino acid sequence of SEQ ID NO: 72, wherein the heavy chain variable domain comprises the HVR-H1, HVR-H2, and HVR-H3 amino acid sequences of antibody AB-64.1.15. In some embodiments, anti-CD33 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.15 or to the amino acid sequence of SEQ ID NO: 86, wherein the light chain variable domain comprises the HVR-L1, HVR-L2, and HVR-L3 amino acid sequences of antibody AB-64.1.15. In some embodiments, the anti-CD33 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AB-64.1.15 or to the amino acid sequence of SEQ ID NO: 72 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain variable domain amino acid sequence of antibody AB-64.1.15 or the amino acid sequence of SEQ ID NO: 72. In certain embodiments, a total of 1 to 5 amino acids have been substituted, inserted and/or deleted in the heavy chain variable domain amino acid sequence of antibody AB-64.1.15 or the amino acid sequence of SEQ ID NO: 72. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VH sequence of antibody CD33 or of SEQ ID NO: 72, including post-translational modifications of that sequence. In a particular embodiment, the VH comprises one, two or three HVRs selected from: (a) the HVR-H1 amino acid sequence of antibody AB-64.1.15, (b) the HVR-H2 amino acid sequence of antibody AB-64.1.15, and (c) the HVR-H3 amino acid sequence of antibody AB-64.1.15. In some embodiments, anti-CD33 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AB-64.1.15 or to the amino acid sequence of SEQ ID NO: 86 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain variable domain amino acid sequence of antibody AB-64.1.15 or the amino acid sequence of SEQ ID NO: 86. In certain embodiments, a total of 1 to 5 amino acids have been substituted, inserted and/or deleted in the light chain variable domain amino acid sequence of antibody AB-64.1.15 or the amino acid sequence of SEQ ID NO: 86. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VL sequence of antibody AB-64.1.15 or of SEQ ID NO: 86, including post-translational modifications of that sequence. In a particular embodiment, the VL comprises one, two or three HVRs selected from: (a) the HVR-L1 amino acid sequence of antibody AB-64.1.15, (b) the HVR-L2 amino acid sequence of antibody AB-64.1.15, and (c) the HVR-L3 amino acid sequence of antibody AB-64.1.15.

In some embodiments, the anti-CD33 antibody is anti-CD33 monoclonal antibody AB-64.1.2. In some embodiments, the anti-CD33 antibody is an isolated antibody which binds essentially the same CD33 epitope as AB-64.1.2. In some embodiments, the anti-CD33 antibody is an isolated antibody comprising the HVR-H1, HVR-H2, and HVR-H3 of the heavy chain variable domain of monoclonal antibody AB-64.1.2. In some embodiments, the anti-CD33 antibody is an isolated antibody comprising the HVR-L1, HVR-L2, and HVR-L3 of the light chain variable domain of monoclonal antibody AB-64.1.2. In some embodiments, the anti-CD33 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody AB-64.1.2.

In some embodiments, the anti-CD33 antibody is anti-CD33 monoclonal antibody AB-64.1.8. In some embodiments, the anti-CD33 antibody is an isolated antibody which binds essentially the same CD33 epitope as AB-64.1.8. In some embodiments, the anti-CD33 antibody is an isolated antibody comprising the HVR-H1, HVR-H2, and HVR-H3 of the heavy chain variable domain of monoclonal antibody AB-64.1.8. In some embodiments, the anti-CD33 antibody is an isolated antibody comprising the HVR-L1, HVR-L2, and HVR-L3 of the light chain variable domain of monoclonal antibody AB-64.1.8. In some embodiments, the anti-CD33 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody AB-64.1.8.

In certain embodiments, the anti-CD33 antibody is an antagonist antibody. In certain embodiments, the anti-CD33 antibody is an agonist antibody or an inert antibody. In some embodiments, anti-CD33 antibodies of the present disclosure are of the IgG class the IgM class, or the IgA class. In some embodiments, anti-CD33 antibodies of the present disclosure are of the IgG class and have an IgG1, IgG2, IgG3, or IgG4 isotype.

Additional anti-CD33 antibodies, e.g., antibodies that specifically bind to a CD33 protein of the present disclosure, may be identified, screened, and/or characterized for their physical/chemical properties and/or biological activities by various assays known in the art.

Anti-CD33 Antibodies Capable of Binding Fc Gamma Receptors

In some embodiments, anti-CD33 antibodies of the present disclosure retain the ability to bind Fc gamma receptors. In some embodiments, such antibodies when they have the correct epitope specificity that is compatible with receptor activation may have features that enable them to cluster and transiently stimulate, for example, the CD33 receptor. In some embodiments, such antibodies may subsequently act as longer-term inhibitors of CD33 expression and/or one or more activities of a CD33 protein by inducing CD33 degradation, CD33 desensitization, CD33 cleavage, CD33 internalization, CD33 shedding, downregulation of CD33 expression, and/or lysosomal degradation of CD33.

In vivo, anti-CD33 antibodies of the present disclosure may cluster receptors and transiently activate CD33 by any one or more of multiple potential mechanisms. Some isotypes of human antibodies such as IgG2 have, due to their unique structure, an intrinsic ability to cluster receptors, or retain receptors in a clustered configuration, thereby transiently activating receptors such as CD33 without binding to an Fc receptor (e.g., White et al., (2015) Cancer Cell 27, 138-148).

In some embodiments, other antibodies may cluster receptors (e.g., CD33) by binding to Fcg receptors on adjacent cells. In some embodiments, binding of the constant IgG Fc region of the antibody to Fcg receptors may lead to aggregation of the antibodies, and the antibodies in turn may aggregate the receptors to which they bind through their variable region (Chu et al (2008) Mol Immunol, 45:3926-3933; and Wilson et al., (2011) Cancer Cell 19, 101-113). In some embodiments, binding to the inhibitory Fcg receptor FcgR (FcgRIIB) that does not elicit cytokine secretion, oxidative burst, increased phagocytosis, and enhanced antibody-dependent, cell-mediated cytotoxicity (ADCC) is a preferred way to cluster antibodies in vivo, since binding to FcgRIIB is not associated with adverse immune response effects.

There are other mechanisms by which anti-CD33 antibodies of the present disclosure can cluster receptors. For example, antibody fragments (e.g., Fab fragments) that are cross-linked together may be used to cluster receptors (e.g., CD33) in a manner similar to antibodies with Fc regions that bind Fcg receptors, as described above. In some embodiments, cross-linked antibody fragments (e.g., Fab fragments) may transiently function as agonist antibodies if they induce receptor clustering on the cell surface and bind an appropriate epitope on the target (e.g., CD33).

Therefore, in some embodiments, antibodies of the present disclosure that bind a CD33 protein may include antibodies that due to their epitope specificity bind CD33 and transiently activate one or more CD33 activities before they, for example, decrease cellular levels of CD33, inhibit one or more CD33 activities, and/or inhibit interaction (e.g., binding) between CD33 and one or more CD33 ligands. In some embodiments, such antibodies may bind to the ligand-binding site on CD33 and transiently mimic the action of a natural ligand, or stimulate the target antigen to transduce signal by binding to one or more domains that are not the ligand-binding sites. In some embodiments, such antibodies would not interfere with ligand binding. In some embodiments, regardless of whether antibodies bind or do not bind to the ligand-binding site on CD33, the antibodies may subsequently act as longer term inhibitors of CD33 expression and/or one or more activities of a CD33 protein by inducing CD33 degradation, CD33 desensitization, CD33 cleavage, CD33 internalization, CD33 shedding, downregulation of CD33 expression, and/or lysosomal degradation of CD33.

In some embodiments, an anti-CD33 antibody of the present disclosure is an antibody that transiently induces one or more activities of a CD33 protein. In some embodiments, the antibody transiently induces the one or more activities after binding to a CD33 protein that is expressed in a cell. In some embodiments, the CD33 protein is expressed on a cell surface. In some embodiments, the one or more activities of a CD33 protein that are transiently induced by anti-CD33 antibodies of the present disclosure may include, without limitation, phosphorylation of Tyr-340 and Tyr-358 by a Src family tyrosine kinase, such as LCK and FYN; recruitment of and binding to the tyrosine-specific protein phosphatases SHP1 and SHP2; recruitment of and binding to PLC-gamma1, which acts as a guanine nucleotide exchange factor for Dynamini-1; recruitment of and binding to SH2-domain containing protein (e.g., Crk1); recruitment of and binding to the spleen tyrosine kinase Syk; recruitment of and binding to SH3-SH2-SH3 growth factor receptor-bound protein 2 (Grb2); recruitment of and binding to multiple SH2-containing proteins; phosphorylation of Ser-307 and Ser-342 by protein kinase C; modulated expression of one or more anti-inflammatory cytokines, IL-4, IL-10, IL-13, IL-35, IL-16, TGF-beta, IL-1Ra, G-CSF, and soluble receptors for TNF, IFN-beta1a, IFN-beta1b, or IL-6 in monocytes, macrophages, T cells, dendritic cells neutrophils, and/or microglia; decreasing intracellular calcium mobilization; modulated expression of one or more pro-inflammatory cytokines IFN-a4, IFN-b, IL-1β, TNF-α, IL-6, IL-8, CRP, IL-20 family members, LIF, IFN-gamma, OSM, CNTF, GM-CSF, IL-11, IL-12, IL-17, IL-18, IL-23, CXCL10, IL-33, CRP, IL-33, MCP-1, and MIP-1-beta in monocytes, macrophages, T cells, dendritic cells, neutrophils, and/or microglia; modulated expression of one or more proteins selected from C1qa, C1qB, C1qC, C1s, C1R, C4, C2, C3, ITGB2, HMOX1, LAT2, CASP1, CSTA, VSIG4, MS4A4A, C3AR1, GPX1, TyroBP, ALOX5AP, ITGAM, SLC7A7, CD4, ITGAX, PYCARD, CD14, CD16, HLA-DR, and CCR2; inhibition of extracellular signal-regulated kinase (ERK) phosphorylation; decreasing tyrosine phosphorylation on multiple cellular proteins; modulated expression of C-C chemokine receptor 7 (CCR7); inhibition of microglial cell chemotaxis toward CCL19 and CCL21 expressing cells; activation of phosphoinositide 3-kinase; reducing cell growth of monocytes, macrophages, T cells, dendritic cells and/or microglia; reducing T cell proliferation induced by dendritic cells, bone marrow-derived dendritic cells, monocytes, microglia, M1 microglia, activated M1 microglia, M2 microglia, macrophages, M1 macrophages, activated M1 macrophages, and/or M2 macrophages; inhibition of osteoclast production, decreased rate of osteoclastogenesis, or both; decreasing survival of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; decreasing proliferation of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; inhibiting migration of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; decreasing one or more functions of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; inhibiting maturation of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; increasing cell death and apoptosis of monocytes, macrophages, T cells, dendritic cells, neutrophils, and/or microglia; reducing phagocytic activity of monocytes, macrophages, T cells, dendritic cells, neutrophils, and/or microglia; reducing proliferation of monocytes, macrophages, T cells, dendritic cells, neutrophils, and/or microglia; reducing the overall functionality of monocytes, macrophages, T cells, dendritic cells, neutrophils, and/or microglia, phosphorylation of an ITAM containing receptor; phosphorylation of a signaling molecules that mediates ITAM signaling; reducing the activation of pattern recognition receptors; reducing the activation of Toll-like receptors; reducing the activation of damage-associated of clearance of cellular and protein debris; interaction between CD33 and one or more of its ligands; interaction between CD33 and a co-receptor such as CD64; reducing one or more types of clearance selected from apoptotic neuron clearance, dysfunctional synapse clearance, nerve tissue debris clearance, non-nerve tissue debris clearance, bacteria or other foreign body clearance, disease-causing protein clearance, and tumor cell clearance; inhibition of phagocytosis of one or more of apoptotic neurons, nerve tissue debris, non-nerve tissue debris, bacteria, other foreign bodies, disease-causing proteins, disease-causing peptides, disease-causing nucleic acid, disease-causing lipids, or tumor cells; inhibition of clearance of a disease-causing nucleic acid, such as the disease-causing nucleic acid is antisense GGCCCC (G2C4) repeat-expansion RNA; activation of clearance of, a disease-causing protein selected from amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides; inhibition of beneficial immune response to different types of cancer selected from bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, acute myeloid leukemia, pancreatic cancer, prostate cancer, ovarian cancer, fibrosarcoma, and thyroid cancer; inhibition of beneficial immune response to different types of neurological disorders selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, essential tremor, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, Sarcoidosis, diseases of aging, seizures, spinal cord injury, traumatic brain injury, age related macular degeneration, glaucoma, retinitis pigmentosa, retinal degeneration, and multiple sclerosis; inhibition of beneficial immune response-to different types of inflammatory and infectious disorders selected from lupus, acute and chronic colitis, wound healing, Crohn's disease, inflammatory bowel disease, ulcerative colitis, obesity, malaria, respiratory tract infection, sepsis, eye infection, systemic infection, lupus, arthritis, low bone density, osteoporosis, osteogenesis, osteopetrotic disease, and Paget's disease of bone; inhibition of phagocytosis of one or more of apoptotic neurons, nerve tissue debris, dysfunctional synapses, non-nerve tissue debris, bacteria, other foreign bodies, disease-causing proteins, disease-causing peptides, disease-causing nucleic acids, or tumor cells, where the disease-causing nucleic acids may be an antisense GGCCCC (G2C4) repeat-expansion RNA, the disease-causing proteins may include amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, and the tumor cells may be from a cancer selected from bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, or thyroid cancer; binding to CD33 ligand on tumor cells; binding to CD33 ligand on dendritic cells, bone marrow-derived dendritic cells, monocytes, microglia, T cells, neutrophils, and/or macrophages; inhibition of tumor cell killing by one or more of microglia, macrophages, dendritic cells, bone marrow-derived dendritic cells, neutrophils, T cells, T helper cells, or cytotoxic T cells; inhibition of anti-tumor cell proliferation activity of one or more of microglia, macrophages, dendritic cells, bone marrow-derived dendritic cells, neutrophils, T cells, T helper cells, or cytotoxic T cells; inhibition of anti-tumor cell metastasis activity of one or more of microglia, macrophages, dendritic cells, bone marrow-derived dendritic cells, neutrophils, T cells, T helper cells, or cytotoxic T cells; promotion of immunosuppressor dendritic cells, immunosuppressor macrophages, myeloid-derived suppressor cells, tumor-associated macrophages, or regulatory T cells; inhibition of one or more ITAM motif containing receptors, such as TREM1, TREM2, FcgR, DAP10, and DAP12; inhibition of one or more receptors containing the motif D/Ex0-2YxxL/IX6-8YxxL/I (SEQ ID NO:165); inhibition of signaling by one or more pattern recognition receptors (PRRs), such as receptors that identify pathogen-associated molecular patterns (PAMPs), and receptors that identify damage-associated molecular patterns (DAMPs); inhibition of signaling by one or more Toll-like receptors; inhibition of the JAK-STAT signaling pathway; inhibition of nuclear factor kappa-light-chain-enhancer of activated B cells (NFκB); inhibition of PLCγ/PKC/calcium mobilization; inhibition of PI3K/Akt, Ras/MAPK signaling; modulated expression of one or more inflammatory receptors, such as CD86, expressed on one or more of microglia, macrophages, dendritic cells, bone marrow-derived dendritic cells, neutrophils, T cells, T helper cells, or cytotoxic T cells; increasing expression of one or more CD33-dependent genes; normalization of disrupted CD33-dependent gene expression; and decreasing expression of one or more ITAM-dependent genes, such as NFAT transcription factors. Anti-CD33 antibodies of the present disclosure may be tested for their ability to transiently induce one or more activities of a CD33 protein utilizing any suitable technique or assay known in the art and disclosed herein. Regardless of the activities that such antibodies transiently induce, such antibodies may subsequently act as longer-term inhibitors of CD33 expression and/or one or more activities of a CD33 protein by inducing CD33 degradation, CD33 desensitization, CD33 cleavage, CD33 internalization, CD33 shedding, downregulation of CD33 expression, and/or lysosomal degradation of CD33. In some embodiments, the CD33 antibody transiently induces one or more activities of a CD33 protein independently of binding to an Fc receptor.

Exemplary antibody Fc isotypes and modifications are provided in Table B below. In some embodiments, an anti-CD33 antibody of the present disclosure that is capable of binding an Fc gamma receptor has an Fc isotype listed in Table B below.

Table B: Exemplary anti-CD33 antibody Fc isotypes that are capable of binding Fc gamma receptor Fc Isotype Mutation (EU numbering scheme) IgG1 N297A IgG1 D265A and N297A IgG1 D270A IgG1 L234A and L235A L234A and G237A L234A and L235A and G237A

TABLE B
Exemplary anti-CD33 antibody Fc isotypes that are capable of binding
Fc gamma receptor

| Fc Isotype | Mutation (EU numbering scheme) |
|---|---|
| IgG1 | N297A |
| IgG1 | D265A and N297A |
| IgG1 | D270A |
| IgG1 | L234A and L235A |
| | L234A and G237A |
| | L234A and L235A and G237A |
| IgG1 | D270A, and/or P238D, and/or L328E, and/or E233D, and/or |
| | G237D and/or H268D, and/or P271G, and/or A330R |
| IgG1 | P238D and L328E and E233D and G237D and |
| | H268D and P271G and A330R |
| IgG1 | P238D and L328E and G237D and H268D and |
| | P271Gand A330R |
| IgG1 | P238D and S267E and L328F and E233D and G237D |
| | and H268D and P271G and A330R |
| IgG1 | P238D and S267E and L328F and G237D and H268D |
| | and P271G and A330R |
| IgG2 | V234A and G237A |
| IgG4 | L235A and G237A and E318A |
| IgG4 | S228P and L236E |
| IgG2/4 | IgG2 aa 118 to 260 and IgG4 aa 261 to 447 |
| hybrid | H268Q and V309L; and A330S and P331S |
| IgG1 | C226S and C229S and E233P and L234V and L235A |
| IgG1 | L234F and L235E and P331S |
| IgG2 | C232S or C233S |
| IgG2 | A330S and P331S |
| IgG1 | S267E and L328F |
| | S267E alone |
| IgG2 | S267E and L328F |
| IgG4 | S267E and L328F |
| IgG2 | WT HC with Kappa (light chain) LC |
| | HC C127S with Kappa LC |
| | Kappa LC C214S |
| | Kappa LC C214S and HC C233S |
| | Kappa LC C214S and HC C232S |
| | Any of the above listed mutations together with P330S and |
| | P331S mutations |
| | F(ab′)2 fragment of WT IgG1 and any of the above listed |
| | mutations |
| IgG1 | Substitute the Constant Heavy 1 (CH1) and hinge region of |
| | IgG1 With CHI and hinge region of IGg2 |
| | ASTKGPSVFP LAPCSRSTSE STAALGCLVK |
| | DYFPEPVTVS WNSGALTSGV HTFPAVLQSS |
| | GLYSLSSVVT VPSSNFGTQT YTCNVDHKPS |
| | NTKVDKTVER KCCVECPPCP (SEQ ID NO: 166) |
| | With a Kappa LC |
| IgG1 | Any of the above listed mutations together with |
| | A330L/A330S and/ or L234F and/or L235E and/or P331S |
| IgG1, | Any of the above listed mutations together with M252Y |
| IgG2, | and/or S254T and/or T256E |
| or IgG4 | |
| Mouse | For mouse disease models |
| IgG1, | |
| mouse | |
| IgG2a, | |
| mouse | |
| IgG2b | |
| IgG4 | WT |
| IgG1 | Any of the above listed mutation together with E430G, |
| | E430S, E430F, E430T, E345K, E345Q, E345R, E345Y, |
| | S440Y, S440W and/or any combination thereof. |
| IgG2 | Any of the above listed mutation together with E430G, |
| | E430S, E430F, E430T, E345K, E345Q, E345R, E345Y, |
| | S440Y, S440W and/or any combination thereof. |

In addition to the isotypes described in Table C, and without wishing to be bound to theory, it is thought that antibodies with human IgG1 or IgG3 isotypes and mutants thereof (e.g. Strohl (2009) Current Opinion in Biotechnology 2009, 20:685-691) that bind the Fcg Receptors I, IIA, IIC, IIIA, IIIB in human and/or Fcg Receptors I, III and IV in mouse, may also act as transient agonist antibodies.

In some embodiments, the Fc gamma receptor-binding antibody is of the IgG class, the IgM class, or the IgA class. In some embodiments, the Fc gamma receptor-binding antibody has an IgG1, IgG2, IgG3, or IgG4 isotype. In some embodiments, the antibody comprises one or more (e.g., one or more, two or more, three or more, four or more, five or more, six or more, seven or more, eight or more, nine or more, 10 or more, 11 or more, 12 or more, or all thirteen) amino acid substitutions in the Fc region at a residue position selected from the group consisting of: C127S, L234A, L234F, L235A, L235E, S267E, K322A, L328F, A330S, P331S, E345R, E430G, S440Y in any combination (residue position according to EU or Kabat numbering). In some embodiments, the Fc region comprises an amino acid substitution at position E430G. In some embodiments, the Fc region comprises an amino acid substitution at positions L243A, L235A, and P331A. In some embodiments, the Fc region comprises an amino acid substitution at positions L243A, L235A, P331A. In some embodiments, the Fc region comprises an amino acid substitution at positions K322A and E430G. In some embodiments, the Fc region comprises an amino acid substitution at positions P331S and E430G. In some embodiments, the Fc region comprises an amino acid substitution at positions A330S, P331S, and E430G. In some embodiments, the Fc region comprises an amino acid substitution at positions K322A, A330S, and P331S. In some embodiments, the Fc region comprises an amino acid substitution at positions K322A, P331S, and E430G. In some embodiments, the Fc region comprises an amino acid substitution at positions A330S, P331S, and E430G. In some embodiments, the Fc region comprises an amino acid substitution at positions S267E and L328F. In some embodiments, the Fc region comprises an amino acid substitution at position C127S. In some embodiments, the Fc region comprises an amino acid substitution at positions E345R, E430G and S440Y. In some embodiments, the Fc region comprises an amino acid substitution at positions L243A, L235A, and P331S.

In certain embodiments, the Fc gamma receptor-binding antibody has an IgG2 isotype. In some embodiments, the Fc gamma receptor-binding antibody contains a human IgG2 constant region. In some embodiments, the human IgG2 constant region includes an Fc region. In some embodiments, the Fc gamma receptor-binding antibody binds an inhibitory Fc receptor. In certain embodiments, the inhibitory Fc receptor is inhibitory Fc-gamma receptor JIB (FcγIIB). In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from V234A (Alegre et al., (1994) Transplantation 57:1537-1543. 31; Xu et al., (2000) Cell Immunol, 200:16-26), G237A (Cole et al. (1999) Transplantation, 68:563-571), H268Q, V309L, A330S, P331S (US 2007/0148167; Armour et al. (1999) Eur J Immunol 29: 2613-2624; Armour et al. (2000) The Haematology Journal 1(Suppl. 1):27; Armour et al. (2000) The Haematology Journal 1(Suppl. 1):27), C232S, and/or C233S (White et al. (2015) Cancer Cell 27, 138-148), S267E, L328F (Chu et al., (2008) Mol Immunol, 45:3926-3933), M252Y, S254T, and/or T256E, where the amino acid position is according to the EU or Kabat numbering convention.

In some embodiments, the Fc gamma receptor-binding antibody has an IgG2 isotype with a heavy chain constant domain that contains a C127S amino acid substitution, where the amino acid position is according to the EU or Kabat numbering convention (White et al., (2015) Cancer Cell 27, 138-148; Lightle et al., (2010) PROTEIN SCIENCE 19:753-762; and WO2008079246).

In some embodiments, the Fc gamma receptor-binding antibody has an IgG2 isotype with a Kappa light chain constant domain that contains a C214S amino acid substitution, where the amino acid position is according to the EU or Kabat numbering convention (White et al., (2015) Cancer Cell 27, 138-148; Lightle et al., (2010) PROTEIN SCIENCE 19:753-762; and WO2008079246).

In certain embodiments, the Fc gamma receptor-binding antibody has an IgG1 isotype. In some embodiments, the Fc gamma receptor-binding antibody contains a mouse IgG1 constant region. In some embodiments, the Fc gamma receptor-binding antibody contains a human IgG1 constant region. In some embodiments, the human IgG1 constant region includes an Fc region. In some embodiments, the Fc gamma receptor-binding antibody binds an inhibitory Fc receptor. In certain embodiments, the inhibitory Fc receptor is inhibitory Fc-gamma receptor IIB (FcγIIB). In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from N297A (Bolt S et al. (1993) Eur J Immunol 23:403-411), D265A (Shields et al. (2001) R. J. Biol. Chem. 276, 6591-6604), D270A, L234A, L235A (Hutchins et al. (1995) Proc Natl Acad Sci USA, 92:11980-11984; Alegre et al., (1994) Transplantation 57:1537-1543. 31; Xu et al., (2000) Cell Immunol, 200:16-26), G237A (Alegre et al. (1994) Transplantation 57:1537-1543. 31; Xu et al. (2000) Cell Immunol, 200:16-26), P238D, L328E, E233D, G237D, H268D, P271G, A330R, C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), S267E, L328F, A330L, M252Y, S254T, T256E, N297Q, P238S, P238A, A327Q, A327G, P329A, K322A, and/or T394D, where the amino acid position is according to the EU or Kabat numbering convention.

In some embodiments, the antibody includes an IgG2 isotype heavy chain constant domain 1(CH1) and hinge region (White et al., (2015) Cancer Cell 27, 138-148). In certain embodiments, the IgG2 isotype CH1 and hinge region contain the amino acid sequence of ASTKGPSVFPLAPCSRSTSESTAALGCLVKDYFPEPVTVSWNSGALTSGVHTFPAVLQSSGLY SLSSVVTVPSSNFGTQTYTCNVDHKPSNTKVDKTVERKCCVECPPCP (SEQ ID NO: 166). In some embodiments, the antibody Fc region contains a S267E amino acid substitution, a L328F amino acid substitution, or both, and/or a N297A or N297Q amino acid substitution, where the amino acid position is according to the EU or Kabat numbering convention.

In certain embodiments, the Fc gamma receptor-binding antibody has an IgG4 isotype. In some embodiments, the Fc gamma receptor-binding antibody contains a human IgG4 constant region. In some embodiments, the human IgG4 constant region includes an Fc region. In some embodiments, the Fc gamma receptor-binding antibody binds an inhibitory Fc receptor. In certain embodiments, the inhibitory Fc receptor is inhibitory Fc-gamma receptor IIB (FcγIIB). In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from L235A, G237A, S228P, L236E (Reddy et al., (2000) J Immunol, 164:1925-1933), S267E, E318A, L328F, M252Y, S254T, and/or T256E, where the amino acid position is according to the EU or Kabat numbering convention.

In certain embodiments, the Fc gamma receptor-binding antibody has a hybrid IgG2/4 isotype. In some embodiments, the Fc gamma receptor-binding antibody includes an amino acid sequence containing amino acids 118 to 260 according to EU or, Kabat numbering of human IgG2 and amino acids 261-447 according to EU or, Kabat numbering of human IgG4 (WO 1997/11971; WO 2007/106585).

In certain embodiments, the antibody contains a mouse IgG4 constant region (Bartholomaeus, et al. (2014). J. Immunol. 192, 2091-2098).

In some embodiments, the Fc region further contains one or more additional amino acid substitutions selected from the group consisting of A330L, L234F; L235E, or P331S according to EU or, Kabat numbering; and any combination thereof.

In certain embodiments, the antibody contains one or more amino acid substitutions in the Fc region at a residue position selected from C127S, L234A, L234F, L235A, L235E, S267E, K322A, L328F, A330S, P331S, E345R, E430G, S440Y, and any combination thereof, where the numbering of the residues is according to EU or Kabat numbering. In some embodiments, the Fc region contains an amino acid substitution at positions E430G, L243A, L235A, and P331S, where the numbering of the residue position is according to EU numbering. In some embodiments, the Fc region contains an amino acid substitution at positions E430G and P331S, where the numbering of the residue position is according to EU numbering. In some embodiments, the Fc region contains an amino acid substitution at positions E430G and K322A, where the numbering of the residue position is according to EU numbering. In some embodiments, the Fc region contains an amino acid substitution at positions E430G, A330S, and P331S, where the numbering of the residue position is according to EU numbering. In some embodiments, the Fc region contains an amino acid substitution at positions E430G, K322A, A330S, and P331S, where the numbering of the residue position is according to EU numbering. In some embodiments, the Fc region contains an amino acid substitution at positions E430G, K322A, and A330S, where the numbering of the residue position is according to EU numbering. In some embodiments, the Fc region contains an amino acid substitution at positions E430G, K322A, and P331S, where the numbering of the residue position is according to EU numbering. In some embodiments, the Fc region contains an amino acid substitution at positions S267E and L328F, where the numbering of the residue position is according to EU numbering. In some embodiments, the Fc region contains an amino acid substitution at position C127S, where the numbering of the residue position is according to EU numbering. In some embodiments, the Fc region contains an amino acid substitution at positions E345R, E430G and S440Y, where the numbering of the residue position is according to EU numbering. In some embodiments, the Fc region comprises an amino acid substitution at positions L243A, L235A, and P331S, wherein the numbering of the residue position is according to EU numbering.

Inert Antibodies

Another class of anti-CD33 antibodies of the present disclosure includes inert antibodies. As used herein, “inert” antibodies refer to antibodies that specifically bind their target antigen (e.g., CD33) but do not modulate (e.g., decrease/inhibit or activate/induce) antigen function. For example, in the case of CD33, inert antibodies do not modulate cellular levels of CD33, do not modulate interaction (e.g., binding) between CD33 and one or more CD33 ligands, or do not modulate one or more activities of a CD33 protein. In some embodiments, antibodies that do not have the ability to cluster CD33 on the cell surface may be inert antibodies even if they have an epitope specificity that is compatible with receptor activation.

In some embodiments, antibodies that bind a CD33 protein may include antibodies that bind CD33 but, due to their epitope specificity, or characteristics, do not decrease cellular levels of CD33 and/or inhibit interaction (e.g., binding) between CD33 and one or more CD33 ligands. In some embodiments, such antibodies can be used as cargo to, for example, transport toxins (e.g., chemotherapeutics) into tumor cells. Therefore, in some embodiments, antibodies of the present disclosure are inert antibodies that bind CD33 but are incapable of decreasing cellular levels of CD33, inhibiting interaction (e.g., binding) between CD33 and one or more CD33 ligands, or inducing one or more activities of a CD33 protein.

Antibodies that either decrease or do not decrease cellular levels of CD33 on cells can be combined with an inert Fc region that displays reduced binding to one or more Fcg Receptor. Examples of such Fc regions and modifications are provided in Table D below. In some embodiments, the antibody with an inert Fc region has an Fc isotype listed in Table D below.

Inhibitory Anti-CD33 Antibodies

A third class of anti-CD33 antibodies of the present disclosure includes antibodies that block or otherwise inhibit one or more CD33 activities. In some embodiments, antibodies that bind a CD33 protein may include antibodies that reduce cellular levels of CD33 (e.g., cell surface levels of CD33, intracellular levels of CD33, and/or total levels of CD33), inhibit interaction (e.g., binding) between CD33 and/or one or more CD33 ligands, and inhibit one or more activities of a CD33 protein. Such antibodies inhibit one or more activities of a CD33 protein either by preventing interaction (e.g., binding) between CD33 and one or more CD33 ligands or by preventing signal transduction from the extracellular domain of CD33 into the cell cytoplasm in the presence of one or more CD33 ligands. Antibodies also can inhibit one or more activities of a CD33 protein by decreasing cell surface levels of CD33 by inducing CD33 degradation, CD33 desensitization, CD33 cleavage, CD33 internalization, CD33 shedding, downregulation of CD33 expression, and/or lysosomal degradation of CD33. In some embodiments, such anti-CD33 antibodies may not transiently activate CD33.

In certain embodiments, the present disclosure provides an anti-CD33 antibody, wherein the anti-CD33 antibody decreases cellular levels of CD33, decreases cell surface levels of CD33, decreases intracellular levels of CD33, decreases total levels of CD33, or any combination thereof. In certain embodiments, the anti-CD33 antibody decreases cellular levels of CD33, decreases cell surface levels of CD33, decreases intracellular levels of CD33, decreases total levels of CD33, or any combination thereof, in monocytes, granulocytes, peripheral blood monocytes, peripheral blood granulocytes, CSF monocytes, and/or CSF granulocytes.

In certain embodiments, the present disclosure provides an anti-CD33 antibody, wherein the anti-CD33 antibody induces CD33 degradation, CD33 cleavage, CD33 internalization, CD33 downregulation, or any combination thereof. In certain embodiments, the anti-CD33 antibody induces CD33 degradation, CD33 cleavage, CD33 internalization, CD33 downregulation, or any combination thereof, in monocytes, granulocytes, peripheral blood monocytes, peripheral blood granulocytes, CSF monocytes, and/or CSF granulocytes.

In certain embodiments, the present disclosure provides an anti-CD33 antibody, wherein the anti-CD33 antibody decreases cellular levels of CD33 and inhibits the interaction between CD33 and a CD33 ligand. In certain embodiments, the anti-CD33 antibody decreases cellular levels of CD33 and inhibits the interaction between CD33 and a CD33 ligand in monocytes, granulocytes, peripheral blood monocytes, peripheral blood granulocytes, CSF monocytes, and/or CSF granulocytes.

In some embodiments, administration of an anti-CD33 antibody according to the methods provided herein reduces the cell surface level of CD33 by at least about 70% (e.g., at least about 70%, at least about 75%, at least about 80%, at least about 85%, at least about 90%, at least about 95%, or at least about 99%) compared to the cell surface level of CD33 prior to administration of the anti-CD33 antibody. In some embodiments, administration of an anti-CD33 antibody according to the methods provided herein reduces the cell surface level of CD33 by at least about 75% compared to the cell surface level of CD33 prior to administration of the anti-CD33 antibody. In some embodiments, administration of an anti-CD33 antibody according to the methods provided herein reduces the cell surface level of CD33 by at least about 80% compared to the cell surface level of CD33 prior to administration of the anti-CD33 antibody. In some embodiments, administration of an anti-CD33 antibody according to the methods provided herein reduces the cell surface level of CD33 by at least about 85% compared to the cell surface level of CD33 prior to administration of the anti-CD33 antibody. In some embodiments, administration of an anti-CD33 antibody according to the methods provided herein reduces the cell surface level of CD33 by at least about 90% compared to the cell surface level of CD33 prior to administration of the anti-CD33 antibody. In some embodiments, the reduction in cell surface levels of CD33 is present for at least about 10 days (e.g., at least 10 days, at least 15 days, at least 20 days, at least 25 days, at least 30 days, at least 35 days, at least 40 days, at least 45 days, at least 50 days, at least 55 days, at least 60 days, at least 70 days, at least 80 days, at least 90 days, or at least 100 days) after administration of the anti-CD33 antibody. In some embodiments, the reduction in cell surface levels of CD33 is present for at least about 12 days after administration of the anti-CD33 antibody. In some embodiments, the reduction in cell surface levels of CD33 is present for at least about 17 days after administration of the anti-CD33 antibody. In some embodiments, the reduction in cell surface levels of CD33 is present for at least about 29 days after administration of the anti-CD33 antibody. In some embodiments, the reduction in cell surface levels of CD33 is present for at least about 42 days after administration of the anti-CD33 antibody. In some embodiments, the reduction in cell surface levels of CD33 is present for at least about 56 days after administration of the anti-CD33 antibody. In some embodiments, the reduction in cell surface levels of CD33 is present for at least about 84 days after administration of the anti-CD33 antibody. In some embodiments, the reduction in cell surface level of CD33 is a reduction in the cell surface level of CD33 on monocytes, granulocytes, peripheral blood monocytes, peripheral blood granulocytes, CSF monocytes, and/or CSF granulocytes of the individual.

In some embodiments, an anti-CD33 antibody of the present disclosure reduces cell surface levels of CD33 by more than about 70% (e.g., about 70% or more, about 75% or more, about 80% or more, about 85% or more, about 90% or more, about 95% or more, or about 99% or more) compared to the cell surface levels of CD33 prior to administration of the anti-CD33 antibody. In some embodiments, an anti-CD33 antibody of the present disclosure reduces cell surface levels of CD33 by more than about 70% after a single intravenous dose of between 1.6 mg/kg and about 15 mg/kg (e.g., about 1.6 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, or about 15 mg/kg) of the antibody compared to the cell surface levels of CD33 prior to administration of the anti-CD33 antibody. In some embodiments, an anti-CD33 antibody of the present disclosure reduces cell surface levels of CD33 by more than about 70% after a single intravenous dose of 1.6 mg/kg of the antibody compared to the cell surface levels of CD33 prior to administration of the anti-CD33 antibody. In some embodiments, an anti-CD33 antibody of the present disclosure reduces cell surface levels of CD33 by more than about 70% after a single intravenous dose of 15 mg/kg of the antibody compared to the cell surface levels of CD33 prior to administration of the anti-CD33 antibody. In some embodiments, an anti-CD33 antibody of the present disclosure reduces cell surface levels of CD33 by more than about 80% after a single intravenous dose of 15 mg/kg of the antibody compared to the cell surface levels of CD33 prior to administration of the anti-CD33 antibody. In some embodiments, an anti-CD33 antibody of the present disclosure reduces cell surface levels of CD33 by more than about 85% after a single intravenous dose of 15 mg/kg of the antibody compared to the cell surface levels of CD33 prior to administration of the anti-CD33 antibody. In some embodiments, an anti-CD33 antibody of the present disclosure reduces cell surface levels of CD33 by more than about 90% after a single intravenous dose of 15 mg/kg of the antibody compared to the cell surface levels of CD33 prior to administration of the anti-CD33 antibody. In some embodiments, the reduction in cell surface levels of CD33 occurs in monocytes from whole blood, granulocytes, peripheral blood monocytes, peripheral blood granulocytes, CSF monocytes, and/or CSF granulocytes from the individual.

In some embodiments, the cell surface level of CD33 may be determined according to any method known in the art. In some embodiments, the cell surface level of CD33 may be determined using flow cytometry. In some embodiments, the cell surface level of CD33 is expressed as Mean Fluorescence Intensity (MFI). In some embodiments, the cell surface level of CD33 is expressed as Molecules of Equivalent Soluble Fluorochrome (MESF) (e.g., see Schawrts et al., (2004) Clin Cytometry 57B:1-6).

In some embodiments, anti-CD33 antibodies of the present disclosure may have the epitope specificity of a transient agonist anti-CD33 antibody of the present disclosure, but have an Fc domain that is not capable of binding Fcg receptors and thus is unable to, for example, transiently clustering and activating CD33.

In some embodiments, anti-CD33 antibodies of the present disclosure have, without limitation, one or more of the following activities: the ability to decrease binding of a CD33 protein to one or more CD33 ligands, such as sialic acid-containing glycolipid s or sialic acid-containing glycoproteins, the ability to decrease the binding of a suppressor of cytokine signaling (SOCS) protein (e.g., SOCS3 protein) to a CD33 protein, the ability to increase the proteasomal degradation of a CD33 protein, the ability to reduce functional expression of CD33 on the surface of circulating dendritic cells, macrophages, monocytes, T cells, and/or microglia, the ability to decrease phosphorylation of Tyr-340 and Tyr-358 by a Src family tyrosine kinase such as LCK and FYN, the ability to decrease recruitment of and binding to the tyrosine-specific protein phosphatases SHP1 and SHP2, the ability to decrease recruitment of and binding to PLC-g1, which acts as a guanine nucleotide, exchange factor for Dynamin-1, the ability to decrease recruitment of and binding to Crk1, the ability to decrease recruitment of and binding to the Spleen tyrosine kinase Syk, the ability to decrease recruitment of and binding to SH3-SH2-SH3 growth factor receptor-bound protein 2 (Grb2), the ability to decrease recruitment of and binding to multiple SH2 containing proteins, the ability to increase intracellular calcium mobilization, the ability to modulate production of pro-inflammatory cytokines IL-1<, IL-8, and TNF-α, the ability to decrease activation of phosphoinositide 3-kinase, the ability to increase the growth of monocytes, macrophages, dendritic cells, T cells, and/or microglia, the ability to increase the survival of monocytes, macrophages, dendritic cells, T cells, and/or microglia, the ability to increase tyrosine phosphorylation on multiple cellular proteins, the ability to increase phagocytic activity of monocytes, macrophages, dendritic cells and/or microglia, the ability to increase cell proliferation of monocytes, macrophages, dendritic cells, T cells, and/or microglia, the ability to increase phosphorylation of signaling molecules that mediates ITAM signaling, the ability to increase the function of pattern recognition receptors, the ability to increase the function of Toll-like receptors, the ability to increases the function of damage-associated molecular pattern (DAMP) receptors, the ability to modulate expression of C-C chemokine receptor 7 (CCR7), and the ability to increase of clearance of cellular and protein debris.

In some embodiments, anti-CD33 antibodies of the present disclosure have an Fc region that displays reduced binding to one or more Fcg Receptor. Examples of such Fc regions and modifications are provided in Table D below. In some embodiments, the antibody has an Fc isotype listed in Table D below.

Antibody Fc Isotypes with Reduced Binding to Fc Gamma Receptors

In some embodiments, anti-CD33 antibodies with reduced binding to Fc gamma receptors have an Fc isotype listed in Table C below.

TABLE C
Exemplary anti-CD33 antibody Fc isotypes with reduced binding to Fc
gamma receptor

| Fc Isotype | Mutation (EU numbering scheme) |
|---|---|
| IgG1 | N297A or N297Q and/or D270A |
| IgG1 | D265A, D270A, and/or N297A |
| IgG1 | L234A and L235A |
| IgG2 | V234A and G237A |
| IgG4 | F235A and G237A and E318A |
| | E233P and/or F234V |
| | N297Aor N297Q |
| IgG4 | S228P and L236E |
| | S241P |
| | S241P and L248E |
| | S228P and F234A and L235A |
| IgG2 | H268Q and V309L and A330S and P331S |
| IgG1 | C220S and C226S and C229S and P238S |
| IgG1 | C226S and C229S and E233P and L234V, and L235A |
| IgG1 | E233P and L234V and L235A and G236-deleted |
| | P238A |
| | D265A |
| | N297A |
| | A327Q or A327G |
| | P329A |
| IgG1 | K322A and L234A and L235A |
| IgG1 | L234Fand L235E and P331S |
| IgG1 or IgG4 | T394D |
| IgG2 | C232S or C233S |
| | N297A or N297Q |
| IgG2 | V234A and G237A and P238S and H268A and V309L |
| | and A330S and P331S |
| IgG1, IgG2, | delta a, b, c, ab, ac, g modifications |
| or IgG4 | |
| IgG1 | Any of the above listed mutations together with A330L |
| | or L234F and/or L235E and/or P331S |
| IgG1, IgG2, | Any of the above listed mutations together with M252Y |
| or IgG4 | and/or S254T and/or T256E |

In certain embodiments, the anti-CD33 antibody has an IgG1 isotype. In some embodiments, the antibody contains a mouse IgG1 constant region. In some embodiments, the antibody contains a human IgG1 constant region. In some embodiments, the human IgG1 constant region includes an Fc region. In some embodiments, the Fc region contains one or more modifications. For example, in some embodiments, the Fc region contains one or more amino acid substitutions (e.g., relative to a wild-type Fc region of the same isotype).

In some embodiments, the one or more amino acid substitutions are selected from N297A, N297Q (Bolt S et al. (1993) Eur J Immunol 23:403-411), D265A, D270A, L234A, L235A (McEarchern et al., (2007) Blood, 109:1185-1192), C226S, C229S (McEarchern et al., (2007) Blood, 109:1185-1192), P238S (Davis et al., (2007) J Rheumatol, 34:2204-2210), E233P, L234V (McEarchern et al., (2007) Blood, 109:1185-1192), P238A, A327Q, A327G, P329A (Shields R L, et al., (2001) J Biol Chem. 276(9):6591-604), K322A, L234F, L235E (Hezareh, et al., (2001) J Virol 75, 12161-12168; Oganesyan et al., (2008). Acta Crystallographica 64, 700-704), P331S (Oganesyan et al., (2008) Acta Crystallographica 64, 700-704), T394D (Wilkinson et al. (2013)MAbs 5(3): 406-417), A330L, M252Y, S254T, and/or T256E, where the amino acid position is according to the EU or Kabat numbering convention. In certain embodiments, the Fc region further includes an amino acid deletion at a position corresponding to glycine 236 according to the EU or Kabat numbering convention.

In some embodiments, the anti-CD33 antibody has an IgG1 isotype with a heavy chain constant region that contains a C220S amino acid substitution according to the EU or Kabat numbering convention. In some embodiments, the Fc region further contains one or more additional amino acid substitutions selected from A330L, L234F; L235E, and/or P331S according to EU or Kabat numbering convention. In certain embodiments, the anti-CD33 antibody has an IgG2 isotype. In some embodiments, the anti-CD33 antibody contains a human IgG2 constant region. In some embodiments, the human IgG2 constant region includes an Fc region. In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from P238S, V234A, G237A, H268A, H268Q, H268E, V309L, N297A, N297Q, V309L, A330S, P331S, C232S, C233S, M252Y, S254T, and/or T256E, where the amino acid position is according to the EU or Kabat numbering convention (Vafa O. et al., (2014) Methods 65:114-126).

In certain embodiments, the anti-CD33 antibody has an IgG4 isotype. In some embodiments, the anti-CD33 antibody contains a human IgG4 constant region. In some embodiments, the human IgG4 constant region includes an Fc region. In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from E233P, F234V, L235A, G237A, E318A (Hutchins et al. (1995) Proc Natl Acad Sci USA, 92:11980-11984), S228P, L234A/F234A, L236E, S241P, L248E (Reddy et al., (2000) J Immunol, 164:1925-1933; Angal et al., (1993) Mol Immunol. 30(1):105-8; U.S. Pat. No. 8,614,299 B2; Vafa O. et al., (2014) Methods 65:114-126), T394D, M252Y, S254T, T256E, N297A, and/or N297Q, where the amino acid position is according to the EU or Kabat numbering convention. In some embodiments the antibody has an IgG4 isotype, and comprises an S228P amino acid substitution at residue position 228, an F234A amino acid substitution at residue position 234, and an L235A amino acid substitution at residue position 235 (residue position according to EU numbering).

In some embodiments, the Fc region further contains one or more additional amino acid substitutions selected from a M252Y, S254T, and/or T256E, where the amino acid position is according to the EU or Kabat numbering convention.

Further IgG Mutations

In some embodiments, one or more of the IgG1 variants described herein may be combined with an A330L mutation (Lazar et al., (2006) Proc Natl Acad Sci USA, 103:4005-4010), or one or more of L234F, L235E, and/or P331S mutations (Sazinsky et al., (2008) Proc Natl Acad Sci USA, 105:20167-20172), where the amino acid position is according to the EU or Kabat numbering convention, to eliminate complement activation. In some embodiments, the IgG variants described herein may be combined with one or more mutations to enhance the anti-CD33 antibody half-life in human serum (e.g. M252Y, S254T, T256E mutations according to the EU or Kabat numbering convention) (Dall'Acqua et al., (2006) J Biol Chem, 281:23514-23524; and Strohl e al., (2009) Current Opinion in Biotechnology, 20:685-691).

In some embodiments, an IgG4 variant of the present disclosure may be combined with an S228P mutation according to the EU or Kabat numbering convention (Angal et al., (1993) Mol Immunol, 30:105-108) and/or with one or more mutations described in Peters et al., (2012) J Biol Chem. 13; 287(29):24525-33) to enhance antibody stabilization.

Bispecific Antibodies

Certain aspects of the present disclosure relate to bispecific antibodies that bind to one or more domains on a CD33 protein of the present disclosure and a second antigen. Methods of generating bispecific antibodies are well known in the art and described herein. In some embodiments, bispecific antibodies of the present disclosure bind to one or more amino acid residues of a CD33 protein of the present disclosure, such as one or more amino acid residues of human CD33 (SEQ ID NO: 1), or amino acid residues on a CD33 protein corresponding to amino acid residues of SEQ ID NO: 1. In some embodiments, bispecific antibodies of the present disclosure recognize a first antigen and a second antigen. In some embodiments, the first antigen is a CD33 protein or a naturally occurring variant thereof. In some embodiments, the second antigen is also a CD33 protein, or a naturally occurring variant thereof. In some embodiments, the second antigen is an antigen facilitating transport across the blood-brain-barrier (see, e.g., Gabathuler R., Neurobiol. Dis. 37 (2010) 48-57). Such second antigens include, without limitation, transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM 30(A), a protein transduction domain, TAT, Syn-B, penetratin, a poly-arginine peptide, Angiopep peptides such as ANG1005 (see, e.g., Gabathuler, 2010), and other cell surface proteins that are enriched on blood-brain barrier endothelial cells (see, e.g., Daneman et al., PLoS One. 2010 Oct. 29; 5(10):e13741). In some embodiments, the second antigen is a disease-causing protein including, without limitation, amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides. In some embodiments, the second antigen is one or more ligands and/or proteins expressed on immune cells, including without limitation, CD40, OX40, ICOS, CD28, CD137/4-1BB, CD27, GITR, PD-L1, CTLA4, PD-L2, PD-1, B7-H3, B7-H4, HVEM, LIGHT, BTLA, CD38, TIGIT, VISTA, KIR, GAL9, TIM1, TIM3, TIM4, A2AR, LAG3, DR5, CD39, CD70, CD73, TREM1, TREM2, Siglec-5, Siglec-7, Siglec-9, Siglec-11, SirpA, CD47, CSF1-receptor, CD3, and phosphatidylserine. In some embodiments, the second antigen is a protein, lipid, polysaccharide, or glycolipid expressed on one or more tumor cells.

Antibody Fragments

Certain aspects of the present disclosure relate to antibody fragments that bind to one or more of a CD33 protein of the present disclosure, a naturally occurring variant of a CD33 protein, and a disease variant of a CD33 protein. In some embodiments, the antibody fragment is an Fab, Fab′, Fab′-SH, F(ab′)2, Fv or scFv fragment.

In some embodiments, the antibody fragment is used in combination with a second CD33 antibody and/or with one or more antibodies that specifically bind a disease-causing protein selected from: amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, and any combination thereof; or with one or more antibodies that bind an immunomodulatory protein selected from the group consisting of: CD40, OX40, ICOS, CD28, CD137/4-1BB, CD27, GITR, PD-L1, CTLA4, PD-L2, PD-1, B7-H3, B7-H4, HVEM, LIGHT, BTLA, CD38, TIGIT, VISTA, KIR, GAL9, TIM1, TIM3, TIM4, A2AR, LAG3, DR5, CD39, CD70, CD73, TREM1, TREM2, CD47, CSF-1 receptor, Siglec-5, Siglec-7, Siglec-9, Siglec-11, phosphatidylserine, and any combination thereof.

In some embodiments, antibody fragments of the present disclosure may be functional fragments that bind the same epitope as any of the anti-CD33 antibodies of the present disclosure. In some embodiments, the antibody fragments are miniaturized versions of the anti-CD33 antibodies or antibody fragments of the present disclosure that have the same epitope of the corresponding full-length antibody, but have much smaller molecule weight. Such miniaturized anti-CD33 antibody fragments may have better brain penetration ability and a shorter half-life, which is advantageous for imaging and diagnostic utilities (see e.g., Lütje S et al., Bioconjug Chem. 2014 Feb. 19; 25(2):335-41; Tavaré R et al., Proc Natl Acad Sci USA. 2014 Jan. 21; 111(3):1108-13; and Wiehr S et al., Prostate. 2014 May; 74(7):743-55). Accordingly, in some embodiments, anti-CD33 antibody fragments of the present disclosure have better brain penetration as compared to their corresponding full-length antibodies and/or have a shorter half-life as compared to their corresponding full-length antibodies.

Antibody Frameworks

Any of the antibodies described herein further include a framework. In some embodiments, the framework is a human immunoglobulin framework. For example, in some embodiments, an antibody (e.g., an anti-CD33 antibody) comprises HVRs as in any of the above embodiments and further comprises an acceptor human framework, e.g., a human immunoglobulin framework or a human consensus framework. Human immunoglobulin frameworks may be part of the human antibody, or a non-human antibody may be humanized by replacing one or more endogenous frameworks with human framework region(s). Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In some embodiments, anti-CD33 antibodies of the present disclosure comprise a heavy chain variable region comprising one or more (e.g., one or more, two or more, three or more, or all four) framework regions selected from VH FR1, VH FR2, VH FR3, and VH FR4 (as shown in Tables 5A-5D). In some embodiments, the VH FR1 comprises a sequence according to Formula VII: QVQLVQSGAEVKKPGX₁SVKX₂SCKAS (SEQ ID NO: 158), wherein X₁ is A or S, and X₂ is V or I. In some embodiments, VH FR1 comprises a sequence selected from the group consisting of SEQ ID NOs: 2-4. In some embodiments, the VH Fr2 comprises the sequence of SEQ ID NO: 5. In some embodiments, the VH FR3 comprises a sequence according to Formula VIII: X₁AX₂X₃X₄X₅X₆RX₇TX₈TVDX₉X₁₀X₁₁STX₁₂YMELSSLRSEDTAVYYCAR (SEQ ID NO: 159), wherein X₁ is Y or S, X₂ is Q or E, X₃ is K or D, X₄ is F or D, X₅ is Q, F, E, or T, X₆ is G, D, or H, X₇ is V or A, X₈ is M or L, X₉ is T, N, or Q, X₁₀ is S or P, X₁₁ is T or A, and X₁₂ is V or A. In some embodiments, VH FR3 comprises a sequence selected from the group consisting of SEQ ID NOs: 6-19. In some embodiments, VH FR4 comprises a sequence according to Formula IX: WGQGTLX₁TVSS (SEQ ID NO: 160), wherein X₁ is V or L. In some embodiments, VH FR4 comprises a sequence selected from the group consisting of SEQ ID NOs: 20-21. In some embodiments, an antibody comprises a heavy chain variable region comprising a VH FR1 according to Formula VII, a VH FR2 comprising the sequence of SEQ ID NO: 5, a VH FR3 according to Formula VIII, and VH FR4 according to Formula IX. In some embodiments, anti-CD33 antibodies of the present disclosure comprise a heavy chain variable region comprising a VH FRI comprising a sequence selected from SEQ ID NOs: 2-4, a VH FR2 comprising the sequence of SEQ ID NO: 5, a VH FR3 comprising a sequence selected from SEQ ID NOs: 6-19, and VH FR4 comprising a sequence selected from SEQ ID NOs: 20-21. In some embodiments, anti-CD33 antibodies of the present disclosure comprise a heavy chain variable region comprising a VH FR1, a VH FR2, a VH FR3, and VH FR4 of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15 (as shown in Table 3).

In some embodiments, anti-CD33 antibodies of the present disclosure comprise a light chain variable region comprising one or more (e.g., one or more, two or more, three or more, or all four) framework regions selected from VL FRI, VL FR2, VL FR3, and VL FR4 (as shown in Tables 6A-6D). In some embodiments, the VL FRI comprises a sequence according to Formula X: X₁IX₂X₃TQSPX₄SLX₅X₆SX₇GXsRX₉TIX₁₀C (SEQ ID NO: 161), wherein X₁ is D or G, X2 is Q or V, X₃ is M or L, X₄ is S or D, X₅ is S, P, or A, X₆ is A or V, X₇ is V or L, X₈ is D or E, X₉ is V or A, and X₁₀ is T, N, or D. In some embodiments, VL FRI comprises a sequence selected from the group consisting of SEQ ID NOs: 22-26. In some embodiments, the VL FR2 comprises a sequence according to Formula XI: WYQQKPGX₁X2PKLLIK (SEQ ID NO: 162), wherein X₁ is K or Q, and X₂ is A or P. In some embodiments, the VL FR2 comprises a sequence selected from the group consisting of SEQ ID NOs: 27-28. In some embodiments, the VL FR3 comprises a sequence according to Formula XII: GVPX₁RFSGSGSGTDFTLTISSLQX₂EDX₃AX₄YYC (SEQ ID NO: 163), wherein X₁ is S or D, X₂ is P or A, X₃ is F, L, or V, and X₄ is T or V. In some embodiments, VL FR3 comprises a sequence selected from the group consisting of SEQ ID NOs: 29-31. In some embodiments, VL FR4 comprises a sequence according to Formula XIII: FGQGTKLEIX₁ (SEQ ID NO: 164), wherein X₁ is K or E. In some embodiments, VL FR4 comprises a sequence selected from the group consisting of SEQ ID NOs: 32-33. In some embodiments, anti-CD33 antibodies of the present disclosure comprise a light chain variable region comprising a VL FR1 according to Formula X, a VL FR2 according to Formula XI, a VL FR3 according to Formula XII, and VL FR4 according to Formula XIII. In some embodiments, anti-CD33 antibodies of the present disclosure comprise a light chain variable region comprising a VL FRI comprising a sequence selected from SEQ ID NOs: 22-26, a VL FR2 comprising a sequence selected from SEQ ID NOs: 27-28, a VL FR3 comprising a sequence selected from SEQ ID NOs: 29-31, and VL FR4 comprising a sequence selected from SEQ ID NOs: 32-33. In some embodiments, anti-CD33 antibodies of the present disclosure comprise a light chain variable region comprising a VL FR1, a VL FR2, a VL FR3, and VL FR4 of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15 (as shown in Table 4).

In some embodiments, anti-CD33 antibodies of the present disclosure comprise a heavy chain variable region comprising one or more (e.g., one or more, two or more, three or more, or all four) framework regions selected from VH FR1, VH FR2, VH FR3, and VH FR4 (as shown in Tables 5A-5D), and a light chain variable region comprising one or more (e.g., one or more, two or more, three or more, or all four) framework regions selected from VL FR1, VL FR2, VL FR3, and VL FR4 (as shown in Tables 6A-6D). In some embodiments, anti-CD33 antibodies of the present disclosure comprise a heavy chain variable region comprising a VH FRI according to Formula VII, a VH FR2 comprising the sequence of SEQ ID NO: 5, a VH FR3 according to Formula VIII, and VH FR4 according to Formula IX, and a light chain variable region comprising a VL FR1 according to Formula X, a VL FR2 according to Formula XI, a VL FR3 according to Formula XII, and VL FR4 according to Formula XIII. In some embodiments, anti-CD33 antibodies of the present disclosure comprise a heavy chain variable region comprising a VH FR1 comprising a sequence selected from SEQ ID NOs: 2-4, a VH FR2 comprising the sequence of SEQ ID NO: 5, a VH FR3 comprising a sequence selected from SEQ ID NOs: 6-19, and VH FR4 comprising a sequence selected from SEQ ID NOs: 20-21, a light chain variable region comprising a VL FRI comprising a sequence selected from SEQ ID NOs: 22-26, a VL FR2 comprising a sequence selected from SEQ ID NOs: 27-28, a VL FR3 comprising a sequence selected from SEQ ID NOs: 29-31, and VL FR4 comprising a sequence selected from SEQ ID NOs: 32-33. In some embodiments, anti-CD33 antibodies of the present disclosure comprise a heavy chain variable region comprising a VH FR1, a VH FR2, a VH FR3, and VH FR4 of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15 (as shown in Table 3), and a light chain variable region comprising a VL FRI, a VL FR2, a VL FR3, and VL FR4 of antibody AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, or AB-64.1.15 (as shown in Table 4).

Pharmacokinetics of Anti-CD33 Antibodies

In some embodiments, the terminal half-life of the anti-CD33 antibody in plasma is between about 4 days and about 12 days (e.g., any of about 4 days, about 5 days, about 6 days, about 7 days, about 8 days, about 9 days, about 10 days, about 11 days, or about 12 days). In some embodiments, the terminal half-life of the anti-CD33 antibody is about 4 days. In some embodiments, the terminal half-life of the anti-CD33 antibody is about 10 days. In some embodiments, the terminal half-life of the anti-CD33 antibody is about 11 days. In some embodiments, the terminal half-life of the anti-CD33 antibody following administration of a single dose of the antibody at 1.6 mg/kg is about 4 days. In some embodiments, the terminal half-life of the anti-CD33 antibody following administration of a single dose of the antibody at 1.6 mg/kg is about 5 days. In some embodiments, the terminal half-life of the anti-CD33 antibody following administration of a single dose of the antibody at 5 mg/kg is about 7 days. In some embodiments, the terminal half-life of the anti-CD33 antibody following administration of a single dose of the antibody at 5 mg/kg is about 8 days. In some embodiments, the terminal half-life of the anti-CD33 antibody following administration of a single dose of the antibody at 15 mg/kg is about 10 days. In some embodiments, the terminal half-life of the anti-CD33 antibody following administration of a single dose of the antibody at 15 mg/kg is about 11 days. In some embodiments, the terminal half-life of the anti-CD33 antibody following administration of a single dose of the antibody at 30 mg/kg is about 9 days. In some embodiments, the terminal half-life of the anti-CD33 antibody following administration of a single dose of the antibody at 30 mg/kg is about 10 days.

In some embodiments, the terminal half-life of an anti-CD33 antibody of the present disclosure is determined using any method known in the art. In some embodiments, the terminal half-life of an anti-CD33 antibody of the present disclosure is determined using enzyme-linked immunosorbent assay (ELISA). In some embodiments, the terminal half-life of an anti-CD33 antibody of the present disclosure is determined in blood of the individual. In some embodiments, the terminal half-life of an anti-CD33 antibody of the present disclosure is determined in plasma of the individual. In some embodiments, the terminal half-life of an anti-CD33 antibody of the present disclosure is determined in serum of the individual. In some embodiments, the terminal half-life of an anti-CD33 antibody of the present disclosure is determined in CSF of the individual.

### CD33 Proteins

In one aspect, the present disclosure provides antibodies, such as isolated (e.g., monoclonal) antibodies, that interact with or otherwise bind to a region, such as an epitope, within a CD33 protein of the present disclosure. In some embodiments, the antibodies interact with or otherwise bind to a region, such as an epitope, within a CD33 protein of the present disclosure with improved/enhanced kinetics (e.g., relative to an anti-CD33 antibody having a heavy chain variable region comprising the sequence of SEQ ID NO: 103 and a light chain variable region comprising the sequence of SEQ ID NO: 104). In some embodiments, the antibodies interact with or otherwise bind to a region, such as an epitope, within a CD33 protein on human cells, such as dendritic cells, with a half-maximal effective concentration (EC₅₀) that is lower than that of a control antibody (e.g., relative to an anti-CD33 antibody having a heavy chain variable region comprising the sequence of SEQ ID NO: 103 and a light chain variable region comprising the sequence of SEQ ID NO: 104). In some embodiments, anti-CD33 antibodies of the present disclosure bind to a CD33 protein and modulate one or more CD33 activities after binding to the CD33 protein, for example, an activity associated with CD33 expression on a cell. CD33 proteins of the present disclosure include, without limitation, a mammalian CD33 protein, human CD33 protein, mouse CD33 protein, and rat CD33 protein.

CD33 is variously referred to as a CD33 molecule, Siglec3, Siglec-3, CD33 antigen (Gp67), P67, Gp67, sialic acid-binding-Ig-like lectin 3, myeloid cell surface antigen CD33, or FLJ00391.

CD33 is an immunoglobulin-like receptor primarily expressed on myeloid lineage cells, including without limitation, macrophages, dendritic cells, osteoclasts, monocytes, and microglia. In some embodiments, CD33 forms a receptor-signaling complex with CD64. In some embodiments, CD33 signaling results in the downstream inhibition of PI3K or other intracellular signals. On myeloid cells, Toll-like receptor (TLR) signals are important for the inhibition of CD33 activities, e.g., in the context of an infection response. TLRs also play a key role in the pathological inflammatory response, e.g., TLRs expressed in macrophages and dendritic cells.

The amino acid sequence of human CD33 is set forth below as SEQ ID NO: 1:

MPLLLLLPLL WAGALAMDPN FWLQVQESVT
VQEGLCVLVP CTFFHPIPY FDKNSPVHGYW
FREGAIISRD SPVATNKLDQ EVQEETQGRF
RLLGDPSRNN CSLSIVDARR RDNGSYFFRM
ERGSTKYSYK SPQLSVHVTD LTHRPKILIP
GTLEPGHSKN LTCSVSWACE QGTPPIFSWL
SAAPTSLGPR TTHSSVLIIT PRPQDHGTNL
TCQVKFAGAG VTTERTIQLN VTYVPQNPTT
GIFPGDGSGK QETRAGVVHG AIGGAGVTAL
LALCLCLIFF IVKTHRRKAA RTAVGRNDTH
PTTGSASPKH QKKSKLHGPT ETSSCSGAAP
TVEMDEELHY ASLNFHGMNP SKDTSTEYSE

In some embodiments, the CD33 is a preprotein that includes a signal sequence. In some embodiments, the CD33 is a mature protein. In some embodiments, the mature CD33 protein does not include a signal sequence. In some embodiments, the mature CD33 protein is expressed on a cell. In some embodiments, the mature CD33 protein is expressed on a cell, such as the surface of a cell, including, without limitation, human dendritic cells, human macrophages, human monocytes, human osteoclasts, human neutrophils, human T cells, human T helper cell, human cytotoxic T cells, human granulocytes, and human microglia. Anti-CD33 antibodies of the present disclosure may bind any of the CD33 proteins of the present disclosure expressed on any cell disclosed herein.

CD33 proteins of the present disclosure, such as human CD33, contain several domains, including without limitation, a signal sequence located at amino acid residues 1-17 of SEQ ID NO: 1, an extracellular immunoglobulin-like variable-type (IgV) domain located at amino acid residues 19-135 of SEQ ID NO: 1, an Ig-like C2-type domain located at amino acid residues 145-228 of SEQ ID NO: 1, a transmembrane domain located at amino acid residues 260-282 of SEQ ID NO: 1, an ITIM motif 1 located at amino acid residues 338-343 of SEQ ID NO: 1, and an ITIM motif 2 located at amino acid residues 356-361 of SEQ ID NO: 1. As one of skill in the art will appreciate, the beginning and ending residues of the domains of the present disclosure may vary depending upon the computer modeling program used or the method used for determining the domain.

Certain aspects of the present disclosure provide anti-CD33 antibodies that bind to a human CD33, or a homolog thereof, including without limitation a mammalian CD33 protein and Cd33 orthologs from other species. In some embodiments, the anti-CD33 antibodies of the present disclosure bind to a human CD33, or homolog thereof, with improved/enhanced binding kinetics (e.g., relative to an anti-CD33 antibody having a heavy chain variable region comprising the sequence of SEQ ID NO: 103 and a light chain variable region comprising the sequence of SEQ ID NO: 104).

Accordingly, as used herein a “CD33” protein of the present disclosure includes, without limitation, a mammalian CD33 protein, human CD33 protein, primate CD33 protein, mouse CD33 protein, and rat CD33 protein. Additionally, anti-CD33 antibodies of the present disclosure may bind an epitope within a human CD33 protein, primate CD33. In some embodiments, anti-CD33 antibodies of the present disclosure may bind specifically to human CD33.

In some embodiments, antibodies of the present disclosure may bind CD33 in a pH dependent manner. In some embodiments, antibodies of the present disclosure can bind to CD33 at a neutral pH and be internalized without dissociating from the CD33 protein. Alternatively, at an acidic pH, antibodies of the present disclosure may dissociate from CD33 once they are internalized and are then degraded by endosome/lysosome pathway. In certain embodiments, an anti-CD33 antibody binds CD33 at a pH that ranges from 5.5 to 8.0, from 5.5 to 7.5, from 5.5 to 7.0, from 5.5 to 6.5, from 5.5 to 6.0, from 6.0 to 8.0, from 6.5 to 8.0, from 7.0 to 8.0, from 7.5 to 8.0, from 6.0 to 7.5, from 6.0 to 7.0, from 6.5 to 7.5. In certain embodiments, an anti-CD33 antibody dissociates from CD33 at a pH of less than 6.0, less than 5.5, less than 5.0, less than 4.5, less than 4.0, less than 3.5, less than 3.0, less than 2.5, or less than 2.0.

In some embodiments, antibodies of the present disclosure, bind to a wild-type CD33 protein of the present disclosure, naturally occurring variants thereof, and/or disease variants thereof.

In some embodiments, antibodies of the present disclosure bind a variant of human CD33, wherein the variant contains a single nucleotide polymorphism (SNP) rs3865444C with a (C) nucleotide. In some embodiments, antibodies of the present disclosure that decrease cellular levels of CD33 and/or that bind or interact with CD33, bind to a variant of human CD33, wherein the variant contains a SNP rs3865444 with an (A) nucleotide. In some embodiments, anti-CD33 antibodies of the present disclosure bind a variant of human CD33, wherein the variant contains a SNP rs3865444^{AC} or rs3865444^{CC}.

In some embodiments, antibodies of the present disclosure that decrease cellular levels of CD33 and/or that bind or interact with CD33, bind a variant of human CD33, wherein the variant contains a SNP rs35112940 with GG nucleotides, AA nucleotides, or AG nucleotides. In some embodiments, antibodies of the present disclosure that decrease cellular levels of CD33 and/or that bind or interact with CD33, bind a variant of human CD33, wherein the variant contains a SNP rs12459419 with CC, CT or TT genotypes. In certain embodiments, the subject has a homozygous or heterozygous for the coding SNPs, rs1803 with GG nucleotides, CG nucleotides, or CC nucleotides.

In some embodiments, antibodies of the present disclosure that decrease cellular levels of CD33 and/or that bind or interact with CD33, bind to a CD33 protein expressed on the surface of a cell including, without limitation, human dendritic cells, human macrophages, human monocytes, human osteoclasts, human neutrophils, human T cells, human T helper cell, human cytotoxic T cells, human granulocytes, and human microglia. In some embodiments, antibodies of the present disclosure that decrease cellular levels of CD33 and/or that bind or interact with CD33, bind to a CD33 protein expressed on the surface of a cell and modulate (e.g., induce or inhibit) at least one CD33 activity of the present disclosure after binding to the surface expressed CD33 protein. In some embodiments of the present disclosure, the anti-CD33 antibody binds specifically to a CD33 protein. In some embodiments of the present disclosure, the anti-CD33 antibody further binds to at least one additional Siglec protein. In some embodiments, the anti-CD33 antibody modulates one or more activities of the at least one additional Siglec protein or of a cell expressing the at least one additional Siglec protein.

### CD33 Ligands

CD33 proteins of the present disclosure can interact with (e.g., bind to) one or more CD33 ligands.

Exemplary CD33 ligands include, without limitation, sialic acid, sialic acid-containing glycolipids, sialic acid-containing glycoproteins, alpha-2,6-linked sialic acid-containing glycolipids, alpha-2,6-linked sialic acid-containing glycoproteins, alpha-2,3-linked sialic acid-containing glycolipids, alpha-2,3-linked sialic acid-containing glycoproteins, alpha-1-acid glycoprotein (AGP), CD24 protein, gangliosides (e.g., glycolipids containing a ceramide linked to a sialylated glycan), secreted mucins, CD33 ligands expressed on red blood cells, CD33 ligands expressed on bacterial cells, CD33 ligands expressed on apoptotic cells, CD33 ligands expressed on tumor cells, CD33 ligands expressed on viruses, CD33 ligands expressed on dendritic cells, CD33 ligands expressed on nerve cells, CD33 ligands expressed on glial cells, CD33 ligands expressed on microglia, CD33 ligands expressed on astrocytes, CD33 ligands on beta amyloid plaques, CD33 ligands on Tau tangles, CD33 ligands on disease-causing proteins, CD33 ligands on disease-causing peptides, CD33 ligands expressed on macrophages, CD33 ligands expressed on natural killer cells, CD33 ligands expressed on T cells, CD33 ligands expressed on T helper cells, CD33 ligands expressed on cytotoxic T cells, CD33 ligands expressed on B cells, CD33 ligands expressed on tumor-imbedded immunosuppressor dendritic cells, CD33 ligands expressed on tumor-imbedded immunosuppressor macrophages, CD33 ligands expressed on myeloid-derived suppressor cells, and CD33 ligands expressed on regulatory T cells. In some embodiments, CD33 ligands of the present disclosure are gangliosides. Gangliosides generally share a common lacto-ceramide core and one or more sialic acid residues.

Further examples of suitable ganglioside ligands are listed in Table A. Generally, a ganglioside is a molecule composed of a glycosphingolipid with one or more sialic acids (e.g., n-acetyl-neuraminic acid, NANA) linked on the sugar chain.

TABLE A
Structures of exemplary ganglioside CD33 ligands

GM2-1 = aNeu5Ac(2-3)bDGalp(1-?)bDGalNAc(1-?)bDGalNAc(1-?)bDGlcp(1-1)Cer
GM3 = aNeu5Ac(2-3)bDGalp(1-4)bDGlcp(1-1)Cer
GM2,GM2a(?) = bDGalpNAc(1-4)[aNeu5Ac(2-3)]bDGalp(1-4)bDGlcp(1-1)Cer
GM2b(?) = aNeu5Ac(2-8)aNeu5Ac(2-3)bDGalp(1-4)bDGlcp(1-1)Cer
GM1,GM1a = bDGalp(1-3)bDGalNAc[aNeu5Ac(2-3)]bDGalp(1-4)bDGlcp(1-1)Cer
asialo-GM1,GA1 =bDGalp(1-3)bDGalpNAc(1-4)bDGalp(1-4)bDGlcp(1-1)Cer
asialo-GM2,GA2 = bDGalpNAc(1-4)bDGalp(1-4)bDGlcp(1-1)Cer
GM1b = aNeu5Ac(2-3)bDGalp(1-3)bDGalNAc(1-4)bDGalp(1-4)bDGlcp(1-1)Cer
GD3 = aNeu5Ac(2-8)aNeu5Ac(2-3)bDGalp(1-4)bDGlcp(1-1)Cer
GD2 = bDGalpNAc(1-4)[aNeu5Ac(2-8)aNeu5Ac(2-3)]bDGalp(1-4)bDGlcp(1-1)Cer
GD1a = aNeu5Ac(2-3)bDGalp(1-3)bDGalNAc(1-4)[aNeu5Ac(2-3)]bDGalp(1-4)bDGlcp(1-1)Cer
GD1alpha = aNeu5Ac(2-3)bDGalp(1-3)bDGalNAc(1-4)[aNeu5Ac(2-6)]bDGalp(1-4)bDGlcp(1-
1)Cer
GD1b = bDGalp(1-3)bDGalNAc(1-4)[aNeu5Ac(2-8)aNeu5Ac(2-3)]bDGalp(1-4)bDGlcp(1-1)Cer
GT1a = aNeu5Ac(2-8)aNeu5Ac(2-3)bDGalp(1-3)bDGalNAc(1-4)[aNeu5Ac(2-3)]bDGalp(1-
4)bDGlcp(1-1)Cer
GT1, GT1b = aNeu5Ac(2-3)bDGalp(1-3)bDGalNAc(1-4)[aNeu5Ac(2-8)aNeu5Ac(2-3)]bDGalp(1-
4)bDGlcp(1-1)Cer
OAc-GT1b = aNeu5Ac(2-3)bDGalp(1-3)bDGalNAc(1-4)aXNeu5Ac9Ac(2-8)aNeu5Ac(2-
3)]bDGalp(1-4)bDGlcp(1-1)Cer
GT1c = bDGalp(1-3)bDGalNAc(1-4)[aNeu5Ac(2-8)aNeu5Ac(2-8)aNeu5Ac(2-3)]bDGalp(1-
4)bDGlcp(1-1)Cer
GT3 = aNeu5Ac(2-8)aNeu5Ac(2-8)aNeu5Ac(2-3)bDGal(1-4)bDGlc(1-1)CerGQ1b = aNeu5Ac(2-
8)aNeu5Ac(2-3)bDGalp(1-3)bDGalNAc(1-4)[aNeu5Ac(2-8)aNeu5Ac(2-3)]bDGalp(1-
4)bDGlcp(1-1)Cer
GGal = aNeu5Ac(2-3)bDGalp(1-1)Cer
where:
aNeu5Ac = 5-acetyl-alpha-neuraminic acid
aNeu5Ac9Ac = 5,9-diacetyl-alpha-neuraminic acid
bDGalp = beta-D-galactopyranose
bDGalpNAc = N-acetyl-beta-D-galactopyranose
bDGlcp = beta-D-glucopyranose
Cer = ceramide (general N-acylated sphingoid)

### CD33 Activities

Modulated Expression of Immune-Related Proteins

In some embodiments, anti-CD33 antibodies of the present disclosure may modulate expression of PD-L1, PD-L2, B7-H2, B7-H3, CD200R, CD163 and/or CD206 after binding to a CD33 protein expressed in a cell. Modulated (e.g., increased or decreased) expression may include, without limitation, modulation in gene expression, modulation in transcriptional expression, or modulation in protein expression. Any method known in the art for determining gene, transcript (e.g., mRNA), and/or protein expression may be used. For example, Northern blot analysis may be used to determine anti-inflammatory mediator gene expression levels, RT-PCR may be used to determine the level of anti-inflammatory mediator transcription, and Western blot analysis may be used to determine anti-inflammatory mediator protein levels.

As used herein, PD-L1, PD-L2, B7-H2, B7-H3, CD200R, CD163 and/or CD206 may have modulated expression if its expression in one or more cells of a subject treated with anti-CD33 antibodies of the present disclosure is modulated (e.g., increased or decreased) as compared to the expression of PD-L1, PD-L2, B7-H2, B7-H3, CD200R, CD163 and/or CD206 expressed in one or more cells of a corresponding subject that is not treated with the antibody. In some embodiments, an anti-CD33 antibody of the present disclosure may modulate PD-L1, PD-L2, B7-H2, B7-H3, CD200R, CD163 and/or CD206 expression in one or more cells of a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to PD-L1, PD-L2, B7-H3, CD200R, CD163 and/or CD206 expression in one or more cells of a corresponding subject that is not treated with the antibody. In other embodiments, an anti-CD33 antibody of the present disclosure modulates PD-L1, PD-L2, B7-H2, B7-H3, CD200R, CD163 and/or CD206 expression in one or more cells of a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to PD-L1, PD-L2, B7-H2, B7-H3, CD200R, CD163 and/or CD206 expression in one or more cells of a corresponding subject that is not treated with the antibody.

In some embodiments, anti-CD33 antibodies of the present disclosure are useful for preventing, lowering the risk of, or treating conditions and/or diseases associated with abnormal levels of PD-L1, PD-L2, B7-H2, B7-H3, CD200R, CD163 and/or CD206, including without limitation, dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, and cancer including bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express CD33, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza.

Enhancement or Normalization of the Ability of Bone Marrow-Derived Dendritic Cells to Induce Antigen-Specific T Cell Proliferation

In some embodiments, anti-CD33 antibodies of the present disclosure may enhance and/or normalize the ability of bone marrow-derived dendritic cells to induce antigen-specific T cell proliferation after binding to a CD33 protein expressed in a cell.

In some embodiments, antagonist anti-CD33 antibodies of the present disclosure may enhance and/or normalize the ability of bone marrow-derived dendritic cells to induce antigen-specific T cell proliferation in one or more bone marrow-derived dendritic cells of a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to the ability of bone marrow-derived dendritic cells to induce antigen-specific T cell proliferation in one or more bone marrow-derived dendritic cells of a corresponding subject that is not treated with the antibody. In other embodiments, an antagonist anti-CD33 antibody may enhance and/or normalize the ability of bone marrow-derived dendritic cells to induce antigen-specific T cell proliferation in one or more bone marrow-derived dendritic cells of a subject by at least at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to the ability of bone marrow-derived dendritic cells to induce antigen-specific T cell proliferation in one or more bone marrow-derived dendritic cells of a corresponding subject that is not treated with the antibody.

In some embodiments, anti-CD33 antibodies of the present disclosure are beneficial for preventing, lowering the risk of, or treating conditions and/or diseases associated with decreased or dysregulated ability of bone marrow-derived dendritic cells to induce antigen-specific T cell proliferation, including without limitation, dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, and cancer including bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express CD33, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza.

Proliferation and Survival of CD33-Expressing Cells

In some embodiments, anti-CD33 antibodies of the present disclosure may increase the proliferation, survival, and/or function of dendritic cells, macrophages, monocytes, neutrophils, osteoclasts, Langerhans cells of skin, Kupffer cells, T cells, T helper cells, cytotoxic T cells, and microglial cells after binding to CD33 protein expressed on a cell.

Microglial cells are a type of glial cell that are the resident macrophages of the brain and spinal cord, and thus act as the first and main form of active immune defense in the central nervous system (CNS). Microglial cells constitute 20% of the total glial cell population within the brain. Microglial cells are constantly scavenging the CNS for plaques, damaged neurons and infectious agents. The brain and spinal cord are considered “immune privileged” organs in that they are separated from the rest of the body by a series of endothelial cells known as the blood-brain barrier, which prevents most pathogens from reaching the vulnerable nervous tissue. In the case where infectious agents are directly introduced to the brain or cross the blood-brain barrier, microglial cells must react quickly to limit inflammation and destroy the infectious agents before they damage the sensitive neural tissue. Due to the unavailability of antibodies from the rest of the body (few antibodies are small enough to cross the blood brain barrier), microglia must be able to recognize foreign bodies, swallow them, and act as antigen-presenting cells activating T cells. Since this process must be done quickly to prevent potentially fatal damage, microglial cells are extremely sensitive to even small pathological changes in the CNS. They achieve this sensitivity in part by having unique potassium channels that respond to even small changes in extracellular potassium.

As used herein, macrophages of the present disclosure include, without limitation, M1 macrophages, activated M1 macrophages, and M2 macrophages. As used herein, microglial cells of the present disclosure include, without limitation, M1 microglial cells, activated M1 microglial cells, and M2 microglial cells.

In some embodiments, anti-CD33 antibodies of the present disclosure may increase the expression of CD80, CD83 and/or CD86 on dendritic cells, monocytes, and/or macrophages.

As used herein, the rate of proliferation, survival, and/or function of macrophages, dendritic cells, monocytes, T cells, neutrophils, and/or microglia may include increased expression if the rate of proliferation, survival, and/or function of dendritic cells, macrophages, monocytes, neutrophils, osteoclasts, Langerhans cells of skin, Kupffer cells, and/or microglia in a subject treated with an anti-CD33 antibody of the present disclosure is greater than the rate of proliferation, survival, and/or function of dendritic cells, macrophages, monocytes, osteoclasts, Langerhans cells of skin, Kupffer cells, T cells, neutrophils, and/or microglia in a corresponding subject that is not treated with the antibody. In some embodiments, an anti-CD33 antibody of the present disclosure may increase the rate of proliferation, survival, and/or function of dendritic cells, macrophages, monocytes, osteoclasts, Langerhans cells of skin, Kupffer cells, T cells, and/or microglia in a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to the rate of proliferation, survival, and/or function of dendritic cells, macrophages, monocytes, osteoclasts, Langerhans cells of skin, Kupffer cells, T cells, and/or microglia in a corresponding subject that is not treated with the antibody. In other embodiments, an anti-CD33 antibody of the present disclosure may increase the rate of proliferation, survival, and/or function of dendritic cells, macrophages, monocytes, osteoclasts, Langerhans cells of skin, Kupffer cells, T cells, and/or microglia in a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to the rate of proliferation, survival, and/or function of dendritic cells, macrophages, monocytes, osteoclasts, Langerhans cells of skin, Kupffer cells, T cells, and/or microglia in a corresponding subject that is not treated with the antibody.

In some embodiments, anti-CD33 antibodies of the present disclosure are beneficial for preventing, lowering the risk of, or treating conditions and/or diseases associated with a reduction in proliferation, survival, increased apoptosis and/or function of dendritic cells, neutrophils, macrophages, monocytes, osteoclasts, Langerhans cells of skin, Kupffer cells, T cells, and/or microglia including without limitation, dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, and cancer including bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express CD33, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza.

CD33-Dependent Activation of Immune Cells

In some embodiments, antagonist anti-CD33 antibodies of the present disclosure may increase the activity of cytotoxic T cells helper T cells or both. In some embodiments, antagonist anti-CD33 antibodies of the present disclosure are beneficial for preventing, lowering the risk of, or treating conditions and/or diseases associated with decreased activity of cytotoxic T cells helper T cells or both, including without limitation, tumors, including solid tumors such as bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, and thyroid cancer.

In some embodiments, antagonist anti-CD33 antibodies of the present disclosure may induce an increase in proliferation, survival, activity, and/or number of T cells, cytotoxic T cells, CD3⁺ T cells, helper T cells, dendritic cells, macrophages, monocytes, neutrophils, osteoclasts, Langerhans cells of skin, Kupffer cells, and/or microglial cells. In some embodiments, antagonist anti-CD33 antibodies of the present disclosure induce an increase in proliferation, survival, activity, and/or number of T cells, cytotoxic T cells, CD3⁺ T cells, helper T cells, dendritic cells, macrophages, monocytes, neutrophils, osteoclasts, Langerhans cells of skin, Kupffer cells, and/or microglial cells in the presence of myeloid-derived suppressor cells (MDSC).

As used herein, the rate of proliferation, survival, activity, and/or number of T cells, cytotoxic T cells, CD3⁺ T cells, helper T cells, dendritic cells, macrophages, monocytes, neutrophils, osteoclasts, Langerhans cells of skin, Kupffer cells, and/or microglial cells may include an increased rate if the rate of proliferation, survival, activity, and/or number of T cells, cytotoxic T cells, CD3⁺ T cells, helper T cells, dendritic cells, macrophages, monocytes, neutrophils, osteoclasts, Langerhans cells of skin, Kupffer cells, and/or microglial cells in a subject treated with an anti-CD33 antibody of the present disclosure is greater than the rate of proliferation, survival, activity, and/or number of T cells, cytotoxic T cells, CD3⁺ T cells, helper T cells, dendritic cells, macrophages, monocytes, neutrophils, osteoclasts, Langerhans cells of skin, Kupffer cells, and/or microglial cells in a corresponding subject that is not treated with the antibody. In some embodiments, an anti-CD33 antibody of the present disclosure may increase proliferation, survival, activity, and/or number of T cells, cytotoxic T cells, CD3⁺ T cells, helper T cells, dendritic cells, macrophages, monocytes, neutrophils, osteoclasts, Langerhans cells of skin, Kupffer cells, and/or microglial cells in a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to the level of proliferation, survival, activity, and/or number of T cells, cytotoxic T cells, CD3⁺ T cells, helper T cells, dendritic cells, macrophages, monocytes, neutrophils, osteoclasts, Langerhans cells of skin, Kupffer cells, and/or microglial cells in a corresponding subject that is not treated with the antibody. In other embodiments, an anti-CD33 antibody of the present disclosure may increase proliferation, survival, activity, and/or number of T cells, cytotoxic T cells, CD3⁺ T cells, helper T cells, dendritic cells, macrophages, monocytes, neutrophils, osteoclasts, Langerhans cells of skin, Kupffer cells, and/or microglial cells in a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to the level of proliferation, survival, activity, and/or number of T cells, cytotoxic T cells, CD3⁺ T cells, helper T cells, dendritic cells, macrophages, monocytes, neutrophils, osteoclasts, Langerhans cells of skin, Kupffer cells, and/or microglial cells in a corresponding subject that is not treated with the antibody.

CD33-Dependent Inhibition of Tumor-Associated Immune Cells

In some embodiments, agonist anti-CD33 antibodies of the present disclosure may decrease the activity, decrease the proliferation, decrease the survival, decrease the functionality, decrease infiltration to tumors or lymphoid organs (e.g., the spleen and lymph nodes), the number of CD14⁺ myeloid cells, decrease tumor growth rate, reduce tumor volume, reduce or inhibit differentiation, survival, and/or one or more functions of myeloid-derived suppressor cells (MDSC), and/or promote apoptosis of T-regulatory cells or inhibitory tumor-imbedded immunosuppressor dendritic cells or, tumor-associated macrophages or, myeloid-derived suppressor cells. In some embodiments, agonist anti-CD33 antibodies of the present disclosure are beneficial for preventing, lowering the risk of, or treating conditions and/or diseases associated with the activity of one or more type of immune suppressor cells, including without limitation, tumors, including solid tumors that do not express CD33 such as bladder cancer, brain cancer, breast cancer, colon cancer, rectal cancer, endometrial cancer, kidney cancer, renal cell cancer, renal pelvis cancer, lung cancer, melanoma, non-Hodgkin's lymphoma, pancreatic cancer, prostate cancer, ovarian cancer, fibrosarcoma, thyroid cancer, and blood tumors that express CD33, such as leukemia cells.

In some embodiments, antagonist anti-CD33 antibodies of the present disclosure may decrease the number of CD14⁺ myeloid cells, decrease tumor growth rate, reduce tumor volume, or reduce or inhibit differentiation, survival, and/or one or more functions of myeloid-derived suppressor cells (MDSC).

In some embodiments, an anti-CD33 antibody of the present disclosure may decrease the number of CD14⁺ myeloid cells, decrease tumor growth rate, reduce tumor volume, or reduce or inhibit differentiation, survival, and/or one or more functions of myeloid-derived suppressor cells (MDSC) in a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to the number of CD14⁺ myeloid cells, tumor growth rate, tumor volume, or level of differentiation, survival, and/or one or more functions of myeloid-derived suppressor cells (MDSC) in a corresponding subject that is not treated with the antibody. In other embodiments, an anti-CD33 antibody of the present disclosure, may decrease the number of CD14⁺ myeloid cells, decrease tumor growth rate, reduce tumor volume, or reduce or inhibit differentiation, survival, and/or one or more functions of myeloid-derived suppressor cells (MDSC) in a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to the number of CD14⁺ myeloid cells, tumor growth rate, tumor volume, or level of differentiation, survival, and/or one or more functions of myeloid-derived suppressor cells (MDSC) in a corresponding subject that is not treated with the antibody.

Increased Efficacy of Checkpoint Inhibitor Therapies

In some embodiments, antagonist anti-CD33 antibodies of the present disclosure may increase the efficacy of one or more checkpoint inhibitor therapies and/or immune-modulating therapies, such as PD-1 inhibitors or therapies that target one or more of CTL4, the adenosine pathway, PD-L1, PD-L2, OX40, TIM3, and/or LAG3.

In some embodiments, an anti-CD33 antibody of the present disclosure may increase the efficacy of one or more checkpoint inhibitor therapies and/or immune-modulating therapies, such as PD-1 inhibitors or therapies that target one or more of CTL4, the adenosine pathway, PD-L1, PD-L2, OX40, TIM3, and/or LAG3 in a subject receiving such one or more therapies by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to the level of effectiveness of one or more checkpoint inhibitor therapies and/or immune-modulating therapies, such as PD-1 inhibitors or therapies that target one or more of CTL4, the adenosine pathway, PD-L1, PD-L2, OX40, TIM3, and/or LAG3 in a corresponding subject receiving such one or more therapies that is not treated with the antibody. In other embodiments, an anti-CD33 antibody of the present disclosure may increase the efficacy of one or more checkpoint inhibitor therapies and/or immune-modulating therapies, such as PD-1 inhibitors or therapies that target one or more of CTL4, the adenosine pathway, PD-L1, PD-L2, OX40, TIM3, and/or LAG3 in a subject receiving such one or more therapies by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to the level of effectiveness of one or more checkpoint inhibitor therapies and/or immune-modulating therapies, such as PD-1 inhibitors or therapies that target one or more of CTL4, the adenosine pathway, PD-L1, PD-L2, OX40, TIM3, and/or LAG3 in a corresponding subject receiving such one or more therapies that is not treated with the antibody.

Increased Efficacy of Chemotherapeutic Agents

In some embodiments, antagonist anti-CD33 antibodies of the present disclosure may increase the efficacy of one or more chemotherapy agents, such as gemcitabine, capecitabine, anthracyclines, doxorubicin (Adriamycin®), epirubicin (Ellence®), taxanes, paclitaxel (Taxol®), docetaxel (Taxotere®), 5-fluorouracil (5-FU), cyclophosphamide (Cytoxan®), carboplatin (Paraplatin®), oxaliplatin (Elotaxin®), leucovorin, and/or temozolomide (Temodar®).

In some embodiments, an anti-CD33 antibody of the present disclosure may increase the efficacy of one or more chemotherapy agents in a subject receiving such one or more therapies by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to the level of effectiveness of one or more chemotherapy agents in a corresponding subject receiving such one or more therapies that is not treated with the antibody. In other embodiments, an anti-CD33 antibody of the present disclosure may increase the efficacy of one or more chemotherapy agents in a subject receiving such one or more therapies by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to the level of effectiveness of one or more chemotherapy agents in a corresponding subject receiving such one or more therapies that is not treated with the antibody.

### Antibody Preparation

Anti-CD33 antibodies of the present disclosure can encompass polyclonal antibodies, monoclonal antibodies, humanized and chimeric antibodies, human antibodies, antibody fragments (e.g., Fab, Fab′-SH, Fv, scFv, and F(ab′)₂), bispecific and polyspecific antibodies, multivalent antibodies, heteroconjugate antibodies, conjugated antibodies, library derived antibodies, antibodies having modified effector functions, fusion proteins containing an antibody portion, and any other modified configuration of the immunoglobulin molecule that includes an antigen recognition site, such as an epitope having amino acid residues of a CD33 protein of the present disclosure, including glycosylation variants of antibodies, amino acid sequence variants of antibodies, and covalently modified antibodies. The anti-CD33 antibodies may be human, murine, rat, or of any other origin (including chimeric or humanized antibodies).

(1) Polyclonal Antibodies

Polyclonal antibodies, such as polyclonal anti-CD33 antibodies, are generally raised in animals by multiple subcutaneous (sc) or intraperitoneal (ip) injections of the relevant antigen and an adjuvant. It may be useful to conjugate the relevant antigen (e.g., a purified or recombinant CD33 protein of the present disclosure) to a protein that is immunogenic in the species to be immunized, e.g., keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are independently lower alkyl groups. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The animals are immunized against the desired antigen, immunogenic conjugates, or derivatives by combining, e.g., 100 μg (for rabbits) or 5 μg (for mice) of the protein or conjugate with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to fourteen days later, the animals are bled and the serum is assayed for antibody titer. Animals are boosted until the titer plateaus. Conjugates also can be made in recombinant cell culture as protein fusions. Also, aggregating agents such as alum are suitable to enhance the immune response.

(2) Monoclonal Antibodies

Monoclonal antibodies, such as monoclonal anti-CD33 antibodies, are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the monoclonal anti-CD33 antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization (e.g., a purified or recombinant CD33 protein of the present disclosure). Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The immunizing agent will typically include the antigenic protein (e.g., a purified or recombinant CD33 protein of the present disclosure) or a fusion variant thereof. Generally peripheral blood lymphocytes (“PBLs”) are used if cells of human origin are desired, while spleen or lymph node cells are used if non-human mammalian sources are desired. The lymphoctyes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, particularly myeloma cells of rodent, bovine or human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors (available from the Salk Institute Cell Distribution Center, San Diego, Calif. USA), as well as SP-2 cells and derivatives thereof (e.g., X63-Ag8-653) (available from the American Type Culture Collection, Manassas, Va. USA). Human myeloma and mouse-human heteromyeloma cell lines have also been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen (e.g., a CD33 protein of the present disclosure). Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed against the desired antigen (e.g., a CD33 protein of the present disclosure). Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose chromatography, hydroxylapatite chromatography, gel electrophoresis, dialysis, affinity chromatography, and other methods as described above.

Anti-CD33 monoclonal antibodies may also be made by recombinant DNA methods, such as those disclosed in U.S. Pat. No. 4,816,567, and as described above. DNA encoding the monoclonal antibodies is readily isolated and sequenced using conventional procedures (e.g., by using oligonucleotide probes that specifically bind to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opin. Immunol., 5:256-262 (1993) and Phückthun, Immunol. Rev. 130:151-188 (1992).

In certain embodiments, anti-CD33 antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) described the isolation of murine and human antibodies, respectively, from phage libraries. Subsequent publications describe the production of high affinity (nanomolar (“nM”) range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies of desired specificity (e.g., those that bind a CD33 protein of the present disclosure).

The DNA encoding antibodies or fragments thereof may also be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein (e.g., anti-CD33 antibodies of the present disclosure or fragments thereof) may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid anti-CD33 antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

(3) Humanized Antibodies

Anti-CD33 antibodies of the present disclosure or antibody fragments thereof may further include humanized or human antibodies. Humanized forms of non-human (e.g., murine) antibodies are chimeric immunoglobulins, immunoglobulin chains or fragments thereof (such as Fab, Fab′-SH, Fv, scFv, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally will also comprise at least a portion of an immunoglobulin constant region (Fc), typically that of a human immunoglobulin. Jones et al., Nature 321: 522-525 (1986); Riechmann et al., Nature 332: 323-329 (1988) and Presta, Curr. Opin. Struct. Biol. 2: 593-596 (1992).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers, Jones et al., Nature 321:522-525 (1986); Riechmann et al., Nature 332:323-327 (1988); Verhoeyen et al., Science 239:1534-1536 (1988), or through substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody. Sims et al., J. Immunol., 151:2296 (1993); Chothia et al., J. Mol. Biol., 196:901 (1987). Another method uses a particular framework derived from the consensus sequence of all human antibodies of a particular subgroup of light or heavy chains. The same framework may be used for several different humanized antibodies. Carter et al., Proc. Nat'l Acad. Sci. USA 89:4285 (1992); Presta et al., J. Immunol. 151:2623 (1993).

Furthermore, it is important that antibodies be humanized with retention of high affinity for the antigen and other favorable biological properties. To achieve this goal, according to a preferred method, humanized antibodies are prepared by a process of analyzing the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen or antigens (e.g., CD33 proteins of the present disclosure), is achieved. In general, the CDR residues are directly and most substantially involved in influencing antigen binding.

Various forms of the humanized anti-CD33 antibody are contemplated. For example, the humanized anti-CD33 antibody may be an antibody fragment, such as an Fab, which is optionally conjugated with one or more cytotoxic agent(s) in order to generate an immunoconjugate. Alternatively, the humanized anti-CD33 antibody may be an intact antibody, such as an intact IgG1 antibody.

(4) Antibody Fragments

In certain embodiments there are advantages to using anti-CD33 antibody fragments, rather than whole anti-CD33 antibodies. Smaller fragment sizes allow for rapid clearance and better brain penetration.

Various techniques have been developed for the production of antibody fragments. Traditionally, these fragments were derived via proteolytic digestion of intact antibodies (see, e.g., Morimoto et al., J. Biochem. Biophys. Method. 24:107-117 (1992); and Brennan et al., Science 229:81 (1985)). However, these fragments can now be produced directly by recombinant host cells, for example, using nucleic acids encoding anti-CD33 antibodies of the present disclosure. Fab, Fv and scFv antibody fragments can all be expressed in and secreted from E. coli, thus allowing the straightforward production of large amounts of these fragments. A anti-CD33 antibody fragments can also be isolated from the antibody phage libraries as discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Production of Fab and F(ab′)₂ antibody fragments with increased in vivo half-lives are described in U.S. Pat. No. 5,869,046. In other embodiments, the antibody of choice is a single chain Fv fragment (scFv). See WO 93/16185; U.S. Pat. Nos. 5,571,894 and 5,587,458. The anti-CD33 antibody fragment may also be a “linear antibody,” e.g., as described in U.S. Pat. No. 5,641,870. Such linear antibody fragments may be monospecific or bispecific.

(5) Bispecific and Polyspecific Antibodies

Bispecific antibodies (BsAbs) are antibodies that have binding specificities for at least two different epitopes, including those on the same or another protein (e.g., one or more CD33 proteins of the present disclosure). Alternatively, one part of a BsAb can be armed to bind to the target CD33 antigen, and another can be combined with an arm that binds to a second protein. Such antibodies can be derived from full length antibodies or antibody fragments (e.g., F(ab′)₂ bispecific antibodies).

Methods for making bispecific antibodies are known in the art. Traditional production of full length bispecific antibodies is based on the coexpression of two immunoglobulin heavy-chain/light chain pairs, where the two chains have different specificities. Millstein et al., Nature, 305:537-539 (1983). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829 and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, C_{H}2, and C_{H}3 regions. It is preferred to have the first heavy-chain constant region (C_{H}1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only half of the bispecific molecules provides for an easy way of separation. This approach is disclosed in WO 94/04690. For further details of generating bispecific antibodies, see, for example, Suresh et al., Methods in Enzymology 121: 210 (1986).

According to another approach described in WO 96/27011 or U.S. Pat. No. 5,731,168, the interface between a pair of antibody molecules can be engineered to maximize the percentage of heterodimers which are recovered from recombinant cell culture. The preferred interface comprises at least a part of the C_{H}3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chains(s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175: 217-225 (1992) describes the production of fully humanized bispecific antibody F(ab′)₂ molecules. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bivalent antibody fragments directly from recombinant cell culture have also been described. For example, bivalent heterodimers have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. The “diabody” technology described by Hollinger et al., Proc. Nat'l Acad. Sci. USA, 90: 6444-6448 (1993) has provided an alternative mechanism for making bispecific/bivalent antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific/bivalent antibody fragments by the use of single-chain Fv (sFv) dimers has also been reported. See Gruber et al., J. Immunol., 152:5368 (1994).

Antibodies with more than two valencies are also contemplated. For example, trispecific antibodies can be prepared. Tutt et al., J. Immunol. 147:60 (1991).

Exemplary bispecific antibodies may bind to two different epitopes on a given molecule (e.g., a CD33 protein of the present disclosure). Alternatively, an arm targeting a CD33 signaling component may be combined with an arm which binds to a triggering molecule on a leukocyte such as a T cell receptor molecule (e.g., CD2, CD3, CD28 or B7), or Fc receptors for IgG (FcγR), such as FcγRI (CD64), FcγRII (CD32) and FcγRIII (CD16) so as to focus cellular defense mechanisms to the cell expressing the particular protein. Bispecific antibodies may also be used to localize cytotoxic agents to cells which express a particular protein. Such antibodies possess a protein-binding arm and an arm which binds a cytotoxic agent or a radionuclide chelator, such as EOTUBE, DPTA, DOTA or TETA. Another bispecific antibody of interest binds the protein of interest and further binds tissue factor (TF).

(6) Multivalent Antibodies

A multivalent antibody may be internalized (and/or catabolized) faster than a bivalent antibody by a cell expressing an antigen to which the antibodies bind. The anti-CD33 antibodies of the present disclosure or antibody fragments thereof can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises an Fc region or a hinge region. In this scenario, the antibody will comprise an Fc region and three or more antigen binding sites amino-terminal to the Fc region. The preferred multivalent antibody herein contains three to about eight, but preferably four, antigen binding sites. The multivalent antibody contains at least one polypeptide chain (and preferably two polypeptide chains), wherein the polypeptide chain or chains comprise two or more variable domains. For instance, the polypeptide chain or chains may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. Similarly, the polypeptide chain or chains may comprise VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain. The multivalent antibodies may recognize the CD33 antigen as well as, without limitation, additional antigens A beta peptide, antigen or an alpha synuclain protein antigen or, Tau protein antigen or, TDP-43 protein antigen or, prion protein antigen or, huntingtin protein antigen, or RAN, translation Products antigen, including the DiPeptide Repeats, (DPRs peptides) composed of glycine-alanine (GA), glycine-proline (GP), glycine-arginine (GR), proline-alanine (PA), or proline-arginine (PR), insulin receptor, insulin like growth factor receptor, transferrin receptor, or any other antigen that facilitates antibody transfer across the blood brain barrier.

(7) Heteroconjugate Antibodies

Heteroconjugate antibodies are also within the scope of the present disclosure. Heteroconjugate antibodies are composed of two covalently joined antibodies (e.g., anti-CD33 antibodies of the present disclosure or antibody fragments thereof). For example, one of the antibodies in the heteroconjugate can be coupled to avidin, the other to biotin. Such antibodies have, for example, been proposed to target immune system cells to unwanted cells, U.S. Pat. No. 4,676,980, and have been used to treat HIV infection. International Publication Nos. WO 91/00360, WO 92/200373 and EP 0308936. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980. Heteroconjugate antibodies may be made using any convenient cross-linking methods. Suitable cross-linking agents are well known in the art, and are disclosed in U.S. Pat. No. 4,676,980, along with a number of cross-linking techniques.

(8) Effector Function Engineering

It may also be desirable to modify an anti-CD33 antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRII, and/or FcγRIII. In some embodiments, the effector function is impaired by removing N-glycosylation of the Fc region (e.g., in the CH 2 domain of IgG) of the antibody. In some embodiments, the effector function is impaired by modifying regions such as 233-236, 297, and/or 327-331 of human IgG as described in PCT WO 99/58572 and Armour et al., Molecular Immunology 40: 585-593 (2003); Reddy et al., J. Immunology 164:1925-1933 (2000).

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

(9) Other Amino Acid Sequence Modifications

Amino acid sequence modifications of anti-CD33 antibodies of the present disclosure, or antibody fragments thereof, are also contemplated. For example, it may be desirable to improve the binding affinity and/or other biological properties of the antibodies or antibody fragments. Amino acid sequence variants of the antibodies or antibody fragments are prepared by introducing appropriate nucleotide changes into the nucleic acid encoding the antibodies or antibody fragments, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics (i.e., the ability to bind or physically interact with a CD33 protein of the present disclosure). The amino acid changes also may alter post-translational processes of the antibody, such as changing the number or position of glycosylation sites.

A useful method for identification of certain residues or regions of the anti-CD33 antibody that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with the target antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at the target codon or region and the expressed antibody variants are screened for the desired activity.

Amino acid sequence insertions include amino- (“N”) and/or carboxy- (“C”) terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in the Table D below under the heading of “preferred substitutions”. If such substitutions result in a change in biological activity, then more substantial changes, denominated “exemplary substitutions” in Table D, or as further described below in reference to amino acid classes, may be introduced and the products screened.

TABLE D
Amino acid substitutions

| Original | | Preferred |
|---|---|---|
| Residue | Exemplary Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln(Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | Ser | ser |
| Trp (W) | tyr; phe | tyr |
| Tyr(Y) | trp; phe; thr; ser | phe |
| Val (V) | ile; leu; met; phe; ala; norleucine | leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment, such as an Fv fragment).

A particularly preferred type of substitutional variant involves substituting one or more hypervariable region residues of a parent antibody (e.g. a humanized or human anti-CD33 antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and the antigen (e.g., a CD33 protein of the present disclosure). Such contact residues and neighboring residues are candidates for substitution according to the techniques elaborated herein. Once such variants are generated, the panel of variants is subjected to screening as described herein and antibodies with superior properties in one or more relevant assays may be selected for further development. Affinity maturation may also be performed by employing a yeast presentation technology such as that disclosed in, for example, WO2009/036379A2; WO2010105256; WO2012009568; and Xu et al., Protein Eng. Des. Sel., 26(10): 663-70 (2013).

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-IgE antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibodies (e.g., anti-CD33 antibodies of the present disclosure) or antibody fragments.

(10) Antibody Conjugates

Anti-CD33 antibodies of the present disclosure, or antibody fragments thereof, can be conjugated to a detectable marker, a toxin, or a therapeutic agent. Any suitable method known in the art for conjugating molecules, such as a detectable marker, a toxin, or a therapeutic agent to antibodies may be used.

For example, drug conjugation involves coupling of a biological active cytotoxic (anticancer) payload or drug to an antibody that specifically targets a certain tumor marker (e.g. a protein that, ideally, is only to be found in or on tumor cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cancer. Due to this targeting, ideally the drug has lower side effects and gives a wider therapeutic window than other chemotherapeutic agents. Technics to conjugate antibodies are disclosed are known in the art (see, e.g., Jane de Lartigue, OncLive Jul. 5, 2012; ADC Review on antibody-drug conjugates; and Ducry et al., (2010). Bioconjugate Chemistry 21 (1): 5-13).

In some embodiments, an anti-CD33 antibody of the present disclosure may be conjugated to a toxin selected from ricin, ricin A-chain, doxorubicin, daunorubicin, a maytansinoid, taxol, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Saponaria officinalis inhibitor, glucocorticoid, auristatin, auromycin, yttrium, bismuth, combrestatin, duocarmycins, dolastatin, cc1065, and a cisplatin.

(11) Other Antibody Modifications

Anti-CD33 antibodies of the present disclosure, or antibody fragments thereof, can be further modified to contain additional non-proteinaceous moieties that are known in the art and readily available. Preferably, the moieties suitable for derivatization of the antibody are water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc. Such techniques and other suitable formulations are disclosed in Remington: The Science and Practice of Pharmacy, 20th Ed., Alfonso Gennaro, Ed., Philadelphia College of Pharmacy and Science (2000).

Binding Assays and Other Assays

Anti-CD33 antibodies of the present disclosure may be tested for antigen binding activity, e.g., by known methods such as ELISA, surface plasmon resonance (SPR), Western blot, etc.

In some embodiments, competition assays may be used to identify an antibody that competes with any of the antibodies described herein. In some embodiments, competition assays may be used to identify an antibody that competes with any of the antibodies listed in Tables 1A-1C, 2A-3C, 3, 4, 5A-5D, and 6A-6D, or selected from AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, AB-64.1.15, AB-H2, AB-H9, AB-H14, AB-H15, AB-H63, AB-H64, AB-H65, or AB-H66 for binding to CD33. In certain embodiments, such a competing antibody binds to the same epitope (e.g., a linear or a conformational epitope) that is bound by any of the antibodies listed in Tables 1A-1C, 2A-2C, 3, 4, 5A-5D, and 6A-6D, or selected from AB-14.1, AB-14.2, AB-14.3, AB-14.4, AB-14.5, AB-14.6, AB-14.7, AB-14.8, AB-14.9, AB-14.10, AB-14.11, AB-63.4, AB-63.5, AB-63.6, AB-63.7, AB-63.8, AB-63.9, AB-63.10, AB-63.11, AB-63.12, AB-63.13, AB-63.14, AB-63.15, AB-63.16, AB-63.17, AB-63.18, AB-64.1, AB-64.2, AB-64.3, AB-64.4, AB-64.5, AB-64.6, AB-64.7, AB-64.8, AB-64.1.1, AB-64.1.2, AB-64.1.3, AB-64.1.4, AB-64.1.5, AB-64.1.6, AB-64.1.7, AB-64.1.8, AB-64.1.9, AB-64.1.10, AB-64.1.11, AB-64.1.12, AB-64.1.13, AB-64.1.14, AB-64.1.15, AB-H2, AB-H9, AB-H14, AB-H15, AB-H63, AB-H64, AB-H65, or AB-H66. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized CD33 or cells expressing CD33 on a cell surface are incubated in a solution comprising a first labeled antibody that binds to CD33 (e.g., human or non-human primate) and a second unlabeled antibody that is being tested for its ability to compete with the first antibody for binding to CD33. The second antibody may be present in a hybridoma supernatant. As a control, immobilized CD33 or cells expressing CD33 is incubated in a solution comprising the first labeled antibody but not the second unlabeled antibody. After incubation under conditions permissive for binding of the first antibody to CD33, excess unbound antibody is removed, and the amount of label associated with immobilized CD33 or cells expressing CD33 is measured. If the amount of label associated with immobilized CD33 or cells expressing CD33 is substantially reduced in the test sample relative to the control sample, then that indicates that the second antibody is competing with the first antibody for binding to CD33. See, Harlow and Lane (1988) Antibodies: A Laboratory Manual ch. 14 (Cold Spring Harbor Laboratory, Cold Spring Harbor, N.Y.).

### Nucleic Acids, Vectors, and Host Cells

Anti-CD33 antibodies of the present disclosure may be produced using recombinant methods and compositions, e.g., as described in U.S. Pat. No. 4,816,567. In some embodiments, isolated nucleic acids having a nucleotide sequence encoding any of the anti-CD33 antibodies of the present disclosure are provided. Such nucleic acids may encode an amino acid sequence containing the VL and/or an amino acid sequence containing the VH of the anti-CD33 antibody (e.g., the light and/or heavy chains of the antibody). In some embodiments, one or more vectors (e.g., expression vectors) containing such nucleic acids are provided. In some embodiments, a host cell containing such nucleic acid is also provided. In some embodiments, the host cell contains (e.g., has been transduced with): (1) a vector containing a nucleic acid that encodes an amino acid sequence containing the VL of the antibody and an amino acid sequence containing the VH of the antibody, or (2) a first vector containing a nucleic acid that encodes an amino acid sequence containing the VL of the antibody and a second vector containing a nucleic acid that encodes an amino acid sequence containing the VH of the antibody. In some embodiments, the host cell is eukaryotic, e.g., a Chinese Hamster Ovary (CHO) cell or lymphoid cell (e.g., Y0, NS0, Sp20 cell).

Methods of making an anti-CD33 antibody of the present disclosure are provided. In some embodiments, the method includes culturing a host cell of the present disclosure containing a nucleic acid encoding the anti-CD33 antibody, under conditions suitable for expression of the antibody. In some embodiments, the antibody is subsequently recovered from the host cell (or host cell culture medium).

For recombinant production of an anti-CD33 antibody of the present disclosure, a nucleic acid encoding the anti-CD33 antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors containing a nucleic acid sequence encoding any of the anti-CD33 antibodies of the present disclosure, or fragments thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid of the present disclosure. The expression vector may replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the nucleic acids of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell. In some embodiments, the vector contains a nucleic acid containing one or more amino acid sequences encoding an anti-CD33 antibody of the present disclosure.

Suitable host cells for cloning or expression of antibody-encoding vectors include prokaryotic or eukaryotic cells. For example, anti-CD33 antibodies of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523; and Charlton, Methods in Molecular Biology, Vol. 248 (B. K. C. Lo, ed., Humana Press, Totowa, N.J., 2003), pp. 245-254, describing expression of antibody fragments in E. coli). After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gerngross, Nat. Biotech. 22:1409-1414 (2004); and Li et al., Nat. Biotech. 24:210-215 (2006)).

Suitable host cells for the expression of glycosylated antibody can also be derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts (e.g., U.S. Pat. Nos. 5,959,177, 6,040,498, 6,420,548, 7,125,978, and 6,417,429, describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CVI); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N. Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### Pharmaceutical Compositions

Anti-CD33 antibodies of the present disclosure can be incorporated into a variety of formulations for therapeutic administration by combining the anti-CD33 antibodies with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols. Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

A pharmaceutical composition of the present disclosure can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and enhance solubility or uptake). Examples of such modifications or complexing agents include, without limitation, sulfate, gluconate, citrate and phosphate. The polypeptides of a composition can also be complexed with molecules that enhance their in vivo attributes. Such molecules include, without limitation, carbohydrates, polyamines, amino acids, other peptides, ions (e.g., sodium, potassium, calcium, magnesium, manganese), and lipids.

Further examples of formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in the brain or central nervous system. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of an anti-CD33 antibody of the present disclosure in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the present disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

Pharmaceutical Dosages

Pharmaceutical compositions of the present disclosure containing an anti-CD33 antibody of the present disclosure may be administered to an individual in need of treatment with the antibody, preferably a human, in accord with known methods, such as intravenous administration as a bolus or by continuous infusion over a period of time, by intramuscular, intraperitoneal, intracerobrospinal, intracranial, intraspinal, subcutaneous, intra-articular, intrasynovial, intrathecal, oral, topical, or inhalation routes.

Dosages and desired drug concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the anti-CD33 antibodies of the present disclosure, normal dosage amounts may vary from about 10 ng/kg up to about 100 mg/kg of an individual's body weight or more per day, preferably about 1 mg/kg/day to 10 mg/kg/day, depending upon the route of administration. For repeated administrations over several days or longer, depending on the severity of the disease, disorder, or condition to be treated, the treatment is sustained until a desired suppression of symptoms is achieved.

In some embodiments, the anti-CD33 antibody is administered at a dose of between about 1.6 mg/kg and about 15 mg/kg about once every twelve weeks or more frequently. In some embodiments, the anti-CD33 antibody is administered at a dose of between about 1.6 mg/kg and about 15 mg/kg about once every two weeks to about once every twelve weeks. In some embodiments, the anti-CD33 antibody is administered at a dose of about 1.6 mg/kg, about 5 mg/kg, about 7.5 mg/kg, about 10 mg/kg, or about 15 mg/kg. In some embodiments, the anti-CD33 antibody is administered once every two weeks, once every four weeks, once every five weeks, once every six weeks, once every seven weeks, once every eight weeks, once every nine weeks, once every ten weeks, once every eleven weeks, or once every twelve weeks. In some embodiments, the anti-CD33 antibody is administered once every two weeks at a dose of about 1.6 mg/kg. In some embodiments, the anti-CD33 antibody is administered once every four weeks at a dose of about 1.6 mg/kg. In some embodiments, the anti-CD33 antibody is administered once every four weeks at a dose of about 15 mg/kg. In some embodiments, the anti-CD33 antibody is administered once every five weeks at a dose of about 15 mg/kg. In some embodiments, the anti-CD33 antibody is administered once every six weeks at a dose of about 15 mg/kg. In some embodiments, the anti-CD33 antibody is administered once every seven weeks at a dose of about 15 mg/kg. In some embodiments, the anti-CD33 antibody is administered once every eight weeks at a dose of about 15 mg/kg.

In some embodiments, the anti-CD33 antibody is administered at a dose of between about 1.6 mg/kg to about 30 mg/kg (e.g., any of about 1.6 mg/kg, about 5 mg/kg, about 7.5 mg/kg, about 10 mg/kg, about 15 mg/kg, or about 30 mg/kg) on day 1 of a treatment period and once every twelve weeks or more frequently (e.g., any of once every two weeks, once every three weeks, once every four weeks, once every five weeks, once every six weeks, once every seven weeks, once every eight weeks, once every nine weeks, once every ten weeks, once every eleven weeks, or once every 12 weeks) thereafter. In some embodiments, the anti-CD33 antibody is administered at a dose of about 1.6 mg/kg on day 1 of a treatment period and once every two weeks thereafter. In some embodiments, the anti-CD33 antibody is administered at a dose of about 1.6 mg/kg on day 1 of a treatment period and once every four weeks thereafter. In some embodiments, the anti-CD33 antibody is administered at a dose of about 15 mg/kg on day 1 of a treatment period and once every four weeks thereafter. In some embodiments, the anti-CD33 antibody is administered at a dose of about 15 mg/kg on day 1 of a treatment period and once every five weeks thereafter. In some embodiments, the anti-CD33 antibody is administered at a dose of about 15 mg/kg on day 1 of a treatment period and once every six weeks thereafter. In some embodiments, the anti-CD33 antibody is administered at a dose of about 15 mg/kg on day 1 of a treatment period and once every eight weeks thereafter. In some embodiments, the anti-CD33 antibody is administered at a dose of about 15 mg/kg on day 1 of a treatment period and once every ten weeks thereafter. In some embodiments, the anti-CD33 antibody is administered at a dose of about 15 mg/kg on day 1 of a treatment period and once every twelve weeks thereafter.

Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg, about 100 μg/kg, about 300 μg/kg, about 1 mg/kg, and about 2/mg/kg) may be used. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the anti-CD33 antibody administered, can vary over time independently of the dose used.

Dosages for a particular anti-CD33 antibody may be determined empirically in individuals who have been given one or more administrations of the anti-CD33 antibody. Individuals are given incremental doses of an anti-CD33 antibody. To assess efficacy of an anti-CD33 antibody, a clinical symptom of any of the diseases, disorders, or conditions of the present disclosure (e.g., frontotemporal dementia, Alzheimer's disease, vascular dementia, seizures, retinal dystrophy, a traumatic brain injury, a spinal cord injury, long-term depression, atherosclerotic vascular diseases, and undesirable symptoms of normal aging) can be monitored.

Administration of an anti-CD33 antibody of the present disclosure can be continuous or intermittent, depending, for example, on the recipient's physiological condition, whether the purpose of the administration is therapeutic or prophylactic, and other factors known to skilled practitioners. The administration of an anti-CD33 antibody, may be essentially continuous over a preselected period of time or may be in a series of spaced doses.

Guidance regarding particular dosages and methods of delivery is provided in the literature; see, for example, U.S. Pat. Nos. 4,657,760; 5,206,344; or 5,225,212. It is within the scope of the present disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Kits/Articles of Manufacture

The present disclosure also provides kits and/or articles of manufacture containing an anti-CD33 antibody described herein, or a functional fragment thereof. Kits and/or articles of manufacture of the present disclosure may include one or more containers comprising a purified antibody of the present disclosure. In some embodiments, the kits and/or articles of manufacture further include instructions for use in accordance with the methods of this disclosure.

In some embodiments, these instructions comprise a description of administration of the anti-CD33 antibody described herein to prevent, reduce risk, or treat an individual having a disease, disorder, or injury selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, and cancer including bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express CD33, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza, according to any methods of this disclosure.

In some embodiments, the instructions comprise a description of how to detect a CD33 protein, for example in an individual, in a tissue sample, or in a cell. The kit and/or article of manufacture may further comprise a description of selecting an individual suitable for treatment based on identifying whether that individual has the disease and the stage of the disease.

In some embodiments, the kits and/or articles of manufacture may further include another antibody of the present disclosure (e.g., at least one antibody that specifically binds to an inhibitory checkpoint molecule, at least one antibody that specifically binds to an inhibitory cytokine, and/or at least one agonistic antibody that specifically binds to a stimulatory checkpoint protein) and/or at least one stimulatory cytokine. In some embodiments, the kits and/or articles of manufacture may further include instructions for using the antibody and/or stimulatory cytokine in combination with an anti-CD33 antibody described herein, instructions for using an anti-CD33 antibody described herein in combination with an antibody and/or stimulatory cytokine, or instructions for using an anti-CD33 antibody described herein and an antibody and/or stimulatory cytokine, according to any methods of this disclosure.

The instructions generally include information as to dosage, dosing schedule, and route of administration for the intended treatment. The containers may be unit doses, bulk packages (e.g., multi-dose packages) or sub-unit doses. Instructions supplied in the kits and/or articles of manufacture of the present disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, e.g., a disease of the present disclosure. Instructions may be provided for practicing any of the methods described herein.

The kits and/or articles of manufacture of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit and/or article of manufacture may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). At least one active agent in the composition is an anti-CD33 antibody described herein. The container may further comprise a second pharmaceutically active agent.

Kits and/or articles of manufacture may optionally provide additional components such as buffers and interpretive information. Normally, the kit comprises a container and a label or package insert(s) on or associated with the container.

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: Anti-CD33 Antibody Pharmacokinetics in Healthy Human Volunteers

This example describes a Phase 1a study according to the protocol described in Example 3 that examined the pharmacokinetics (PK) of intravenously administered anti-CD33 antibody AB-64.1.2 in humans.

### Materials and Methods

Healthy human volunteer subjects were administered a single-dose of anti-CD33 antibody AB-64.1.2 (or placebo control) as an intravenous infusion over approximately one hour. Antibody AB-64.1.2 dose levels used for these studies were 1.6 mg/kg, 5 mg/kg, 15 mg/kg, and 30 mg/kg. Each AB-64.1.2 dose cohort included 6 subjects.

Blood was drawn from the human subjects at multiple time-points to obtain anti-CD33 antibody concentrations in serum for measurement of pharmacokinetics. Anti-CD33 antibody concentrations were available up to 84 days post-dose for all cohorts up to and including the 5 mg/kg cohort; up to 56-days post-dose for the 15 mg/kg cohort; and up to 29-days post-dose for the 30 mg/kg cohort. Anti-CD33 antibody serum concentrations were assayed using an ELISA assay.

### Results

Serum PK data for anti-CD33 antibody AB-64.1.2 in healthy volunteers from each of the dose cohorts are provided in Table E.

TABLE E
Serum PK data for anti-CD33 antibody AB-64.1.2.

| | Cₘₐₓ | Vz | AUC_{inf} | CL | |
|---|---|---|---|---|---|
| Dose | (μg/mL) | (mL/kg) | (h*μg/mL) | (mL/h/kg) | T_{1/2} (h) |
| level | (CV %) | (CV %) | (CV %) | (CV %) | (CV %) |
| 1.6 mg/kg | 28.5 | 80.7 | 3276.5 | 0.508 | 113.5 |
| | (18.2) | (30.3) | (20.9) | (23.3) | (33.0) |
| 5 mg/kg | 86.2 | 92.2 | 13,731.1 | 0.375 | 180.0 |
| | (12.7) | (34.4) | (20.7) | (17.2) | (46.2) |
| 15 mg/kg | 386.5 | 92.9 | 57,706.9 | 0.264 | 246.4 |
| | (14.8) | (16.3) | (13.4) | (12.9) | (18.2) |
| 30 mg/kg | 773.1 | 86.8 | 106,670.6 | 0.286 | 217.4 |
| | (19.0) | (24.1) | (14.1) | (14.0) | (34.4) |

Cₘₐₓ: maximum concentration; Vz: apparent volume of distribution; AUC_{inf}: AUC from time 0 extrapolated to infinity; CL: clearance; T_{1/2}: terminal half-life; CV = coefficient of variation.

As shown in Table E, anti-CD33 antibody AB-64.1.2 administered to healthy human volunteers displayed an approximate dose proportional Cₘₐₓ. The data also showed that plasma terminal half-life of anti-CD33 antibody AB-64.1.2 was short at all doses tested, ranging from 113.5 hours (4.73 days) at the 1.6 mg/kg dose to 246.4 hours (10.27 days) at the 15 mg/kg dose.

### Conclusions

The results presented in this Example indicated that at the doses tested, anti-CD33 antibody AB-64.1.2 was cleared more rapidly than other therapeutic antibodies of similar class, thus demonstrating that, unexpectedly, anti-CD33 antibody AB-64.1.2 showed a short terminal half-life compared to other antibodies of similar class (Ovacik, M and Lin, L, (2018) Clin Transl Sci 11, 540-552). The short terminal half-life of AB-64.1.2 suggested that the antibody may not be useful therapeutically.

### Example 2: Effect of Anti-CD33 Antibody AB-64.1.2 on Monocyte CD33 Levels in Healthy Human Volunteers

This example describes a Phase 1a study according to the protocol described in Example 3 that examined the effect of anti-CD33 antibody AB-64.1.2 on monocyte CD33 expression levels in humans.

### Materials and Methods

Healthy human volunteers were administered a single intravenous dose of anti-CD33 antibody AB-64.1.2 at a dose of either 1.6 mg/kg or 15 mg/kg. Six individuals were administered the anti-CD33 antibody at each dose and 2 individuals were administered placebo control antibody at each dose.

Whole blood was collected from each individual on days 1, 4, 7, 12, 17, 29, 42, 56, and 84 after administration of the anti-CD33 antibody.

Changes in CD33 expression levels on monocytes were determined using flow cytometry. Since Mean Fluorescence Intensity (MFI) can be variable across instruments or across measurements made on the same instrument over time, Molecules of Equivalent Soluble Fluorochrome (MESF) was used as a standardized unit of fluorescence intensity. MESF accounts for variability observed in mean fluorescence intensity (e.g., quenching, spectra shifts, extinction coefficients) and allows flow cytometry measurements to be compared quantitatively over time and across different instruments (See Schwartz et al., 2004, Cytometry Part B (Clinical Cytometry), 57B:1-6.) In the flow cytometry analyses presented in this Example, monocytes were gated as CD11b+ CD14+ CD16- HLA-DR+ and were selected as single, viable cells. Granulocytes were gated as CD11b+ CD16+ CD66+. Flow cytometry of the blood samples was performed at 360BioLabs (Melbourne, Australia).

### Results

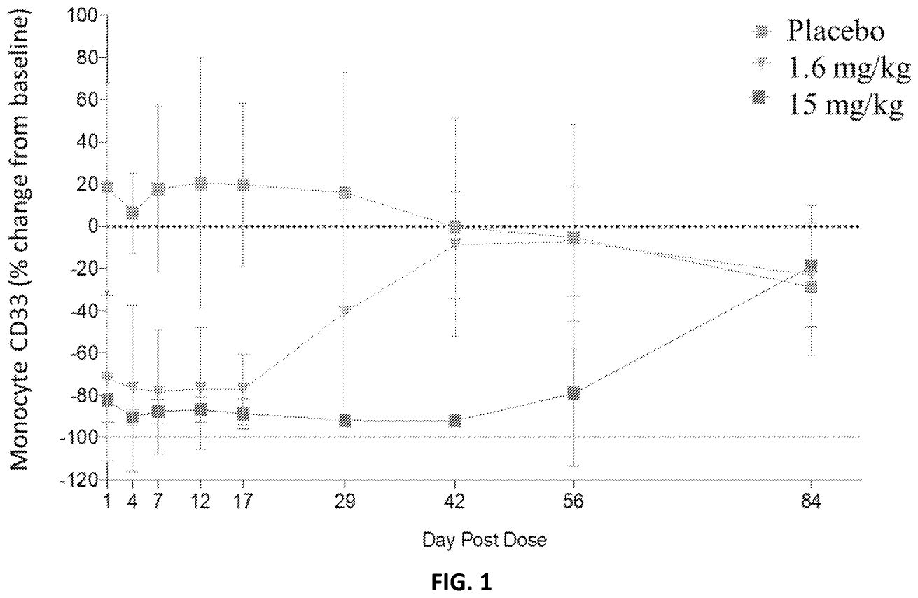

The percent change from baseline of monocyte CD33 levels from day 1 to day 84 after administration of the anti-CD33 antibody to healthy human volunteers was measured from samples of whole blood. As shown in FIG. 1, monocyte CD33 levels decreased rapidly following administration of anti-CD33 antibody at either the 1.6 mg/kg or 15 mg/kg doses.

In particular, monocyte CD33 levels decreased by approximately 70% by day 1 post-administration from baseline in human subjects given a single dose of 1.6 mg/kg of the antibody. The reduction in monocyte CD33 levels remained at levels of approximately 70-75% below that of baseline levels for at least 17 days post-administration of the single 1.6 mg/kg dose. Additionally, healthy human subjects administered a single anti-CD33 antibody AB-64.1.2 dose of 15 mg/kg exhibited a decrease in monocyte CD33 levels of approximately 80-90% below baseline levels for at least 56 days post-administration. The total number of monocytes in whole blood samples did not decrease, indicating that the reduction in CD33 levels observed was not due to a reduction in monocyte numbers by, for example, monocyte death.

### Conclusions

The results presented in this Example show that anti-CD33 antibody AB-64.1.2 causes a dose-dependent and long-lasting decrease in peripheral monocyte CD33 levels.

In view of the short terminal half-life of anti-CD33 antibody AB-64.1.2 of approximately 113.5 hours (4.73 days) after a single dose of 1.6 mg/kg, or of approximately 246.6 hours (10.27 days) after a single dose of 15 mg·kg (See Example 1 and Table E), the long-lasting reduction of monocyte CD33 levels of about 70-90% below that of baseline levels following administration of a single dose of AB-64.1.2 to healthy human subjects was unexpected and surprising.

### Example 3: A Phase 1 Study Evaluating the Safety, Tolerability, Pharmacokinetics, Pharmacodynamics, and Immunogenicity of Single and Multiple Doses of Anti-CD33 Antibody AB-64.1.2 in Healthy Participants and in Participants with Mild to Moderate Alzheimer's Disease

This Example describes a multi-center, randomized, double-blind, placebo-controlled, dose escalation first in human (FIH) study in healthy adults and in patients with mild to moderate Alzheimer's disease. The study is designed to systematically assess the safety (including immunogenicity) and tolerability, pharmacokinetics (PK), and pharmacodynamics (PD) of anti-CD33 antibody AB-64.1.2 when administered as single ascending doses in healthy participants and as multiple doses in patients with mild to moderate Alzheimer's disease.

### Primary Objective

The primary objective of this study is to evaluate safety, tolerability, pharmacokinetics, and pharmacodynamics of anti-CD33 antibody AB-64.1.2 administered in single ascending doses in healthy participants and multiple doses in patients with mild to moderate Alzheimer's disease.

### Study Design

The study is conducted in 2 phases, as described below.

### Single Ascending Dose Phase

In the single ascending dose (SAD) phase, up to 49 healthy adult participants are sequentially enrolled in 8 predefined cohorts (cohorts A to H). Three initial cohorts are conducted in 1-3 healthy volunteer participants (all receive active drug, i.e., anti-CD33 antibody AB-64.1.2), then each subsequent cohort has 8 healthy volunteer participants per cohort (6 active: 2 placebo). Additional open label cohorts are added to further assess safety, tolerability, and PD effects in the cerebrospinal fluid (CSF) at alternate timepoints, with up to 8 participants per cohort.

Dosing of the SAD cohort is as shown in Table F.

TABLE F
Dosing regimen for single ascending dose (SAD) cohorts A-H.

| | | Number of Participants | |
|---|---|---|---|
| | | Active (anti-CD33 | |
| SAD | | antibody AB- | |
| Cohort* | Dose (mg/kg) | 64.1.2) | Placebo |
| A | 0.05 | 1-3 | 0 |
| B | 0.2 | 1-3 | 0 |
| C | 0.6 | 1-3 | 0 |
| D | 1.6 | 6 | 2 |
| E | 5 | 6 | 2 |
| F | 15 | 6 | 2 |
| G | 30 | 6 | 2 |
| H | 60 | 6 | 2 |

*Additional open label cohorts are added to assess further safety and tolerability, with up to 8 participants per cohort.

The SAD healthy volunteer phase of the study consists of a screening period, study (treatment) period, follow up visits, and a final follow up/study completion visit. Screening occurs within 4 weeks prior to enrollment and the first administered dose of study drug on Day 1. All SAD participants are followed for 12 weeks after infusion. Participants in a designated cerebrospinal fluid (CSF) cohort (i.e., SAD cohorts E, F, G and H) undergo lumbar punctures at screening, on Day 8 and on Day 18 (±1 day), or on a day determined by PK and PD data from previous single dose cohorts where applicable.

Additional single dose cohorts are added as open label cohorts of up to 8 participants per cohort at dose levels not exceeding 15 mg/kg. Participants in these cohorts undergo lumbar punctures at screening (at least 4 days prior to study drug infusion), on Day 8 and on Day 18 (+1 day), or on a day determined by preliminary PK and PD data from previous single dose cohorts where applicable.

### Multiple Dose Phase

In the multiple dose (MD) phase, approximately 12 patients with mild to moderate Alzheimer's disease are enrolled and randomized in 1 cohort (Cohort I; 10 active: 2 placebo).

The MD cohort is initiated only after safety and tolerability up to and including the Day 13 visit for the last participant in the SAD phase has been evaluated. Anti-CD33 antibody AB-64.1.2 is administered via IV infusion in two doses given 4 weeks apart (q4w×2). The dose level used for the 2 study drug infusions is 15 mg/kg, which has been deemed tolerable and is expected to not exceed minimum exposures seen in the 30 mg/kg HV cohort (Cohort G).

The MD phase of the study in patients with mild to moderate Alzheimer's disease consists of a screening period, study (treatment) period, follow up visits and a final follow up/study completion visit.

Screening occurs within 6 weeks prior to enrolment and the first administered dose of study drug on Day 1.

Patients are administered the Mini-Mental State Examination (MMSE), Repeatable Battery for the Assessment of Neuropsychological Status (RBANS), and Clinical Dementia Rating (CDR) and undergo an MRI (including but not limited to fluid-attenuated inversion recovery [FLAIR] and T2* weighted GRE sequences) assessment of the brain. The screening MRI occurs as close to the beginning of the screening window as possible and at least 10 days prior to randomization on Day 1.

A lumbar puncture to obtain a CSF baseline sample is performed.

Amyloid-positron emission tomography (PET) imaging is performed in all participants. Participants may also participate in an optional exploratory assessment to evaluate changes in the brain as measured by translocator protein (TSPO)-PET.

Following the first IV infusion of study drug on Day 1 and discharge on Day 2, participants return to the study site as outpatients on Days 8, 15 and 22 (+2 days) for safety assessment follow up and on Day 29 (+1 day) to be administered the second dose of study drug.

Lumbar puncture to obtain CSF is performed on Day 50 and Day 64 (+2 days), or on a day determined by PK and PD data from previous single dose cohorts. Participants are followed for 16 weeks after the last infusion day.

Amyloid-PET imaging is performed in all participants on Day 106 (−2 days; +14 days). TSPO-PET imaging may be performed as an optional assessment on Day 36 (+10 days). A brain MRI is scheduled for all participants on Day 43 (+2 days).

### Eligibility Criteria

Inclusion Criteria

Subjects that meet the following criteria are included in the SAD Phase of this study:
Patients in the SAD cohorts are healthy adults ages 18-65 years.

Subjects that meet the following criteria are included in the MD Phase of this study:
Adults ages 50-85 years.
Clinical diagnosis of probable Alzheimer's disease dementia based on National Institute on Aging Alzheimer's Association criteria.
Screening MMSE score of 16-28 points, inclusive.
Screening Clinical Dementia Rating-Global Score (CDR-GS) of 0.5, 1.0, or 2.0.
Positive amyloid-PET scan by qualitative read, as defined in the PET Imaging Charter.
If already taking cholinesterase inhibitor and/or memantine therapy for Alzheimer's disease, on a stable dose for at least 4 weeks prior to screening, with no intent to initiate, discontinue, or alter the dose of any therapy for Alzheimer's disease for the duration of the study.

### Exclusion Criteria

Subjects are excluded from this study if they meet the following criteria:
Carriers of two copies of the minor allele rs12459419T.
History or presence of CNS or systemic autoimmune disorders including but not limited to rheumatoid arthritis, multiple sclerosis, lupus erythematosus, anti-phospholipid antibody syndrome, Behçet disease.
Dementia due to a condition other than Alzheimer's disease, including, but not limited to, Frontotemporal Dementia, Parkinson's disease, dementia with Lewy bodies, Huntington disease, or vascular dementia.
History or presence of clinically evident vascular disease potentially affecting the brain (e.g., clinically significant carotid, vertebral stenosis or plaque; aortic aneurysm; intracranial aneurysm; cerebral hemorrhage; arteriovenous malformation) that has the potential to affect cognitive function.
History or presence of stroke within the past 2 years or documented history of transient ischemic attack within the last 12 months.
History of severe, clinically significant (persistent neurologic deficit or structural brain damage) CNS trauma (e.g., cerebral contusion).
MRI evidence of
   More than two lacunar infarcts;
   Any territorial infarct >1 cm³; or
   Significant FLAIR hyperintense lesions in the cerebral white matter that may contribute to cognitive dysfunction.

### Study Duration

The duration of study participation for each participant in the SAD cohorts is about 16 weeks, including up to 4 weeks of screening, a single treatment on Day 1 and follow up period, culminating in a final study completion visit on Day 85 (+5 days).

The duration of study participation for each participant in the MD cohort is about 26 weeks, including up to 6 weeks of screening, multiple treatments (two doses administered 4 weeks apart [q4w×2]) and a follow up period, culminating in a final study completion visit on Day 141 (±5 days).

### Administration of Study Drug

Anti-CD33 antibody AB-64.1.2 is administered by intravenous (IV) infusion over about 60 minutes. The rate of infusion is adjusted in the event of an infusion related reaction. Placebo for IV infusion is commercially available normal saline.

### Study Outcome Assessments

Pharmacokinetics Outcomes

Pharmacokinetic outcome measures for the study include:
Serum concentration of anti-CD33 antibody AB-64.1.2 at specified time points.
Relationship between serum concentration or PK parameters for anti-CD33 antibody AB-64.1.2 and safety endpoints.
Relationship between serum, CSF concentration, or PK parameters for anti-CD33 antibody AB-64.1.2 and activity or PD endpoints (relationship with activity is an endpoint only for the MD cohort i.e., patients with Alzheimer's disease).

Clinical Outcomes

Exploratory clinical outcome measures (for the MD cohort only, i.e., patients with Alzheimer's disease) include:
Clinical Dementia Rating (CDR) Sum of Boxes (CDR-SB) score (change after dosing relative to baseline).
Mini-Mental State Examination (MMSE) score (change after dosing relative to baseline).
Repeatable Battery for the Assessment of Neuropsychological Status (RBANS) score (change after dosing relative to baseline).

Washington University's CDR is a global assessment instrument that yields global scores (i.e., CDR-GS). The sum of boxes (i.e., CDR-SB) score is a detailed quantitative general index that provides more information than the CDR-GS in patients with mild dementia (O'Bryant et al., (2010) Arch Neurol, 67(6):746-49). The CDR characterizes 6 domains of cognitive and functional performance applicable to Alzheimer's disease and related dementias: memory, orientation, judgment and problem solving, community affairs, home and hobbies, and personal care. The necessary information to make each rating is obtained through a semi-structured interview of the patient and a reliable informant or collateral source (e.g., a caregiver).

The MMSE is a brief test used to screen for cognitive impairment. It is routinely used for estimating the severity of cognitive impairment and tracking cognitive changes in an individual over time. The MMSE assesses orientation (time and place), registration, attention and calculation, recent memory, language (naming, comprehension and repetition), and constructional praxis (copying a FIGURE). The maximum total score is 30, with a higher score indicating better cognitive performance.

The RBANS is a collection of 12 subtests representing 5 neurocognitive domains: Immediate Memory, Visuospatial/Constructional, Language, Attention, and Delayed Memory. The raw scores from each subtest within a domain are converted to a summary score, or Index Score, for the domain by consulting normative data tables. The RBANS also provides an overall Index Score that summarizes the patient's overall level of performance on this measure.

Pharmacodynamics Outcomes

Pharmacodynamics biomarkers are assessed, including the following:
Blood-based biomarkers:
   Cell surface expression of CD33.
   Soluble CD33 (sCD33) in plasma.
   Markers of neuroinflammation in blood.
   Cell surface expression of relevant biomarkers/antigens.
CSF-based biomarkers:
   sCD33.
   CSF biomarkers relevant to Alzheimer's disease, e.g., Abeta, Tau, p-Tau, neurofilament light chain [NF-L], neurogranin, and YKL-40).
   Other relevant markers of neuroinflammation.
Genetic markers relevant to the disease indication including the following:
   ApoE4.
   TREM2 variants, CD33 variants, TMEM106b variants, CLUSTERIN variants.
Imaging biomarkers (for MD cohort only, i.e., patients with Alzheimer's disease):
   Magnetic resonance imaging (MRI).
   Amyloid-positon emission tomography (PET) (in all Alzheimer's disease patients).
   Translocator protein (TSPO)-PET.
Analysis of exploratory biomarker endpoints for the study include:
   Changes in expression levels of cell surface CD33.
   Changes in levels of sCD33 in plasma and CSF after dosing relative to baseline concentration.
   Changes in expression of cell surface antigens.
   Relationship between biomarkers at baseline, including common and rare genetic variants, identified through whole genome sequencing (WGS) performed on deoxyribonucleic acid (DNA) extracted from blood, and safety, PK, activity, immunogenicity, or other biomarker endpoints (relationship with activity is an endpoint only for the MD patient cohort, i.e., patients with Alzheimer's disease).
   Changes in brain amyloid burden as assessed by Amyloid-PET in the MD patient cohort only, i.e., patients with Alzheimer's disease.
   Changes in brain inflammation as assessed by translocator protein (TSPO)-PET.
   Changes in markers of neuroinflammation and disease process in CSF and plasma.
Biomarkers analyzed by whole genome sequencing include the following:
   Apolipoprotein E4 (ApoE4).
   TREM2 variants, CD33 variants, TMEM106b variants, CLUSTERIN variants.

Safety and Tolerability

The safety and tolerability endpoints of this study include:
Incidence, nature, and severity of serious adverse events (SAEs)
Incidence of dose limiting adverse events (DLAEs).
Incidence of adverse events (AEs) of Special Interest (AESIs), including the following:
   New or worsening brain edema.
   New cerebral micro-hemorrhages.
   Grade 2 or higher AEs considered potentially CD33-mediated.
Incidence of treatment discontinuations due to AEs.
Incidence of dose reductions due to AEs.
Mean changes in clinical laboratory tests from baseline over time; incidence of treatment emergent abnormal laboratory values and abnormal laboratory values reported as AEs.
Physical and neurologic examination abnormalities.
Mean change in vital signs from baseline over time and incidence of abnormal vital sign measurements.
Suicidal ideation, suicidal behavior, and self-injurious behavior without suicidal intent, as determined using the Sheehan-STS (for the MD patient cohort only).
Incidence of anti-drug antibodies (ADAs) during the study relative to the prevalence of ADAs at baseline (in SAD healthy adult participant cohorts and in the MD patient cohort).

### Statistical Methods

Analysis Populations

The statistical analysis populations include the following:

Treatment Received population: The treatment received population includes all randomized participants and is based on the treatment/dose level received.

Safety population: The Safety population includes all randomized participants who receive any amount of study drug (anti-CD33 antibody AB-64.1.2 or placebo) and is based on the actual treatment/dose level received, if this differs to what the participant is randomized to.

PK population: The PK population includes all randomized participants who receive any amount of active study drug (anti-CD33 antibody AB-64.1.2) with sufficient plasma concentration-time data to determine at least one PK parameter. Participants who receive only placebo are excluded from the PK population.

PD population: The PD population includes all randomized participants who receive any amount of study drug (anti-CD33 antibody AB-64.1.2 or placebo), with results from baseline and from at least one post-baseline PD assessment and is based on the actual treatment/dose level received, if this differs from what the participant was randomized to.

Pharmacokinetics

Individual and mean serum anti-CD33 antibody AB-64.1.2 concentration-time data is tabulated and plotted by cohort/dose level. PK parameters are computed from the individual serum anti-CD33 antibody AB-64.1.2 concentrations using a non-compartmental approach. The PK parameters estimated include:
Maximum drug concentration (Cₘₐₓ).
Time to reach Cmax (Tₘₐₓ).
Area under the drug concentration-time curve from time zero to the last quantifiable concentration (AUC₍₀₋ₗₐₛₜ₎).
Area under the drug concentration-time curve from time zero to infinity (AUC_{(0-inf)}) calculated as the sum of AUC₍₀₋ₗₐₛₜ₎ plus the last measurable plasma concentration divided by elimination rate constant (kₑₗ).
Areas under the drug concentration-time curve over the inter-dosing interval (AUCₜₐᵤ) where tau is the time over the inter-dosing interval. Calculated for the MD cohort only.
Apparent terminal elimination rate constant (kₑₗ) calculated by linear regression of the terminal linear portion of the log concentration vs. time curve.
Apparent terminal half-life (t_{1/2}).
Apparent total body clearance after extravascular administration (SAD cohorts: CL; MD cohort CLss), calculated as Dose/AUC_{0-inf} for single/first dose and Dose/AUCₜₐᵤ after multiple dose administrations.
Apparent total volume of distribution at the terminal phase after extravascular administration (SAD cohorts: Vz; MD cohort: Vzss), calculated as Dose/(kₑₗ×AUC_{0-inf}) after single/first dose and Dose/(kₑₗ×AUCₜₐᵤ) after multiple dose administrations.

Values for kₑₗ, t_{1/2}, AUC_{0-inf}, CL or Vz are not reported for cases that fail to exhibit a terminal log-linear phase in the concentration versus time profile.

Estimates for PK parameters are tabulated and summarized by descriptive statistics (mean, standard deviation [SD], median, minimum, and maximum, coefficient of variation [CV %], geometric mean and 90% confidence interval [CI], and geometric CV %).

Individual and mean anti-CD33 antibody AB-64.1.2 CSF concentration-time data are tabulated by cohort/dose level.

Potential correlations of relevant PK parameters with dose, demographics, safety (including QT changes), and PD measures are explored. Additional modelling, including population PK analysis, to characterize these correlations is performed.

Exploratory Clinical Outcomes

Individual exploratory clinical outcome measures for CDR-SB, MMSE and RBANS are presented in a data listing for all participants (for the MD cohort only, i.e. patients with Alzheimer's disease). CDR-SB, MMSE and RBANS are summarized by time point and treatment group (active or placebo) and a summary of change from baseline by treatment group, is presented.

Pharmacodynamics and Exploratory Biomarkers

All individual PD biomarker data are presented in data listings and summarized by nominal sampling time point, treatment group and cohort with descriptive statistics (e.g., number of non-missing observations, arithmetic mean, SD, median, minimum, maximum and % CV). The number of values below the limit of quantitation (BLQ) are presented. Observed change from baseline and percent changes from baseline for PD biomarker parameters are summarized separately for the single dosing cohorts and the multiple dosing cohort.

Exploratory analyses of biomarkers are conducted to evaluate the effect of anti-CD33 antibody AB-64.1.2 on exploratory biomarkers. In addition, exploratory biomarkers are analyzed before and after dosing with anti-CD33 antibody AB-64.1.2 to determine the relationship between PK exposure and biomarker levels.

TABLE 1A
EU or Kabat heavy chain HVR H1
sequences of anti-CD33 antibodies

| | | SEQ |
|---|---|---|
| | | ID |
| Ab(s) | HVR H1 | NO: |
| AB-H14; AB-H63; | GYTFTDYNLH | 105 |
| AB-63.6; AB-63.7; | | |
| AB-63.13; AB-63.14; | | |
| AB-H64; AB-64.1; | | |
| AB-64.2; AB-64.3; | | |
| AB-64.4; AB-64.1.1; | | |
| AB-64.1.2; AB-64.1.3; | | |
| AB-64.1.4; AB-64.1.5; | | |
| AB-64.1.6; AB-64.1.7; | | |
| AB-64.1.8; AB-64.1.9; | | |
| AB-64.1.10; AB-64.1.11; | | |
| AB-64.1.12; AB-64.1.13; | | |
| AB-64.1.14; and | | |
| AB-64.1.15 | | |
| AB-14.1 | GATFTDYNFH | 106 |
| AB-14.2 | GATFTDYNYH | 107 |
| AB-14.3; AB-14.4; | GYTFTDYNYH | 108 |
| AB-14.5; AB-14.6; | | |
| AB-14.7; AB-14.8; | | |
| AB-14.9; AB-14.10; | | |
| AB-14.11; AB-63.4; | | |
| AB-63.15; AB-63.16; | | |
| AB-63.17; AB-64.5; | | |
| AB-64.6; AB-64.7; | | |
| and AB-64.8 | | |
| AB-63.5 | GYTFTDYNNH | 109 |
| AB-63.8 | GVTFTDYNYH | 110 |
| AB-63.9 | GYAFTDYNLH | ill |
| AB-63.10 | GYTETDYNLH | 112 |
| AB-63.11 and AB-63.12 | GYTFTDYNFH | 113 |
| AB-63.18 | GYTHTDYNLH | 114 |
| Formula I | GX₁X₂X₃TDYNX₄H | 152 |
| | X₁ is Y, A, or V | |
| | X₂ is T or A | |
| | X₃ is F, E, or H | |
| | X₄ is L, F, Y, or | |
| | N | |

TABLE IB
EU or Kabat heavy chain HVR H2
sequences of anti-CD33 antibodies

| | | SEQ |
|---|---|---|
| | | ID |
| Ab(s) | HVR H2 | NO: |
| AB-H14; AB-14.1; | FIYPSNGITG | 115 |
| AB-14.2; AB-14.3; | | |
| AB-14.4; AB-14.5; | | |
| AB-14.6; AB-14.7; | | |
| AB-14.8; AB-14.9; | | |
| AB-14.10; AB-14.11; | | |
| AB-H63; AB-63.4; | | |
| AB-63.5; AB-63.6; | | |
| AB-63.7; AB-63.8; | | |
| AB-63.9; AB-63.10; | | |
| AB-63.12; AB-63.14; | | |
| AB-63.15; AB-63.16; | | |
| AB-63.17; AB-63.18; | | |
| AB-H64; AB-64.1; | | |
| AB-64.2; AB-64.3; | | |
| AB-64.4; AB-64.5; | | |
| AB-64.6; AB-64.7; | | |
| AB-64.8; AB-64.1.4; | | |
| AB-64.1.5; AB-64.1.6; | | |
| and AB-64.1.7 | | |
| AB-63.11 | FIYPANGITG | 116 |
| AB-63.13 | FIYPSNGIRG | 117 |
| AB-64.1.1; AB-64.1.8; | FIYPSNQITG | 118 |
| and AB-64.1.9 | | |
| AB-64.1.2; AB-64.1.10; | FIYPSNRITG | 119 |
| AB-64.1.11; and | | |
| AB-64.1.12 | | |
| AB-64.1.3; AB-64.1.13; | FIYPSNVITG | 120 |
| AB-64.1.14; and AB- | | |
| 64.1.15 | | |
| Formula II | FIYPX₁NX₂IX₃G | 153 |
| | X₁ is S or A | |
| | X₂ is G, Q, | |
| | R, or V | |
| | X₃ is T or R | |

TABLE 1C
EU or Kabat heavy chain HVR H3
sequences of anti-CD33 antibodies

| | | SEQ |
|---|---|---|
| | | ID |
| Ab(s) | HVR H3 | NO: |
| AB-H14; AB-14.4; AB-H63; | STVDYFDY | 121 |
| AB-63.4; AB-63.6; AB-63.7; | | |
| AB-63.8; AB-H64; and | | |
| AB-64.2 | | |
| AB-14.1; AB-14.3; | SDVDYFDY | 122 |
| AB-14.5; AB-14.6; | | |
| AB-14.7; AB-14.8; | | |
| AB-14.9; AB-14.10; | | |
| AB-63.5; AB-63.9; | | |
| AB-63.10; AB-63.11; | | |
| AB-63.13; AB-63.14; | | |
| AB-63.16; AB-63.17; | | |
| AB-63.18; AB-64.1; | | |
| AB-64.4; AB-64.5; | | |
| AB-64.6; AB-64.7; | | |
| AB-64.8; AB-64.1.1; | | |
| AB-64.1.2; AB-64.1.3; | | |
| AB-64.1.4; AB-64.1.5; | | |
| AB-64.1.6; AB-64.1.7; | | |
| AB-64.1.8; AB-64.1.9; | | |
| AB-64.1.10; AB-64.1.11; | | |
| AB-64.1.12; AB-64.1.13; | | |
| AB-64.1.14; | | |
| and AB-64.1.15 | | |
| AB-14.2 and AB-64.3 | SFVDYFDY | 123 |
| AB-14.11 | SSVDYFDY | 124 |
| AB-63.12 | STVDYFDD | 125 |
| AB-63.15 | SDVDYFDL | 126 |
| Formula III | SX₁VDYFDX₂ | 154 |
| | X₁ is T, D, | |
| | F, or S | |
| | X₂ is Y, | |
| | D, or L | |

TABLE 2A
EU or Kabat light chain HVR L1
sequences of anti-CD33 antibodies

| | | SEQ |
|---|---|---|
| | | ID |
| Ab(s) | HVR L1 | NO: |
| AB-H14; AB-14.1; | RASQSVSTSTYSYMH | 127 |
| AB-H63; AB-63.4; | | |
| AB-63.5; AB-63.13; | | |
| AB-63.18; AB-H64; | | |
| AB-64.1; AB-64.1.1; | | |
| AB-64.1.2; AB-64.1.3; | | |
| AB-64.1.4; AB-64.1.5; | | |
| AB-64.1.6; AB-64.1.7; | | |
| AB-64.1.8; AB-64.1.9; | | |
| AB-64.1.10; AB-64.1.11; | | |
| AB-64.1.12; AB-64.1.13; | | |
| AB-64.1.14; and | | |
| AB-64.1.15 | | |
| AB-14.6; AB-14.7; | RASQSVGTSTYSYMH | 128 |
| AB-14.8; AB-14.9; | | |
| AB-64.5; | | |
| and AB-64.7 | | |
| AB-14.10 | RASQSVSASTYSYMH | 129 |
| AB-14.2, AB-14.3; | RASQDVSTSTYSYMH | 130 |
| AB-14.4; AB-14.5; AB- | | |
| 14.11; AB-63.6; | | |
| AB-63.7; AB-63.8; | | |
| AB-63.9; | | |
| AB-63.10; AB-63.11; | | |
| AB-63.12; AB-63.15; | | |
| AB-64.3; AB-64.4; | | |
| AB-64.6; and AB-64.8 | | |
| AB-63.14 | KASQDVSTSTYSYMH | 131 |
| AB-63.16 | RASQSVHTSTYSYMH | 132 |
| AB-63.17 | RGSQSVSTSTYSYMH | 133 |
| AB-64.2 | RVSQDVSTSTYSYMH | 134 |
| Formula IV | X₁X₂SQX₃VX₄X₅S | 155 |
| | TYSYMH | |
| | X₁ is R or K | |
| | X₂ is A, G, or V | |
| | X₃ is S or D | |
| | X₄ is S, G, or H | |
| | X₅ is T or A | |

TABLE 2B
EU or Kabat light chain HVR L2
sequences of anti-CD33 antibodies

| | | SEQ |
|---|---|---|
| | | ID |
| Ab(s) | HVR L2 | NO: |
| AB-H14; AB-14.1; | YASNLES | 135 |
| AB-14.2; AB-14.5; | | |
| AB-14.7; AB-14.10; | | |
| AB-H63; AB-63.4; | | |
| AB-63.5; AB-63.6; | | |
| AB-63.8; AB-63.10; | | |
| AB-63.11; AB-63.13; | | |
| AB-63.14; AB-63.16; | | |
| AB-H64; AB-64.1; | | |
| AB-64.2; AB-64.5; | | |
| AB-64.8; AB-64.1.1; | | |
| AB-64.1.2; AB-64.1.3; | | |
| AB-64.1.4; AB-64.1.5; | | |
| AB-64.1.6; AB-64.1.7; | | |
| AB-64.1.8; AB-64.1.9; | | |
| AB-64.1.10; AB-64.1.11; | | |
| AB-64.1.12; AB-64.1.13; | | |
| AB-64.1.14; | | |
| and AB-64.1.15 | | |
| AB-14.3; AB-14.4; | YVSNLES | 136 |
| and AB-14.11 | | |
| AB-14.6 | YASALES | 137 |
| AB-14.8 | YASNLGS | 138 |
| AB-14.9 | YAVNLES | 139 |
| AB-63.7 | YAFNLES | 140 |
| AB-63.9; AB-64.3; | YASYLES | 141 |
| and AB-64.4 | | |
| AB-63.12 and AB-63.15 | YASNVES | 142 |
| AB-63.17 and AB-63.18 | YESNLES | 143 |
| AB-64.6 | YASFLES | 144 |
| AB-64.7 | YASNLNS | 145 |
| Formula V | YX₁X2X₃X₄X₅S | 156 |
| | X₁ is A, V, or E | |
| | X₂ is S, V, or F | |
| | X₃ is N, A, Y, or F | |
| | X₄ is L or V | |
| | X₅ is E, G, or N | |

TABLE 2C
EU or Kabat light chain HVR L3
sequences of anti-CD33 antibodies

| | | SEQ |
|---|---|---|
| | | ID |
| Ab(s) | HVR L3 | NO: |
| AB-H14; AB-14.1; | QHSWEIPLT | 146 |
| AB-14.2; AB-14.3; | | |
| AB-14.4; AB-14.5; | | |
| AB-14.6; AB-14.7; | | |
| AB-14.8; AB-14.9; | | |
| AB-14.10; AB-14.11; | | |
| AB-H63; AB-63.4; | | |
| AB-63.5; AB-63.6, | | |
| AB-63.8; AB-63.11; | | |
| AB-63.14; AB-63.15; | | |
| AB-63.16; AB-63.17; | | |
| AB-63.18; AB-H64; | | |
| AB-64.1; AB-64.2; | | |
| AB-64.5; AB-64.6; | | |
| AB-64.8; AB-64.1.1; | | |
| AB-64.1.2; AB-64.1.3; | | |
| AB-64.1.4; AB-64.1.5; | | |
| AB-64.1.6; AB-64.1.7; | | |
| AB-64.1.8; AB-64.1.9; | | |
| AB-64.1.10; AB-64.1.11; | | |
| AB-64.1.12; AB-64.1.13; | | |
| AB-64.1.14; and | | |
| AB-64.1.15 | | |
| AB-63.7 | QHSWEIPLE | 147 |
| AB-63.9; AB-64.3; | EHSWEIPLT | 148 |
| and AB-64.4 | | |
| AB-63.10 | QHSWELPLT | 149 |
| AB-63.12 | QHSWAIPLT | 150 |
| AB-63.13 and | QHSEEIPLT | 151 |
| AB-64.7 | | |
| Formula VI | X₁HSX₂X₃X₄ | 157 |
| | PLX₅ | |
| | X₁ is Q or E | |
| | X₂ is W or E | |
| | X₃ is E or A | |
| | X₄ is I or L | |
| | X₅ is T or E | |

TABLE 3
EU or Kabat heavy chain variable region sequences of anti-CD33 antibodies

| Ab(s) | HCVR | SEQ ID NO: |
|---|---|---|
| AB-14.1 | QVQLVQSGAEVKKPGASVKVSCKASGATFTDYNFHWVRQAPGQG | 34 |
| | LEWIGFIYPSNGITGYAQDFQGRVTMTVDTSTSTVYMELSSLRS | |
| | EDTAVYYCARSDVDYFDYWGQGTLVTVSS | |
| AB-14.2 | QVQLVQSGAEVKKPGASVKVSCKASGATFTDYNYHWVRQAPGQG | 35 |
| | LEWIGFIYPSNGITGYAQKFQDRVTMTVDTSTSTVYMELSSLRS | |
| | EDTAVYYCARSFVDYFDYWGQGTLVTVSS | |
| AB-14.3 | QVQLVQSGAEVKKPGASVKVSCKASGYTFTDYNYHWVRQAPGQG | 36 |
| | LEWIGFIYPSNGITGSAQKFQGRVTMTVDTSTSTVYMELSSLRS | |
| | EDTAVYYCARSDVDYFDYWGQGTLVTVSS | |
| AB-14.4 | QVQLVQSGAEVKKPGASVKVSCKASGYTFTDYNYHWVRQAPGQG | 37 |
| | LEWIGFIYPSNGITGYAQKFQGRVTMTVDTSTSTVYMELSSLRS | |
| | EDTAVYYCADSTVDYFDYWGQGTLVTVSS | |
| AB-14.5; AB-14.6; | QVQLVQSGAEVKKPGASVKVSCKASGYTFTDYNYHWVRQAPGQG | 38 |
| AB-14.7; | LEWIGFIYPSNGITGYAQKFQGRVTMTVDTSTSTVYMELSSLRS | |
| AB-14.8; AB-14.9; | EDTAVYYCARSDVDYFDYWGQGTLVTVSS | |
| AB-14.10, AB-63.16; | | |
| and AB-63.17 | | |
| AB-14.11 | QVQLVQSGAEVKKPGASVKVSCKASGYTFTDYNYHWVRQAPGQG | 39 |
| | LEWIGFIYPSNGITGYAQKFQGRVTMTVDTSTSTVYMELSSLRS | |
| | EDTAVYYCARSSVDYFDYWGQGTLVTVSS | |
| AB-63.4 | QVQLVQSGAEVKKPGASVKVSCKASGYTFTDYNYHWVRQAPGQG | 40 |
| | LEWIGFIYPSNGITGYAQKFQGRVTMTVDTSTSTVYMELSSLRS | |
| | EDTAVYYCARSTVDYFDYWGQGTLVTVSS | |
| AB-63.5 | QVQLVQSGAEVKKPGASVKVSCKASGYTFTDYNNHWVRQAPGQG | 41 |
| | LEWIGFIYPSNGITGYAQKFQGRVTMTVDTSTSTVYMELSSLRS | |
| | EDTAVYYCARSDVDYFDYWGQGTLVTVSS | |
| AB-H14; AB-H63; | QVQLVQSGAEVKKPGASVKVSCKASGYTFTDYNLHWVRQAPGQG | 42 |
| AB-63.6; AB-63.7; | LEWIGFIYPSNGITGYAQKFQGRVTMTVDTSTSTVYMELSSLRS | |
| and AB-H2 | EDTAVYYCARSTVDYFDYWGQGTLVTVSS | |
| AB-63.8 | QVQLVQSGAEVKKPGSSVKVSCKASGVTFTDYNYHWVRQAPGQG | 43 |
| | LEWIGFIYPSNGITGYAQKFQDRVTMTVDTSTSTVYMELSSLRS | |
| | EDTAVYYCARSTVDYFDYWGQGTLVTVSS | |
| AB-63.9 | QVQLVQSGAEVKKPGASVKVSCKASGYAFTDYNLHWVRQAPGQG | 44 |
| | LEWIGFIYPSNGITGYAQKFQGRVTMTVDTSTSTVYMELSSLRS | |
| | EDTAVYYCARSDVDYFDYWGQGTLVTVSS | |
| AB-63.10 | QVQLVQSGAEVKKPGASVKVSCKASGYTETDYNLHWVRQAPGQG | 45 |
| | LEWIGFIYPSNGITGYAQKFQGRVTMTVDTSTSTVYMELSSLRS | |
| | EDTAVYYCARSDVDYFDYWGQGTLVTVSS | |
| AB-63.11 | QVQLVQSGAEVKKPGASVKVSCKASGYTFTDYNFHWVRQAPGQG | 46 |
| | LEWIGFIYPANGITGYAQKDQGRVTMTVDTSTSTVYMELSSLRS | |
| | EDTAVYYCARSDVDYFDYWGQGTLVTVSS | |
| AB-63.12 | QVQLVQSGAEVKKPGASVKVSCKASGYTFTDYNFHWVRQAPGQG | 47 |
| | LEWIGFIYPSNGITGYAQKFTGRVTMTVDTSTSTVYMELSSLRS | |
| | EDTAVYYCARSTVDYFDDWGQGTLVTVSS | |
| AB-63.13 | QVQLVQSGAEVKKPGASVKVSCKASGYTFTDYNLHWVRQAPGQG | 48 |
| | LEWIGFIYPSNGIRGYAQKFQGRVTMTVDTSTSTVYMELSSLRS | |
| | EDTAVYYCARSDVDYFDYWGQGTLVTVSS | |
| AB-63.14 | QVQLVQSGAEVKKPGSSVKVSCKASGYTFTDYNLHWVRQAPGQG | 49 |
| | LEWIGFIYPSNGITGYAQKFQGRVTMTVDTSTSTVYMELSSLRS | |
| | EDTAVYYCARSDVDYFDYWGQGTLVTVSS | |
| AB-63.15 | QVQLVQSGAEVKKPGASVKVSCKASGYTFTDYNYHWVRQAPGQG | 50 |
| | LEWIGFIYPSNGITGYAQKFQGRVTMTVDTSTSTVYMELSSLRS | |
| | EDTAVYYCARSDVDYFDLWGQGTLVTVSS | |
| AB-63.18 | QVQLVQSGAEVKKPGSSVKVSCKASGYTHTDYNLHWVRQAPGQG | 51 |
| | LEWIGFIYPSNGITGYAQKFQGRVTMTVDTSTSTVYMELSSLRS | |
| | EDTAVYYCARSDVDYFDYWGQGTLVTVSS | |
| AB-64.1; AB-64.4 | QVQLVQSGAEVKKPGASVKISCKASGYTFTDYNLHWVRQAPGQG | 52 |
| | LEWIGFIYPSNGITGYAQKFQGRATLTVDNSTSTAYMELSSLRS | |
| | EDTAVYYCARSDVDYFDYWGQGTLLTVSS | |
| AB-H64 and AB- | QVQLVQSGAEVKKPGASVKISCKASGYTFTDYNLHWVRQAPGQG | 53 |
| 64.2 | LEWIGFIYPSNGITGYAQKFQGRATLTVDNSTSTAYMELSSLRS | |
| | EDTAVYYCARSTVDYFDYWGQGTLLTVSS | |
| AB-64.3 | QVQLVQSGAEVKKPGASVKISCKASGYTFTDYNLHWVRQAPGQG | 54 |
| | LEWIGFIYPSNGITGYAEKFEGRATLTVDNSTSTAYMELSSLRS | |
| | EDTAVYYCARSFVDYFDYWGQGTLLTVSS | |
| AB-64.5 | QVQLVQSGAEVKKPGASVKISCKASGYTFTDYNYHWVRQAPGQG | 55 |
| | LEWIGFIYPSNGITGYAQKFFGRATLTVDNSTSTAYMELSSLRS | |
| | EDTAVYYCARSDVDYFDYWGQGTLLTVSS | |
| AB-64.6 and AB- | QVQLVQSGAEVKKPGASVKISCKASGYTFTDYNYHWVRQAPGQG | 56 |
| 64.7 | LEWIGFIYPSNGITGYAQKFQGRATLTVDNSTSTAYMELSSLRS | |
| | EDTAVYYCARSDVDYFDYWGQGTLLTVSS | |
| AB-64.8 | QVQLVQSGAEVKKPGASVKISCKASGYTFTDYNYHWVRQAPGQG | 57 |
| | LEWIGFIYPSNGITGYAQKFQHRATLTVDNSTSTAYMELSSLRS | |
| | EDTAVYYCARSDVDYFDYWGQGTLLTVSS | |
| AB-64.1.1 | QVQLVQSGAEVKKPGASVKISCKASGYTFTDYNLHWVRQAPGQG | 58 |
| | LEWIGFIYPSNQITGYAQKFQGRATLTVDNSTSTAYMELSSLRS | |
| | EDTAVYYCARSDVDYFDYWGQGTLLTVSS | |
| AB-64.1.2 | QVQLVQSGAEVKKPGASVKISCKASGYTFTDYNLHWVRQAPGQG | 59 |
| | LEWIGFIYPSNRITGYAQKFQGRATLTVDNSTSTAYMELSSLRS | |
| | EDTAVYYCARSDVDYFDYWGQGTLLTVSS | |
| AB-64.1.3 | QVQLVQSGAEVKKPGASVKISCKASGYTFTDYNLHWVRQAPGQG | 60 |
| | LEWIGFIYPSNVITGYAQKFQGRATLTVDNSTSTAYMELSSLRS | |
| | EDTAVYYCARSDVDYFDYWGQGTLLTVSS | |
| AB-64.1.4 | QVQLVQSGAEVKKPGASVKISCKASGYTFTDYNLHWVRQAPGQG | 61 |
| | LEWIGFIYPSNGITGYAQKFQGRATLTVDTSTSTAYMELSSLRS | |
| | EDTAVYYCARSDVDYFDYWGQGTLLTVSS | |
| AB-64.1.5 | QVQLVQSGAEVKKPGASVKISCKASGYTFTDYNLHWVRQAPGQG | 62 |
| | LEWIGFIYPSNGITGYAQKFQGRATLTVDQSTSTAYMELSSLRS | |
| | EDTAVYYCARSDVDYFDYWGQGTLLTVSS | |
| AB-64.1.6 | QVQLVQSGAEVKKPGASVKISCKASGYTFTDYNLHWVRQAPGQG | 63 |
| | LEWIGFIYPSNGITGYAQKFQGRATLTVDNSASTAYMELSSLRS | |
| | EDTAVYYCARSDVDYFDYWGQGTLLTVSS | |
| AB-64.1.7 | QVQLVQSGAEVKKPGASVKISCKASGYTFTDYNLHWVRQAPGQG | 64 |
| | LEWIGFIYPSNGITGYAQKFQGRATLTVDNPTSTAYMELSSLRS | |
| | EDTAVYYCARSDVDYFDYWGQGTLLTVSS | |
| AB-64.1.8 | QVQLVQSGAEVKKPGASVKISCKASGYTFTDYNLHWVRQAPGQG | 65 |
| | LEWIGFIYPSNQITGYAQKFQGRATLTVDNSASTAYMELSSLRS | |
| | EDTAVYYCARSDVDYFDYWGQGTLLTVSS | |
| AB-64.1.9 | QVQLVQSGAEVKKPGASVKISCKASGYTFTDYNLHWVRQAPGQG | 66 |
| | LEWIGFIYPSNQITGYAQKFQGRATLTVDNPTSTAYMELSSLRS | |
| | EDTAVYYCARSDVDYFDYWGQGTLLTVSS | |
| AB-64.1.10 | QVQLVQSGAEVKKPGASVKISCKASGYTFTDYNLHWVRQAPGQG | 67 |
| | LEWIGFIYPSNRITGYAQKFQGRATLTVDTSTSTAYMELSSLRS | |
| | EDTAVYYCARSDVDYFDYWGQGTLLTVSS | |
| AB-64.1.11 | QVQLVQSGAEVKKPGASVKISCKASGYTFTDYNLHWVRQAPGQG | 68 |
| | LEWIGFIYPSNRITGYAQKFQGRATLTVDQSTSTAYMELSSLRS | |
| | EDTAVYYCARSDVDYFDYWGQGTLLTVSS | |
| AB-64.1.12 | QVQLVQSGAEVKKPGASVKISCKASGYTFTDYNLHWVRQAPGQG | 69 |
| | LEWIGFIYPSNRITGYAQKFQGRATLTVDNSASTAYMELSSLRS | |
| | EDTAVYYCARSDVDYFDYWGQGTLLTVSS | |
| AB-64.1.13 | QVQLVQSGAEVKKPGASVKISCKASGYTFTDYNLHWVRQAPGQG | 70 |
| | LEWIGFIYPSNVITGYAQKFQGRATLTVDTSTSTAYMELSSLRS | |
| | EDTAVYYCARSDVDYFDYWGQGTLLTVSS | |
| AB-64.1.14 | QVQLVQSGAEVKKPGASVKISCKASGYTFTDYNLHWVRQAPGQG | 71 |
| | LEWIGFIYPSNVITGYAQKFQGRATLTVDQSTSTAYMELSSLRS | |
| | EDTAVYYCARSDVDYFDYWGQGTLLTVSS | |
| AB-64.1.15 | QVQLVQSGAEVKKPGASVKISCKASGYTFTDYNLHWVRQAPGQG | 72 |
| | LEWIGFIYPSNVITGYAQKFQGRATLTVDNSASTAYMELSSLRS | |
| | EDTAVYYCARSDVDYFDYWGQGTLLTVSS | |
| AB-H9 and AB-H71 | QVQLVQSGAELKKPGASVKVSCKASGYTFTDYNLHWVRQAPGQR | 73 |
| | LEWIGFIYPSNGITGYSQKFQGKATLTVDTSASTAYMELSSLRS | |
| | EDTAVYYCARSTVDYFDYWGQGTTVTVSS | |
| AB-H3 and AB-H15 | QVQLVQSGAEVKKPGASVKISCKASGYTFTDYNLHWVRQAPGQG | 74 |
| | LEWIGFIYPSNGITGYAQKFQGRATLTVDTSTSTAYMELSSLRS | |
| | EDTAVYYCARSTVDYFDYWGQGTLLTVSS | |
| AB-H65 | QVQLVQSGAEVKKPGASVKVSCKASGYTFTDYNLHWVRQAPGQG | 75 |
| | LEWMGFIYPSNGITGYAQKFQGRVTMTRDTSTSTVYMELSSLRS | |
| | EDTAVYYCARSTVDYFDYWGQGTTVTVSS | |
| AB-H66 | QVQLVQSGAEVKKPGASVKISCKASGYTFTDYNLHWVRQAPGQG | 76 |
| | LEWIGFIYPSNGITGYAQKFQGRATLTVDTSTSTAYMELSSLRS | |
| | EDTAVYYCARSTVDYFDYWGQGTTVTVSS | |
| AB-H1 | QVQLVQSGAEVKKPGASVKVSCKASGYTFTDYNLHWVRQAPGQG | 170 |
| | LEWMGFIYPSNGITGYAQKFQGRVTMTRDTSTSTVYMELSSLRS | |
| | EDTAVYYCARSTVDYFDYWGQGTLVTVSS | |
| AB-H6 | QVQLVQSGAEVKKPGASVKVSCKASGYTFTDYNLHWVRQAPGQG | 171 |
| | LEWMGFIYPSNGITGYAQKFQGRVTMTVDTSTSTAYMELSSLRS | |
| | EDTAVYYCARSTVDYFDYWGQGTTVTVSS | |
| AB-H11 | QVQLVQSGAEVKKPGASVKVSCKASGYTFTDYNLHWVRQAPGQS | 172 |
| | LEWIGFIYPSNGITGYSQKFQGKATLTVDTSASTAYMELSSLRS | |
| | EDTAVYYCARSTVDYFDYWGQGTTVTVSS | |
| AB-H22 | QVQLVQSGAEVKKPGASVKISCKASGYTFTDYNLHWVRQAPGQR | 173 |
| | LEWIGFIYPSNGITGYNQKFKNKATLTVDTSASTAYMELSSLRS | |
| | EDTAVYYCARSTVDYFDYWGQGTTVTVSS | |
| AB-H24 | QVQLVQSGAEVKKPGASVKISCKASGYTFTDYNLHWVRQAPGQR | 174 |
| | LEWIGFIYPSNGITGYSQKFQGKATLTVDTSASTAYMELSSLRS | |
| | EDTAVYYCARSTVDYFDYWGQGTTVTVSS | |
| AB-H26 | QVQLVQSGAEVKKPGASVKVSCKASGYTFTDYNLHWVRQAPGQR | 175 |
| | LEWIGFIYPSNGITGYSQKFQGRATLTVDTSASTAYMELSSLRS | |
| | EDTAVYYCARSTVDYFDYWGQGTTVTVSS | |

TABLE 4
EU or Kabat light chain variable region sequences of anti-CD33 antibodies

| Ab(s) | LCVR | SEQ ID NO: |
|---|---|---|
| AB-H14; AB-14.1; | DIQMTQSPSSLSASVGDRVTITCRASQSVSTSTYSYMHWYQQKP | 77 |
| AB-H15; AB-22; | GKAPKLLIKYASNLESGVPSRFSGSGSGTDFTLTISSLQPEDFA | |
| AB-24; and AB-26 | TYYCQHSWEIPLTFGQGTKLEIK | |
| AB-14.2 | DIQMTQSPSSLSASVGDRVTITCRASQDVSTSTYSYMHWYQQKP | 78 |
| | GKAPKLLIKYASNLESGVPSRFSGSGSGTDFTLTISSLQPEDFA | |
| | TYYCQHSWEIPLTFGQGTKLEIK | |
| AB-14.3; AB-14.4; | DIQMTQSPSSLSASVGDRVTITCRASQDVSTSTYSYMHWYQQKP | 79 |
| and AB-14.11 | GKAPKLLIKYVSNLESGVPSRFSGSGSGTDFTLTISSLQPEDFA | |
| | TYYCQHSWEIPLTFGQGTKLEIK | |
| AB-14.5 | DIQMTQSPSSLSASVGDRVTITCRASQDVSTSTYSYMHWYQRKP | 80 |
| | GKAPKLLIKYASNLESGVPSRFSGSGSGTDFTLTISSLQPEDFA | |
| | TYYCQHSWEIPLTFGQGTKLEIK | |
| AB-14.6 | DIQMTQSPSSLSASVGDRVTITCRASQSVGTSTYSYMHWYQQKP | 81 |
| | GKAPKLLIKYASALESGVPSRFSGSGSGTDFTLTISSLQPEDFA | |
| | TYYCQHSWEIPLTFGQGTKLEIK | |
| AB-14.7 | DIQMTQSPSSLSASVGDRVTITCRASQSVGTSTYSYMHWYQQKP | 82 |
| | GKAPKLLIKYASNLESGVPSRFSGSGSGTDFTLTISSLQPEDFA | |
| | TYYCQHSWEIPLTFGQGTKLEIK | |
| AB-14.8 | DIQMTQSPSSLPASVGDRVTITCRASQSVGTSTYSYMHWYQQKP | 83 |
| | GKAPKLLIKYASNLGSGVPSRFSGSGSGTDFTLTISSLQPEDFA | |
| | TYYCQHSWEIPLTFGQGTKLEIK | |
| AB-14.9 | GIQMTQSPSSLSASVGDRVTITCRASQSVGTSTYSYMHWYQQKP | 84 |
| | GKAPKLLIKYAVNLESGVPSRFSGSGSGTDFTLTISSLQPEDFA | |
| | TYYCQHSWEIPLTFGQGTKLEIK | |
| AB-14.10 | DIQMTQSPSSLSASVGDRVTITCRASQSVSASTYSYMHWYQQKP | 85 |
| | GKAPKLLIKYASNLESGVPSRFSGSGSGTDFTLTISSLQPEDLA | |
| | TYYCQHSWEIPLTFGQGTKLEIK | |
| AB-H63; AB-63.4; | DIVLTQSPDSLAVSLGERATINCRASQSVSTSTYSYMHWYQQKP | 86 |
| AB-63.5; AB-H64; | GQPPKLLIKYASNLESGVPDRFSGSGSGTDFTLTISSLQAEDVA | |
| AB-64.1 | VYYCQHSWEIPLTFGQGTKLEIK | |
| AB-64.1.1; AB- | | |
| 64.1.2; AB-64.1.3; | | |
| AB-64.1.4; AB- | | |
| 64.1.5; AB-64.1.6; | | |
| AB-64.1.7; AB- | | |
| 64.1.8; AB-64.1.9; | | |
| AB-64.1.10; AB- | | |
| 64.1.11; AB-64.1.12; | | |
| AB-64.1.13 | | |
| AB-64.1.14; AB- | | |
| 64.1.15; AB-H65, | | |
| AB-H66; and AB-71 | | |
| AB-63.6; AB-63.8; | DIVLTQSPDSLAVSLGERATINCRASQDVSTSTYSYMHWYQQKP | 87 |
| AB-63.11; and AB- | GQPPKLLIKYASNLESGVPDRFSGSGSGTDFTLTISSLQAEDVA | |
| 64.8 | VYYCQHSWEIPLTFGQGTKLEIK | |
| AB-63.7 | DIVLTQSPDSLAVSLGERATINCRASQDVSTSTYSYMHWYQQKP | 88 |
| | GQPPKLLIKYAFNLESGVPDRFSGSGSGTDFTLTISSLQAEDVA | |
| | VYYCQHSWEIPLEFGQGTKLEIK | |
| AB-63.9; AB-64.3; | DIVLTQSPDSLAVSLGERATINCRASQDVSTSTYSYMHWYQQKP | 89 |
| and AB-64.4 | GQPPKLLIKYASYLESGVPDRFSGSGSGTDFTLTISSLQAEDVA | |
| | VYYCEHSWEIPLTFGQGTKLEIK | |
| AB-63.10 | DIVLTQSPDSLAVSLGERATINCRASQDVSTSTYSYMHWYQQKP | 90 |
| | GQPPKLLIKYASNLESGVPDRFSGSGSGTDFTLTISSLQAEDVA | |
| | VYYCQHSWELPLTFGQGTKLEIK | |
| AB-63.12 | DIVLTQSPDSLAVSLGERATINCRASQDVSTSTYSYMHWYQQKP | 91 |
| | GQPPKLLIKYASNVESGVPDRFSGSGSGTDFTLTISSLQAEDVA | |
| | VYYCQHSWAIPLTFGQGTKLEIK | |
| AB-63.13 | DIVLTQSPDSLAVSLGERATINCRASQSVSTSTYSYMHWYQQKP | 92 |
| | GQPPKLLIKYASNLESGVPDRFSGSGSGTDFTLTISSLQAEDVA | |
| | VYYCQHSEEIPLTFGQGTKLEIK | |
| AB-63.14 | DIVLTQSPDSLAVSLGERATIDCKASQDVSTSTYSYMHWYQQKP | 93 |
| | GQPPKLLIKYASNLESGVPDRFSGSGSGTDFTLTISSLQAEDVA | |
| | VYYCQHSWEIPLTFGQGTKLEIK | |
| AB-63.15 | DIVLTQSPDSLAVSLGERATINCRASQDVSTSTYSYMHWYQQKP | 94 |
| | GQPPKLLIKYASNVESGVPDRFSGSGSGTDFTLTISSLQAEDVA | |
| | VYYCQHSWEIPLTFGQGTKLEIE | |
| AB-63.16 | DIVLTQSPDSLAVSLGERATINCRASQSVHTSTYSYMHWYQQKP | 95 |
| | GQPPKLLIKYASNLESGVPDRFSGSGSGTDFTLTISSLQAEDVA | |
| | VYYCQHSWEIPLTFGQGTKLEIK | |
| AB-63.17 | DIVLTQSPDSLAVSLGERATINCRGSQSVSTSTYSYMHWYQQKP | 96 |
| | GQPPKLLIKYESNLESGVPDRFSGSGSGTDFTLTISSLQAEDVA | |
| | VYYCQHSWEIPLTFGQGTKLEIK | |
| AB-63.18 | DIVLTQSPDSLAVSLGERATINCRASQSVSTSTYSYMHWYQQKP | 97 |
| | GQPPKLLIKYESNLESGVPDRFSGSGSGTDFTLTISSLQAEDVA | |
| | VYYCQHSWEIPLTFGQGTKLEIK | |
| AB-64.2 | DIVLTQSPDSLAVSLGERATINCRVSQDVSTSTYSYMHWYQQKP | 98 |
| | GQPPKLLIKYASNLESGVPDRFSGSGSGTDFTLTISSLQAEDVA | |
| | VYYCQHSWEIPLTFGQGTKLEIK | |
| AB-64.5 | DIVLTQSPDSLAVSLGERATINCRASQSVGTSTYSYMHWYQQKP | 99 |
| | GQPPKLLIKYASNLESGVPDRFSGSGSGTDFTLTISSLQAEDVA | |
| | VYYCQHSWEIPLTFGQGTKLEIK | |
| AB-64.6 | DIVLTQSPDSLAVSLGERATINCRASQDVSTSTYSYMHWYQQKP | 100 |
| | GQPPKLLIKYASFLESGVPDRFSGSGSGTDFTLTISSLQAEDVA | |
| | VYYCQHSWEIPLTFGQGTKLEIK | |
| AB-64.7 | DIVLTQSPDSLAVSLGERATINCRASQSVGTSTYSYMHWYQQKP | 101 |
| | GQPPKLLIKYASNLNSGVPDRFSGSGSGTDFTLTISSLQAEDVA | |
| | VYYCQHSEEIPLTFGQGTKLEIK | |
| AB-H1; AB- | DIQMTQSPSSLSASVGDRVTITCRASQSVSTSTYSYMHWYQQKP | 102 |
| H2; AB-H3; AB-H6; | GKAPKLLIYYASNLESGVPSRFSGSGSGTDFTLTISSLQPEDFA | |
| AB-H9; and AB-H11 | TYYCQHSWEIPLTFGQGTKLEIK | |

TABLE 5A
EU or Kabat heavy chain
framework 1 sequences of anti-CD33
antibodies

| | | SEQ |
|---|---|---|
| | | ID |
| Ab(s) | VH FR1 | NO: |
| AB-H14; AB-14.1; | QVQLVQSGAEVKKP | 2 |
| AB-14.2; AB-14.3; | GASVKVSCKAS | |
| AB-14.4; AB-14.5; | | |
| AB-14.6; AB-14.7; | | |
| AB-14.8; AB-14.9; | | |
| AB-14.10; AB-14.11; | | |
| AB-H63; AB-63.4; | | |
| AB-63.5; AB-63.6; | | |
| AB-63.7; AB-63.9; | | |
| AB-63.10; AB-63.11; | | |
| AB-63.12; AB-63.13; | | |
| AB-63.15; AB-63.16; | | |
| and AB-63.17 | | |
| AB-63.8; AB-63.14; | QVQLVQSGAEVKK | 3 |
| and AB-63.18 | PGSSVKVSCKAS | |
| AB-H64; AB-64.1; | QVQLVQSGAEVKK | 4 |
| AB-64.2; AB-64.3; | PGASVKISCKAS | |
| AB-64.4; AB-64.5; | | |
| AB-64.6; AB-64.7; | | |
| AB-64.8; AB-64.1.1; | | |
| AB-64.1.2; AB-64.1.3; | | |
| AB-64.1.4; AB-64.1.5; | | |
| AB-64.1.6; AB-64.1.7; | | |
| AB-64.1.8; | | |
| AB-64.1.9; AB-64.1.10; | | |
| AB-64.1.11; AB- | | |
| 64.1.12; AB-64.1.13; | | |
| AB-64.1.14; and AB- | | |
| 64.1.15 | | |
| Formula VII | QVQLVQSGAEVKKPG | 158 |
| | X₁SVKX₂SCKAS | |
| | X₁ is A or S | |
| | X₂ is V or I | |

TABLE 5B
EU or Kabat heavy chain framework 2
sequences of anti-CD33 antibodies

| | | SEQ |
|---|---|---|
| | | ID |
| Ab(s) | VH FR2 | NO: |
| AB-H14; AB-14.1; AB-14.2; | WVRQAPG | 5 |
| AB-14.3; AB-14.4; AB-14.5; | QGLEWIG | |
| AB-14.6; AB-14.7; AB-14.8; | | |
| AB-14.9; AB-14.10; AB-14.11; | | |
| AB-H63; AB-63.4; AB-63.5; | | |
| AB-63.6; AB-63.7; AB-63.8; | | |
| AB-63.9; AB-63.10; | | |
| AB-63.11; AB-63.12; | | |
| AB-63.13; AB-63.14; | | |
| AB-63.15; AB-63.16; | | |
| AB-63.17; AB-63.18; | | |
| AB-H64; AB-64.1; | | |
| AB-64.2; AB-64.3; | | |
| AB-64.4; AB-64.5; | | |
| AB-64.6; AB-64.7; | | |
| AB-64.8; AB-64.1.1; | | |
| AB-64.1.2; AB-64.1.3; | | |
| AB-64.1.4; AB-64.1.5; | | |
| AB-64.1.6; AB-64.1.7; | | |
| AB-64.1.8; AB-64.1.9; | | |
| AB-64.1.10; AB-64.1.11; | | |
| AB-64.1.12; AB-64.1.13; | | |
| AB-64.1.14; | | |
| and AB-64.1.15 | | |

TABLE 5C
EU or Kabat heavy chain framework 3
sequences of anti-CD33 antibodies

| | | SEQ |
|---|---|---|
| | | ID |
| Ab(s) | VH FR3 | NO: |
| AB-H14; AB-14.4; | YAQKFQGRVTMT | 6 |
| AB-14.5; AB-14.6; | VDTSTSTVYMEL | |
| AB-14.7; AB-14.8; | SSLRSEDTAVYY | |
| AB-14.9; AB-14.10; | CAR | |
| AB-14.11; AB-H63; | | |
| AB-63.4; AB-63.5; | | |
| AB-63.6; AB-63.7; | | |
| AB-63.9; AB-63.10; | | |
| AB-63.13; AB-63.14; | | |
| AB-63.15; AB- | | |
| 63.16; AB-63.17; | | |
| and AB-63.18 | | |
| AB-14.1 | YAQDFQGRVTMTV | 7 |
| | DTSTSTVYMELSS | |
| | LRSEDTAVYYCAR | |
| AB-14.2 and AB-63.8 | YAQKFQDRVTMTV | 8 |
| | DTSTSTVYMELSS | |
| | LRSEDTAVYYCAR | |
| AB-14.3 | SAQKFQGRVTMTV | 9 |
| | DTSTSTVYMELSS | |
| | LRSEDTAVYYCAR | |
| AB-63.11 | YAQKDQGRVTMTV | 10 |
| | DTSTSTVYMELSS | |
| | LRSEDTAVYYCAR | |
| AB-63.12 | YAQKFTGRVTMTV | 11 |
| | DTSTSTVYMELSS | |
| | LRSEDTAVYYCAR | |
| AB-H64; AB-64.1; | YAQKFQGRATLTV | 12 |
| AB-64.2; AB-64.4; | DNSTSTAYMELSS | |
| AB-64.6; AB-64.7; | LRSEDTAVYYCAR | |
| AB-64.1.1; | | |
| AB-64.1.2; and | | |
| AB-64.1.3 | | |
| AB-64.3 | YAEKFEGRATLTV | 13 |
| | DNSTSTAYMELSS | |
| | LRSEDTAVYYCAR | |
| AB-64.5 | YAQKFFGRATLTV | 14 |
| | DNSTSTAYMELSS | |
| | LRSEDTAVYYCAR | |
| AB-64.8 | YAQKFQHRATLTV | 15 |
| | DNSTSTAYMELSS | |
| | LRSEDTAVYYCAR | |
| AB-64.1.4; | YAQKFQGRATLTV | 16 |
| AB-64.1.10; | DTSTSTAYMELSS | |
| and | LRSEDTAVYYCAR | |
| AB-64.1.13 | | |
| AB-64.1.5; | YAQKFQGRATLTV | 17 |
| AB-64.1.11; and | DQSTSTAYMELSS | |
| | LRSEDTAVYYCAR | |
| AB-64.1.14 | | |
| AB-64.1.6; | YAQKFQGRATLTV | 18 |
| AB-64.1.8; | DNSASTAYMELSS | |
| | LRSEDTAVYYCAR | |
| AB-64.1.12; | | |
| and AB-64.1.15 | | |
| AB-64.1.7 and | YAQKFQGRATLTV | 19 |
| AB-64.1.9 | DNPTSTAYMELSS | |
| | LRSEDTAVYYCAR | |
| Formula VIII | X₁AX₂X₃X₄X₅X₆ | 159 |
| | RX₇TX₈TVDX₉X₁₀ | |
| | X₁₁STX₁₂YMELSS | |
| | LRSEDTAVYYCAR | |
| | X₁ is Y or S | |
| | X₂ is Q or E | |
| | X₃ is K or D | |
| | X₄ is F or D | |
| | X₅ is Q, F, E, | |
| | or T | |
| | X₆ is G, D, or H | |
| | X₇ is V or A | |
| | X₈ is M or L | |
| | X₉ is T, N, or Q | |
| | X₁₀ is S or P | |
| | X₁₁ is T or A | |
| | X₁₂ is V or A | |

TABLE 5D
EU or Kabat heavy chain framework 4
sequences of anti-CD33 antibodies

| | | SEQ |
|---|---|---|
| | | ID |
| Ab(s) | VH FR4 | NO: |
| AB-H14; AB-14.1; | WGQGTLVTVSS | 20 |
| AB-14.2; AB-14.3; | | |
| AB-14.4; AB-14.5; | | |
| AB-14.6; AB-14.7; | | |
| AB-14.8; AB-14.9; | | |
| AB-14.10; AB-14.11; | | |
| AB-H63; AB-63.4; | | |
| AB-63.5; AB-63.6; | | |
| AB-63.7; AB-63.8; | | |
| AB-63.9; AB-63.10; | | |
| AB-63.11; AB-63.12; | | |
| AB-63.13; AB-63.14; | | |
| AB-63.15; AB-63.16; | | |
| AB-63.17; and | | |
| AB-63.18 | | |
| AB-H64; AB-64.1; | WGQGTLLTVSS | 21 |
| AB-64.2; AB-64.3; | | |
| AB-64.4; AB-64.5; | | |
| AB-64.6; AB-64.7; | | |
| AB-64.8; AB-64.1.1; | | |
| AB-64.1.2; AB-64.1.3; | | |
| AB-64.1.4; AB-64.1.5; | | |
| AB-64.1.6; AB-64.1.7; | | |
| AB-64.1.8; AB-64.1.9; | | |
| AB-64.1.10; AB-64.1.11; | | |
| AB-64.1.12; AB-64.1.13; | | |
| AB-64.1.14; and | | |
| AB-64.1.15 | | |
| Formula IX | WGQGTLX₁TVSS | 160 |
| | X₁ is V or L | |

TABLE 6A
EU or Kabat light chain framework 1
sequences of anti-CD33 antibodies

| | | SEQ |
|---|---|---|
| | | ID |
| Ab(s) | VL FR1 | NO: |
| AB-H14; AB-14.1; | DIQMTQSPSS | 22 |
| AB-14.2; AB-14.3; | LSASVGDRVT | |
| AB-14.4; AB-14.5; | ITC | |
| AB-14.6; AB-14.7; | | |
| AB-14.10; and | | |
| AB-14.11 | | |
| AB-14.8 | DIQMTQSPSSL | 23 |
| | PASVGDRVTIT | |
| | C | |
| AB-14.9 | GIQMTQSPSSL | 24 |
| | SASVGDRVTIT | |
| | C | |
| AB-H63; AB-63.4; | DIVLTQSPDSL | 25 |
| AB-63.5; AB-63.6; | AVSLGERATIN | |
| AB-63.7; AB-63.8; | C | |
| AB-63.9; AB-63.10; | | |
| AB-63.11; AB-63.12; | | |
| AB-63.13; AB-63.14; | | |
| AB-63.15; AB-63.16; | | |
| AB-63.17; AB-63.18; | | |
| AB-H64; AB-64.1; | | |
| AB-64.2; AB-64.3; | | |
| AB-64.4; AB-64.5; | | |
| AB-64.6; AB-64.7; | | |
| AB-64.8; AB-64.1.1; | | |
| AB-64.1.2; AB-64.1.3; | | |
| AB-64.1.4; AB-64.1.5; | | |
| AB-64.1.6; AB-64.1.7; | | |
| AB-64.1.8; AB-64.1.9; | | |
| AB-64.1.10; AB-64.1.11; | | |
| AB-64.1.12; AB-64.1.13; | | |
| AB-64.1.14; and | | |
| AB-64.1.15 | | |
| AB-63.14 | DIVLTQSPDSL | 26 |
| | AVSLGERATID | |
| | C | |
| Formula X | X₁X₂X₃TQSPX₄ | 161 |
| | SLX₅X₆SX₇GX₈ | |
| | RX₉TIX₁₀C | |
| | X₁ is D or G | |
| | X₂ is Q or V | |
| | X₃ is M or L | |
| | X₄ is S or D | |
| | X₅ is S, P, | |
| | or A | |
| | X₆ is A or V | |
| | X₇ is V or L | |
| | X₈ is D or E | |
| | X₉ is V or A | |
| | X₁₀ is T, N, | |
| | or D | |

TABLE 6B
EU or Kabat light chain framework 2
sequences of anti-CD33 antibodies

| | | SEQ |
|---|---|---|
| | | ID |
| Ab(s) | VL FR2 | NO: |
| AB-H14; AB-14.1; | WYQQKPGKAPKLLIK | 27 |
| AB-14.2; AB-14.3; | | |
| AB-14.4; AB-14.6; | | |
| AB-14.7; AB-14.8; | | |
| AB-14.9; AB-14.10; | | |
| and AB-14.11 | | |
| AB-H63; AB-63.4; | WYQQKPGQPPKLLIK | 28 |
| AB-63.5; AB-63.6; | | |
| AB-63.7; AB-63.8; | | |
| AB-63.9; AB-63.10; | | |
| AB-63.11; AB-63.12; | | |
| AB-63.13; AB- | | |
| 63.14; AB-63.15; | | |
| AB-63.16; AB-63.17; | | |
| AB-63.18; AB-H64; | | |
| AB-64.1; AB-64.2; | | |
| AB-64.3; AB-64.4; | | |
| AB-64.5; AB-64.6; | | |
| AB-64.7; AB-64.8; | | |
| AB-64.1.1; AB-64.1.2; | | |
| AB-64.1.3; AB-64.1.4; | | |
| AB-64.1.5; AB-64.1.6; | | |
| AB-64.1.7; AB-64.1.8; | | |
| AB-64.1.9; AB-64.1.10; | | |
| AB-64.1.11; AB-64.1.12; | | |
| AB-64.1.13; AB-64.1.14; | | |
| and AB-64.1.15 | | |
| AB-14.5 | WYQRKPGKAPKLLIK | 168 |
| Formula XI | WYQQKPGX₁X₂PKLLIK | 162 |
| | X₁ is K or Q | |
| | X₂ is A or P | |
| Formula XIV | WYQX₁KPGX₂X₃PKL | 169 |
| | LIK | |
| | X₁ is Q or R | |
| | X₂ is K or Q | |
| | X₃ is A or P | |

TABLE 6C
EU or Kabat light chain framework 3
sequences of anti-CD33 Mantibodies

| | | SEQ |
|---|---|---|
| | | ID |
| Ab(s) | VL FR3 | NO: |
| AB-H14; AB-14.1; | GVPSRFSGSGSGT | 29 |
| AB-14.2; AB-14.3; | DFTLTISSLQPED | |
| AB-14.4; AB-14.5; | FATYYC | |
| AB-14.6; AB-14.7; | | |
| AB-14.8; AB-14.9; | | |
| and AB-14.11 | | |
| AB-14.10 | GVPSRFSGSGSGT | 30 |
| | DFTLTISSLQPED | |
| | LATYYC | |
| AB-H63; AB-63.4; | GVPDRFSGSGSGT | 31 |
| AB-63.5; AB-63.6; | DFTLTISSLQAED | |
| AB-63.7; AB-63.8; | VAVYYC | |
| AB-63.9; AB-63.10; | | |
| AB-63.11; AB-63.12; | | |
| AB-63.13; AB-63.14; | | |
| AB-63.15; AB-63.16; | | |
| AB-63.17; AB-63.18; | | |
| AB-H64; AB-64.1; | | |
| AB-64.2; AB-64.3; | | |
| AB-64.4; AB-64.5; | | |
| AB-64.6; AB-64.7; | | |
| AB-64.8; AB-64.1.1; | | |
| AB-64.1.2; AB-64.1.3; | | |
| AB-64.1.4; AB-64.1.5; | | |
| AB-64.1.6; AB-64.1.7; | | |
| AB-64.1.8; AB-64.1.9; | | |
| AB-64.1.10;AB-64.1.11; | | |
| AB-64.1.12; AB-64.1.13; | | |
| AB-64.1.14; and | | |
| AB-64.1.15 | | |
| Formula XII | GVPX₁RFSGSGSG | 163 |
| | TDFTLTISSLQX₂ | |
| | EDX₃AX₄ | |
| | YYC | |
| | X₁ is S or D | |
| | X₂ is P or A | |
| | X₃ is F, L, or V | |
| | X₄ is T or V | |

TABLE 6D
EU or Kabat light chain framework 4
sequences of anti-CD33 antibodies

| | | SEQ |
|---|---|---|
| | | ID |
| Ab(s) | VL FR4 | NO: |
| AB-H14; AB-14.1; | FGQGTKLEIK | 32 |
| AB-14.2; AB-14.3; | | |
| AB-14.4; AB-14.5; | | |
| AB-14.6; AB-14.7; | | |
| AB-14.8; AB-14.9; | | |
| AB-14.10; AB-14.11; | | |
| AB-H63; AB-63.4; | | |
| AB-63.5; AB-63.6; | | |
| AB-63.7; AB-63.8; | | |
| AB-63.9; AB-63.10; | | |
| AB-63.11; AB-63.12; | | |
| AB-63.13; AB-63.14; | | |
| AB-63.16;AB-63.17; | | |
| AB-63.18; AB-H64; | | |
| AB-64.1; AB-64.2; | | |
| AB-64.3; AB-64.4; | | |
| AB-64.5; AB-64.6; | | |
| AB-64.7; AB-64.8; | | |
| AB-64.1.1; AB-64.1.2; | | |
| AB-64.1.3; AB-64.1.4; | | |
| AB-64.1.5; AB-64.1.6; | | |
| AB-64.1.7; AB-64.1.8; | | |
| AB-64.1.9; AB-64.1.10; | | |
| AB-64.1.11; AB-64.1.12; | | |
| AB-64.1.13; AB-64.1.14; | | |
| and AB-64.1.15 | | |
| AB-63.15 | FGQGTKLEIE | 33 |
| Formula XIII | FGQGTKLEIX₁ | 164 |
| | X₁ is K or E | |

### SEQUENCES

All polypeptide sequences are presented N-terminal to C-terminal unless otherwise noted.

Parental mouse antibody heavy chain variable
region:
(SEQ ID NO: 103)
EVQLQQSGPELVKPGASVKISCKASGYTFTDYNLHWVKLSHGKSL
EWIGFIYPSNGITGYNQKFKNKATLTVDNSSSTAYMELRSLTSED
SAVYYCARSTVDYFDYWGQGTTLTVSS
Parental mouse antibody light chain variable
region:
(SEQ ID NO: 104)
DIVLTQSPASLAVSLGQRATMSCRASQSVSTSTYSYMHWYQQKPG
QPPKLLIKYASNLESGVPARFSGSGSGTDFTLNIHPVEEEDTATY
YCQHSWEIPLTFGAGTKLELK
Receptor motif:
(SEQ ID NO: 165)
D/Ex0-2YxxL/IX6-8YxxL/I
CH1 and hinge region of IGg2:
(SEQ ID NO: 166)
ASTKGPSVFPLAPCSRSTSESTAALGCLVKDYFPEPVTVSWNSGA
LTSGVHTFPAVLQSSGLYSLSSVVTVPSSNFGTQTYTCNVDHKPS
NTKVDKTVERKCCVECPPCP
AB-64.1 huIgG1 full-length antibody
sequence:
Heavy chain:
(SEQ ID NO: 176)
QVQLVQSGAEVKKPGASVKISCKASGYTFTDYNLHWVRQAPGQGL
EWIGFIYPSNGITGYAQKFQGRATLTVDNSTSTAYMELSSLRSED
TAVYYCARSDVDYFDYWGQGTLLTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPELLGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPSRDELTK
NQVSLTCLVKGFYPSDIAVEWESNGQPENNYKTTPPVLDSDGSFF
LYSKLTVDKSRWQQGNVFSCSVMHEALHNHYTQKSLSLSPGK
(SEQ ID NO: 197)
QVQLVQSGAEVKKPGASVKISCKASGYTFTDYNLHWVRQAPGQGL
EWIGFIYPSNGITGYAQKFQGRATLTVDNSTSTAYMELSSLRSED
TAVYYCARSDVDYFDYWGQGTLLTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPELLGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPSRDELTK
NQVSLTCLVKGFYPSDIAVEWESNGQPENNYKTTPPVLDSDGSFF
LYSKLTVDKSRWQQGNVFSCSVMHEALHNHYTQKSLSLSPG
Light chain:
(SEQ ID NO: 185)
DIVLTQSPDSLAVSLGERATINCRASQSVSTSTYSYMHWYQQKPG
QPPKLLIKYASNLESGVPDRFSGSGSGTDFTLTISSLQAEDVAVY
YCQHSWEIPLTFGQGTKLEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
AB-64.1.2 huIgG1 full-length antibody
sequence
Heavy chain:
(SEQ ID NO: 177)
QVQLVQSGAEVKKPGASVKISCKASGYTFTDYNLHWVRQAPGQGL
EWIGFIYPSNRITGYAQKFQGRATLTVDNSTSTAYMELSSLRSED
TAVYYCARSDVDYFDYWGQGTLLTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPELLGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPSRDELTK
NQVSLTCLVKGFYPSDIAVEWESNGQPENNYKTTPPVLDSDGSFF
LYSKLTVDKSRWQQGNVFSCSVMHEALHNHYTQKSLSLSPGK
(SEQ ID NO: 198)
QVQLVQSGAEVKKPGASVKISCKASGYTFTDYNLHWVRQAPGQGL
EWIGFIYPSNRITGYAQKFQGRATLTVDNSTSTAYMELSSLRSED
TAVYYCARSDVDYFDYWGQGTLLTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPELLGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPSRDELTK
NQVSLTCLVKGFYPSDIAVEWESNGQPENNYKTTPPVLDSDGSFF
LYSKLTVDKSRWQQGNVFSCSVMHEALHNHYTQKSLSLSPG
Light chain:
(SEQ ID NO: 185)
DIVLTQSPDSLAVSLGERATINCRASQSVSTSTYSYMHWYQQKPG
QPPKLLIKYASNLESGVPDRFSGSGSGTDFTLTISSLQAEDVAVY
YCQHSWEIPLTFGQGTKLEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
AB-64.1.8 huIgGl full-length antibody
sequence
Heavy chain:
(SEQ ID NO: 178)
QVQLVQSGAEVKKPGASVKISCKASGYTFTDYNLHWVRQAPGQGL
EWIGFIYPSNQITGYAQKFQGRATLTVDNSASTAYMELSSLRSED
TAVYYCARSDVDYFDYWGQGTLLTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPELLGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPSRDELTK
NQVSLTCLVKGFYPSDIAVEWESNGQPENNYKTTPPVLDSDGSFF
LYSKLTVDKSRWQQGNVFSCSVMHEALHNHYTQKSLSLSPGK
(SEQ ID NO: 199)
QVQLVQSGAEVKKPGASVKISCKASGYTFTDYNLHWVRQAPGQGL
EWIGFIYPSNQITGYAQKFQGRATLTVDNSASTAYMELSSLRSED
TAVYYCARSDVDYFDYWGQGTLLTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPELLGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPSRDELTK
NQVSLTCLVKGFYPSDIAVEWESNGQPENNYKTTPPVLDSDGSFF
LYSKLTVDKSRWQQGNVFSCSVMHEALHNHYTQKSLSLSPG
Light chain:
(SEQ ID NO: 185)
DIVLTQSPDSLAVSLGERATINCRASQSVSTSTYSYMHWYQQKPG
QPPKLLIKYASNLESGVPDRFSGSGSGTDFTLTISSLQAEDVAVY
YCQHSWEIPLTFGQGTKLEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
AB-64.1 huIgG2 full-length antibody
sequence
Heavy chain:
(SEQ ID NO: 179)
QVQLVQSGAEVKKPGASVKISCKASGYTFTDYNLHWVRQAPGQGL
EWIGFIYPSNGITGYAQKFQGRATLTVDNSTSTAYMELSSLRSED
TAVYYCARSDVDYFDYWGQGTLLTVSSASTKGPSVFPLAPCSRST
SESTAALGCLVKDYFPEPVTVSWNSGALTSGVHTFPAVLQSSGLY
SLSSVVTVPSSNFGTQTYTCNVDHKPSNTKVDKTVERKCCVECPP
CPAPPVAGPSVFLFPPKPKDTLMISRTPEVTCVVVDVSHEDPEVQ
FNWYVDGVEVHNAKTKPREEQFNSTFRVVSVLTVVHQDWLNGKEY
KCKVSNKGLPAPIEKTISKTKGQPREPQVYTLPPSREEMTKNQVS
LTCLVKGFYPSDIAVEWESNGQPENNYKTTPPMLDSDGSFFLYSK
LTVDKSRWQQGNVFSCSVMHEALHNHYTQKSLSLSPGK
(SEQ ID NO: 200)
QVQLVQSGAEVKKPGASVKISCKASGYTFTDYNLHWVRQAPGQGL
EWIGFIYPSNGITGYAQKFQGRATLTVDNSTSTAYMELSSLRSED
TAVYYCARSDVDYFDYWGQGTLLTVSSASTKGPSVFPLAPCSRST
SESTAALGCLVKDYFPEPVTVSWNSGALTSGVHTFPAVLQSSGLY
SLSSVVTVPSSNFGTQTYTCNVDHKPSNTKVDKTVERKCCVECPP
CPAPPVAGPSVFLFPPKPKDTLMISRTPEVTCVVVDVSHEDPEVQ
FNWYVDGVEVHNAKTKPREEQFNSTFRVVSVLTVVHQDWLNGKEY
KCKVSNKGLPAPIEKTISKTKGQPREPQVYTLPPSREEMTKNQVS
LTCLVKGFYPSDIAVEWESNGQPENNYKTTPPMLDSDGSFFLYSK
LTVDKSRWQQGNVFSCSVMHEALHNHYTQKSLSLSPG
Light chain:
(SEQ ID NO: 185)
DIVLTQSPDSLAVSLGERATINCRASQSVSTSTYSYMHWYQQKPG
QPPKLLIKYASNLESGVPDRFSGSGSGTDFTLTISSLQAEDVAVY
YCQHSWEIPLTFGQGTKLEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
AB-64.1.2 huQG2 full-length antibody
sequence
Heavy chain:
(SEQ ID NO: 180)
QVQLVQSGAEVKKPGASVKISCKASGYTFTDYNLHWVRQAPGQGL
EWIGFIYPSNRITGYAQKFQGRATLTVDNSTSTAYMELSSLRSED
TAVYYCARSDVDYFDYWGQGTLLTVSSASTKGPSVFPLAPCSRST
SESTAALGCLVKDYFPEPVTVSWNSGALTSGVHTFPAVLQSSGLY
SLSSVVTVPSSNFGTQTYTCNVDHKPSNTKVDKTVERKCCVECPP
CPAPPVAGPSVFLFPPKPKDTLMISRTPEVTCVVVDVSHEDPEVQ
FNWYVDGVEVHNAKTKPREEQFNSTFRVVSVLTVVHQDWLNGKEY
KCKVSNKGLPAPIEKTISKTKGQPREPQVYTLPPSREEMTKNQVS
LTCLVKGFYPSDIAVEWESNGQPENNYKTTPPMLDSDGSFFLYSK
LTVDKSRWQQGNVFSCSVMHEALHNHYTQKSLSLSPGK
(SEQ ID NO: 201)
QVQLVQSGAEVKKPGASVKISCKASGYTFTDYNLHWVRQAPGQGL
EWIGFIYPSNRITGYAQKFQGRATLTVDNSTSTAYMELSSLRSED
TAVYYCARSDVDYFDYWGQGTLLTVSSASTKGPSVFPLAPCSRST
SESTAALGCLVKDYFPEPVTVSWNSGALTSGVHTFPAVLQSSGLY
SLSSVVTVPSSNFGTQTYTCNVDHKPSNTKVDKTVERKCCVECPP
CPAPPVAGPSVFLFPPKPKDTLMISRTPEVTCVVVDVSHEDPEVQ
FNWYVDGVEVHNAKTKPREEQFNSTFRVVSVLTVVHQDWLNGKEY
KCKVSNKGLPAPIEKTISKTKGQPREPQVYTLPPSREEMTKNQVS
LTCLVKGFYPSDIAVEWESNGQPENNYKTTPPMLDSDGSFFLYSK
LTVDKSRWQQGNVFSCSVMHEALHNHYTQKSLSLSPG
Light chain:
(SEQ ID NO: 185)
DIVLTQSPDSLAVSLGERATINCRASQSVSTSTYSYMHWYQQKPG
QPPKLLIKYASNLESGVPDRFSGSGSGTDFTLTISSLQAEDVAVY
YCQHSWEIPLTFGQGTKLEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
AB-64.1.8 huIgG2 full-length antibody
sequence
Heavy chain:
(SEQ ID NO: 181)
QVQLVQSGAEVKKPGASVKISCKASGYTFTDYNLHWVRQAPGQGL
EWIGFIYPSNQITGYAQKFQGRATLTVDNSASTAYMELSSLRSED
TAVYYCARSDVDYFDYWGQGTLLTVSSASTKGPSVFPLAPCSRST
SESTAALGCLVKDYFPEPVTVSWNSGALTSGVHTFPAVLQSSGLY
SLSSVVTVPSSNFGTQTYTCNVDHKPSNTKVDKTVERKCCVECPP
CPAPPVAGPSVFLFPPKPKDTLMISRTPEVTCVVVDVSHEDPEVQ
FNWYVDGVEVHNAKTKPREEQFNSTFRVVSVLTVVHQDWLNGKEY
KCKVSNKGLPAPIEKTISKTKGQPREPQVYTLPPSREEMTKNQVS
LTCLVKGFYPSDIAVEWESNGQPENNYKTTPPMLDSDGSFFLYSK
LTVDKSRWQQGNVFSCSVMHEALHNHYTQKSLSLSPGK
(SEQ ID NO: 202)
QVQLVQSGAEVKKPGASVKISCKASGYTFTDYNLHWVRQAPGQGL
EWIGFIYPSNQITGYAQKFQGRATLTVDNSASTAYMELSSLRSED
TAVYYCARSDVDYFDYWGQGTLLTVSSASTKGPSVFPLAPCSRST
SESTAALGCLVKDYFPEPVTVSWNSGALTSGVHTFPAVLQSSGLY
SLSSVVTVPSSNFGTQTYTCNVDHKPSNTKVDKTVERKCCVECPP
CPAPPVAGPSVFLFPPKPKDTLMISRTPEVTCVVVDVSHEDPEVQ
FNWYVDGVEVHNAKTKPREEQFNSTFRVVSVLTVVHQDWLNGKEY
KCKVSNKGLPAPIEKTISKTKGQPREPQVYTLPPSREEMTKNQVS
LTCLVKGFYPSDIAVEWESNGQPENNYKTTPPMLDSDGSFFLYSK
LTVDKSRWQQGNVFSCSVMHEALHNHYTQKSLSLSPG
Light chain:
(SEQ ID NO: 185)
DIVLTQSPDSLAVSLGERATINCRASQSVSTSTYSYMHWYQQKPG
QPPKLLIKYASNLESGVPDRFSGSGSGTDFTLTISSLQAEDVAVY
YCQHSWEIPLTFGQGTKLEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
AB-64.1 huIgG1 LALAPS full-length antibody
sequence
Heavy chain:
(SEQ ID NO: 182)
QVQLVQSGAEVKKPGASVKISCKASGYTFTDYNLHWVRQAPGQGL
EWIGFIYPSNGITGYAQKFQGRATLTVDNSTSTAYMELSSLRSED
TAVYYCARSDVDYFDYWGQGTLLTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPASIEKTISKAKGQPREPQVYTLPPSRDELTK
NQVSLTCLVKGFYPSDIAVEWESNGQPENNYKTTPPVLDSDGSFF
LYSKLTVDKSRWQQGNVFSCSVMHEALHNHYTQKSLSLSPGK
(SEQ ID NO: 203)
QVQLVQSGAEVKKPGASVKISCKASGYTFTDYNLHWVRQAPGQGL
EWIGFIYPSNGITGYAQKFQGRATLTVDNSTSTAYMELSSLRSED
TAVYYCARSDVDYFDYWGQGTLLTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPASIEKTISKAKGQPREPQVYTLPPSRDELTK
NQVSLTCLVKGFYPSDIAVEWESNGQPENNYKTTPPVLDSDGSFF
LYSKLTVDKSRWQQGNVFSCSVMHEALHNHYTQKSLSLSPG
Light chain:
(SEQ ID NO: 185)
DIVLTQSPDSLAVSLGERATINCRASQSVSTSTYSYMHWYQQKPG
QPPKLLIKYASNLESGVPDRFSGSGSGTDFTLTISSLQAEDVAVY
YCQHSWEIPLTFGQGTKLEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
AB-64.1.2 huIgG1 LALAPS full-length
antibody sequence
Heavy chain:
(SEQ ID NO: 183)
QVQLVQSGAEVKKPGASVKISCKASGYTFTDYNLHWVRQAPGQGL
EWIGFIYPSNRITGYAQKFQGRATLTVDNSTSTAYMELSSLRSED
TAVYYCARSDVDYFDYWGQGTLLTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPASIEKTISKAKGQPREPQVYTLPPSRDELTK
NQVSLTCLVKGFYPSDIAVEWESNGQPENNYKTTPPVLDSDGSFF
LYSKLTVDKSRWQQGNVFSCSVMHEALHNHYTQKSLSLSPGK
(SEQ ID NO: 204)
QVQLVQSGAEVKKPGASVKISCKASGYTFTDYNLHWVRQAPGQGL
EWIGFIYPSNRITGYAQKFQGRATLTVDNSTSTAYMELSSLRSED
TAVYYCARSDVDYFDYWGQGTLLTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPASIEKTISKAKGQPREPQVYTLPPSRDELTK
NQVSLTCLVKGFYPSDIAVEWESNGQPENNYKTTPPVLDSDGSFF
LYSKLTVDKSRWQQGNVFSCSVMHEALHNHYTQKSLSLSPG
Light chain:
(SEQ ID NO: 185)
DIVLTQSPDSLAVSLGERATINCRASQSVSTSTYSYMHWYQQKPG
QPPKLLIKYASNLESGVPDRFSGSGSGTDFTLTISSLQAEDVAVY
YCQHSWEIPLTFGQGTKLEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
AB-64.1.8 huIgG1 LALAPS full-length antibody
sequence
Heavy chain:
(SEQ ID NO: 184)
QVQLVQSGAEVKKPGASVKISCKASGYTFTDYNLHWVRQAPGQGL
EWIGFIYPSNQITGYAQKFQGRATLTVDNSASTAYMELSSLRSED
TAVYYCARSDVDYFDYWGQGTLLTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPASIEKTISKAKGQPREPQVYTLPPSRDELTK
NQVSLTCLVKGFYPSDIAVEWESNGQPENNYKTTPPVLDSDGSFF
LYSKLTVDKSRWQQGNVFSCSVMHEALHNHYTQKSLSLSPGK
(SEQ ID NO: 205)
QVQLVQSGAEVKKPGASVKISCKASGYTFTDYNLHWVRQAPGQGL
EWIGFIYPSNQITGYAQKFQGRATLTVDNSASTAYMELSSLRSED
TAVYYCARSDVDYFDYWGQGTLLTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPEAAGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPASIEKTISKAKGQPREPQVYTLPPSRDELTK
NQVSLTCLVKGFYPSDIAVEWESNGQPENNYKTTPPVLDSDGSFF
LYSKLTVDKSRWQQGNVFSCSVMHEALHNHYTQKSLSLSPG
Light chain:
(SEQ ID NO: 185)
DIVLTQSPDSLAVSLGERATINCRASQSVSTSTYSYMHWYQQKPG
QPPKLLIKYASNLESGVPDRFSGSGSGTDFTLTISSLQAEDVAVY
YCQHSWEIPLTFGQGTKLEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC

1. A method of treating and/or delaying the progression of a disease or injury in an individual, comprising administering to the individual an anti-CD33 antibody intravenously at a dose of at least about 1.6 mg/kg,
wherein the antibody is administered about once every twelve weeks or more frequently; and
wherein the antibody comprises: a heavy chain variable region that comprises
an HVR-H1 comprising the amino acid sequence GYTFTDYNLH (SEQ ID NO: 105),
an HVR-H2 comprising the amino acid sequence FIYPSNRITG (SEQ ID NO: 119), and
an HVR-H3 comprising the amino acid sequence SDVDYFDY (SEQ ID NO: 122); and
a light chain variable region that comprises
an HVR-L1 comprising the amino acid sequence RASQSVSTSTYSYMH (SEQ ID NO: 127),
an HVR-L2 comprising the amino acid sequence YASNLES (SEQ ID NO: 135), and
an HVR-L3 comprising the amino acid sequence QHSWEIPLT (SEQ ID NO: 146).

2. The method of claim 1, wherein the anti-CD33 antibody is administered at a dose of between about 1.6 mg/kg and about 15 mg/kg.

3. The method of claim 1, wherein the anti-CD33 antibody is administered at a dose of about 1.6 mg/kg, about 5 mg/kg, about 7.5 mg/kg, about 10 mg/kg, or about 15 mg/kg.

4. The method of claim 1, wherein the anti-CD33 antibody is administered once every two weeks, once every four weeks, once every five weeks, once every six weeks, once every seven weeks, once every eight weeks, once every nine weeks, once every ten weeks, once every eleven weeks, or once every twelve weeks.

5. The method of claim 1, wherein the anti-CD33 antibody is administered once every two weeks at a dose of about 1.6 mg/kg.

6. The method of claim 1, wherein the anti-CD33 antibody is administered once every four weeks at a dose of about 1.6 mg/kg.

7. The method of claim 1, wherein the anti-CD33 antibody is administered once every four weeks at a dose of about 15 mg/kg.

8. (canceled)

9. (canceled)

10. (canceled)

11. (canceled)

12. The method of claim 1, wherein the cell surface level of CD33 is reduced by at least about 70% compared to the cell surface level of CD33 prior to administration of the anti-CD33 antibody.

13. (canceled)

14. (canceled)

15. (canceled)

16. (canceled)

17. The method of claim 12, wherein the reduction in the cell surface level of CD33 is present for at least about 17 days after administration of the anti-CD33 antibody.

18. (canceled)

19. (canceled)

20. The method of claim 12, wherein the reduction in the cell surface level of CD33 is present for at least about 56 days after administration of the anti-CD33 antibody.

21. The method of claim 12, wherein the reduction in cell surface level of CD33 comprises a reduction in the cell surface level of CD33 on peripheral blood monocytes of the individual.

22. The method of claim 1, wherein the antibody comprises a heavy chain variable region comprising the amino acid sequence of SEQ ID NO: 59 and a light chain variable region comprising the amino acid sequence of SEQ ID NO: 86.

23. The method of claim 1, wherein the antibody has an IgG2 isotype.

24. The method of claim 1, wherein the antibody comprises a heavy chain comprising the amino acid sequence of SEQ ID NO: 180 or SEQ ID NO: 201, and a light chain comprising the amino acid sequence of SEQ ID NO: 185.

25. The method of claim 1, wherein the terminal half-life of the anti-CD33 antibody in plasma is about 4 days, about 5 days, about 6 days, about 7 days, about 8 days, about 9 days, about 10 days, about 11 days, or about 12 days.

26. The method of claim 1, wherein the terminal half-life of the anti-CD33 antibody in plasma is about 10 days.

27. The method of claim 1, wherein the disease or injury is selected from the group consisting of dementia, frontotemporal dementia, Alzheimer's disease, vascular dementia, mixed dementia, tauopathy disease, infections, and cancer.

28. The method of claim 1, wherein the disease or injury is Alzheimer's disease.

29. The method of claim 1, wherein the individual is diagnosed with Alzheimer's disease, or has a clinical diagnosis of probable Alzheimer's disease dementia.

30. The method of claim 1, wherein the individual has a Mini-Mental State Examination (MMSE) score of between about 16 points to about 28 points.

31. The method of claim 1, wherein the individual has a Clinical Dementia Rating-Global Score (CDR-GS) of about 0.5, about 1.0, or about 2.0.

32. The method of claim 1, wherein the individual has a positive amyloid-PET scan.

33. The method of claim 1, wherein the individual is taking a stable dose of a cholinesterase inhibitor and/or a memantine therapy for Alzheimer's disease.

34. The method of claim 1, wherein the individual does not carry two copies of the rs12459419^{T} allele.

35. The method of claim 1, wherein the disease or injury is Alzheimer's disease, and wherein treatment and/or delay of the progression of Alzheimer's disease is assessed using one or more clinical assessments selected from the group consisting of the Mini-Mental State Examination (MMSE), the Repeatable Battery for the Assessment of Neuropsychological Status (RBANS), the Clinical Dementia Rating (CDR) assessment, amyloid brain positron emission tomography (PET), translocator protein (TSPO)-PET imaging, and any combination thereof.


SEQUENCE LISTING (205 sequences)

SEQ ID NO: 1 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 2 - protein, 25 aa, Artificial Sequence [sequence omitted]

SEQ ID NO: 3 - protein, 25 aa, Artificial Sequence [sequence omitted]

SEQ ID NO: 4 - protein, 25 aa, Artificial Sequence
  Synthetic Construct
QVQLVQSGAEVKKPGASVKISCKAS

SEQ ID NO: 5 - protein, 14 aa, Artificial Sequence
  Synthetic Construct
WVRQAPGQGLEWIG

SEQ ID NO: 6 - protein, 39 aa, Artificial Sequence
  Synthetic Construct
YAQKFQGRVTMTVDTSTSTVYMELSSLRSEDTAVYYCAR

SEQ ID NO: 7 - protein, 39 aa, Artificial Sequence
  Synthetic Construct
YAQDFQGRVTMTVDTSTSTVYMELSSLRSEDTAVYYCAR

SEQ ID NO: 8 - protein, 39 aa, Artificial Sequence
  Synthetic Construct
YAQKFQDRVTMTVDTSTSTVYMELSSLRSEDTAVYYCAR

SEQ ID NO: 9 - protein, 39 aa, Artificial Sequence
  Synthetic Construct
SAQKFQGRVTMTVDTSTSTVYMELSSLRSEDTAVYYCAR

SEQ ID NO: 10 - protein, 39 aa, Artificial Sequence
  Synthetic Construct
YAQKDQGRVTMTVDTSTSTVYMELSSLRSEDTAVYYCAR

SEQ ID NO: 11 - protein, 39 aa, Artificial Sequence
  Synthetic Construct
YAQKFTGRVTMTVDTSTSTVYMELSSLRSEDTAVYYCAR

SEQ ID NO: 12 - protein, 39 aa, Artificial Sequence
  Synthetic Construct
YAQKFQGRATLTVDNSTSTAYMELSSLRSEDTAVYYCAR

SEQ ID NO: 13 - protein, 39 aa, Artificial Sequence
  Synthetic Construct
YAEKFEGRATLTVDNSTSTAYMELSSLRSEDTAVYYCAR

SEQ ID NO: 14 - protein, 39 aa, Artificial Sequence
  Synthetic Construct
YAQKFFGRATLTVDNSTSTAYMELSSLRSEDTAVYYCAR

SEQ ID NO: 15 - protein, 39 aa, Artificial Sequence
  Synthetic Construct
YAQKFQHRATLTVDNSTSTAYMELSSLRSEDTAVYYCAR

SEQ ID NO: 16 - protein, 39 aa, Artificial Sequence
  Synthetic Construct
YAQKFQGRATLTVDTSTSTAYMELSSLRSEDTAVYYCAR

SEQ ID NO: 17 - protein, 39 aa, Artificial Sequence
  Synthetic Construct
YAQKFQGRATLTVDQSTSTAYMELSSLRSEDTAVYYCAR

SEQ ID NO: 18 - protein, 39 aa, Artificial Sequence
  Synthetic Construct
YAQKFQGRATLTVDNSASTAYMELSSLRSEDTAVYYCAR

SEQ ID NO: 19 - protein, 39 aa, Artificial Sequence
  Synthetic Construct
YAQKFQGRATLTVDNPTSTAYMELSSLRSEDTAVYYCAR

SEQ ID NO: 20 - protein, 11 aa, Artificial Sequence
  Synthetic Construct
WGQGTLVTVSS

SEQ ID NO: 21 - protein, 11 aa, Artificial Sequence
  Synthetic Construct
WGQGTLLTVSS

SEQ ID NO: 22 - protein, 23 aa, Artificial Sequence
  Synthetic Construct
DIQMTQSPSSLSASVGDRVTITC

SEQ ID NO: 23 - protein, 23 aa, Artificial Sequence
  Synthetic Construct
DIQMTQSPSSLPASVGDRVTITC

SEQ ID NO: 24 - protein, 23 aa, Artificial Sequence
  Synthetic Construct
GIQMTQSPSSLSASVGDRVTITC

SEQ ID NO: 25 - protein, 23 aa, Artificial Sequence
  Synthetic Construct
DIVLTQSPDSLAVSLGERATINC

SEQ ID NO: 26 - protein, 23 aa, Artificial Sequence
  Synthetic Construct
DIVLTQSPDSLAVSLGERATIDC

SEQ ID NO: 27 - protein, 15 aa, Artificial Sequence
  Synthetic Construct
WYQQKPGKAPKLLIK

SEQ ID NO: 28 - protein, 15 aa, Artificial Sequence
  Synthetic Construct
WYQQKPGQPPKLLIK

SEQ ID NO: 29 - protein, 32 aa, Artificial Sequence
  Synthetic Construct
GVPSRFSGSGSGTDFTLTISSLQPEDFATYYC

SEQ ID NO: 30 - protein, 32 aa, Artificial Sequence
  Synthetic Construct
GVPSRFSGSGSGTDFTLTISSLQPEDLATYYC

SEQ ID NO: 31 - protein, 32 aa, Artificial Sequence
  Synthetic Construct
GVPDRFSGSGSGTDFTLTISSLQAEDVAVYYC

SEQ ID NO: 32 - protein, 10 aa, Artificial Sequence
  Synthetic Construct
FGQGTKLEIK

SEQ ID NO: 33 - protein, 10 aa, Artificial Sequence
  Synthetic Construct
FGQGTKLEIE

SEQ ID NO: 34 - protein, 117 aa, Artificial Sequence
  Synthetic Construct
QVQLVQSGAEVKKPGASVKVSCKASGATFTDYNFHWVRQAPGQGLEWIGFIYPSNGITGYAQDFQGRVTMTVDTSTSTVYMELSSLRSEDTAVYYCARSDVDYFDYWGQGTLVTVSS

SEQ ID NO: 35 - protein, 117 aa, Artificial Sequence
  Synthetic Construct
QVQLVQSGAEVKKPGASVKVSCKASGATFTDYNYHWVRQAPGQGLEWIGFIYPSNGITGYAQKFQDRVTMTVDTSTSTVYMELSSLRSEDTAVYYCARSFVDYFDYWGQGTLVTVSS

SEQ ID NO: 36 - protein, 117 aa, Artificial Sequence
  Synthetic Construct
QVQLVQSGAEVKKPGASVKVSCKASGYTFTDYNYHWVRQAPGQGLEWIGFIYPSNGITGSAQKFQGRVTMTVDTSTSTVYMELSSLRSEDTAVYYCARSDVDYFDYWGQGTLVTVSS

SEQ ID NO: 37 - protein, 117 aa, Artificial Sequence
  Synthetic Construct
QVQLVQSGAEVKKPGASVKVSCKASGYTFTDYNYHWVRQAPGQGLEWIGFIYPSNGITGYAQKFQGRVTMTVDTSTSTVYMELSSLRSEDTAVYYCADSTVDYFDYWGQGTLVTVSS

SEQ ID NO: 38 - protein, 117 aa, Artificial Sequence
  Synthetic Construct
QVQLVQSGAEVKKPGASVKVSCKASGYTFTDYNYHWVRQAPGQGLEWIGFIYPSNGITGYAQKFQGRVTMTVDTSTSTVYMELSSLRSEDTAVYYCARSDVDYFDYWGQGTLVTVSS

SEQ ID NO: 39 - protein, 117 aa, Artificial Sequence
  Synthetic Construct
QVQLVQSGAEVKKPGASVKVSCKASGYTFTDYNYHWVRQAPGQGLEWIGFIYPSNGITGYAQKFQGRVTMTVDTSTSTVYMELSSLRSEDTAVYYCARSSVDYFDYWGQGTLVTVSS

SEQ ID NO: 40 - protein, 117 aa, Artificial Sequence
  Synthetic Construct
QVQLVQSGAEVKKPGASVKVSCKASGYTFTDYNYHWVRQAPGQGLEWIGFIYPSNGITGYAQKFQGRVTMTVDTSTSTVYMELSSLRSEDTAVYYCARSTVDYFDYWGQGTLVTVSS

SEQ ID NO: 41 - protein, 117 aa, Artificial Sequence
  Synthetic Construct
QVQLVQSGAEVKKPGASVKVSCKASGYTFTDYNNHWVRQAPGQGLEWIGFIYPSNGITGYAQKFQGRVTMTVDTSTSTVYMELSSLRSEDTAVYYCARSDVDYFDYWGQGTLVTVSS

SEQ ID NO: 42 - protein, 117 aa, Artificial Sequence
  Synthetic Construct
QVQLVQSGAEVKKPGASVKVSCKASGYTFTDYNLHWVRQAPGQGLEWIGFIYPSNGITGYAQKFQGRVTMTVDTSTSTVYMELSSLRSEDTAVYYCARSTVDYFDYWGQGTLVTVSS

SEQ ID NO: 43 - protein, 117 aa, Artificial Sequence
  Synthetic Construct
QVQLVQSGAEVKKPGSSVKVSCKASGVTFTDYNYHWVRQAPGQGLEWIGFIYPSNGITGYAQKFQDRVTMTVDTSTSTVYMELSSLRSEDTAVYYCARSTVDYFDYWGQGTLVTVSS

SEQ ID NO: 44 - protein, 117 aa, Artificial Sequence
  Synthetic Construct
QVQLVQSGAEVKKPGASVKVSCKASGYAFTDYNLHWVRQAPGQGLEWIGFIYPSNGITGYAQKFQGRVTMTVDTSTSTVYMELSSLRSEDTAVYYCARSDVDYFDYWGQGTLVTVSS

SEQ ID NO: 45 - protein, 117 aa, Artificial Sequence
  Synthetic Construct
QVQLVQSGAEVKKPGASVKVSCKASGYTETDYNLHWVRQAPGQGLEWIGFIYPSNGITGYAQKFQGRVTMTVDTSTSTVYMELSSLRSEDTAVYYCARSDVDYFDYWGQGTLVTVSS

SEQ ID NO: 46 - protein, 117 aa, Artificial Sequence
  Synthetic Construct
QVQLVQSGAEVKKPGASVKVSCKASGYTFTDYNFHWVRQAPGQGLEWIGFIYPANGITGYAQKDQGRVTMTVDTSTSTVYMELSSLRSEDTAVYYCARSDVDYFDYWGQGTLVTVSS

SEQ ID NO: 47 - protein, 117 aa, Artificial Sequence
  Synthetic Construct
QVQLVQSGAEVKKPGASVKVSCKASGYTFTDYNFHWVRQAPGQGLEWIGFIYPSNGITGYAQKFTGRVTMTVDTSTSTVYMELSSLRSEDTAVYYCARSTVDYFDDWGQGTLVTVSS

SEQ ID NO: 48 - protein, 117 aa, Artificial Sequence
  Synthetic Construct
QVQLVQSGAEVKKPGASVKVSCKASGYTFTDYNLHWVRQAPGQGLEWIGFIYPSNGIRGYAQKFQGRVTMTVDTSTSTVYMELSSLRSEDTAVYYCARSDVDYFDYWGQGTLVTVSS

SEQ ID NO: 49 - protein, 117 aa, Artificial Sequence
  Synthetic Construct
QVQLVQSGAEVKKPGSSVKVSCKASGYTFTDYNLHWVRQAPGQGLEWIGFIYPSNGITGYAQKFQGRVTMTVDTSTSTVYMELSSLRSEDTAVYYCARSDVDYFDYWGQGTLVTVSS

SEQ ID NO: 50 - protein, 117 aa, Artificial Sequence
  Synthetic Construct
QVQLVQSGAEVKKPGASVKVSCKASGYTFTDYNYHWVRQAPGQGLEWIGFIYPSNGITGYAQKFQGRVTMTVDTSTSTVYMELSSLRSEDTAVYYCARSDVDYFDLWGQGTLVTVSS

SEQ ID NO: 51 - protein, 117 aa, Artificial Sequence
  Synthetic Construct
QVQLVQSGAEVKKPGSSVKVSCKASGYTHTDYNLHWVRQAPGQGLEWIGFIYPSNGITGYAQKFQGRVTMTVDTSTSTVYMELSSLRSEDTAVYYCARSDVDYFDYWGQGTLVTVSS

SEQ ID NO: 52 - protein, 117 aa, Artificial Sequence
  Synthetic Construct
QVQLVQSGAEVKKPGASVKISCKASGYTFTDYNLHWVRQAPGQGLEWIGFIYPSNGITGYAQKFQGRATLTVDNSTSTAYMELSSLRSEDTAVYYCARSDVDYFDYWGQGTLLTVSS

SEQ ID NO: 53 - protein, 117 aa, Artificial Sequence
  Synthetic Construct
QVQLVQSGAEVKKPGASVKISCKASGYTFTDYNLHWVRQAPGQGLEWIGFIYPSNGITGYAQKFQGRATLTVDNSTSTAYMELSSLRSEDTAVYYCARSTVDYFDYWGQGTLLTVSS

SEQ ID NO: 54 - protein, 117 aa, Artificial Sequence
  Synthetic Construct
QVQLVQSGAEVKKPGASVKISCKASGYTFTDYNLHWVRQAPGQGLEWIGFIYPSNGITGYAEKFEGRATLTVDNSTSTAYMELSSLRSEDTAVYYCARSFVDYFDYWGQGTLLTVSS

SEQ ID NO: 55 - protein, 117 aa, Artificial Sequence
  Synthetic Construct
QVQLVQSGAEVKKPGASVKISCKASGYTFTDYNYHWVRQAPGQGLEWIGFIYPSNGITGYAQKFFGRATLTVDNSTSTAYMELSSLRSEDTAVYYCARSDVDYFDYWGQGTLLTVSS

SEQ ID NO: 56 - protein, 117 aa, Artificial Sequence
  Synthetic Construct
QVQLVQSGAEVKKPGASVKISCKASGYTFTDYNYHWVRQAPGQGLEWIGFIYPSNGITGYAQKFQGRATLTVDNSTSTAYMELSSLRSEDTAVYYCARSDVDYFDYWGQGTLLTVSS

SEQ ID NO: 57 - protein, 117 aa, Artificial Sequence
  Synthetic Construct
QVQLVQSGAEVKKPGASVKISCKASGYTFTDYNYHWVRQAPGQGLEWIGFIYPSNGITGYAQKFQHRATLTVDNSTSTAYMELSSLRSEDTAVYYCARSDVDYFDYWGQGTLLTVSS

SEQ ID NO: 58 - protein, 117 aa, Artificial Sequence
  Synthetic Construct
QVQLVQSGAEVKKPGASVKISCKASGYTFTDYNLHWVRQAPGQGLEWIGFIYPSNQITGYAQKFQGRATLTVDNSTSTAYMELSSLRSEDTAVYYCARSDVDYFDYWGQGTLLTVSS

SEQ ID NO: 59 - protein, 117 aa, Artificial Sequence
  Synthetic Construct
QVQLVQSGAEVKKPGASVKISCKASGYTFTDYNLHWVRQAPGQGLEWIGFIYPSNRITGYAQKFQGRATLTVDNSTSTAYMELSSLRSEDTAVYYCARSDVDYFDYWGQGTLLTVSS

SEQ ID NO: 60 - protein, 117 aa, Artificial Sequence
  Synthetic Construct
QVQLVQSGAEVKKPGASVKISCKASGYTFTDYNLHWVRQAPGQGLEWIGFIYPSNVITGYAQKFQGRATLTVDNSTSTAYMELSSLRSEDTAVYYCARSDVDYFDYWGQGTLLTVSS

SEQ ID NO: 61 - protein, 117 aa, Artificial Sequence
  Synthetic Construct
QVQLVQSGAEVKKPGASVKISCKASGYTFTDYNLHWVRQAPGQGLEWIGFIYPSNGITGYAQKFQGRATLTVDTSTSTAYMELSSLRSEDTAVYYCARSDVDYFDYWGQGTLLTVSS

SEQ ID NO: 62 - protein, 117 aa, Artificial Sequence
  Synthetic Construct
QVQLVQSGAEVKKPGASVKISCKASGYTFTDYNLHWVRQAPGQGLEWIGFIYPSNGITGYAQKFQGRATLTVDQSTSTAYMELSSLRSEDTAVYYCARSDVDYFDYWGQGTLLTVSS

SEQ ID NO: 63 - protein, 117 aa, Artificial Sequence
  Synthetic Construct
QVQLVQSGAEVKKPGASVKISCKASGYTFTDYNLHWVRQAPGQGLEWIGFIYPSNGITGYAQKFQGRATLTVDNSASTAYMELSSLRSEDTAVYYCARSDVDYFDYWGQGTLLTVSS

SEQ ID NO: 64 - protein, 117 aa, Artificial Sequence
  Synthetic Construct
QVQLVQSGAEVKKPGASVKISCKASGYTFTDYNLHWVRQAPGQGLEWIGFIYPSNGITGYAQKFQGRATLTVDNPTSTAYMELSSLRSEDTAVYYCARSDVDYFDYWGQGTLLTVSS

SEQ ID NO: 65 - protein, 117 aa, Artificial Sequence
  Synthetic Construct
QVQLVQSGAEVKKPGASVKISCKASGYTFTDYNLHWVRQAPGQGLEWIGFIYPSNQITGYAQKFQGRATLTVDNSASTAYMELSSLRSEDTAVYYCARSDVDYFDYWGQGTLLTVSS

SEQ ID NO: 66 - protein, 117 aa, Artificial Sequence
  Synthetic Construct
QVQLVQSGAEVKKPGASVKISCKASGYTFTDYNLHWVRQAPGQGLEWIGFIYPSNQITGYAQKFQGRATLTVDNPTSTAYMELSSLRSEDTAVYYCARSDVDYFDYWGQGTLLTVSS

SEQ ID NO: 67 - protein, 117 aa, Artificial Sequence
  Synthetic Construct
QVQLVQSGAEVKKPGASVKISCKASGYTFTDYNLHWVRQAPGQGLEWIGFIYPSNRITGYAQKFQGRATLTVDTSTSTAYMELSSLRSEDTAVYYCARSDVDYFDYWGQGTLLTVSS

SEQ ID NO: 68 - protein, 117 aa, Artificial Sequence
  Synthetic Construct
QVQLVQSGAEVKKPGASVKISCKASGYTFTDYNLHWVRQAPGQGLEWIGFIYPSNRITGYAQKFQGRATLTVDQSTSTAYMELSSLRSEDTAVYYCARSDVDYFDYWGQGTLLTVSS

SEQ ID NO: 69 - protein, 117 aa, Artificial Sequence
  Synthetic Construct
QVQLVQSGAEVKKPGASVKISCKASGYTFTDYNLHWVRQAPGQGLEWIGFIYPSNRITGYAQKFQGRATLTVDNSASTAYMELSSLRSEDTAVYYCARSDVDYFDYWGQGTLLTVSS

SEQ ID NO: 70 - protein, 117 aa, Artificial Sequence
  Synthetic Construct
QVQLVQSGAEVKKPGASVKISCKASGYTFTDYNLHWVRQAPGQGLEWIGFIYPSNVITGYAQKFQGRATLTVDTSTSTAYMELSSLRSEDTAVYYCARSDVDYFDYWGQGTLLTVSS

SEQ ID NO: 71 - protein, 117 aa, Artificial Sequence
  Synthetic Construct
QVQLVQSGAEVKKPGASVKISCKASGYTFTDYNLHWVRQAPGQGLEWIGFIYPSNVITGYAQKFQGRATLTVDQSTSTAYMELSSLRSEDTAVYYCARSDVDYFDYWGQGTLLTVSS

SEQ ID NO: 72 - protein, 117 aa, Artificial Sequence
  Synthetic Construct
QVQLVQSGAEVKKPGASVKISCKASGYTFTDYNLHWVRQAPGQGLEWIGFIYPSNVITGYAQKFQGRATLTVDNSASTAYMELSSLRSEDTAVYYCARSDVDYFDYWGQGTLLTVSS

SEQ ID NO: 73 - protein, 117 aa, Artificial Sequence
  Synthetic Construct
QVQLVQSGAELKKPGASVKVSCKASGYTFTDYNLHWVRQAPGQRLEWIGFIYPSNGITGYSQKFQGKATLTVDTSASTAYMELSSLRSEDTAVYYCARSTVDYFDYWGQGTTVTVSS

SEQ ID NO: 74 - protein, 117 aa, Artificial Sequence
  Synthetic Construct
QVQLVQSGAEVKKPGASVKISCKASGYTFTDYNLHWVRQAPGQGLEWIGFIYPSNGITGYAQKFQGRATLTVDTSTSTAYMELSSLRSEDTAVYYCARSTVDYFDYWGQGTLLTVSS

SEQ ID NO: 75 - protein, 117 aa, Artificial Sequence
  Synthetic Construct
QVQLVQSGAEVKKPGASVKVSCKASGYTFTDYNLHWVRQAPGQGLEWMGFIYPSNGITGYAQKFQGRVTMTRDTSTSTVYMELSSLRSEDTAVYYCARSTVDYFDYWGQGTTVTVSS

SEQ ID NO: 76 - protein, 117 aa, Artificial Sequence
  Synthetic Construct
QVQLVQSGAEVKKPGASVKISCKASGYTFTDYNLHWVRQAPGQGLEWIGFIYPSNGITGYAQKFQGRATLTVDTSTSTAYMELSSLRSEDTAVYYCARSTVDYFDYWGQGTTVTVSS

SEQ ID NO: 77 - protein, 111 aa, Artificial Sequence
  Synthetic Construct
DIQMTQSPSSLSASVGDRVTITCRASQSVSTSTYSYMHWYQQKPGKAPKLLIKYASNLESGVPSRFSGSGSGTDFTLTISSLQPEDFATYYCQHSWEIPLTFGQGTKLEIK

SEQ ID NO: 78 - protein, 111 aa, Artificial Sequence
  Synthetic Construct
DIQMTQSPSSLSASVGDRVTITCRASQDVSTSTYSYMHWYQQKPGKAPKLLIKYASNLESGVPSRFSGSGSGTDFTLTISSLQPEDFATYYCQHSWEIPLTFGQGTKLEIK

SEQ ID NO: 79 - protein, 111 aa, Artificial Sequence
  Synthetic Construct
DIQMTQSPSSLSASVGDRVTITCRASQDVSTSTYSYMHWYQQKPGKAPKLLIKYVSNLESGVPSRFSGSGSGTDFTLTISSLQPEDFATYYCQHSWEIPLTFGQGTKLEIK

SEQ ID NO: 80 - protein, 111 aa, Artificial Sequence
  Synthetic Construct
DIQMTQSPSSLSASVGDRVTITCRASQDVSTSTYSYMHWYQRKPGKAPKLLIKYASNLESGVPSRFSGSGSGTDFTLTISSLQPEDFATYYCQHSWEIPLTFGQGTKLEIK

SEQ ID NO: 81 - protein, 111 aa, Artificial Sequence
  Synthetic Construct
DIQMTQSPSSLSASVGDRVTITCRASQSVGTSTYSYMHWYQQKPGKAPKLLIKYASALESGVPSRFSGSGSGTDFTLTISSLQPEDFATYYCQHSWEIPLTFGQGTKLEIK

SEQ ID NO: 82 - protein, 111 aa, Artificial Sequence
  Synthetic Construct
DIQMTQSPSSLSASVGDRVTITCRASQSVGTSTYSYMHWYQQKPGKAPKLLIKYASNLESGVPSRFSGSGSGTDFTLTISSLQPEDFATYYCQHSWEIPLTFGQGTKLEIK

SEQ ID NO: 83 - protein, 111 aa, Artificial Sequence
  Synthetic Construct
DIQMTQSPSSLPASVGDRVTITCRASQSVGTSTYSYMHWYQQKPGKAPKLLIKYASNLGSGVPSRFSGSGSGTDFTLTISSLQPEDFATYYCQHSWEIPLTFGQGTKLEIK

SEQ ID NO: 84 - protein, 111 aa, Artificial Sequence
  Synthetic Construct
GIQMTQSPSSLSASVGDRVTITCRASQSVGTSTYSYMHWYQQKPGKAPKLLIKYAVNLESGVPSRFSGSGSGTDFTLTISSLQPEDFATYYCQHSWEIPLTFGQGTKLEIK

SEQ ID NO: 85 - protein, 111 aa, Artificial Sequence
  Synthetic Construct
DIQMTQSPSSLSASVGDRVTITCRASQSVSASTYSYMHWYQQKPGKAPKLLIKYASNLESGVPSRFSGSGSGTDFTLTISSLQPEDLATYYCQHSWEIPLTFGQGTKLEIK

SEQ ID NO: 86 - protein, 111 aa, Artificial Sequence
  Synthetic Construct
DIVLTQSPDSLAVSLGERATINCRASQSVSTSTYSYMHWYQQKPGQPPKLLIKYASNLESGVPDRFSGSGSGTDFTLTISSLQAEDVAVYYCQHSWEIPLTFGQGTKLEIK

SEQ ID NO: 87 - protein, 111 aa, Artificial Sequence
  Synthetic Construct
DIVLTQSPDSLAVSLGERATINCRASQDVSTSTYSYMHWYQQKPGQPPKLLIKYASNLESGVPDRFSGSGSGTDFTLTISSLQAEDVAVYYCQHSWEIPLTFGQGTKLEIK

SEQ ID NO: 88 - protein, 111 aa, Artificial Sequence
  Synthetic Construct
DIVLTQSPDSLAVSLGERATINCRASQDVSTSTYSYMHWYQQKPGQPPKLLIKYAFNLESGVPDRFSGSGSGTDFTLTISSLQAEDVAVYYCQHSWEIPLEFGQGTKLEIK

SEQ ID NO: 89 - protein, 111 aa, Artificial Sequence
  Synthetic Construct
DIVLTQSPDSLAVSLGERATINCRASQDVSTSTYSYMHWYQQKPGQPPKLLIKYASYLESGVPDRFSGSGSGTDFTLTISSLQAEDVAVYYCEHSWEIPLTFGQGTKLEIK

SEQ ID NO: 90 - protein, 111 aa, Artificial Sequence
  Synthetic Construct
DIVLTQSPDSLAVSLGERATINCRASQDVSTSTYSYMHWYQQKPGQPPKLLIKYASNLESGVPDRFSGSGSGTDFTLTISSLQAEDVAVYYCQHSWELPLTFGQGTKLEIK

SEQ ID NO: 91 - protein, 111 aa, Artificial Sequence
  Synthetic Construct
DIVLTQSPDSLAVSLGERATINCRASQDVSTSTYSYMHWYQQKPGQPPKLLIKYASNVESGVPDRFSGSGSGTDFTLTISSLQAEDVAVYYCQHSWAIPLTFGQGTKLEIK

SEQ ID NO: 92 - protein, 111 aa, Artificial Sequence
  Synthetic Construct
DIVLTQSPDSLAVSLGERATINCRASQSVSTSTYSYMHWYQQKPGQPPKLLIKYASNLESGVPDRFSGSGSGTDFTLTISSLQAEDVAVYYCQHSEEIPLTFGQGTKLEIK

SEQ ID NO: 93 - protein, 111 aa, Artificial Sequence
  Synthetic Construct
DIVLTQSPDSLAVSLGERATIDCKASQDVSTSTYSYMHWYQQKPGQPPKLLIKYASNLESGVPDRFSGSGSGTDFTLTISSLQAEDVAVYYCQHSWEIPLTFGQGTKLEIK

SEQ ID NO: 94 - protein, 111 aa, Artificial Sequence
  Synthetic Construct
DIVLTQSPDSLAVSLGERATINCRASQDVSTSTYSYMHWYQQKPGQPPKLLIKYASNVESGVPDRFSGSGSGTDFTLTISSLQAEDVAVYYCQHSWEIPLTFGQGTKLEIE

SEQ ID NO: 95 - protein, 111 aa, Artificial Sequence
  Synthetic Construct
DIVLTQSPDSLAVSLGERATINCRASQSVHTSTYSYMHWYQQKPGQPPKLLIKYASNLESGVPDRFSGSGSGTDFTLTISSLQAEDVAVYYCQHSWEIPLTFGQGTKLEIK

SEQ ID NO: 96 - protein, 111 aa, Artificial Sequence
  Synthetic Construct
DIVLTQSPDSLAVSLGERATINCRGSQSVSTSTYSYMHWYQQKPGQPPKLLIKYESNLESGVPDRFSGSGSGTDFTLTISSLQAEDVAVYYCQHSWEIPLTFGQGTKLEIK

SEQ ID NO: 97 - protein, 111 aa, Artificial Sequence
  Synthetic Construct
DIVLTQSPDSLAVSLGERATINCRASQSVSTSTYSYMHWYQQKPGQPPKLLIKYESNLESGVPDRFSGSGSGTDFTLTISSLQAEDVAVYYCQHSWEIPLTFGQGTKLEIK

SEQ ID NO: 98 - protein, 111 aa, Artificial Sequence
  Synthetic Construct
DIVLTQSPDSLAVSLGERATINCRVSQDVSTSTYSYMHWYQQKPGQPPKLLIKYASNLESGVPDRFSGSGSGTDFTLTISSLQAEDVAVYYCQHSWEIPLTFGQGTKLEIK

SEQ ID NO: 99 - protein, 111 aa, Artificial Sequence
  Synthetic Construct
DIVLTQSPDSLAVSLGERATINCRASQSVGTSTYSYMHWYQQKPGQPPKLLIKYASNLESGVPDRFSGSGSGTDFTLTISSLQAEDVAVYYCQHSWEIPLTFGQGTKLEIK

SEQ ID NO: 100 - protein, 111 aa, Artificial Sequence
  Synthetic Construct
DIVLTQSPDSLAVSLGERATINCRASQDVSTSTYSYMHWYQQKPGQPPKLLIKYASFLESGVPDRFSGSGSGTDFTLTISSLQAEDVAVYYCQHSWEIPLTFGQGTKLEIK

SEQ ID NO: 101 - protein, 111 aa, Artificial Sequence
  Synthetic Construct
DIVLTQSPDSLAVSLGERATINCRASQSVGTSTYSYMHWYQQKPGQPPKLLIKYASNLNSGVPDRFSGSGSGTDFTLTISSLQAEDVAVYYCQHSEEIPLTFGQGTKLEIK

SEQ ID NO: 102 - protein, 111 aa, Artificial Sequence
  Synthetic Construct
DIQMTQSPSSLSASVGDRVTITCRASQSVSTSTYSYMHWYQQKPGKAPKLLIYYASNLESGVPSRFSGSGSGTDFTLTISSLQPEDFATYYCQHSWEIPLTFGQGTKLEIK

SEQ ID NO: 103 - protein, 117 aa, Artificial Sequence
  Synthetic Construct
EVQLQQSGPELVKPGASVKISCKASGYTFTDYNLHWVKLSHGKSLEWIGFIYPSNGITGYNQKFKNKATLTVDNSSSTAYMELRSLTSEDSAVYYCARSTVDYFDYWGQGTTLTVSS

SEQ ID NO: 104 - protein, 111 aa, Artificial Sequence
  Synthetic Construct
DIVLTQSPASLAVSLGQRATMSCRASQSVSTSTYSYMHWYQQKPGQPPKLLIKYASNLESGVPARFSGSGSGTDFTLNIHPVEEEDTATYYCQHSWEIPLTFGAGTKLELK

SEQ ID NO: 105 - protein, 10 aa, Artificial Sequence
  Synthetic Construct
GYTFTDYNLH

SEQ ID NO: 106 - protein, 10 aa, Artificial Sequence
  Synthetic Construct
GATFTDYNFH

SEQ ID NO: 107 - protein, 10 aa, Artificial Sequence
  Synthetic Construct
GATFTDYNYH

SEQ ID NO: 108 - protein, 10 aa, Artificial Sequence
  Synthetic Construct
GYTFTDYNYH

SEQ ID NO: 109 - protein, 10 aa, Artificial Sequence
  Synthetic Construct
GYTFTDYNNH

SEQ ID NO: 110 - protein, 10 aa, Artificial Sequence
  Synthetic Construct
GVTFTDYNYH

SEQ ID NO: 111 - protein, 10 aa, Artificial Sequence
  Synthetic Construct
GYAFTDYNLH

SEQ ID NO: 112 - protein, 10 aa, Artificial Sequence
  Synthetic Construct
GYTETDYNLH

SEQ ID NO: 113 - protein, 10 aa, Artificial Sequence
  Synthetic Construct
GYTFTDYNFH

SEQ ID NO: 114 - protein, 10 aa, Artificial Sequence
  Synthetic Construct
GYTHTDYNLH

SEQ ID NO: 115 - protein, 10 aa, Artificial Sequence
  Synthetic Construct
FIYPSNGITG

SEQ ID NO: 116 - protein, 10 aa, Artificial Sequence
  Synthetic Construct
FIYPANGITG

SEQ ID NO: 117 - protein, 10 aa, Artificial Sequence
  Synthetic Construct
FIYPSNGIRG

SEQ ID NO: 118 - protein, 10 aa, Artificial Sequence
  Synthetic Construct
FIYPSNQITG

SEQ ID NO: 119 - protein, 10 aa, Artificial Sequence
  Synthetic Construct
FIYPSNRITG

SEQ ID NO: 120 - protein, 10 aa, Artificial Sequence
  Synthetic Construct
FIYPSNVITG

SEQ ID NO: 121 - protein, 8 aa, Artificial Sequence
  Synthetic Construct
STVDYFDY

SEQ ID NO: 122 - protein, 8 aa, Artificial Sequence
  Synthetic Construct
SDVDYFDY

SEQ ID NO: 123 - protein, 8 aa, Artificial Sequence
  Synthetic Construct
SFVDYFDY

SEQ ID NO: 124 - protein, 8 aa, Artificial Sequence
  Synthetic Construct
SSVDYFDY

SEQ ID NO: 125 - protein, 8 aa, Artificial Sequence
  Synthetic Construct
STVDYFDD

SEQ ID NO: 126 - protein, 8 aa, Artificial Sequence
  Synthetic Construct
SDVDYFDL

SEQ ID NO: 127 - protein, 15 aa, Artificial Sequence
  Synthetic Construct
RASQSVSTSTYSYMH

SEQ ID NO: 128 - protein, 15 aa, Artificial Sequence
  Synthetic Construct
RASQSVGTSTYSYMH

SEQ ID NO: 129 - protein, 15 aa, Artificial Sequence
  Synthetic Construct
RASQSVSASTYSYMH

SEQ ID NO: 130 - protein, 15 aa, Artificial Sequence
  Synthetic Construct
RASQDVSTSTYSYMH

SEQ ID NO: 131 - protein, 15 aa, Artificial Sequence
  Synthetic Construct
KASQDVSTSTYSYMH

SEQ ID NO: 132 - protein, 15 aa, Artificial Sequence
  Synthetic Construct
RASQSVHTSTYSYMH

SEQ ID NO: 133 - protein, 15 aa, Artificial Sequence
  Synthetic Construct
RGSQSVSTSTYSYMH

SEQ ID NO: 134 - protein, 15 aa, Artificial Sequence
  Synthetic Construct
RVSQDVSTSTYSYMH

SEQ ID NO: 135 - protein, 7 aa, Artificial Sequence
  Synthetic Construct
YASNLES

SEQ ID NO: 136 - protein, 7 aa, Artificial Sequence
  Synthetic Construct
YVSNLES

SEQ ID NO: 137 - protein, 7 aa, Artificial Sequence
  Synthetic Construct
YASALES

SEQ ID NO: 138 - protein, 7 aa, Artificial Sequence
  Synthetic Construct
YASNLGS

SEQ ID NO: 139 - protein, 7 aa, Artificial Sequence
  Synthetic Construct
YAVNLES

SEQ ID NO: 140 - protein, 7 aa, Artificial Sequence
  Synthetic Construct
YAFNLES

SEQ ID NO: 141 - protein, 7 aa, Artificial Sequence
  Synthetic Construct
YASYLES

SEQ ID NO: 142 - protein, 7 aa, Artificial Sequence
  Synthetic Construct
YASNVES

SEQ ID NO: 143 - protein, 7 aa, Artificial Sequence
  Synthetic Construct
YESNLES

SEQ ID NO: 144 - protein, 7 aa, Artificial Sequence
  Synthetic Construct
YASFLES

SEQ ID NO: 145 - protein, 7 aa, Artificial Sequence
  Synthetic Construct
YASNLNS

SEQ ID NO: 146 - protein, 9 aa, Artificial Sequence
  Synthetic Construct
QHSWEIPLT

SEQ ID NO: 147 - protein, 9 aa, Artificial Sequence
  Synthetic Construct
QHSWEIPLE

SEQ ID NO: 148 - protein, 9 aa, Artificial Sequence
  Synthetic Construct
EHSWEIPLT

SEQ ID NO: 149 - protein, 9 aa, Artificial Sequence
  Synthetic Construct
QHSWELPLT

SEQ ID NO: 150 - protein, 9 aa, Artificial Sequence
  Synthetic Construct
QHSWAIPLT

SEQ ID NO: 151 - protein, 9 aa, Artificial Sequence
  Synthetic Construct
QHSEEIPLT

SEQ ID NO: 152 - protein, 10 aa, Artificial Sequence
  Synthetic Construct
  VARIANT 2: Xaa = Tyr, Ala, or Val
  VARIANT 3: Xaa = Thr or Ala
  VARIANT 4: Xaa = Phe, Glu, or His
  VARIANT 9: Xaa = Leu, Phe, Tyr, or Asn
GXXXTDYNXH

SEQ ID NO: 153 - protein, 10 aa, Artificial Sequence
  Synthetic Construct
  VARIANT 5: Xaa = Ser or Ala
  VARIANT 7: Xaa = Gly, Gln, Arg, or Val
  VARIANT 9: Xaa = Thr or Arg
FIYPXNXIXG

SEQ ID NO: 154 - protein, 8 aa, Artificial Sequence
  Synthetic Construct
  VARIANT 2: Xaa = Thr, Asp, Phe, or Ser
  VARIANT 8: Xaa = Tyr, Asp, or Leu
SXVDYFDX

SEQ ID NO: 155 - protein, 15 aa, Artificial Sequence
  Synthetic Construct
  VARIANT 1: Xaa = Arg or Lys
  VARIANT 2: Xaa = Ala, Gly, or Val
  VARIANT 5: Xaa = Ser or Asp
  VARIANT 7: Xaa = Ser, Gly, or His
  VARIANT 8: Xaa = Thr or Ala
XXSQXVXXSTYSYMH

SEQ ID NO: 156 - protein, 7 aa, Artificial Sequence
  Synthetic Construct
  VARIANT 2: Xaa = Ala, Val, or Glu
  VARIANT 3: Xaa = Ser, Val, or Phe
  VARIANT 4: Xaa = Asn, Ala, Tyr, or Phe
  VARIANT 5: Xaa = Leu or Val
  VARIANT 6: Xaa = Glu, Gly, or Asn
YXXXXXS

SEQ ID NO: 157 - protein, 9 aa, Artificial Sequence
  Synthetic Construct
  VARIANT 1: Xaa = Gln or Glu
  VARIANT 4: Xaa = Trp or Glu
  VARIANT 5: Xaa = Glu or Ala
  VARIANT 6: Xaa = Ile or Leu
  VARIANT 9: Xaa = Thr or Glu
XHSXXXPLX

SEQ ID NO: 158 - protein, 25 aa, Artificial Sequence
  Synthetic Construct
  VARIANT 16: Xaa = Ala or Ser
  VARIANT 20: Xaa = Val or Ile
QVQLVQSGAEVKKPGXSVKXSCKAS

SEQ ID NO: 159 - protein, 39 aa, Artificial Sequence
  Synthetic Construct
  VARIANT 1: Xaa = Tyr or Ser
  VARIANT 3: Xaa = Gln or Glu
  VARIANT 4: Xaa = Lys or Asp
  VARIANT 5: Xaa = Phe or Asp
  VARIANT 6: Xaa = Gln, Phe, Glu, or Thr
  VARIANT 7: Xaa = Gly, Asp, or His
  VARIANT 9: Xaa = Val or Ala
  VARIANT 11: Xaa = Met or Leu
  VARIANT 15: Xaa = Thr, Asn, or Gln
  VARIANT 16: Xaa = Ser or Pro
  VARIANT 17: Xaa = Thr or Ala
  VARIANT 20: Xaa = Val or Ala
XAXXXXXRXTXTVDXXXSTXYMELSSLRSEDTAVYYCAR

SEQ ID NO: 160 - protein, 11 aa, Artificial Sequence
  Synthetic Construct
  VARIANT 7: Xaa = Val or Leu
WGQGTLXTVSS

SEQ ID NO: 161 - protein, 23 aa, Artificial Sequence
  Synthetic Construct
  VARIANT 1: Xaa = Asp or Gly
  VARIANT 3: Xaa = Gln or Val
  VARIANT 4: Xaa = Met or Leu
  VARIANT 9: Xaa = Ser or Asp
  VARIANT 12: Xaa = Ser, Pro, or Ala
  VARIANT 13: Xaa = Ala or Val
  VARIANT 15: Xaa = Val or Leu
  VARIANT 17: Xaa = Asp or Glu
  VARIANT 19: Xaa = Val or Ala
  VARIANT 22: Xaa = Thr, Asn, or Asp
XIXXTQSPXSLXXSXGXRXTIXC

SEQ ID NO: 162 - protein, 15 aa, Artificial Sequence
  Synthetic Construct
  VARIANT 8: Xaa = Lys or Gln
  VARIANT 9: Xaa = Ala or Pro
WYQQKPGXXPKLLIK

SEQ ID NO: 163 - protein, 32 aa, Artificial Sequence
  Synthetic Construct
  VARIANT 4: Xaa = Ser or Asp
  VARIANT 24: Xaa = Pro or Ala
  VARIANT 27: Xaa = Phe, Leu, or Val
  VARIANT 29: Xaa = Thr or Val
GVPXRFSGSGSGTDFTLTISSLQXEDXAXYYC

SEQ ID NO: 164 - protein, 10 aa, Artificial Sequence
  Synthetic Construct
  VARIANT 10: Xaa = Lys or Glu
FGQGTKLEIX

SEQ ID NO: 165 - protein, 19 aa, Artificial Sequence
  Synthetic Construct
  VARIANT 1: Xaa = Asp or Glu
  VARIANT 2, 3: Xaa = Any amino acid, and up to two can be present or absent
  VARIANT 5, 6: Xaa = Any amino acid
  VARIANT 7: Xaa = Leu or Ile
  VARIANT 8, 9, 10, 11, 12, 13, 14, 15: Xaa = Any amino acid, and up to two can be present or absent
  VARIANT 17, 18: Xaa = Any amino acid
  VARIANT 19: Xaa = Leu or Ile
XXXYXXXXXXXXXXXYXXX

SEQ ID NO: 166 - protein, 110 aa, Artificial Sequence
  Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCP

SEQ ID NO: 167 - skipped

SEQ ID NO: 168 - protein, 15 aa, Artificial Sequence
  Synthetic Construct
WYQRKPGKAPKLLIK

SEQ ID NO: 169 - protein, 15 aa, Artificial Sequence
  Synthetic Construct
  VARIANT 4: Xaa = Gln or Arg
  VARIANT 8: Xaa = Lys or Gln
  VARIANT 9: Xaa = Ala or Pro
WYQXKPGXXPKLLIK

SEQ ID NO: 170 - protein, 117 aa, Artificial Sequence
  Synthetic Construct
QVQLVQSGAEVKKPGASVKVSCKASGYTFTDYNLHWVRQAPGQGLEWMGFIYPSNGITGYAQKFQGRVTMTRDTSTSTVYMELSSLRSEDTAVYYCARSTVDYFDYWGQGTLVTVSS

SEQ ID NO: 171 - protein, 117 aa, Artificial Sequence
  Synthetic Construct
QVQLVQSGAEVKKPGASVKVSCKASGYTFTDYNLHWVRQAPGQGLEWMGFIYPSNGITGYAQKFQGRVTMTVDTSTSTAYMELSSLRSEDTAVYYCARSTVDYFDYWGQGTTVTVSS

SEQ ID NO: 172 - protein, 117 aa, Artificial Sequence
  Synthetic Construct
QVQLVQSGAEVKKPGASVKVSCKASGYTFTDYNLHWVRQAPGQSLEWIGFIYPSNGITGYSQKFQGKATLTVDTSASTAYMELSSLRSEDTAVYYCARSTVDYFDYWGQGTTVTVSS

SEQ ID NO: 173 - protein, 117 aa, Artificial Sequence
  Synthetic Construct
QVQLVQSGAEVKKPGASVKISCKASGYTFTDYNLHWVRQAPGQRLEWIGFIYPSNGITGYNQKFKNKATLTVDTSASTAYMELSSLRSEDTAVYYCARSTVDYFDYWGQGTTVTVSS

SEQ ID NO: 174 - protein, 117 aa, Artificial Sequence
  Synthetic Construct
QVQLVQSGAEVKKPGASVKISCKASGYTFTDYNLHWVRQAPGQRLEWIGFIYPSNGITGYSQKFQGKATLTVDTSASTAYMELSSLRSEDTAVYYCARSTVDYFDYWGQGTTVTVSS

SEQ ID NO: 175 - protein, 117 aa, Artificial Sequence
  Synthetic Construct
QVQLVQSGAEVKKPGASVKVSCKASGYTFTDYNLHWVRQAPGQRLEWIGFIYPSNGITGYSQKFQGRATLTVDTSASTAYMELSSLRSEDTAVYYCARSTVDYFDYWGQGTTVTVSS

SEQ ID NO: 176 - protein, 447 aa, Artificial Sequence
  Synthetic Construct
QVQLVQSGAEVKKPGASVKISCKASGYTFTDYNLHWVRQAPGQGLEWIGFIYPSNGITGYAQKFQGRATLTVDNSTSTAYMELSSLRSEDTAVYYCARSDVDYFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 447 aa, Artificial Sequence
  Synthetic Construct
QVQLVQSGAEVKKPGASVKISCKASGYTFTDYNLHWVRQAPGQGLEWIGFIYPSNRITGYAQKFQGRATLTVDNSTSTAYMELSSLRSEDTAVYYCARSDVDYFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 447 aa, Artificial Sequence
  Synthetic Construct
QVQLVQSGAEVKKPGASVKISCKASGYTFTDYNLHWVRQAPGQGLEWIGFIYPSNQITGYAQKFQGRATLTVDNSASTAYMELSSLRSEDTAVYYCARSDVDYFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 443 aa, Artificial Sequence
  Synthetic Construct
QVQLVQSGAEVKKPGASVKISCKASGYTFTDYNLHWVRQAPGQGLEWIGFIYPSNGITGYAQKFQGRATLTVDNSTSTAYMELSSLRSEDTAVYYCARSDVDYFDYWGQGTLLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 180 - protein, 443 aa, Artificial Sequence
  Synthetic Construct
QVQLVQSGAEVKKPGASVKISCKASGYTFTDYNLHWVRQAPGQGLEWIGFIYPSNRITGYAQKFQGRATLTVDNSTSTAYMELSSLRSEDTAVYYCARSDVDYFDYWGQGTLLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 181 - protein, 443 aa, Artificial Sequence
  Synthetic Construct
QVQLVQSGAEVKKPGASVKISCKASGYTFTDYNLHWVRQAPGQGLEWIGFIYPSNQITGYAQKFQGRATLTVDNSASTAYMELSSLRSEDTAVYYCARSDVDYFDYWGQGTLLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 182 - protein, 447 aa, Artificial Sequence
  Synthetic Construct
QVQLVQSGAEVKKPGASVKISCKASGYTFTDYNLHWVRQAPGQGLEWIGFIYPSNGITGYAQKFQGRATLTVDNSTSTAYMELSSLRSEDTAVYYCARSDVDYFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 447 aa, Artificial Sequence
  Synthetic Construct
QVQLVQSGAEVKKPGASVKISCKASGYTFTDYNLHWVRQAPGQGLEWIGFIYPSNRITGYAQKFQGRATLTVDNSTSTAYMELSSLRSEDTAVYYCARSDVDYFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 447 aa, Artificial Sequence
  Synthetic Construct
QVQLVQSGAEVKKPGASVKISCKASGYTFTDYNLHWVRQAPGQGLEWIGFIYPSNQITGYAQKFQGRATLTVDNSASTAYMELSSLRSEDTAVYYCARSDVDYFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 218 aa, Artificial Sequence
  Synthetic Construct
DIVLTQSPDSLAVSLGERATINCRASQSVSTSTYSYMHWYQQKPGQPPKLLIKYASNLESGVPDRFSGSGSGTDFTLTISSLQAEDVAVYYCQHSWEIPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 186-196 - skipped (11 entries)

SEQ ID NO: 197 - protein, 446 aa, Artificial Sequence
  Synthetic Construct
QVQLVQSGAEVKKPGASVKISCKASGYTFTDYNLHWVRQAPGQGLEWIGFIYPSNGITGYAQKFQGRATLTVDNSTSTAYMELSSLRSEDTAVYYCARSDVDYFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 198 - protein, 446 aa, Artificial Sequence
  Synthetic Construct
QVQLVQSGAEVKKPGASVKISCKASGYTFTDYNLHWVRQAPGQGLEWIGFIYPSNRITGYAQKFQGRATLTVDNSTSTAYMELSSLRSEDTAVYYCARSDVDYFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 199 - protein, 446 aa, Artificial Sequence
  Synthetic Construct
QVQLVQSGAEVKKPGASVKISCKASGYTFTDYNLHWVRQAPGQGLEWIGFIYPSNQITGYAQKFQGRATLTVDNSASTAYMELSSLRSEDTAVYYCARSDVDYFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 200 - protein, 442 aa, Artificial Sequence
  Synthetic Construct
QVQLVQSGAEVKKPGASVKISCKASGYTFTDYNLHWVRQAPGQGLEWIGFIYPSNGITGYAQKFQGRATLTVDNSTSTAYMELSSLRSEDTAVYYCARSDVDYFDYWGQGTLLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 201 - protein, 442 aa, Artificial Sequence
  Synthetic Construct
QVQLVQSGAEVKKPGASVKISCKASGYTFTDYNLHWVRQAPGQGLEWIGFIYPSNRITGYAQKFQGRATLTVDNSTSTAYMELSSLRSEDTAVYYCARSDVDYFDYWGQGTLLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 202 - protein, 442 aa, Artificial Sequence
  Synthetic Construct
QVQLVQSGAEVKKPGASVKISCKASGYTFTDYNLHWVRQAPGQGLEWIGFIYPSNQITGYAQKFQGRATLTVDNSASTAYMELSSLRSEDTAVYYCARSDVDYFDYWGQGTLLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 203 - protein, 446 aa, Artificial Sequence
  Synthetic Construct
QVQLVQSGAEVKKPGASVKISCKASGYTFTDYNLHWVRQAPGQGLEWIGFIYPSNGITGYAQKFQGRATLTVDNSTSTAYMELSSLRSEDTAVYYCARSDVDYFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 204 - protein, 446 aa, Artificial Sequence
  Synthetic Construct
QVQLVQSGAEVKKPGASVKISCKASGYTFTDYNLHWVRQAPGQGLEWIGFIYPSNRITGYAQKFQGRATLTVDNSTSTAYMELSSLRSEDTAVYYCARSDVDYFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 205 - protein, 446 aa, Artificial Sequence
  Synthetic Construct
QVQLVQSGAEVKKPGASVKISCKASGYTFTDYNLHWVRQAPGQGLEWIGFIYPSNQITGYAQKFQGRATLTVDNSASTAYMELSSLRSEDTAVYYCARSDVDYFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG